(19) United States
(12) Patent Application Publication
(10) Pub. No.: US 2023/0310551 A1
(43) Pub. Date: Oct. 5, 2023

(54) INSULIN RECEPTOR PARTIAL AGONISTS

(71) Applicant: Merck Sharp & Dohme LLC, Rahway, NJ (US)

(72) Inventors: Christina B. Madsen-Duggan, Scotch Plains, NJ (US); Danqing Feng, Green Brook, NJ (US); Pei Huo, Millburn, NJ (US); Ahmet Kekec, Hoboken, NJ (US); Songnian Lin, Holmdel, NJ (US); Ravi Nargund, Skillman, NJ (US); Christopher R. Moyes, Westfield, NJ (US); Dmitri A. Pissarnitski, Scotch Plains, NJ (US); Zhicai Shi, Monmouth Junction, NJ (US); Zhicai Wu, Montvale, NJ (US); Lin Yan, East Bruswick, NJ (US); Yuping Zhu, Basking Ridge, NJ (US)

(73) Assignee: Merck Sharp & Dohme LLC, Rahway, NJ (US)

(21) Appl. No.: 18/041,825

(22) PCT Filed: Aug. 24, 2021
(86) PCT No.: PCT/US21/47304

§ 371 Date: Feb. 16, 2023

Related U.S. Application Data
(60) Provisional application No. 63/070,384, filed on Aug. 26, 2020.

(51) Int. Cl. A61K 38/28; C07K 14/62; A61P 3/10

(52) U.S. Cl. CPC A61K 38/28; C07K 14/62; A61P 3/10

(57) ABSTRACT

Insulin dimers and insulin analog dimers that act as partial agonists at the insulin receptor are disclosed.

### BACKGROUND OF THE INVENTION

### Field of the Invention

The present invention relates to insulin dimers and insulin analog dimers that act as partial agonists at the insulin receptor.

### Description of Related Art

Insulin is the most effective anti-diabetic therapy for glycemic control in diabetic patients, but high hypoglycemia risk limits its treatment efficacy. The key reason that diabetic patients on insulin are not attaining their HbA1C goal is because administration doses are often intentionally lowered to avoid potentially life-threatening hypoglycemia. To improve the narrow therapeutic index (TI) of insulin may allow for further lowering glucose level to attain glycemic control with lower hypoglycemic risk, reducing health care cost associated with hypoglycemia treatment. Among various active research areas of developing insulins with improved TI, covalently linked insulin dimers have been reported in the literature to function as partial agonists of insulin receptor. It is believed that partial agonism of the insulin receptor by these covalent dimers may elicit a desired submaximal activation of the insulin receptor while it may also reduce overactivation of the insulin receptor by excess amount of the endogeneous insulin, leading to an increased therapeutic index in vivo.

Insulin is an essential therapy for type 1 diabetes mellitus (T1DM) patients and many type 2 mellitus diabetics (T2DMs), prescribed to close to one third of U.S. patients among all anti-diabetic drug users in the past decade. However, challenges of current insulin therapies, including narrow TI to hypoglycemia and body weight gain, limit their wider adoption and potential for patients to achieve ideal glycemic control.

In addition to prandial insulin secretion in response to meals, the pancreas releases insulin at a “basal” rate, governed largely by plasma glucose levels to maintain appropriate fasting glucose regulation. This is achieved mainly by controlling hepatic glucose release, through endogenous insulin's hepato-preferring action. Modern insulin analogs include rapid acting and basal insulins, as well as mixtures of these two. Rapid-acting insulin analogs (RAA) are developed to control post-prandial hyperglycemia while insulins with extended duration of action regulate basal glucose levels. Long-acting insulins are used by all T1DM (in combination with prandial injections) and the majority of T2DM patients start their insulin therapy from a basal product. Basal insulin consumption is growing rapidly as the worldwide diabetes population (particularly T2DM) soars.

Insulin dimers have been disclosed in Brandenburg et al. in U.S. Pat. No. 3,907,763 (1973); Tatnell et al., Biochem J. 216: 687-694 (1983); Shuttler and Brandenburg, Hoppe-Seyler's Z. Physiol. Chem, 363, 317-330, 1982; Weiland et al., Proc Natl. Acad. Sci. (USA) 87: 1154-1158 (1990); Deppe et al., Naunyn-Schmiedeberg's Arch Pharmacol (1994) 350:213-217; Brandenburg and Havenith in U.S. Pat. No. 6,908,897(B2) (2005); Knudsen et al., PLOS ONE 7: e51972 (2012); DiMarchi et al in WO2011/159895; DiMarchi et al. in WO 2014/052451; and Herrera et al., WO2014141165. More recently, insulin dimers have been described in Brant-Synthesis and Characterization of Insulin Receptor Partial Agonists as a Route to Improved Diabetes Therapy, Ph.D. Dissertation, Indiana University (April 2015) and Zaykov and DiMarchi, Poster P212-Exploration of the structural and mechanistic basis for partial agonism of insulin dimers, American Peptide Symposium, Orlando FL (June 20-25 (2015).

Despite continuous development efforts over the past several decades, available long-acting insulins are still not optimized compared to physiological basal insulin. This is partially because major focus was on improving PK flatness of these analogs but not fixing the relative over-insulinization of peripheral tissues, which contributes to increased hypoglycemia risk. As a result, hypoglycemia remains a key medical risk with huge burden on patients and causes significant morbidity and mortality.

### REFERENCE TO SEQUENCE LISTING SUBMITTED ELECTRONICALLY

The sequence listing of the present application is submitted electronically via EFS-Web as an ASCII formatted sequence listing with a file name 25085WOPCT-SEQLIST-17JUN2021.txt, creation date of Jun. 17, 2021, and a size of 6.96 kb. This sequence listing submitted via EFS-Web is part of the specification and is herein incorporated by reference in its entirety.

### BRIEF SUMMARY OF THE INVENTION

The present invention provides compounds comprising two insulin molecules covalently linked to form an insulin molecule dimer that may activate the insulin receptor with regular insulin-like potency but with reduced maximum activity. These compounds are insulin receptor partial agonists (IPRAs): they behave like other insulin analogs to lower glucose effectively but with lower risk of hypoglycemia.

Provided are insulin receptor partial agonist covalent insulin dimers formulated as novel and transformative basal insulins (once daily administration) that manifest an improved therapeutic index (TI) over current standard of care (SOC) basal insulins. In one embodiment, the IPRAs of the present invention may lower glucose effectively with reduced risk of hypoglycemia in diabetic minipig and has the property of a once daily (QD) basal insulin. The improved TI may empower practitioners to more aggressively dose IRPAs of the present invention to achieve target goals for control of fasting glucose. Tight control of fasting glucose and HbA c by an IRPA may allow it to serve as 1) a stand-alone long-acting insulin with an enhanced efficacy and safety profile in T2DM and 2) an improved foundational basal insulin in T1DM (and some T2DM) for use with additional prandial rapid-acting insulin analogs (RAA) doses. Thus, the present invention provides the following embodiments.

The present invention relates to two insulin molecules dimerized by covalently linking the a-amino groups of the A1 residue of each insulin molecule via a linker moiety, wherein the linker moiety is selected from the group consisting Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, Linker 23, Linker 24, Linker 25, Linker 26, Linker 27, Linker 28, Linker 29, Linker 30, Linker 31, Linker 32, Linker 33, Linker 34, Linker 35, Linker 36, Linker 37, Linker 38, Linker 39, Linker 40, Linker 41, Linker 42, Linker 43, Linker 44, Linker 45, and Linker 46.

In particular aspects of the insulin dimer, the 8-amine of the B-29 or B28 lysine of each insulin molecule B chain and the N-terminal amino acid of of the B-chains of each of the two insulins may be optionally and independently conjugated with a capping group. An embodiment of this aspect of the invention is realized when the (ε)-amine is from the B-28 lysine in the case of LISPRO insulin.

Another aspect of the insulin dimer is realized when capping groups include acyl moieties comprising carbamates, PEG-containing chains, sugar-containing groups, carboxylic acid containing groups, phosphonate groups, aryl groups (including but not limited to unsubstituted and substituted phenyl groups), and aromatic or non-aromatic heterocycles (including but not limited to morpholinyl, tetrahydrofuranyl, and fused versions of thereof), or a mixture thereof. A subembodiment of this aspect of the invention is realized when the capping group is an acyl moiety bearing one or more carbamates. Another subembodiment of this aspect of the invention is realized when the capping group is an acyl moiety bearing one or more PEG-containing chains. A subembodiment of this aspect of the invention is realized when the PEG in the PEG-containing chains is selected from PEG2 through PEG25. A subembodiment of this aspect of the invention is realized when the capping group is an acyl moiety bearing one or more sugar-containing groups. A subembodiment of this aspect of the invention is realized when the capping group is an acyl moiety bearing one or more carboxylic acid containing groups. A subembodiment of this aspect of the invention is realized when the capping group is an acyl moiety bearing one or more amines. A subembodiment of this aspect of the invention is realized when the capping group is an acyl moiety bearing one or more amides. A subembodiment of this aspect of the invention is realized when the capping group is an acyl moiety bearing one or more hydroxyls. A subembodiment of this aspect of the invention is realized when the capping group is an acyl moiety bearing one or more phosphonates. A subembodiment of this aspect of the invention is realized when the capping group is an acyl moiety bearing one or more heterocycle groups.

Another aspect of the insulin dimer is realized when the capping group is a linear or branch C₁₋₆ alkyl, or has the general formula RC(O)—, where R is:
a) a peptide,
b) PEG,
c) linear or branched C₁-6 alkyl chain,
d) R′NH—, or
e) R′O—,
   wherein R′ is H (when R is R′NH—), peptide, PEG, or linear or branched alkyl chain, and wherein each said peptide, PEG and linear or branched alkyl may be unsubstituted or substituted with 1 to 3 groups selected from amino-, phosphono-, hydroxy-, carboxylic acid, amino acid, PEG, and saccharides.

In aspects of this invention the capping group is, for example dimethyl, isobutyl, or is a group RC(O) that may be exemplified as acetyl, phenylacetyl, isobutyl, methoxyacetyl, 2-(carboxymethoxy)acetyl, 2-[bis(carboxymethylamino)]acetyl, glutaryl, trifluoroacetyl, glycyl, aminoethylglucose (AEG), AEG-C6, PEG (e.g., PEG1, PEG2, PEG3, PEG4, PEG5, PEG8, PEG24), phosphonoacetyl, morpholinohexanoyl, and alkoxycarbonyl.

A particular aspect of the insulin dimer is realized when the capping group is selected from Capping Group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31, or a mixture thereof from Table II. A subembodiment of this aspect of the invention is realized when the capping group is selected from 6, 7, 9, 10, 11, 12, 15, 19, 20, and 21-28, or a mixture thereof from Table II. A further subembodiment of this aspect of the invention is realized when at least one of the capping group of Table II is 6. A further subembodiment of this aspect of the invention is realized when at least one of the capping group of Table II is 7. A further subembodiment of this aspect of the invention is realized when at least one of the capping group of Table II is 9. A further subembodiment of this aspect of the invention is realized when at least one of the capping group of Table II is 10. A further subembodiment of this aspect of the invention is realized when at least one of the capping group of Table II is 11. A further subembodiment of this aspect of the invention is realized when at least one of the capping group of Table II is 12. A further subembodiment of this aspect of the invention is realized when at least one of the capping group of Table II is 15. A further subembodiment of this aspect of the invention is realized when at least one of the capping group of Table II is 19. A further subembodiment of this aspect of the invention is realized when at least one of the capping group of Table II is 20. A further subembodiment of this aspect of the invention is realized when at least one of the capping group of Table II is 21. A further subembodiment of this aspect of the invention is realized when at least one of the capping group of Table II is 22. A further subembodiment of this aspect of the invention is realized when at least one of the capping group of Table II is 23. A further subembodiment of this aspect of the invention is realized when at least one of the capping group of Table II is 24. A further subembodiment of this aspect of the invention is realized when at least one of the capping group of Table II is 25. A further subembodiment of this aspect of the invention is realized when at least one of the capping group of Table II is 26. A further subembodiment of this aspect of the invention is realized when at least one of the capping group of Table II is 27. A further subembodiment of this aspect of the invention is realized when at least one of the capping group of Table II is 28.

In particular aspects of the insulin dimer, the first insulin and the second insulin heterodimers are independently native human insulin, insulin lispro, insulin aspart, desB30 insulin, or insulin glargine.

In a further aspect of the invention, the insulin dimer may be symmetrical, consisting of two identical insulins, or unsymmetrical, consisting of two insulins which differ either in the capping groups at the lysines and N-terminals of B-chains or in the amino acid sequence of the peptidic backbone.

The present invention further provides a composition comprising a first insulin or insulin analog heterodimer and a second insulin or insulin analog heterodimer each heterodimer including an A-chain polypeptide and a B-chain polypeptide, wherein the A-chain polypeptide and the B-chain polypeptide are linked together through interchain disulfide bonds; wherein the first and second insulin or insulin analog heterodimers are covalently linked together through a linking moiety joining the α-amino groups at the A1 position of the two respective A-chain polypeptides, wherein the linking moiety is selected from the group consisting of Linking moiety Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, Linker 23, Linker 24, Linker 25, Linker 26, Linker 27, Linker 28, Linker 29, Linker 30, Linker 31, Linker 32, Linker 33, Linker 34, Linker 35, Linker 36, Linker 37, Linker 38, Linker 39, Linker 40, Linker 41, Linker 42, Linker 43, Linker 44, Linker 45, and Linker 46, wherein each of the ε-amines of the B-29 or B-28 lysine and the N-terminal amino acids of each of the B-chain of the two insulins optionally and independently are conjugated with a capping group selected from Capping Group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31. An embodiment of this aspect of the invention is realized when the insulin is recombinant human insulin and the insulin analog is selected from the group consisting of insulin lispro, insulin aspart, and insulin glargine; and wherein the amino terminus of the B-chain polypeptides of the first insulin polypeptide and second insulin polypeptide is covalently linked to a capping group.

Exemplary insulin dimers of the present invention are represented by Formula I.

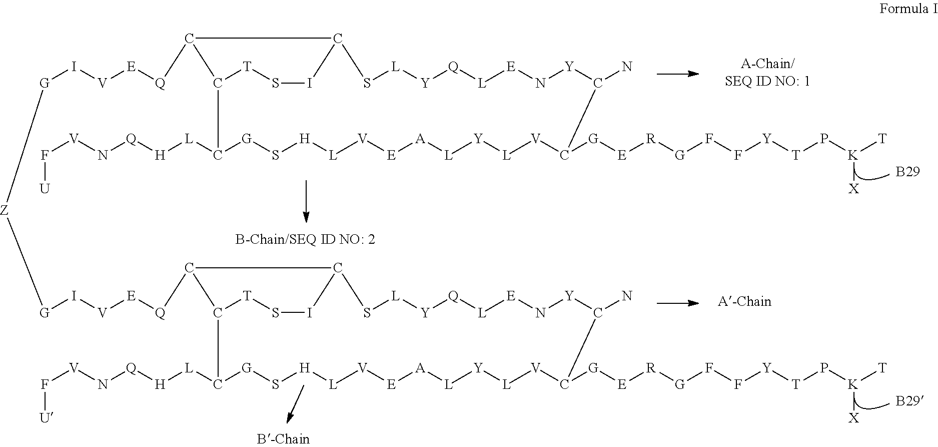

wherein X, U, X′ and U′ are capping groups on lysines and N-terminals at B1 of each B-chain and Z is the linker moiety, a first insulin heterodimer molecule having a first A-chain polypeptide and first B-chain polypeptide and a second insulin heterodimer having a second A′-chain polypeptide and second B′-chain polypeptide that is conjugated together at the α-amino groups of the first and second heterodimer, respectively, by a bifunctional linking moiety represented by Z, the A-chain and A′-chain peptides have the amino acid sequence shown in SEQ ID NO: 1 and the B-chain and B′-chain peptides have the amino acid sequence shown in SEQ ID NO: 2, and wherein the cysteine residues at positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

The present invention further provides a composition comprising an insulin dimer selected from the group consisting of Dimers 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, and 144 from Table III.

The present invention further provides a method for treating diabetes comprising administering to an individual with diabetes a therapeutically effective amount of a composition comprising the insulin receptor partial agonist of any one of insulin dimers disclosed herein. In particular aspects the diabetes is Type 1 diabetes, Type 2 diabetes, or gestational diabetes.

The present invention further provides a composition for the treatment of diabetes comprising the any one of the above insulin dimers. In particular aspects the diabetes is Type 1 diabetes, Type 2 diabetes, or gestational diabetes.

The present invention further provides for the use of any one of the above the insulin dimers for the manufacture of a medicament for the treatment of diabetes. In particular aspects the diabetes is Type 1 diabetes, Type 2 diabetes, or gestational diabetes.

The present invention further provides a composition comprising any one of the aforementioned insulin dimers and a glucagon-like protein 1 (GLP-1) receptor agonist. In particular aspects, the GLP-1 agonist is liraglutide, dulaglutide, or albiglutide.

### BRIEF DESCRIPTION OF THE DRAWINGS

FIG. 1 shows the change in plasma glucose in diabetic minipigs over time for Dimers 17, 30, and 44 compared to recombinant human insulin (RHI). Dimers and RHI were administered at 0.69 nmol/kg.

FIG. 2 shows the change in plasma glucose in diabetic minipigs over time for Dimers 49, 57, and 62 compared to recombinant human insulin (RHI). Dimers and RHI were administered at 0.69 nmol/kg.

FIG. 3 shows the change in plasma glucose in diabetic minipigs over time for Dimers 84, 85, and 86 compared to recombinant human insulin (RHI). Dimers and RHI were administered at 0.69 nmol/kg.

FIG. 4 shows the change in plasma glucose in diabetic minipigs over time for Dimers 132, 134, and 139 compared to recombinant human insulin (RHI). Dimers and RHI were administered at 0.69 nmol/kg.

### DETAILED DESCRIPTION OF THE INVENTION

The present invention provides compounds comprising two insulin molecules covalently linked to form a covalently-linked insulin dimer that may activate the insulin receptor with regular insulin-like potency and reduced maximum activity. These compounds are insulin receptor partial agonists (IRPA): they behave like other insulin analogs to lower glucose effectively but with lower risk of hypoglycemia.

### Definitions

Insulin—as used herein, the term means the active principle of the pancreas that affects the metabolism of carbohydrates in the animal body and which is of value in the treatment of diabetes mellitus. The term includes synthetic and biotechnologically derived products that are the same as, or similar to, naturally occurring insulins in structure, use, and intended effect and are of value in the treatment of diabetes mellitus. The term is a generic term that designates the 51 amino acid heterodimer comprising the A-chain peptide having the amino acid sequence shown in SEQ ID NO: 1 and the B-chain peptide having the amino acid sequence shown in SEQ ID NO: 2, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

Insulin analog or analogue—the term as used herein includes any heterodimer analogue or single-chain analogue that comprises one or more modification(s) of the native A-chain peptide and/or B-chain peptide. Modifications include but are not limited to substituting an amino acid for the native amino acid at a position selected from A4, A5, A8, A9, A10, A12, A13, A14, A15, A16, A17, A18, A19, A21, B1, B2, B3, B4, B5, B9, B10, B13, B14, B15, B16, B17, B18, B20, B21, B22, B23, B26, B27, B28, B29, and B30; deleting any or all of positions B1-4 and B26-30; or conjugating directly or by a polymeric or non-polymeric linker one or more acyl, polyethylglycine (PEG), or saccharide moiety (moieties); or any combination thereof. As exemplified by the N-linked glycosylated insulin analogues disclosed herein, the term further includes any insulin heterodimer and single-chain analogue that has been modified to have at least one N-linked glycosylation site and in particular, embodiments in which the N-linked glycosylation site is linked to or occupied by an N-glycan. Examples of insulin analogues include but are not limited to the heterodimer and single-chain analogues disclosed in published international application WO20100080606, WO2009/099763, and WO2010080609, the disclosures of which are incorporated herein by reference. Examples of single-chain insulin analogues also include but are not limited to those disclosed in published International Applications WO9634882, WO95516708, WO2005054291, WO2006097521, WO2007104734, WO2007104736, WO2007104737, WO2007104738, WO2007096332, WO2009132129; U.S. Pat. Nos. 5,304,473, 6,630,348 and 8,273,361; and Kristensen et al., Biochem. J. 305: 981-986 (1995), the disclosures of which are each incorporated herein by reference.

The term further includes single-chain and heterodimer polypeptide molecules that have little or no detectable activity at the insulin receptor but which have been modified to include one or more amino acid modifications or substitutions to have an activity at the insulin receptor that has at least 1%, 10%, 50%, 75%, or 90% of the activity at the insulin receptor as compared to native insulin and which further includes at least one N-linked glycosylation site. In particular aspects, the insulin analogue is a partial agonist that has less than 80% (or 70%) activity at the insulin receptor as does native insulin. These insulin analogues, which have reduced activity at the insulin growth hormone receptor and enhanced activity at the insulin receptor, include both heterodimers and single-chain analogues.

Single-chain insulin or single-chain insulin analog as used herein, the term encompasses a group of structurally-related proteins wherein the A-chain peptide or functional analogue and the B-chain peptide or functional analogue are covalently linked by a peptide or polypeptide of 2 to 35 amino acids or non-peptide polymeric or non-polymeric linker and which has at least 1%, 10%, 50%, 75%, or 90% of the activity of insulin at the insulin receptor as compared to native insulin. The single-chain insulin or insulin analogue further includes three disulfide bonds: the first disulfide bond is between the cysteine residues at positions 6 and 11 of the A-chain or functional analogue thereof, the second disulfide bond is between the cysteine residues at position 7 of the A-chain or functional analogue thereof and position 7 of the B-chain or functional analogue thereof, and the third disulfide bond is between the cysteine residues at position 20 of the A-chain or functional analogue thereof and position 19 of the B-chain or functional analogue thereof.

Insulin dimer—as used herein, the term refers to a dimer comprising two insulin heterodimers (insulin molecules comprising an A chain and a B chain) linked together via their respective α-amino groups at position A1 residue of each insulin via a linking moiety as disclosed herein.

Boc—as used herein, the term refers to tert-butoxycarbonyl.

Heterocycles—as used herein, the term refers to a nonaromatic 3-10 membered monocyclic, 8-12 membered bicyclic, or 11-14 membered tricyclic ring system having 1-3 heteroatoms of monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heterocyclyl groups herein described may also contain fused rings. Fused rings are rings that share a common carbon-carbon bond or a common carbon atom (e.g., spiro-fused rings). Examples of heterocyclyl include, but are not limited to tetrahydrofuranyl, tetrahydropyranyl, piperidinyl, morpholino, pyrrolinyl and pyrrolidinyl.

Amino acid modification—as used herein, the term refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, WI), ChemPep Inc. (Miami, FL), and Genzyme Pharmaceuticals (Cambridge, MA). Atypical amino acids may be purchased from commercial suppliers, synthesized de novo, or chemically modified or derivatized from naturally occurring amino acids.

Amino acid substitution—as used herein refers to the replacement of one amino acid residue by a different amino acid residue.

Conservative amino acid substitution—as used herein, the term is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln, cysteic acid and homocysteic acid;
III. Polar, positively charged residues:
   His, Arg, Lys; Omithine (Orn)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine
   Treat—As used herein, the term “treat” (or “treating”, “treated”, “treatment”, etc.)
      refers to the administration of an IRPA of the present disclosure to a subject in need thereof with the purpose to alleviate, relieve, alter, ameliorate, improve or affect a condition (e.g., diabetes), a symptom or symptoms of a condition (e.g., hyperglycemia), or the predisposition toward a condition. For example, as used herein the term “treating diabetes” will refer in general to maintaining glucose blood levels near normal levels and may include increasing or decreasing blood glucose levels depending on a given situation.

The term AEG-C6 is depicted as:

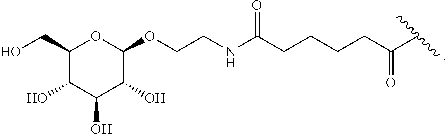 [SMILES: *C(=O)CCCCC(=O)NCCO[C@@H]1O[C@H](CO)[C@@H](O)[C@H](O)[C@H]1O]

Pharmaceutically acceptable carrier—as used herein, the term includes any of the standard pharmaceutical carriers, such as a phosphate buffered saline solution, water, emulsions such as an oil/water or water/oil emulsion, and various types of wetting agents suitable for administration to or by an individual in need. The term also encompasses any of the agents approved by a regulatory agency of the US Federal government or listed in the US Pharmacopeia for use in animals, including humans.

Pharmaceutically acceptable salt—as used herein, the term refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium, zinc, and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

Effective or therapeutically effective amount—as used herein refers to a nontoxic but sufficient amount of an insulin analog to provide the desired effect. For example one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” It is not always possible to determine the optimal effective amount prior to administration to or by an individual in need thereof. However, an appropriate “effective” amount in any individual case may be determined by one of ordinary skill in the art using routine experimentation.

Parenteral—as used herein, the term means not through the alimentary canal but by some other route such as intranasal, inhalation, subcutaneous, intramuscular, intraspinal, or intravenous.

The instant invention relates to insulin dimers where the level of insulin activity and partial agonist activity of the dimers is a function of the dimeric structure that involves the sequence of the insulin analog, the length of the dimerization linker, and the site of dimerization that connects the two insulin polypeptides. The insulin dimers of the present invention have reduced risk of promoting hypoglycemia when administered in high doses than native insulin or other insulin analogs when administered at high doses. The instant invention further relates to insulin dimers with capping groups X, X′, U, U′ optionally and independently introduced at ε-amines of lysine at B29 positions (B-28 lysine in case of LISPRO insulin) and N-terminal amino acids (B1) of B-chain insulin of Formula I. The instant invention further relates to insulin dimers with at least one capping group X, X′, U, or U′ introduced at B1, B29 (B28 in the case of LISPRO insulin), B1′, or B29′ (B28′ in the case of LISPRO insulin) positions of Formula I. A subembodiment of this aspect of the invention is realized when the insulin dimer has one capping group independently introduced at any of B1, B29 and B-28 positions, B1′, or B29′ and B-28′ positions positions of Formula I. Another subembodiment of this aspect of the invention is realized when the insulin dimer has two capping groups independently introduced at any of B1, B29 and B-28 positions, B1′, or B29′ and B-28′ positions positions of Formula I. Still another subembodiment of this aspect of the invention is realized when the insulin dimer has three capping groups independently introduced at any of B1, B29 and B-28 positions, B1′, or B29′ and B-28′ positions positions of Formula I. Yet another subembodiment of this aspect of the invention is realized when the insulin dimer has four capping groups independently introduced at any of B1, B29 and B-28 positions, B1′, or B29′ and B-28′ positions positions of Formula I.

The present invention provides partial agonist covalently-linked insulin dimers formulated as a novel and transformative basal insulin (once daily administration) that manifests improved therapeutic index (TI) over current standard of care (SOC) basal insulins. These molecules may lower glucose effectively with reduced risk of hypoglycemia in diabetic minipig and have the property of a once daily (QD) basal insulin. The improved TI may enable practitioners to more aggressively dose IRPA insulin dimer to achieve target goals for control of fasting glucose. Tight control of fasting glucose and HbAlc may allow these molecules to serve as 1) a stand-alone long-acting insulin with an enhanced efficacy and safety profile in Type 2 diabetes mellitus (T2DM) and 2) an improved foundational basal insulin in Type 1 diabetes mellitus (T1DM) (and some T2DM) for use with additional prandial rapid-acting insulin analogs (RAA) doses.

An ideal long-acting insulin provides continuous control of fasting glucose in diabetics with highly stable and reproducible PK/PD. However, currently available basal insulins, even those with improved stability and reproducibility of PK/PD continue to have a narrow therapeutic index and hypoglycemia incidents increase as glucose levels approach euglycemia target. This can often lead to underdosing to avoid hypoglycemia. Treatment with an IRPA of the present invention is expected to be safer with respect to hypoglycemia due to reduced maximal effect of the drug.

### Insulin A and B Chains

Disclosed herein are insulin or insulin analog dimers that have insulin receptor agonist activity. The level of insulin activity of the dimers is a function of the dimeric structure, the sequence of the insulin analog, the length of the dimerization linker, and the site of dimerization that connects the two insulin polypeptides. The insulin polypeptides of the present invention may comprise the native B and A chain sequences of human insulin (SEQ ID NOs: 1 and 2, respectively) or any of the known analogs or derivatives thereof that exhibit insulin agonist activity when linked to one another in a heteroduplex. Such analogs include, for example, proteins that having an A-chain and a B-chain that differ from the A-chain and B-chain of human insulin by having one or more amino acid deletions, one or more amino acid substitutions, and/or one or more amino acid insertions that do not destroy the insulin activity of the insulin analog.

One type of insulin analog, “monomeric insulin analog,” is well known in the art. These are fast-acting analogs of human insulin, including, for example, insulin analogs wherein:
(a) the amino acid residue at position B28 is substituted with Asp, Lys, Leu, Val, or Ala, and the amino acid residue at position B29 is Lys or Pro;
(b) the amino acid residues at any of positions B27 and B30 are deleted or substituted with a nonnative amino acid.

In one embodiment an insulin analog is provided comprising an Asp substituted at position B28 (e.g., insulin aspart (NOVOLOG); see SEQ ID NO:9) or a Lys substituted at position 28 and a proline substituted at position B29 (e.g., insulin lispro (HUMALOG); see SEQ ID NO:6). Additional monomeric insulin analogs are disclosed in Chance, et al., U.S. Pat. No. 5,514,646; Chance, et al., U.S. patent application Ser. No. 08/255,297; Brems, et al., Protein Engineering, 5:527-533 (1992); Brange, et al., EPO Publication No. 214,826 (published Mar. 18, 1987); and Brange, et al., Current Opinion in Structural Biology, 1:934-940 (1991). These disclosures are expressly incorporated herein by reference for describing monomeric insulin analogs.

Insulin analogs may also have replacements of the amidated amino acids with acidic forms. For example, Asn may be replaced with Asp or Glu. Likewise, Gln may be replaced with Asp or Glu. In particular, Asn(A18), Asn(A21), or Asp(B3), or any combination of those residues, may be replaced by Asp or Glu. Also, Gln(A15) or Gln(B4), or both, may be replaced by either Asp or Glu.

As disclosed herein insulin single chain analogs are provided comprising a B chain and A chain of human insulin, or analogs or derivative thereof, wherein the carboxy terminus of the B chain is linked to the amino terminus of the A chain via a linking moiety. In one embodiment the A chain is amino acid sequence GIVEQCCTSICSLYQLENYCN (SEQ ID NO: 1 and the B chain comprises amino acid sequence FVNQHLCGSH LVEALYLVCGERGFFYTPKT (SEQ ID NO: 2) or a carboxy shortened sequence thereof having B30 deleted, and analogs of those sequences wherein each sequence is modified to comprise one to five amino acid substitutions at positions corresponding to native insulin positions selected from A5, A8, A9, A10, A14, A15, A17, A18, A21, B1, B2, B3, B4, B5, B9, B10, B13, B14, B20, B22, B23, B26, B27, B28, B29 and B30, with the proviso that at least one of B28 or B29 is lysine. In one embodiment the amino acid substitutions are conservative amino acid substitutions. Suitable amino acid substitutions at these positions that do not adversely impact insulin's desired activities are known to those skilled in the art, as demonstrated, for example, in Mayer, et al., Insulin Structure and Function, Biopolymers. 2007; 88(5):687-713, the disclosure of which is incorporated herein by reference.

In accordance with one embodiment, the insulin analog peptides may comprise an insulin A chain and an insulin B chain or analogs thereof, wherein the A chain comprises an amino acid sequence that shares at least 70% sequence identity (e.g., 70%, 75%, 80%, 85%, 90%, 95%) over the length of the native peptide, with GIVEQCCTSICSLYQLENYCN (SEQ ID NO: 1) and the B chain comprises an amino acid sequence that shares at least 60% sequence identity (e.g., 60%, 65%, 70%, 75%, 80%, 85%, 90%, 95%) over the length of the native peptide, with FVNQHLCGSHLVEALYLVCGERGFFYTPKT (SEQ ID NO: 2) or a carboxy shortened sequence thereof having B30 deleted.

Additional amino acid sequences can be added to the amino terminus of the B chain or to the carboxy terminus of the A chain of the insulin polypeptides of the present invention. For example, a series of negatively charged amino acids can be added to the amino terminus of the B chain, including for example a peptide of 1 to 12, 1 to 10, 1 to 8 or 1 to 6 amino acids in length and comprising one or more negatively charged amino acids including for example glutamic acid and aspartic acid. In one embodiment the B chain amino terminal extension comprises 1 to 6 charged amino acids. In accordance with one embodiment, the insulin polypeptides disclosed comprise a C-terminal amide or ester in place of a C-terminal carboxylate on the A chain.

In various embodiments, the insulin analog has an isoelectric point that has been shifted relative to human insulin. In some embodiments, the shift in isoelectric point is achieved by adding one or more arginine, lysine, or histidine residues to the N-terminus of the insulin A-chain peptide and/or the C-terminus of the insulin B-chain peptide. Examples of such insulin polypeptides include Arg^{A0}-human insulin, Arg^{B31}Arg^{B32}-human insulin, Gly^{A21}Arg^{B31}Arg^{B32}-human insulin, Arg^{A0}Arg^{B31}Arg^{B32}-human insulin, and Arg^{A0}Gly^{A21}Arg^{B31}Arg^{B32}-human insulin. By way of further example, insulin glargine (LANTUS; see SEQ ID NOs: 7 and 8) is an exemplary long-acting insulin analog in which Asn^{A21} has been replaced by glycine, and two arginine residues have been covalently linked to the C-terminus of the B-peptide. The effect of these amino acid changes was to shift the isoelectric point of the molecule, thereby producing a molecule that is soluble at acidic pH (e.g., pH 4 to 6.5) but insoluble at physiological pH. When a solution of insulin glargine is injected into the muscle, the pH of the solution is neutralized and the insulin glargine forms microprecipitates that slowly release the insulin glargine over the 24 hour period following injection with no pronounced insulin peak and thus a reduced risk of inducing hypoglycemia. This profile allows a once-daily dosing to provide a patient's basal insulin. Thus, in some embodiments, the insulin analog comprises an A-chain peptide wherein the amino acid at position A21 is glycine and a B-chain peptide wherein the amino acids at position B31 and B32 are arginine. The present disclosure encompasses all single and multiple combinations of these mutations and any other mutations that are described herein (e.g., Gly^{A21}-human insulin, Gly^{A21}Arg^{B31}-human insulin, Arg^{B31}Arg^{B32}-human insulin, Arg^{B31}-human insulin).

In particular aspects of the insulin receptor partial agonists, one or more amidated amino acids of the insulin analog are replaced with an acidic amino acid, or another amino acid. For example, asparagine may be replaced with aspartic acid or glutamic acid, or another residue. Likewise, glutamine may be replaced with aspartic acid or glutamic acid, or another residue. In particular, Asn^{A18}, Asn^{A21}, or Asn^{B3}, or any combination of those residues, may be replaced by aspartic acid or glutamic acid, or another residue. Gln^{A15} or Gln^{B4}, or both, may be replaced by aspartic acid or glutamic acid, or another residue. In particular aspects of the insulin receptor partial agonists, the insulin analogs have an aspartic acid, or another residue, at position A21 or aspartic acid, or another residue, at position B3, or both.

One skilled in the art will recognize that it is possible to replace yet other amino acids in the insulin analog with other amino acids while retaining biological activity of the molecule. For example, without limitation, the following modifications are also widely accepted in the art: replacement of the histidine residue of position B10 with aspartic acid (His^{B10} to AspB10); replacement of the phenylalanine residue at position B1 with aspartic acid (PheBI to AspB1); replacement of the threonine residue at position B30 with alanine (ThrB30 toAlaB30); replacement of the tyrosine residue at position B26 with alanine (TyrB26 to AlaB26); and replacement of the serine residue at position B9 with aspartic acid (SerB9 to AspB9).

In various embodiments, the insulin analog has a protracted profile of action. Thus, in certain embodiments, the insulin analog may be acylated with a fatty acid. That is, an amide bond is formed between an amino group on the insulin analog and the carboxylic acid group of the fatty acid. The amino group may be the alpha-amino group of an N-terminal amino acid of the insulin analog, or may be the epsilon-amino group of a lysine residue of the insulin analog. The insulin analog may be acylated at one or more of the three amino groups that are present in wild-type human insulin may be acylated on lysine residue that has been introduced into the wild-type human insulin sequence. In particular aspects of the insulin receptor partial agonists, the insulin analog may be acylated at position B1, B1′, or both B1 and B1′.

Examples of insulin analogs can be found for example in published International Application WO9634882, WO95516708; WO20100080606, WO2009/099763, and WO2010080609, U.S. Pat. No. 6,630,348, and Kristensen et al., Biochem. J. 305: 981-986 (1995), the disclosures of which are incorporated herein by reference). In further embodiments, the in vitro glycosylated or in vivo N-glycosylated insulin analogs may be acylated and/or pegylated.
In particular aspects of the insulin dimer, each A-chain polypeptide independently comprises the amino acid sequenceGX₂X₃EQCCX₈SICSLYQLX₁₇NX₁₉CX₂₃ (SEQ ID NO:3) and each B-chain polypeptide independently comprises the amino acid sequence X₂₅LCGX₂₉X₃₀LVEALYLVCGERGFX₂₇YTX₃₁X₃₂ (SEQ ID NO:4) or
X₂₂VNQX₂₅X₂₆CGX₂₉X₃₀LVEALYLVCGERGFX₂₇YTX₃₁X₃₂X₃₃X₃₄X₃₅ (SEQ ID NO:5) wherein X₂ is isoleucine or threonine; X₃ is valine, glycine, or leucine; X₈ is threonine or histidine; X₁₇ is glutamic acid or glutamine; X₁₉ is tyrosine, 4-methoxy-phenylalanine, alanine, or 4-amino phenylalanine; X₂₃ is asparagine or glycine; X₂₂ is or phenylalanine and desamino-phenylalanine; X₂₅ is histidine or threonine; X₂₆ is leucine or glycine; X₂₇ is phenylalanine or aspartic acid; X₂₉ is alanine, glycine, or serine; X₃₀ is histidine, aspartic acid, glutamic acid, homocysteic acid, or cysteic acid; X₃₁ is aspartic acid, proline, or lysine; X₃₂ is lysine or proline; X₃₃ is threonine, alanine, or absent; X₃₄ is arginine or absent; and X₃₅ is arginine or absent; with the proviso at least one of X₃₁ or X₃₂ is lysine.

### Linking Moiety

The insulin dimers disclosed herein are formed between a first and second insulin polypeptide wherein each insulin polypeptide comprises an A chain and a B chain. The first and second insulin polypeptides may be two chain insulin analogs (i.e., wherein the A and B chains are linked only via inter-chain disulfide bonds between internal cysteine residues) wherein the first and second insulin polypeptides are linked to one another to form the dimer by a covalent bond, bifunctional linker, or other means known in the art to link linking moieties on the respective B chains. In accordance with one embodiment the first and second insulin polypeptides are linked to one another by a bifunctional linker joining the side chain of the A1□-amino group of the A chain of the first insulin polypeptide to the side chain of the A1 □-amino group of the A′ chain of the second insulin polypeptide.

The following Table I shows exemplary linker structures, which may be used to construct the dimers of the present invention. The linker reagent shown comprise 2,5-dioxopyrrolidin-1yl groups for conjugating to the alpha amino group of the A1 residue of each insulin. Also shown are exemplary linking moieties of the invention.

TABLE I

| | | Linking Moiety (Wavy lines indicate |
|---|---|---|
| Linker | | attachment to A1 and A1′ sites of two |
| No. | Linking Reagent | insulins) |
| 1 | 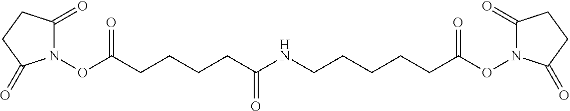 [SMILES: O=C(CCCCC(=O)ON1C(=O)CCC1=O)NCCCCCC(=O)ON1C(=O)CCC1=O] | 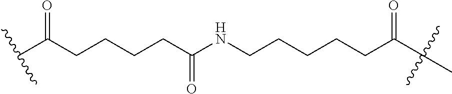 [SMILES: *C(=O)CCCCCNC(=O)CCCCC(*)=O] |
| 2 | 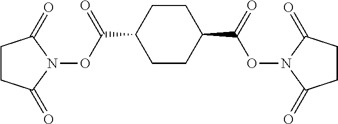 [SMILES: O=C1CCC(=O)N1OC(=O)[C@H]1CC[C@H](C(=O)ON2C(=O)CCC2=O)CC1] | 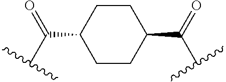 [SMILES: *C(=O)[C@H]1CC[C@H](C(*)=O)CC1] |
| 3 | 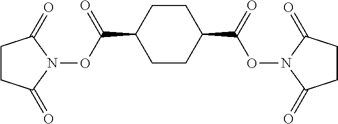 [SMILES: O=C1CCC(=O)N1OC(=O)[C@H]1CC[C@@H](C(=O)ON2C(=O)CCC2=O)CC1] | 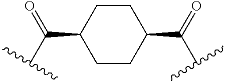 [SMILES: *C(=O)[C@H]1CC[C@@H](C(*)=O)CC1] |
| 4 | 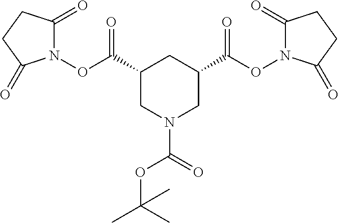 [SMILES: CC(C)(C)OC(=O)N1C[C@H](C(=O)ON2C(=O)CCC2=O)C[C@H](C(=O)ON2C(=O)CCC2=O)C1] | 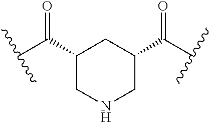 [SMILES: *C(=O)[C@@H]1CNC[C@H](C(*)=O)C1] |
| 5 | 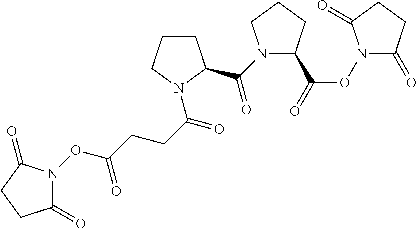 [SMILES: O=C(CCC(=O)N1CCC[C@H]1C(=O)N1CCC[C@H]1C(=O)ON1C(=O)CCC1=O)ON1C(=O)CCC1=O] | 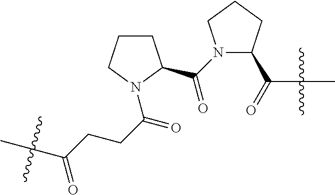 [SMILES: *C(=O)CCC(=O)N1CCC[C@H]1C(=O)N1CCC[C@H]1C(*)=O] |
| 6 | 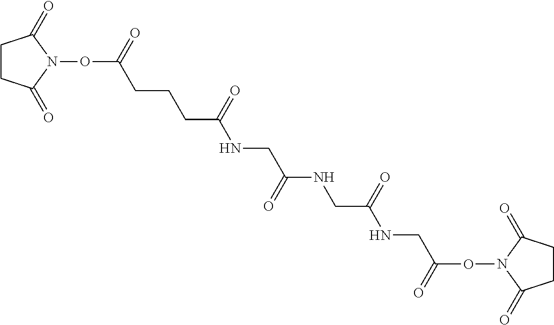 [SMILES: O=C(CCCC(=O)ON1C(=O)CCC1=O)NCC(=O)NCC(=O)NCC(=O)ON1C(=O)CCC1=O] | 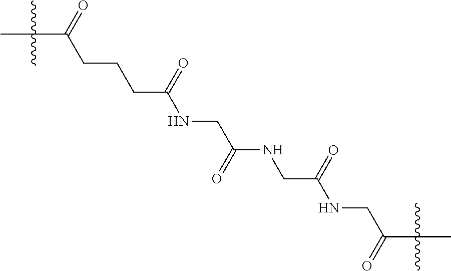 [SMILES: *C(=O)CCCC(=O)NCC(=O)NCC(=O)NCC(*)=O] |
| 7 | 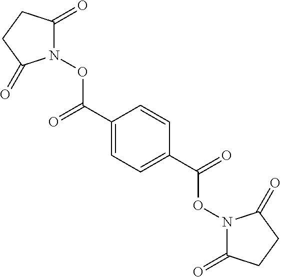 [SMILES: O=C(ON1C(=O)CCC1=O)c1ccc(C(=O)ON2C(=O)CCC2=O)cc1] | 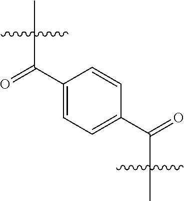 [SMILES: *C(=O)c1ccc(C(*)=O)cc1] |
| 8 | 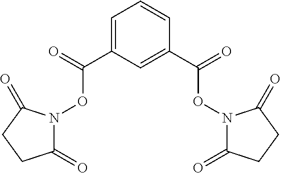 [SMILES: O=C(ON1C(=O)CCC1=O)c1cccc(C(=O)ON2C(=O)CCC2=O)c1] | 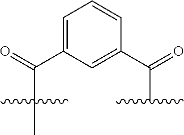 [SMILES: *C(=O)c1cccc(C(*)=O)c1] |
| 9 | 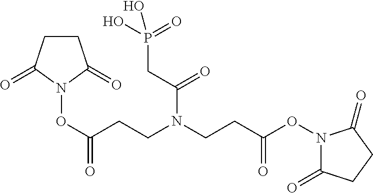 [SMILES: O=C(CCN(CCC(=O)ON1C(=O)CCC1=O)C(=O)CP(=O)(O)O)ON1C(=O)CCC1=O] | 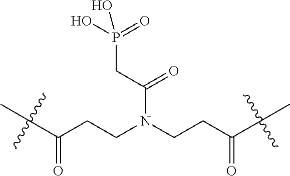 [SMILES: *C(=O)CCN(CCC(*)=O)C(=O)CP(=O)(O)O] |
| 10 | 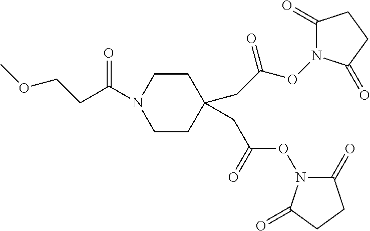 [SMILES: COCCC(=O)N1CCC(CC(=O)ON2C(=O)CCC2=O)(CC(=O)ON2C(=O)CCC2=O)CC1] | 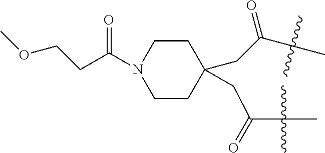 [SMILES: *C(=O)CC1(CC(*)=O)CCN(C(=O)CCOC)CC1] |
| 11 | 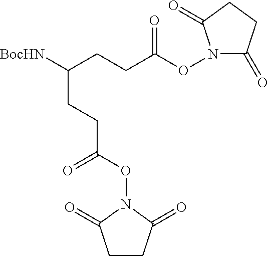 [SMILES: CC(C)(C)OC(=O)NC(CCC(=O)ON1C(=O)CCC1=O)CCC(=O)ON1C(=O)CCC1=O] | 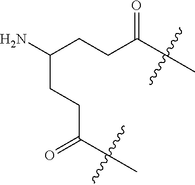 [SMILES: *C(=O)CCC(N)CCC(*)=O] |
| 12 | 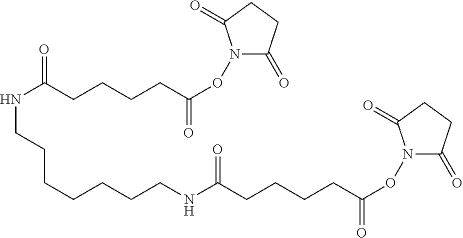 [SMILES: O=C(CCCCC(=O)ON1C(=O)CCC1=O)NCCCCCCCNC(=O)CCCCC(=O)ON1C(=O)CCC1=O] | 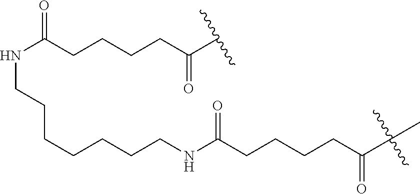 [SMILES: *C(=O)CCCCC(=O)NCCCCCCCNC(=O)CCCCC(*)=O] |
| 13 | 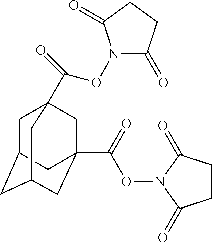 [SMILES: O=C1CCC(=O)N1OC(=O)C12CC3CC(C1)CC(C(=O)ON1C(=O)CCC1=O)(C3)C2] | 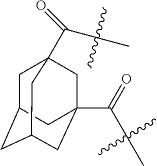 [SMILES: *C(=O)C12CC3CC(C1)CC(C(*)=O)(C3)C2] |
| 14 | 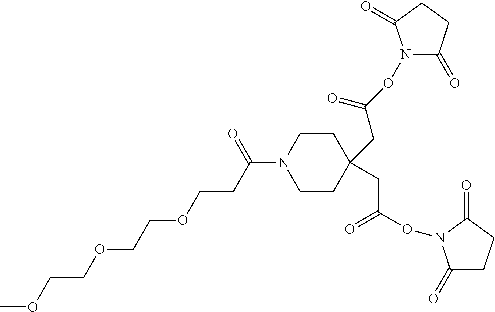 [SMILES: COCCOCCOCCC(=O)N1CCC(CC(=O)ON2C(=O)CCC2=O)(CC(=O)ON2C(=O)CCC2=O)CC1] | 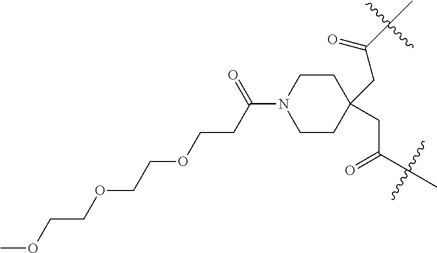 [SMILES: *C(=O)CC1(CC(*)=O)CCN(C(=O)CCOCCOCCOC)CC1] |
| 15 | 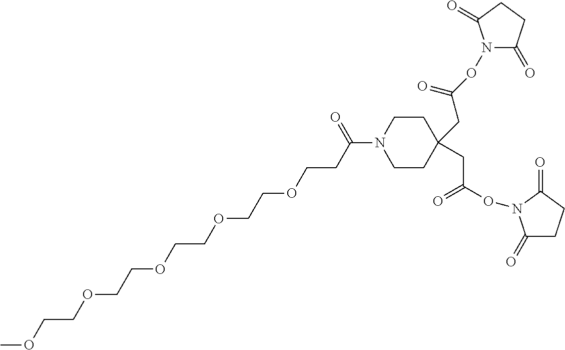 [SMILES: COCCOCCOCCOCCOCCC(=O)N1CCC(CC(=O)ON2C(=O)CCC2=O)(CC(=O)ON2C(=O)CCC2=O)CC1] | 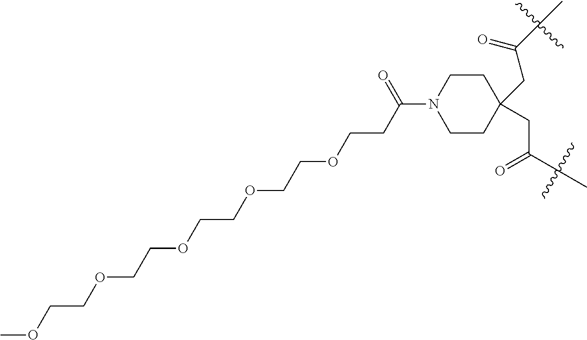 [SMILES: *C(=O)CC1(CC(*)=O)CCN(C(=O)CCOCCOCCOCCOCCOC)CC1] |
| 16 | 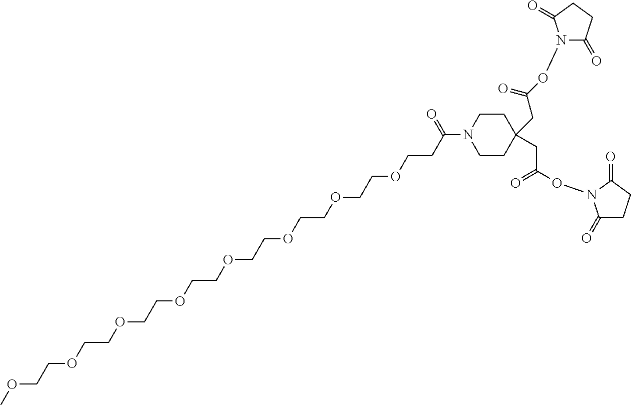 [SMILES: COCCOCCOCCOCCOCCOCCOCCOCCC(=O)N1CCC(CC(=O)ON2C(=O)CCC2=O)(CC(=O)ON2C(=O)CCC2=O)CC1] | 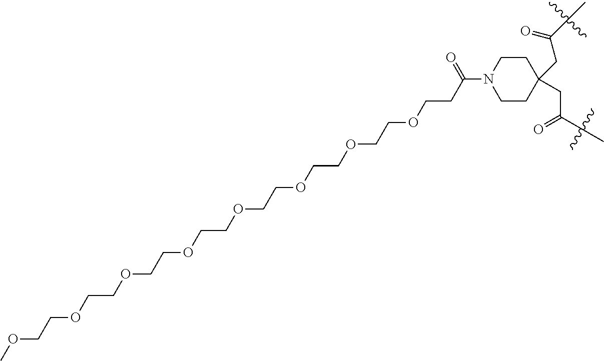 [SMILES: *C(=O)CC1(CC(*)=O)CCN(C(=O)CCOCCOCCOCCOCCOCCOCCOCCOC)CC1] |
| 17 | 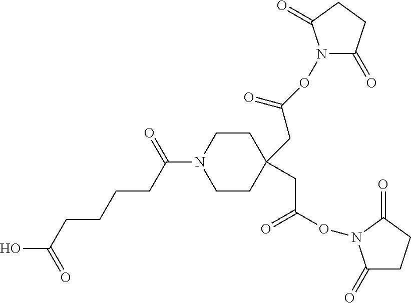 [SMILES: O=C(O)CCCCC(=O)N1CCC(CC(=O)ON2C(=O)CCC2=O)(CC(=O)ON2C(=O)CCC2=O)CC1] | 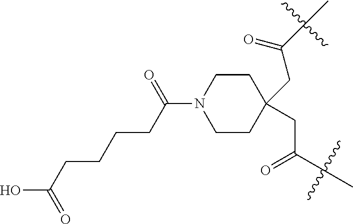 [SMILES: *C(=O)CC1(CC(*)=O)CCN(C(=O)CCCCC(=O)O)CC1] |
| 18 | 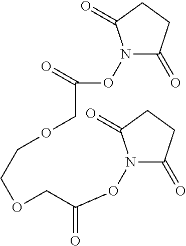 [SMILES: O=C(COCCOCC(=O)ON1C(=O)CCC1=O)ON1C(=O)CCC1=O] | 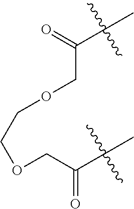 [SMILES: *C(=O)COCCOCC(*)=O] |
| 19 | 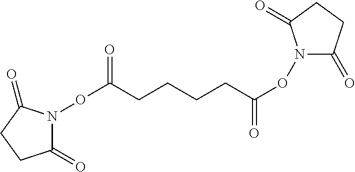 [SMILES: O=C(CCCCC(=O)ON1C(=O)CCC1=O)ON1C(=O)CCC1=O] | 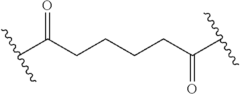 [SMILES: *C(=O)CCCCC(*)=O] |
| 20 | 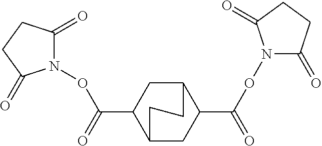 [SMILES: O=C(ON1C(=O)CCC1=O)C1CC2CCC1CC2C(=O)ON1C(=O)CCC1=O] | 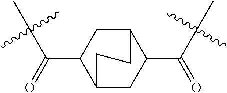 [SMILES: *C(=O)C1CC2CCC1CC2C(*)=O] |
| 21 | 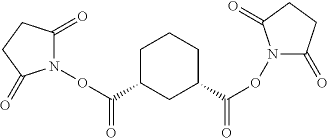 [SMILES: O=C(ON1C(=O)CCC1=O)[C@@H]1CCC[C@H](C(=O)ON2C(=O)CCC2=O)C1] | 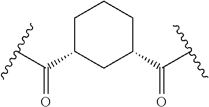 [SMILES: *C(=O)[C@@H]1CCC[C@H](C(*)=O)C1] |
| 22 | 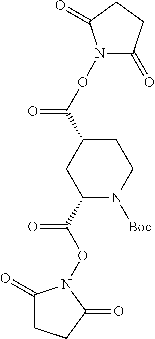 [SMILES: CC(C)(C)OC(=O)N1CC[C@@H](C(=O)ON2C(=O)CCC2=O)C[C@H]1C(=O)ON1C(=O)CCC1=O] | 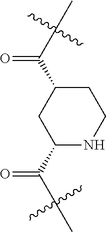 [SMILES: *C(=O)[C@@H]1CCN[C@H](C(*)=O)C1] |
| 23 | 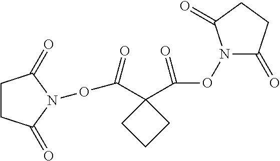 [SMILES: O=C1CCC(=O)N1OC(=O)C1(C(=O)ON2C(=O)CCC2=O)CCC1] | 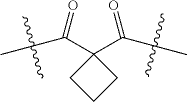 [SMILES: *C(=O)C1(C(*)=O)CCC1] |
| 24 | 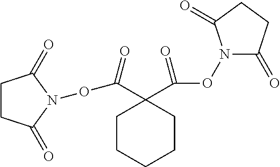 [SMILES: O=C1CCC(=O)N1OC(=O)C1(C(=O)ON2C(=O)CCC2=O)CCCCC1] | 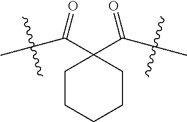 [SMILES: *C(=O)C1(C(*)=O)CCCCC1] |
| 25 | 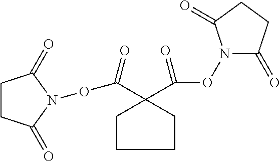 [SMILES: O=C1CCC(=O)N1OC(=O)C1(C(=O)ON2C(=O)CCC2=O)CCCC1] | 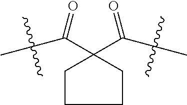 [SMILES: *C(=O)C1(C(*)=O)CCCC1] |
| 26 | 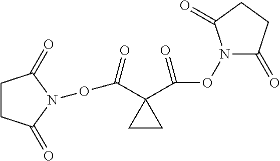 [SMILES: O=C1CCC(=O)N1OC(=O)C1(C(=O)ON2C(=O)CCC2=O)CC1] | 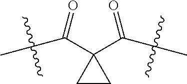 [SMILES: *C(=O)C1(C(*)=O)CC1] |
| 27 | 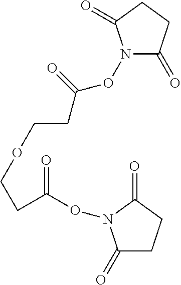 [SMILES: O=C(CCOCCC(=O)ON1C(=O)CCC1=O)ON1C(=O)CCC1=O] | 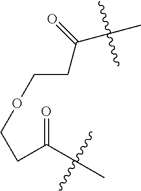 [SMILES: *C(=O)CCOCCC(*)=O] |
| 28 | 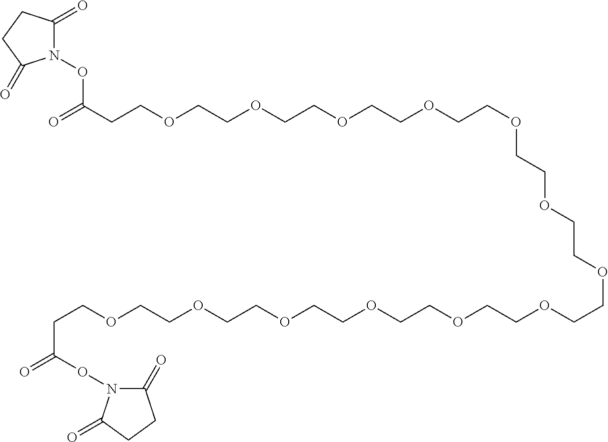 [SMILES: O=C(CCOCCOCCOCCOCCOCCOCCOCCOCCOCCOCCOCCOCCOCCC(=O)ON1C(=O)CCC1=O)ON1C(=O)CCC1=O] | 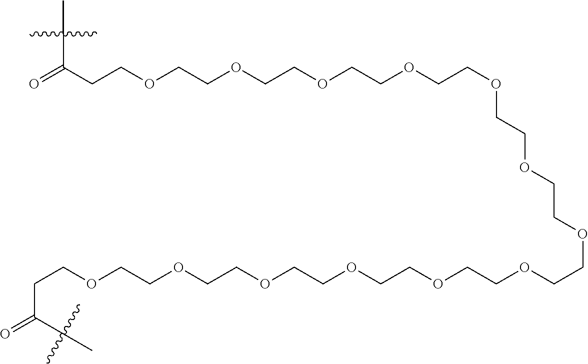 [SMILES: *C(=O)CCOCCOCCOCCOCCOCCOCCOCCOCCOCCOCCOCCOCCOCCC(*)=O] |
| 29 | 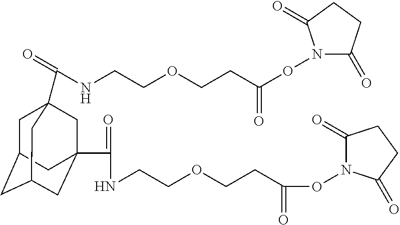 [SMILES: O=C(CCOCCNC(=O)C12CC3CC(C1)CC(C(=O)NCCOCCC(=O)ON1C(=O)CCC1=O)(C3)C2)ON1C(=O)CCC1=O] | 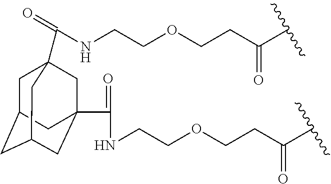 [SMILES: *C(=O)CCOCCNC(=O)C12CC3CC(C1)CC(C(=O)NCCOCCC(*)=O)(C3)C2] |
| 30 | 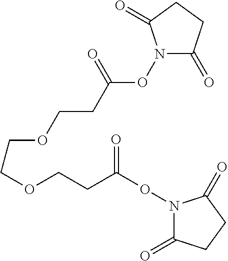 [SMILES: O=C(CCOCCOCCC(=O)ON1C(=O)CCC1=O)ON1C(=O)CCC1=O] | 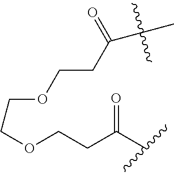 [SMILES: *C(=O)CCOCCOCCC(*)=O] |
| 31 | 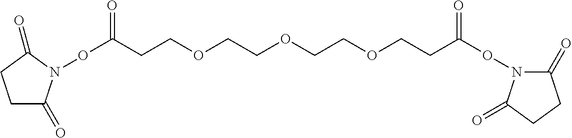 [SMILES: O=C(CCOCCOCCOCCC(=O)ON1C(=O)CCC1=O)ON1C(=O)CCC1=O] | 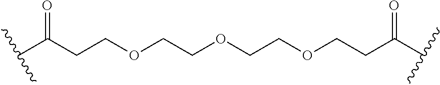 [SMILES: *C(=O)CCOCCOCCOCCC(*)=O] |
| 32 | 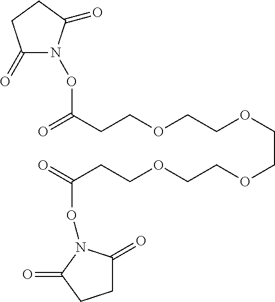 [SMILES: O=C(CCOCCOCCOCCOCCC(=O)ON1C(=O)CCC1=O)ON1C(=O)CCC1=O] | 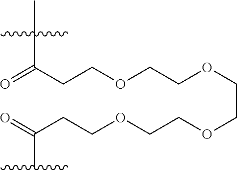 [SMILES: *C(=O)CCOCCOCCOCCOCCC(*)=O] |
| 33 | 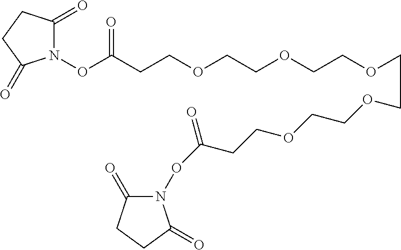 [SMILES: O=C(CCOCCOCCOCCOCCOCCC(=O)ON1C(=O)CCC1=O)ON1C(=O)CCC1=O] | 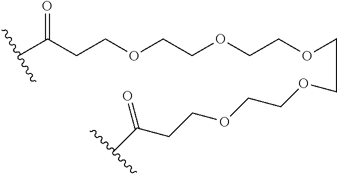 [SMILES: *C(=O)CCOCCOCCOCCOCCOCCC(*)=O] |
| 34 | 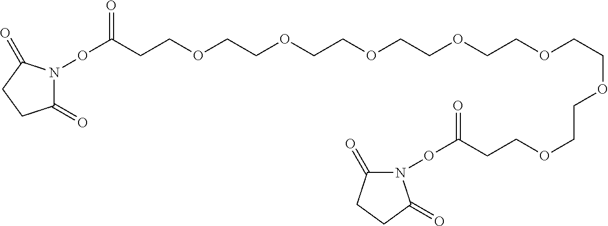 [SMILES: O=C(CCOCCOCCOCCOCCOCCOCCOCCC(=O)ON1C(=O)CCC1=O)ON1C(=O)CCC1=O] | 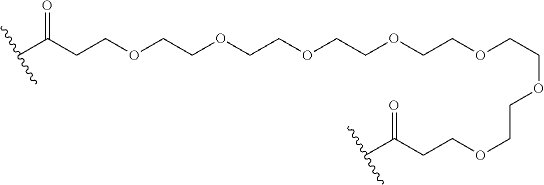 [SMILES: *C(=O)CCOCCOCCOCCOCCOCCOCCOCCC(*)=O] |
| 35 | 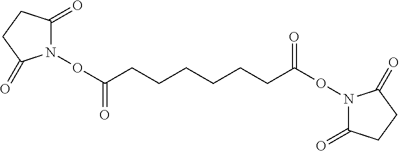 [SMILES: O=C(CCCCCCC(=O)ON1C(=O)CCC1=O)ON1C(=O)CCC1=O] | 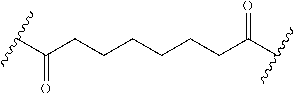 [SMILES: *C(=O)CCCCCCC(*)=O] |
| 36 | 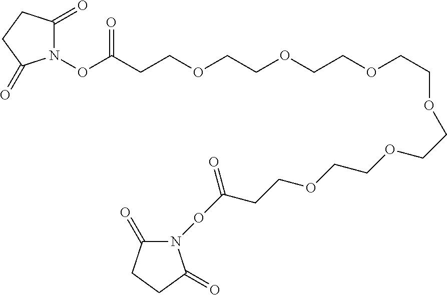 [SMILES: O=C(CCOCCOCCOCCOCCOCCOCCC(=O)ON1C(=O)CCC1=O)ON1C(=O)CCC1=O] | 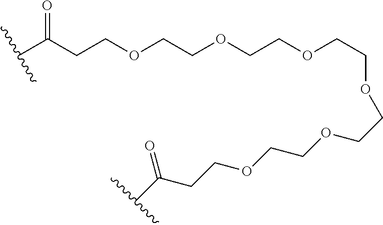 [SMILES: *C(=O)CCOCCOCCOCCOCCOCCOCCC(*)=O] |
| 37 | 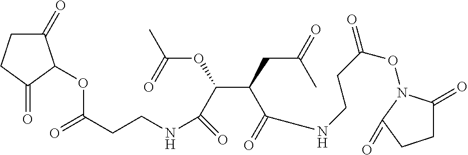 [SMILES: CC(=O)C[C@@H](C(=O)NCCC(=O)ON1C(=O)CCC1=O)[C@@H](OC(C)=O)C(=O)NCCC(=O)OC1C(=O)CCC1=O] | 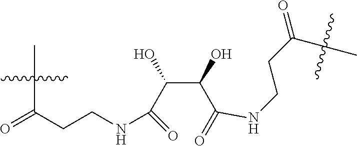 [SMILES: *C(=O)CCNC(=O)[C@H](O)[C@@H](O)C(=O)NCCC(*)=O] |
| 38 | 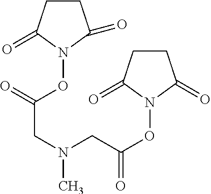 [SMILES: CN(CC(=O)ON1C(=O)CCC1=O)CC(=O)ON1C(=O)CCC1=O] | 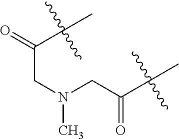 [SMILES: *C(=O)CN(C)CC(*)=O] |
| 39 | 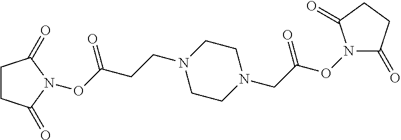 [SMILES: O=C(CCN1CCN(CC(=O)ON2C(=O)CCC2=O)CC1)ON1C(=O)CCC1=O] | 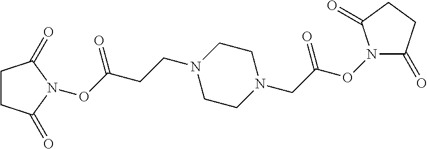 [SMILES: O=C(CCN1CCN(CC(=O)ON2C(=O)CCC2=O)CC1)ON1C(=O)CCC1=O] |
| 40 | 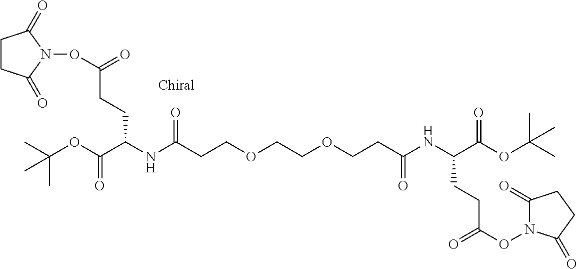 [SMILES: CC(C)(C)OC(=O)[C@H](CCC(=O)ON1C(=O)CCC1=O)NC(=O)CCOCCOCCC(=O)N[C@@H](CCC(=O)ON1C(=O)CCC1=O)C(=O)OC(C)(C)C] | 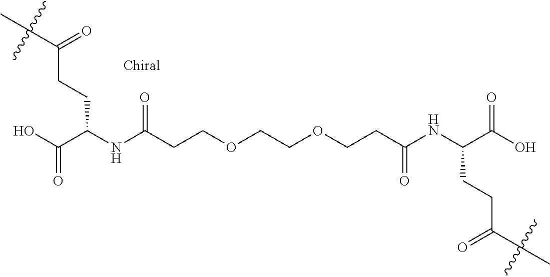 [SMILES: *C(=O)CC[C@H](NC(=O)CCOCCOCCC(=O)N[C@@H](CCC(*)=O)C(=O)O)C(=O)O] |
| 41 | 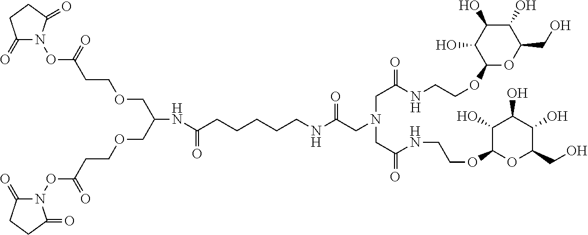 [SMILES: O=C(CN(CC(=O)NCCO[C@@H]1O[C@H](CO)[C@@H](O)[C@H](O)[C@H]1O)CC(=O)NCCO[C@@H]1O[C@H](CO)[C@@H](O)[C@H](O)[C@H]1O)NCCCCCC(=O)NC(COCCC(=O)ON1C(=O)CCC1=O)COCCC(=O)ON1C(=O)CCC1=O] | 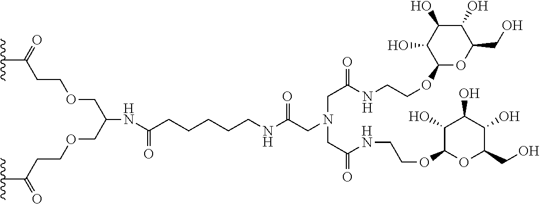 [SMILES: *C(=O)CCOCC(COCCC(*)=O)NC(=O)CCCCCNC(=O)CN(CC(=O)NCCO[C@@H]1O[C@H](CO)[C@@H](O)[C@H](O)[C@H]1O)CC(=O)NCCO[C@@H]1O[C@H](CO)[C@@H](O)[C@H](O)[C@H]1O] |
| 42 | 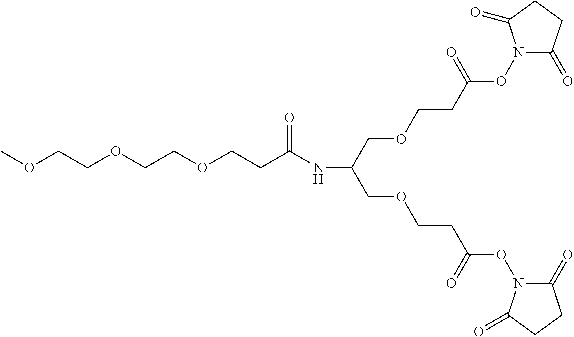 [SMILES: COCCOCCOCCC(=O)NC(COCCC(=O)ON1C(=O)CCC1=O)COCCC(=O)ON1C(=O)CCC1=O] | 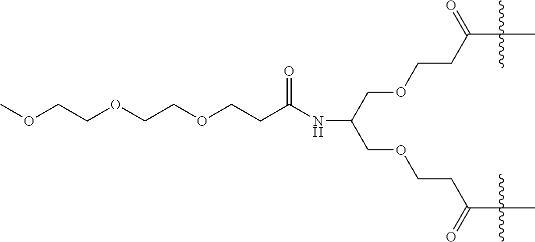 [SMILES: *C(=O)CCOCC(COCCC(*)=O)NC(=O)CCOCCOCCOC] |
| 43 | 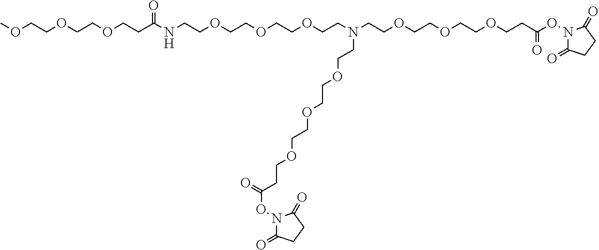 [SMILES: COCCOCCOCCC(=O)NCCOCCOCCOCCN(CCOCCOCCOCCC(=O)ON1C(=O)CCC1=O)CCOCCOCCOCCC(=O)ON1C(=O)CCC1=O] | 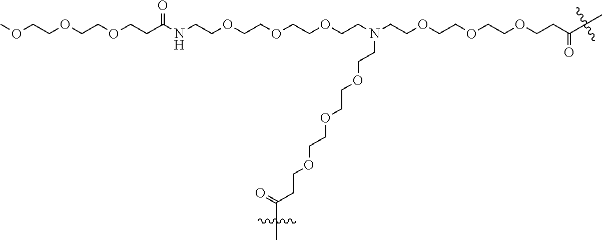 [SMILES: *C(=O)CCOCCOCCOCCN(CCOCCOCCOCCNC(=O)CCOCCOCCOC)CCOCCOCCOCCC(*)=O] |
| 44 | 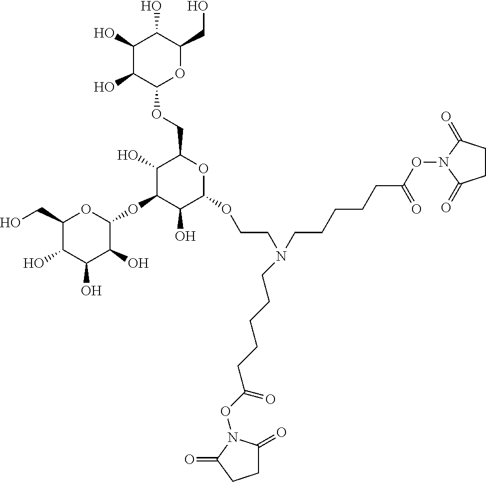 [SMILES: O=C(CCCCCN(CCCCCC(=O)ON1C(=O)CCC1=O)CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)ON1C(=O)CCC1=O] | 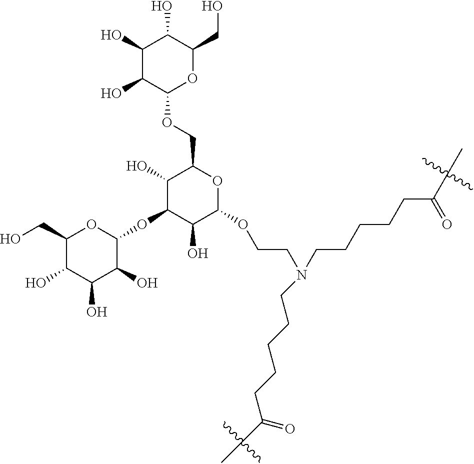 [SMILES: *C(=O)CCCCCN(CCCCCC(*)=O)CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O] |

Linking moieties prepared by click reaction starting from A1-azide and A1′-alkyne substituted insulins:

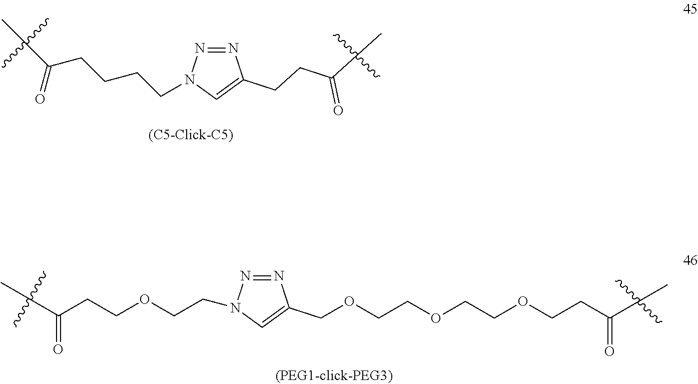 [SMILES: *C(=O)CCCCn1cc(CCC(*)=O)nn1 ; *C(=O)CCOCCOCCOCc1cn(CCOCCC(*)=O)nn1]

In one embodiment, the linking moiety comprises a PEG linker, a short linear polymer of about 2-25 ethylene glycol units or 2, 3, 4, 5, 6, 7, 8, 9, 10, 11, 12, 13, 14, 15, 16, 24, or 25 ethylene glycol units and optionally one or more amino acids. In particular aspects of the insulin receptor partial agonists, the PEG linker comprises the structure (PEG)₂, (PEG)₃, (PEG)₄, (PEG)₅, (PEG)₆, (PEG)₇, (PEG)₈, (PEG)₉, (PEG)₁₀, (PEG)₁₁, (PEG)₁₂, (PEG)₁₃, (PEG)₁₄, (PEG)₁₅, (PEG)₁₆, (PEG)₁₇, (PEG)₂₄, or (PEG)₂₅. The PEG linker may be a bifunctional linker that may be covalently conjugated or linked to alpha amino group of the position A1 residues of the first and second insulin polypeptides. The structure of a bifunctional PEG linker conjugated to the alpha amino group at position A1 residue of the first and second insulin polypeptides may be represented by the following general formula

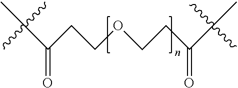

wherein n=1, 2, 3, 4, 5, 6, 7, 8, 9, 10, 11, 12, 13, 14, 15, 16, 17, 24 or 25 and the wavy line indicates the bond between the linker and the alpha amino group. Methods for conjugating PEG to the epsilon amino group of lysine are well known in the art, see for example, Veronese, Biomaterials 22: 405-417 (2001).

In particular aspects of the insulin receptor partial agonists, PEG linking moiety conjugating the alpha amino group of the A1 residue of the first insulin polypeptide to the alpha amino acid at position A1 of the second insulin polypeptide is

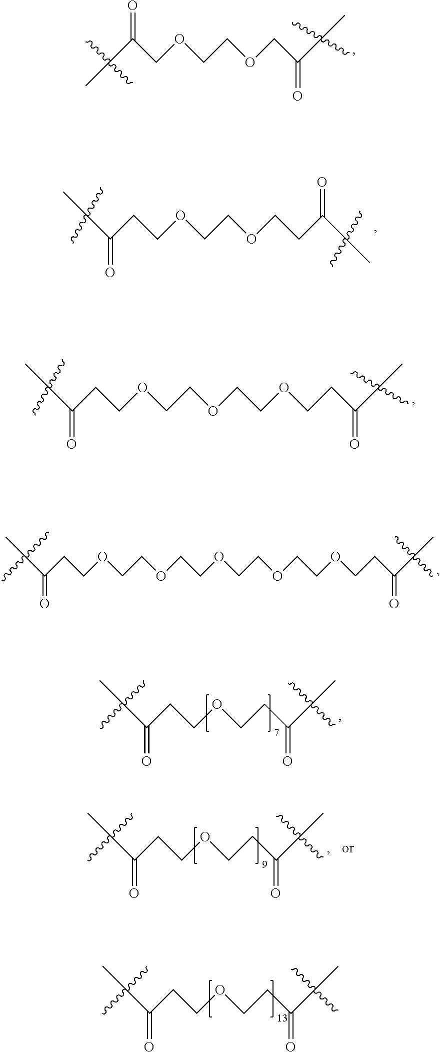

wherein the wavy lines indicate the bond between the linker and the alpha amino group of the at position A1 and A1′ of the insulin polypeptides.

In another embodiment, the linking moiety comprises an acyl moiety comprising 4, 5, 6, 7, 8, 9, 10, 11, 12, 13, 14 15, or 16 carbons. In particular aspects of the insulin receptor partial agonists, the acyl moiety is a succinyl (4), adipoyl (C6), suberyol (C8), or hexadecanedioyl (C16) moiety. The acyl moiety may comprise a bifunctional linker that may be covalently conjugated or linked to alpha amino group of the position A1 residues of the first and second insulin polypeptides. The structure of a bifunctional acyl linker conjugated to the alpha amino group of the at position A1 residue of the first and second insulin polypeptides may be represented by the following general formula

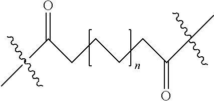

wherein n=0, 1, 2, 3, 4, 5, 6, 7, 8, 9, 10, 11, or 12 and the wavy lines indicate the bond between the linker and the alpha amino group at position A1 of the insulin polypeptides.

In particular aspects of the insulin receptor partial agonists, acyl linking moiety conjugating the alpha amino group at position A1 of the first insulin polypeptide to the alpha amino acid at position A1 of the second insulin polypeptide is

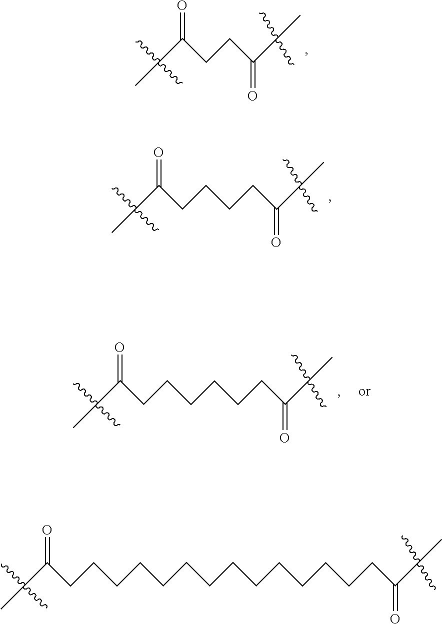 [SMILES: *C(=O)CCC(*)=O ; *C(=O)CCCCC(*)=O ; *C(=O)CCCCCCC(*)=O ; *C(=O)CCCCCCCCCCCCCCC(*)=O]

wherein the wavy lines indicate the bond between the linker and the alpha amino group of the lysine at position A1 of the insulin polypeptides.

Conjugation of a bifunctional linker to the alpha amino group at position A1 of the A-chain polypeptide of two insulin or insulin analog molecules to form the insulin dimer linked by a linking moiety may be schematically shown as:

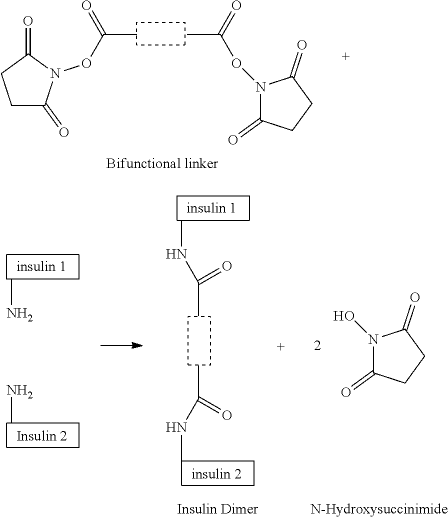

wherein the insulin 1 and insulin 2 molecules may be the same or different and the bifunctional linker and resulting linking moiety following conjugation may have the structure of any linker and resulting linking moiety disclosed herein.

Modification of insulin polypeptides In some embodiments, at least one of the B-chain polypeptides of the insulin receptor partial agonist is modified to comprise an acyl group. The acyl group can be covalently linked directly to an amino acid of the insulin polypeptide, or indirectly to an amino acid of the insulin polypeptide via a spacer, wherein the spacer is positioned between the amino acid of the insulin polypeptide and the acyl group. For example, acylation may occur at any position including any amino acid of the B-chain polypeptides as well as a position within the linking moiety, provided that the activity exhibited by the non-acylated insulin polypeptide is retained upon acylation. Non-limiting examples include acylation at position B1 of the B chain.

In one specific aspect of the invention, the first and/or second insulin polypeptide (or derivative or conjugate thereof) is modified to comprise an acyl group by direct acylation of an amine, hydroxyl, or thiol of a side chain of an amino acid of the insulin polypeptide. In some embodiments, the first and/or second insulin polypeptide is directly acylated through the side chain amine, hydroxyl, or thiol of an amino acid. In this regard, an insulin polypeptide may be provided that has been modified by one or more amino acid substitutions in the B-chain polypeptide sequence, including for example at positions B1, B10, or B22 or at any position of the linking moiety with an amino acid comprising a side chain amine, hydroxyl, or thiol. An example of a spacer in illustrated in Scheme X below:

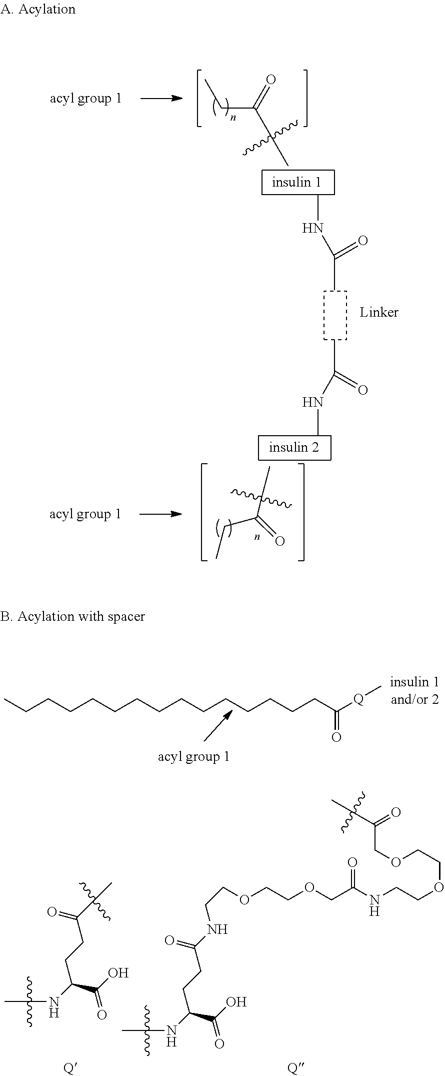

wherein in A. the wavy line illustrates point of attachment to side chain of insulin peptide and in B. the wavy line at the amino group of the spacer illustrates point of attachment to the acyl group 1 and the wavy line at the carbonyl group of the spacer illustrates point of attachment to the insulin peptide, Q is a spacer represented, for example, as Q′ and Q″ and n is a C₁₅ alkyl.chain.

In some embodiments, the spacer between the first and/or second insulin polypeptide and the acyl group is an amino acid comprising a side chain amine, hydroxyl, or thiol (or a dipeptide or tripeptide comprising an amino acid comprising a side chain amine, hydroxyl, or thiol). In some embodiments, the spacer comprises a hydrophilic bifunctional spacer. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, KY). In one embodiment, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate.

In some embodiments, the spacer between the first and/or second insulin polypeptide and the acyl group is a hydrophobic bifunctional spacer. Hydrophobic bifunctional spacers are known in the art. See, e.g., Bioconjugate Techniques, G. T. Hermanson (Academic Press, San Diego, C A, 1996), which is incorporated by reference in its entirety. In certain embodiments, the hydrophobic bifunctional spacer comprises two or more reactive groups, e.g., an amine, a hydroxyl, a thiol, and a carboxyl group or any combinations thereof. In certain embodiments, the hydrophobic bifunctional spacer comprises a hydroxyl group and a carboxylate. In other embodiments, the hydrophobic bifunctional spacer comprises an amine group and a carboxylate. In other embodiments, the hydrophobic bifunctional spacer comprises a thiol group and a carboxylate. Suitable hydrophobic bifunctional spacers comprising a carboxylate and a hydroxyl group or a thiol group are known in the art and include, for example, 8-hydroxyoctanoic acid and 8-mercaptooctanoic acid.

In accordance with certain embodiments the bifunctional spacer can be a synthetic or naturally occurring amino acid comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. Each amino acid of the dipeptide or tripeptide spacer attached to the insulin polypeptide can be independently selected from the group consisting of: naturally-occurring and/or non-naturally occurring amino acids, including, for example, any of the D or L isomers of the naturally-occurring amino acids (Ala, Cys, Asp, Glu, Phe, Gly, His, Ile, Lys, Leu, Met, Asn, Pro, Arg, Ser, Thr, Val, Trp, Tyr), or any D or L isomers of the non-naturally occurring amino acids selected from the group consisting of: β-alanine (β-Ala), N-α-methyl-alanine (Me-Ala), aminobutyric acid (Abu), α-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (MeIle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O2)), norleucine (Nle), methyl-norleucine (Me-Nle), norvaline (Nva), omithine (Om), para-aminobenzoic acid (PABA), penicillamine (Pen), methylphenylalanine (MePhe), 4-Chlorophenylalanine (Phe(4-Cl)), 4-fluorophenylalanine (Phe(4-F)), 4-nitrophenylalanine (Phe(4-NO2)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), U-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4-tetrahydro-isoquinoline-3-carboxylic acid (Tic), tetrahydropyranglycine, thienylalanine (Thi), U-Benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), 1-amino-1-cyclohexane carboxylic acid (Acx), aminovaleric acid, beta-cyclopropyl-alanine (Cpa), propargylglycine (Prg), allylglycine (Alg), 2-amino-2-cyclohexyl-propanoic acid (2-Cha), tertbutylglycine (Tbg), vinylglycine (Vg), 1-amino-1-cyclopropane carboxylic acid (Acp), 1-amino-1-cyclopentane carboxylic acid (Acpe), alkylated 3-mercaptopropionic acid, 1-amino-1-cyclobutane carboxylic acid (Acb). In some embodiments the dipeptide spacer is selected from the group consisting of. Ala-Ala, β-Ala, β-Ala, Leu-Leu, Pro-Pro, γ-aminobutyric acid-γ-aminobutyric acid, and γ-Glu-γ-Glu.

The first and/or second insulin polypeptide may be modified to comprise an acyl group by acylation of a long chain alkane. In specific aspects, the long chain alkane comprises an amine, hydroxyl, or thiol group (e.g. octadecylamine, tetradecanol, and hexadecanethiol) which reacts with a carboxyl group, or activated form thereof, of the insulin polypeptide. The carboxyl group, or activated form thereof, of the insulin polypeptide can be part of a side chain of an amino acid (e.g., glutamic acid, aspartic acid) of the insulin polypeptide or can be part of the peptide backbone.

In certain embodiments, the first and/or second insulin polypeptide is modified to comprise an acyl group by acylation of the long chain alkane by a spacer which is attached to the insulin polypeptide. In specific aspects, the long chain alkane comprises an amine, hydroxyl, or thiol group which reacts with a carboxyl group, or activated form thereof, of the spacer. Suitable spacers comprising a carboxyl group, or activated form thereof, are described herein and include, for example, bifunctional spacers, e.g., amino acids, dipeptides, tripeptides, hydrophilic bifunctional spacers and hydrophobic bifunctional spacers. As used herein, the term “activated form of a carboxyl group” refers to a carboxyl group with the general formula R(C═O)X, wherein X is a leaving group and R is the insulin polypeptide or the spacer. For example, activated forms of a carboxyl groups may include, but are not limited to, acyl chlorides, anhydrides, and esters. In some embodiments, the activated carboxyl group is an ester with an N-hydroxysuccinimide (NHS) leaving group.

With regard to these aspects of the invention, in which a long chain alkane is acylated by the peptide, the insulin polypeptide or the spacer, the long chain alkane may be of any size and can comprise any length of carbon chain. The long chain alkane can be linear or branched. In certain aspects, the long chain alkane is a C₄ to C₃₀ alkane. For example, the long chain alkane can be any of a C₄ alkane, C₆ alkane, C₈ alkane, C₁₀ alkane, C₁₂ alkane, C₁₄ alkane, C₁₆ alkane, C₁₈ alkane, C₂₀ alkane, C₂₂ alkane, C₂₄ alkane, C₂₆ alkane, C₂₈ alkane, or a C₃₀ alkane. In some embodiments, the long chain alkane comprises a C₈ to C₂₀ alkane, e.g., a C₁₄ alkane, C₁₆ alkane, or a C₁₈ alkane.

In some embodiments, an amine, hydroxyl, or thiol group of the first and/or second insulin polypeptide is acylated with a cholesterol acid. In a specific embodiment, the peptide is linked to the cholesterol acid through an alkylated des-amino Cys spacer, i.e., an alkylated 3-mercaptopropionic acid spacer. Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmacuetical Res. “Synthesis of Palmitoyl Derivatives of Insulin and their Biological Activity” Vol. 6, No: 2 pp. 171-176 (1989).

The acyl group of the acylated peptide the first and/or second insulin polypeptide can be of any size, e.g., any length carbon chain, and can be linear or branched. In some specific embodiments of the invention, the acyl group is a C₄ to C₃₀ fatty acid. For example, the acyl group can be any of a C₄ fatty acid, C₆ fatty acid, C₈ fatty acid, C₁₀ fatty acid, C₁₂ fatty acid, C₁₄ fatty acid, C₁₆ fatty acid, C₁₈ fatty acid, C₂₀ fatty acid, C₂₂ fatty acid, C₂₄ fatty acid, C₂₆ fatty acid, C₂₈ fatty acid, or a C₃₀ fatty acid. In some embodiments, the acyl group is a C₈ to C₂₀ fatty acid, e.g., a C₁₄ fatty acid or a C₁₆ fatty acid. In some embodiments, the acyl group is carbamoyl.

In an alternative embodiment, the acyl group is a bile acid. The bile acid can be any suitable bile acid, including, but not limited to, cholic acid, chenodeoxycholic acid, deoxycholic acid, lithocholic acid, taurocholic acid, glycocholic acid, and cholesterol acid.

The acylated first and/or second insulin polypeptide described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. In some embodiments the acylated single chain analog comprises an amino acid selected from the group consisting of a Cys, Lys, Orn, homo-Cys, or Ac-Phe, and the side chain of the amino acid is covalently bonded to a hydrophilic moiety (e.g., PEG). In one embodiment, the acyl group is attached to position B1, B2, B10, or B22 (according to the amino acid numbering of the A and B chains of native insulin), optionally via a spacer comprising Cys, Lys, Orn, homo-Cys, or Ac-Phe.

Alternatively, the acylated first and/or second insulin polypeptide comprises a spacer, wherein the spacer is both acylated and modified to comprise the hydrophilic moiety. Non-limiting examples of suitable spacers include a spacer comprising one or more amino acids selected from the group consisting of Cys, Lys, Orn, homo-Cys, and Ac-Phe.

In some embodiments, at least one of the ε-amines of B29 or B28 lysine and/or N-terminal amino acids B1 of B-chain is modified to comprise a capping group. In another embodiment, the capping group is at ε-amines of B29 or B28 lysine of the B-chain polypeptides of the insulin receptor partial agonist. In another embodiment, the capping group is at N-terminal amino acids B1 of the B-chain polypeptides of the insulin receptor partial agonist. The capping group may be covalently linked directly to the amino group of the N-terminal amino acid or indirectly to the amino group via a spacer, wherein the spacer is positioned between the amino group of the N-terminal amino acid of the insulin polypeptide and the capping group. The capping group may be an acyl moiety as discussed supra. The substituent may have the general formula RC(O)—, where R can be peptide, PEG, linear or branched alkyl chain, said peptide, PEG and alkyl optionally substituted with fluoro, amino-, phosphono-, hydroxy-, carboxylic acid, amino acid, PEG, and saccharides, or R can be R′NH, or R′O, wherein R′ can be H (when R is R′NH), peptide, PEG, linear or branched alkyl chain, said peptide, PEG and alkyl optionally substituted with fluoro, amino-, phosphono-, hydroxy-, or carboxylic acid, amino acid, PEG, and saccharides. Examples of some RC(O) capping groups are realized where some —CH₂-groups may be replaced with —O— groups, or nitrogen atoms of the said amino- and amido-groups can be iteratively alkylated with R groups as defined above, or acylated with RC(O)—.

In aspects of this invention the capping group is N-dimethyl, or RC(O) that may be exemplified as acetyl, phenylacetyl, isobutyl, methoxyacetyl, 2-(carboxymethoxy)acetyl, 2-[bis(carboxymethylamino)]acetyl, glutaryl, trifluoroacetyl, glycyl, aminoethylglucose (AEG), AEG-C6, PEG (e.g., PEG1, PEG2, PEG3, PEG4, PEG5, PEG8, PEG24), and alkoxycarbonyl.

In a particular aspect of the insulin dimer is realized when the capping group is selected from Capping Group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31, or a mixture thereof from Table II. A subembodiment of this aspect of the invention is realized when the capping group is selected from 6, 7, 9, 10, 11, 12, 15, 19, 20, and 21-28, or a mixture thereof from Table II (see Table II for structures of the capping group). Carbamolyation of insulin has been disclosed by Oimoni et al., Nephron 46: 63-66 (1987) and insulin dimers comprising a carbamoyl groups at the N-terminus has been disclosed in disclosed in published PCT Application No. WO2014052451 (E.g., MIU-90).

Exemplary capping groups conjugated to the N-terminal amino group are illustrated in Table II

| Capping | | |
|---|---|---|
| Group | | |
| No. | Capping Reagent | Capping Group |
| 1 | 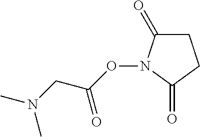 | 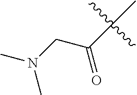 [SMILES: *C(=O)CN(C)C] |
| 2 | 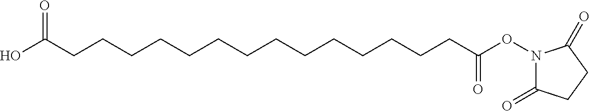 [SMILES: O=C(O)CCCCCCCCCCCCCCC(=O)ON1C(=O)CCC1=O] | 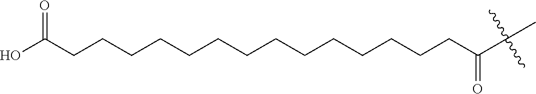 [SMILES: *C(=O)CCCCCCCCCCCCCCC(=O)O] |
| 3 | 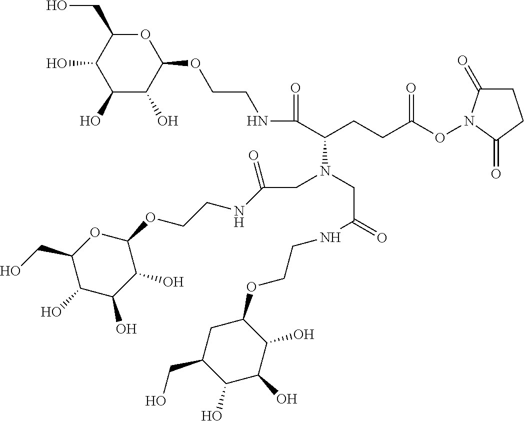 [SMILES: O=C(CN(CC(=O)NCCO[C@@H]1C[C@H](CO)[C@@H](O)[C@H](O)[C@H]1O)[C@@H](CCC(=O)ON1C(=O)CCC1=O)C(=O)NCCO[C@@H]1O[C@H](CO)[C@@H](O)[C@H](O)[C@H]1O)NCCO[C@@H]1O[C@H](CO)[C@@H](O)[C@H](O)[C@H]1O] | 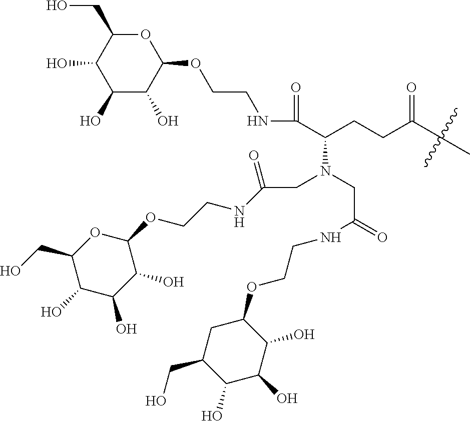 [SMILES: *C(=O)CC[C@@H](C(=O)NCCO[C@@H]1O[C@H](CO)[C@@H](O)[C@H](O)[C@H]1O)N(CC(=O)NCCO[C@@H]1O[C@H](CO)[C@@H](O)[C@H](O)[C@H]1O)CC(=O)NCCO[C@@H]1C[C@H](CO)[C@@H](O)[C@H](O)[C@H]1O] |
| 4 | 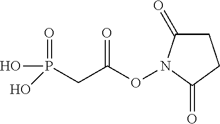 [SMILES: O=C(CP(=O)(O)O)ON1C(=O)CCC1=O] | 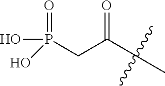 [SMILES: *C(=O)CP(=O)(O)O] |
| 5 | 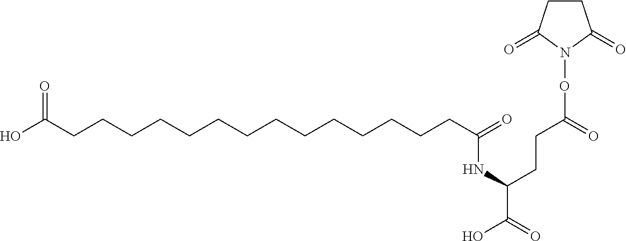 [SMILES: O=C(O)CCCCCCCCCCCCCCC(=O)N[C@@H](CCC(=O)ON1C(=O)CCC1=O)C(=O)O] | 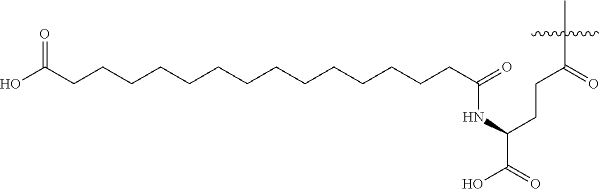 [SMILES: *C(=O)CC[C@H](NC(=O)CCCCCCCCCCCCCCC(=O)O)C(=O)O] |
| 6 | 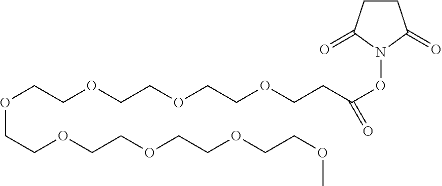 [SMILES: COCCOCCOCCOCCOCCOCCOCCOCCC(=O)ON1C(=O)CCC1=O] | 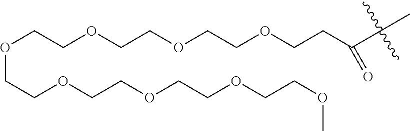 [SMILES: *C(=O)CCOCCOCCOCCOCCOCCOCCOCCOC] |
| 7 | 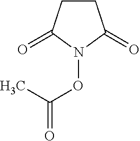 | 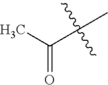 [SMILES: *C(C)=O] |
| 8 | 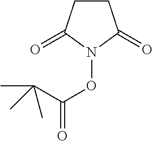 | 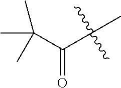 [SMILES: *C(=O)C(C)(C)C] |
| 9 | 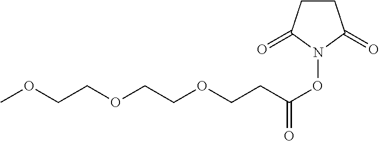 [SMILES: COCCOCCOCCC(=O)ON1C(=O)CCC1=O] | 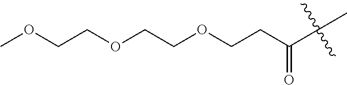 [SMILES: *C(=O)CCOCCOCCOC] |
| 10 | 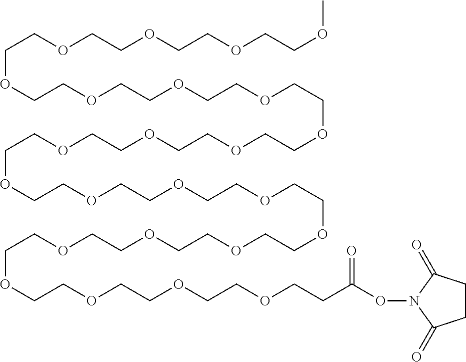 [SMILES: COCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O] | 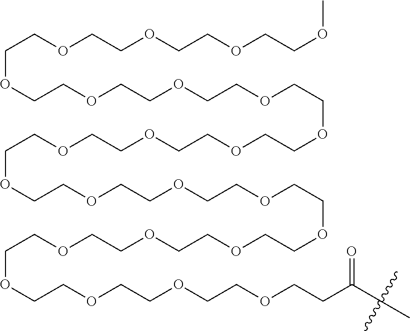 [SMILES: *C(=O)CCOCCOCCOCCOCCOCCOCCOCCOCCOCCOCCOCCOCCOCCOCCOCCOCCOCCOCCOCCOCCOCCOCCOCCOC] |
| 11 | 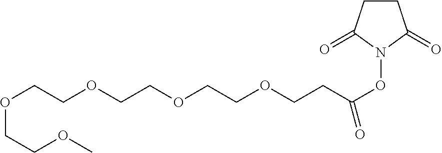 [SMILES: COCCOCCOCCOCCOCCC(=O)ON1C(=O)CCC1=O] | 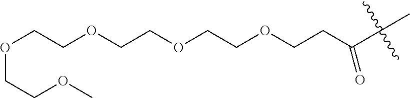 [SMILES: *C(=O)CCOCCOCCOCCOCCOC] |
| 12 | 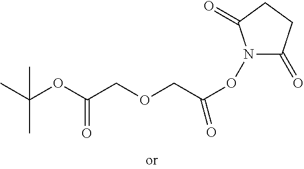 [SMILES: CC(C)(C)OC(=O)COCC(=O)ON1C(=O)CCC1=O] | 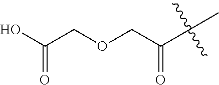 [SMILES: *C(=O)COCC(=O)O] |
| | 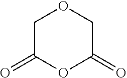 | |
| 13 | 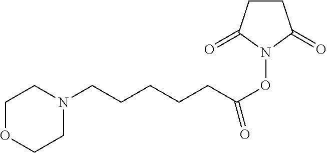 [SMILES: O=C(CCCCCN1CCOCC1)ON1C(=O)CCC1=O] | 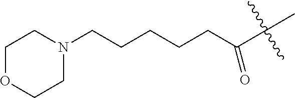 [SMILES: *C(=O)CCCCCN1CCOCC1] |
| 14 | 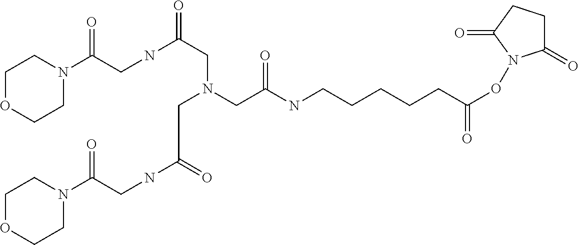 [SMILES: O=C(CN(CC(=O)NCC(=O)N1CCOCC1)CC(=O)NCC(=O)N1CCOCC1)NCCCCCC(=O)ON1C(=O)CCC1=O] | 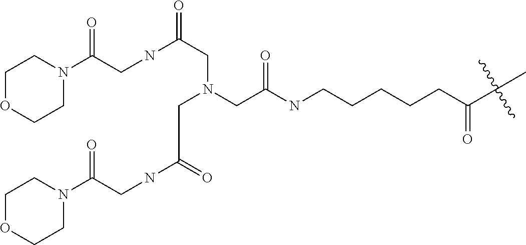 [SMILES: *C(=O)CCCCCNC(=O)CN(CC(=O)NCC(=O)N1CCOCC1)CC(=O)NCC(=O)N1CCOCC1] |
| 15 | 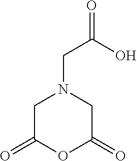 [SMILES: O=C(O)CN1CC(=O)OC(=O)C1] | 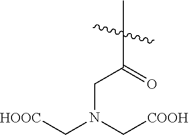 [SMILES: *C(=O)CN(CC(=O)O)CC(=O)O] |
| 16 | 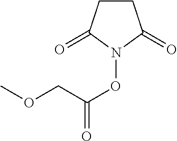 | 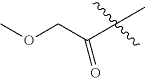 [SMILES: *C(=O)COC] |
| 17 | 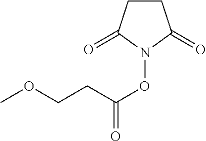 | 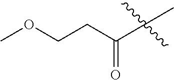 [SMILES: *C(=O)CCOC] |
| 18 | 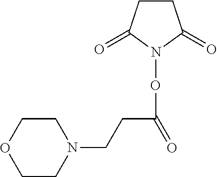 [SMILES: O=C(CCN1CCOCC1)ON1C(=O)CCC1=O] | 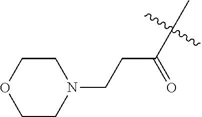 [SMILES: *C(=O)CCN1CCOCC1] |
| 19 | 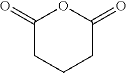 | 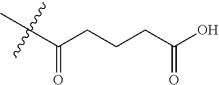 [SMILES: *C(=O)CCCC(=O)O] |
| 20 | 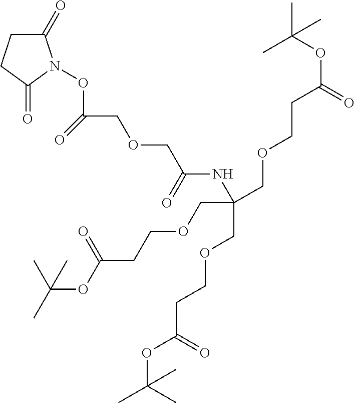 [SMILES: CC(C)(C)OC(=O)CCOCC(COCCC(=O)OC(C)(C)C)(COCCC(=O)OC(C)(C)C)NC(=O)COCC(=O)ON1C(=O)CCC1=O] | 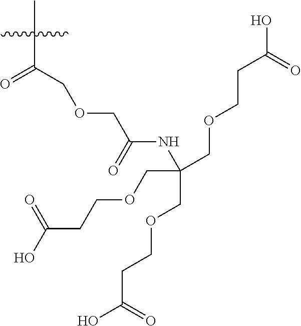 [SMILES: *C(=O)COCC(=O)NC(COCCC(=O)O)(COCCC(=O)O)COCCC(=O)O] |
| 21 | 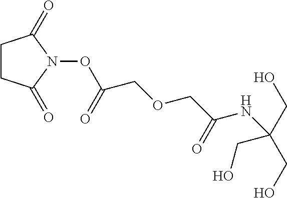 [SMILES: O=C(COCC(=O)ON1C(=O)CCC1=O)NC(CO)(CO)CO] | 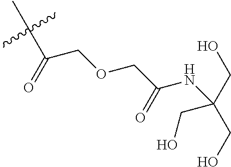 [SMILES: *C(=O)COCC(=O)NC(CO)(CO)CO] |
| 22 | 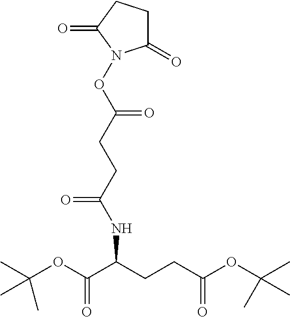 [SMILES: CC(C)(C)OC(=O)CC[C@H](NC(=O)CCC(=O)ON1C(=O)CCC1=O)C(=O)OC(C)(C)C] | 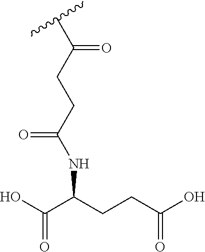 [SMILES: *C(=O)CCC(=O)N[C@@H](CCC(=O)O)C(=O)O] |
| 23 | 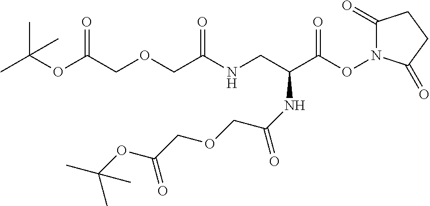 [SMILES: CC(C)(C)OC(=O)COCC(=O)NC[C@H](NC(=O)COCC(=O)OC(C)(C)C)C(=O)ON1C(=O)CCC1=O] | 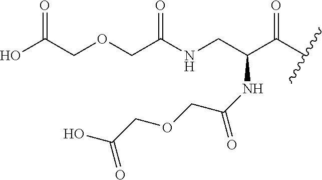 [SMILES: *C(=O)[C@H](CNC(=O)COCC(=O)O)NC(=O)COCC(=O)O] |
| 24 | 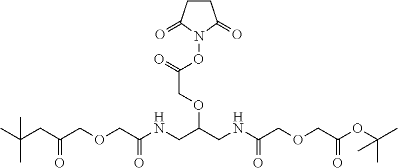 [SMILES: CC(C)(C)CC(=O)COCC(=O)NCC(CNC(=O)COCC(=O)OC(C)(C)C)OCC(=O)ON1C(=O)CCC1=O] | 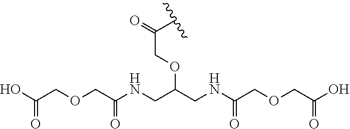 [SMILES: *C(=O)COC(CNC(=O)COCC(=O)O)CNC(=O)COCC(=O)O] |
| 25 | 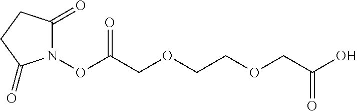 [SMILES: O=C(O)COCCOCC(=O)ON1C(=O)CCC1=O] | 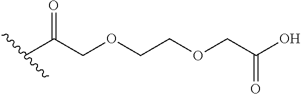 [SMILES: *C(=O)COCCOCC(=O)O] |
| 26 | 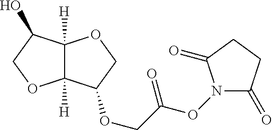 [SMILES: O=C(CO[C@H]1CO[C@H]2[C@@H]1OC[C@H]2O)ON1C(=O)CCC1=O] | 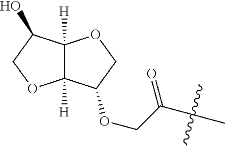 [SMILES: *C(=O)CO[C@H]1CO[C@H]2[C@@H]1OC[C@H]2O] |
| 27 | 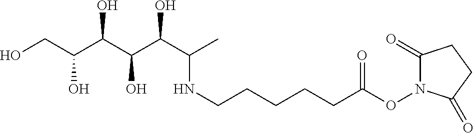 [SMILES: CC(NCCCCCC(=O)ON1C(=O)CCC1=O)[C@H](O)[C@@H](O)[C@H](O)[C@H](O)CO] | 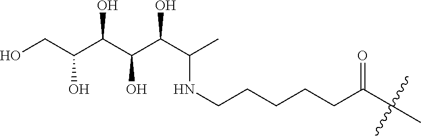 [SMILES: *C(=O)CCCCCNC(C)[C@H](O)[C@@H](O)[C@H](O)[C@H](O)CO] |
| 28 | 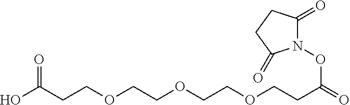 [SMILES: O=C(O)CCOCCOCCOCCC(=O)ON1C(=O)CCC1=O] | 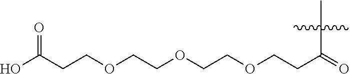 [SMILES: *C(=O)CCOCCOCCOCCC(=O)O] |
| 29 | 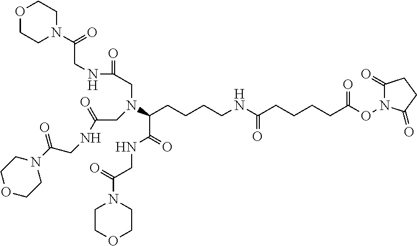 [SMILES: O=C(CCCCC(=O)ON1C(=O)CCC1=O)NCCCC[C@@H](C(=O)NCC(=O)N1CCOCC1)N(CC(=O)NCC(=O)N1CCOCC1)CC(=O)NCC(=O)N1CCOCC1] | 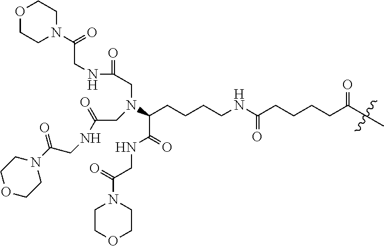 [SMILES: *C(=O)CCCCC(=O)NCCCC[C@@H](C(=O)NCC(=O)N1CCOCC1)N(CC(=O)NCC(=O)N1CCOCC1)CC(=O)NCC(=O)N1CCOCC1] |
| 30 | 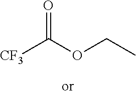 | 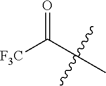 [SMILES: *C(=O)C(F)(F)F] |
| | 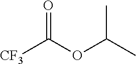 | |
| 31 | 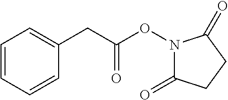 [SMILES: O=C(Cc1ccccc1)ON1C(=O)CCC1=O] | 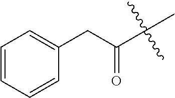 [SMILES: *C(=O)Cc1ccccc1] |

Thus, an embodiment of this invention is realized when the insulin dimer comprises zero to four capping groups independently conjugated to N-terminal amino acid B1 and ε-amine of B-29 lysine (B-28 lysine in the case of LISRO insulin) of the B-chain insulins and independently selected from the group consisting of RC(O)—, where R can be peptide, PEG, linear or branched alkyl chain, said peptide, PEG and alkyl optionally substituted with amino-, phosphono-, hydroxy-, carboxylic acid, amino acid, PEG, and saccharides, or R can be R′NH, or R′O, wherein R′ can be H (when R is R′NH), peptide, PEG, linear or branched alkyl chain, said peptide, PEG and alkyl optionally substituted with fluoro, amino-, phosphono-, hydroxy-, or carboxylic acid, amino acid, PEG, and saccharides. An embodiment of this aspect of the invention is realized when the capping group is N-dimethyl. An embodiment of this aspect of the invention is realized when RC(O) is a capping group selected from acetyl, phenylacetyl, isobutyl, methoxyacetyl, 2-(carboxymethoxy)acetyl, 2-[bis(carboxymethylamino)]acetyl, glutaryl, trifluoroacetyl, glycyl, aminoethylglucose (AEG), AEG-C6, PEG (e.g., PEG1, PEG2, PEG3, PEG4, PEG5, PEG8, PEG24), and alkoxycarbonyl. Another embodiment of this aspect of the insulin dimer is realized when 0-4 capping groups are independently selected from Capping Group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31, or a mixture thereof from Table II. Still another subembodiment of this aspect of the invention is realized when 0-4 capping groups are independently selected from 6, 7, 9, 10, 11, 12, 15, 19, 20, and 21-28, or a mixture thereof from Table II.

Yet another aspect of the invention is realized when capping groups are independently selected from the group consisting of Capping Group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31, or a mixture thereof and the bifunctional linker moiety is selected from the group consisting of Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, Linker 23, Linker 24, Linker 25, Linker 26, Linker 27, Linker 28, Linker 29, Linker 30, Linker 31, Linker 32, Linker 33, Linker 34, Linker 35, Linker 36, Linker 37, Linker 38, Linker 39, Linker 40, Linker 41, Linker 42, Linker 43, Linker 44, Linker 45, and Linker 46. A subembodiment of this aspect of the invention is realized when the capping groups are independently selected from 6, 7, 9, 10, 11, 12, 15, 19, 20, and 21-28, or a mixture thereof.

### Exemplary Insulin Dimers

A complete structural depiction of the compounds of this invention is illustrated with insulin dimer 49 in Formula II below, wherein the the α-amino group of the A1 residue of one A-chain insulin heterodimer is conjugated to the α-amino group of the A1 residue of the other A′-chain insulin heterodimer via a bis-functional PEG5 linker moiety, disulfide linkages between the Cys₆ and Cys₁₁ residues of the A-chain polypeptide and disulfide linkages between the Cys₇ and Cys₂₀ of the A-chain to the Cys₇ and Cys₁₉ of the B-chain polypeptide, respectively exists; the linking moieties are covalently linked to the alpha amino acid of the A1 residue, wherein the A-chain and A′-chain polypeptides for Dimers 1-132 and 134-1 (Table III) has the amino acid sequence shown in SEQ ID NO:1, Dimer 133 has the amino acid sequence shown in SEQ ID NO:10; the B-chain and B′-chain polypeptide Dimers 1-144 (Table III) has the amino acid sequence shown in SEQ ID NO:2; and where 0-4 capping groups are independently conjugated to N-terminal amino acid B1 and □□-amine of B-29 lysine (B-28 lysine in the case of LISRO insulin) of the B-chain insulins It should be noted that the linking moiety and capping groups in insulin dimer 49 independently may differ from other insulin dimers of the present invention as shown in Table III.

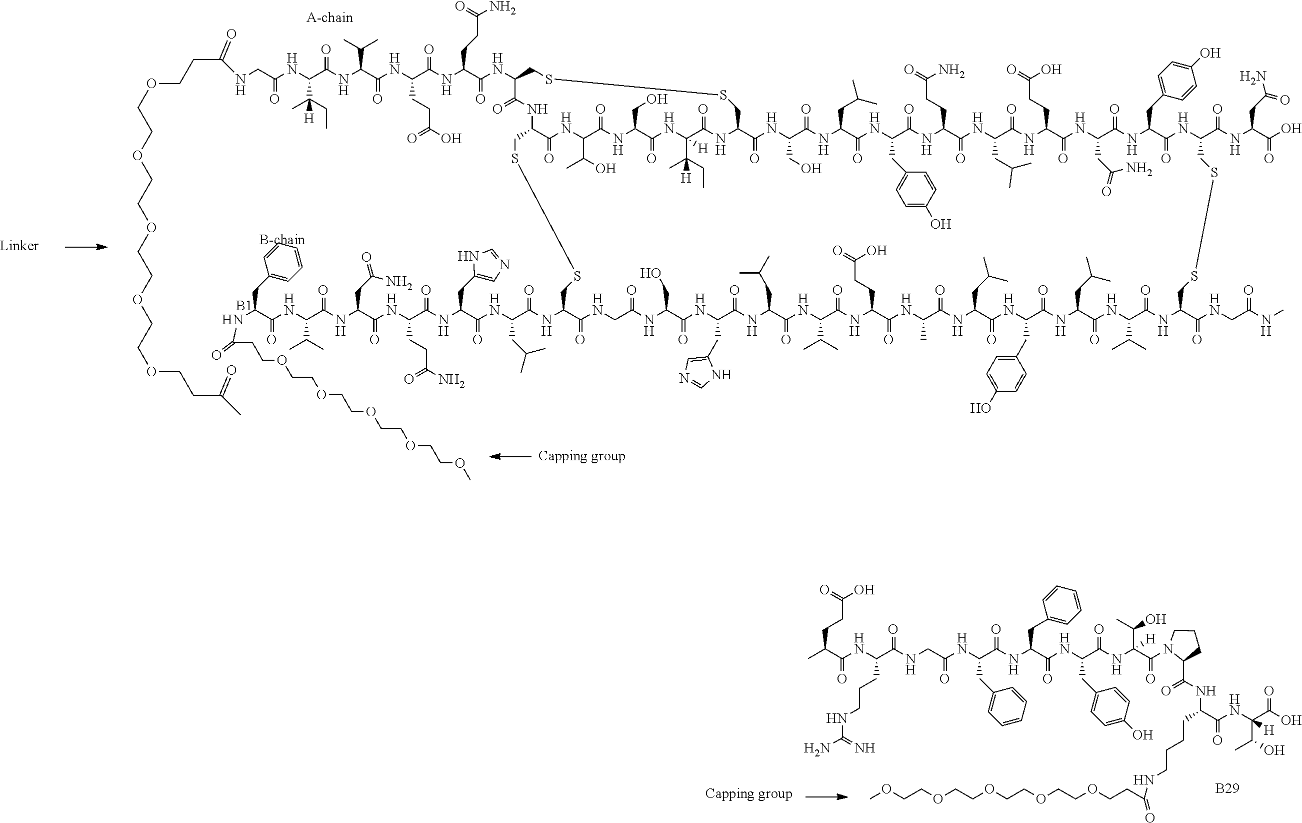

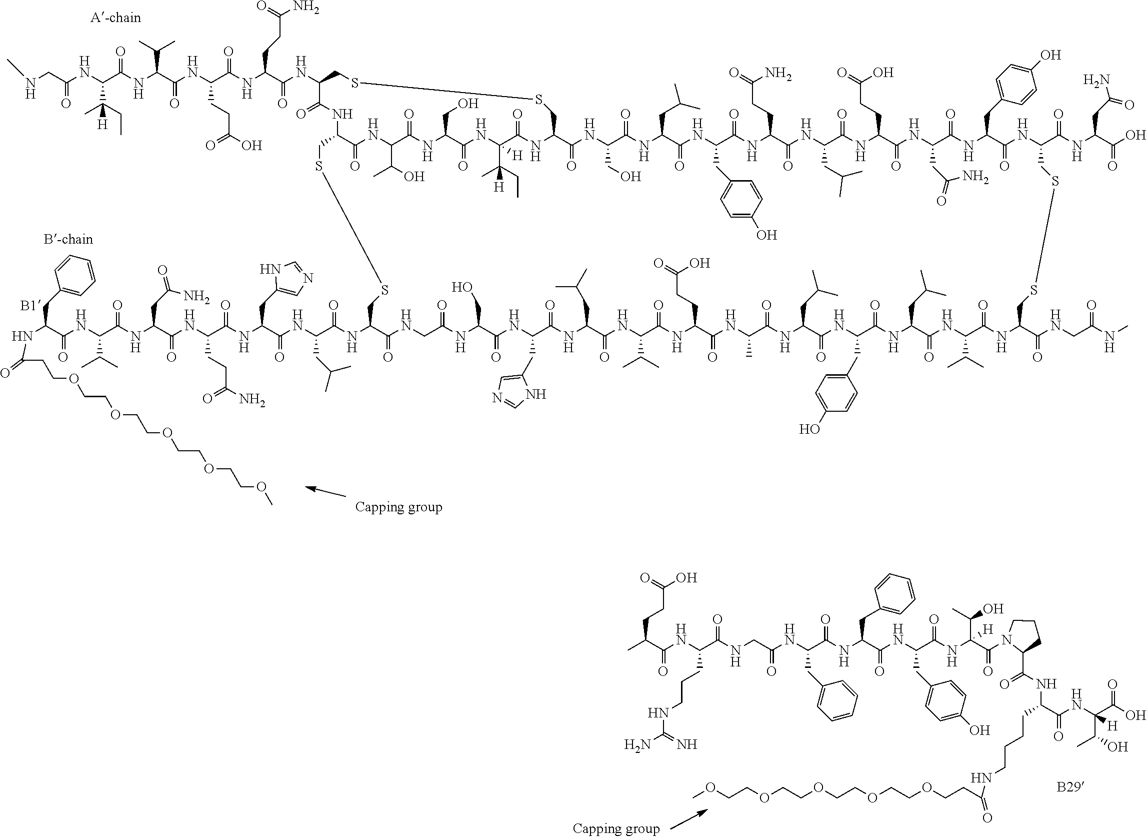

Exemplary insulin dimers include those in Table III:

### Table III

| Dimer | Structural Representation |
|---|---|
| 1 | 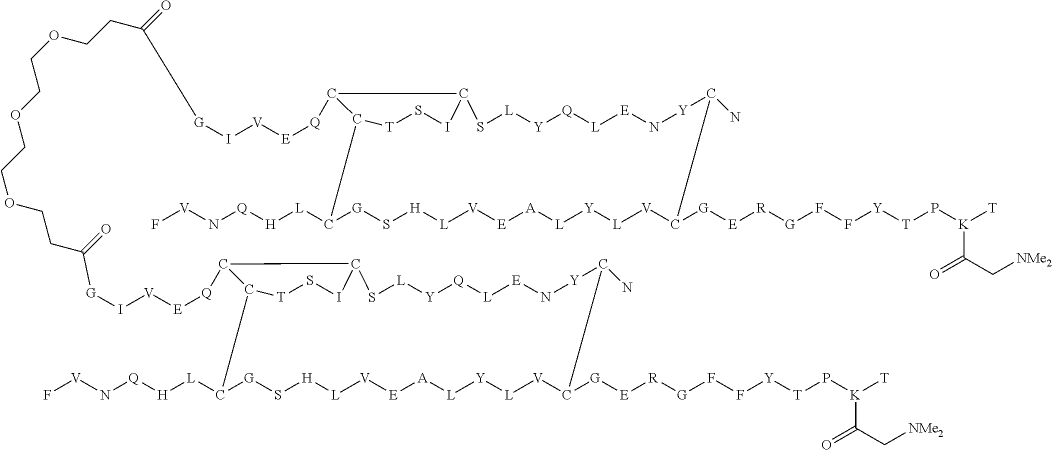 |
| 2 | 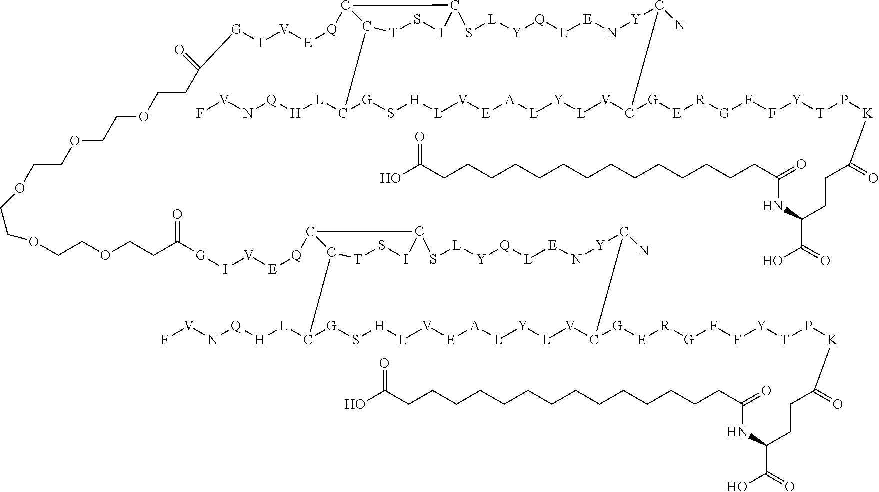 |
| 3 | 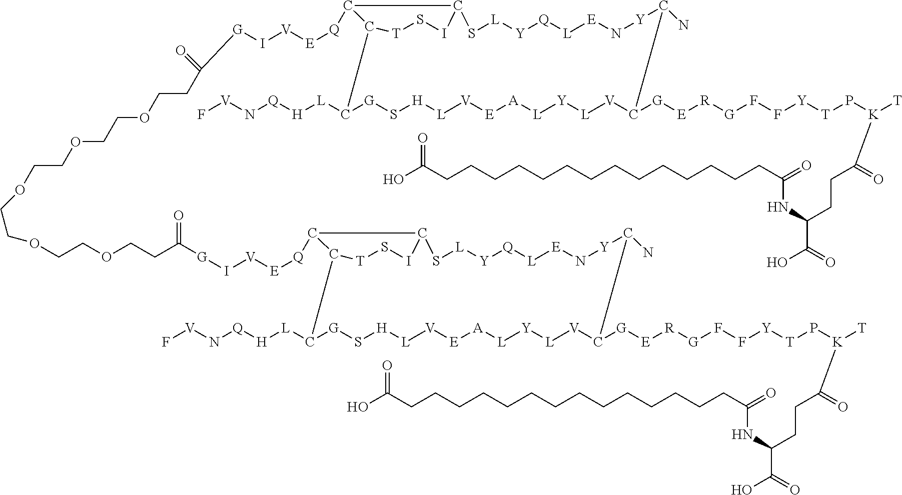 |
| 4 | 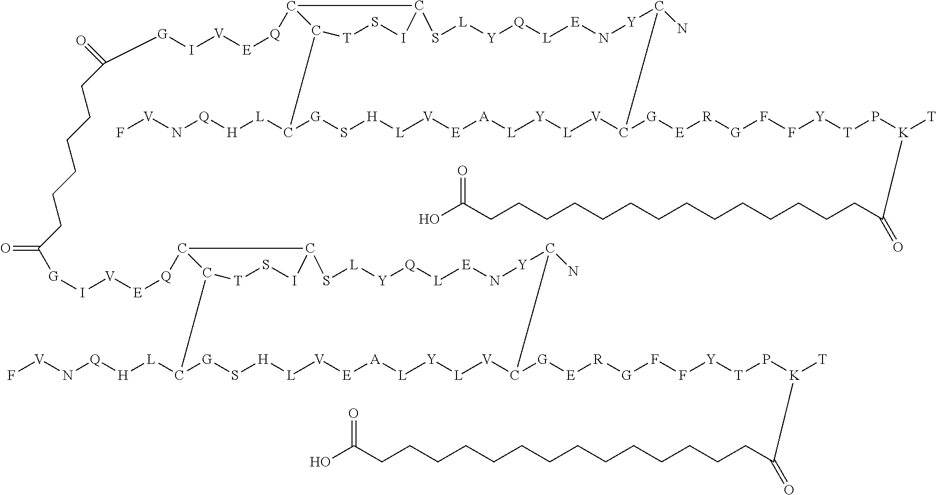 |
| 5 | 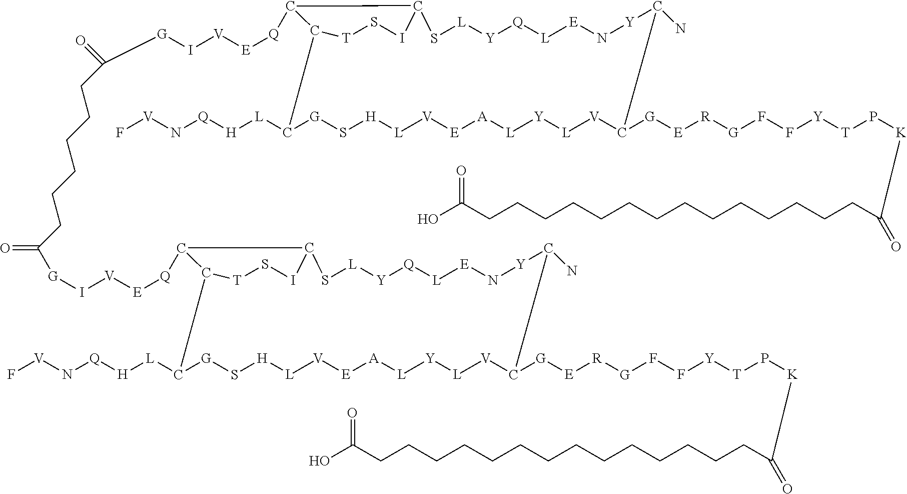 |
| 6 | 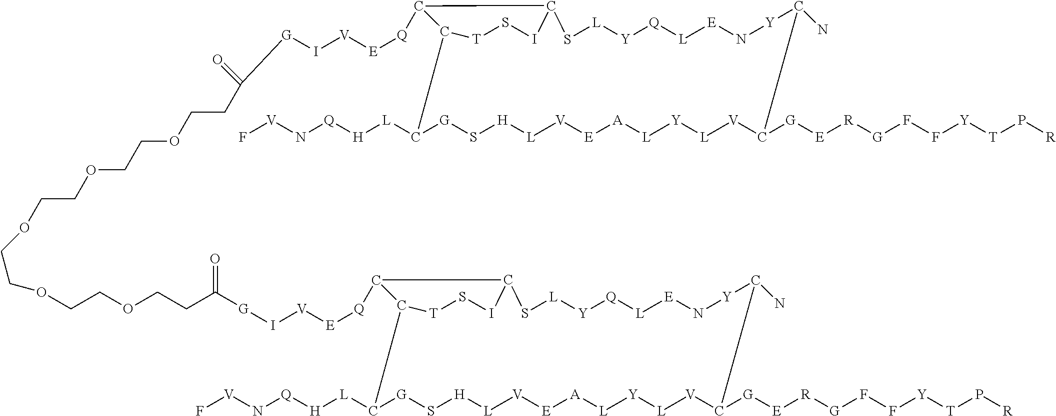 |
| 7 | 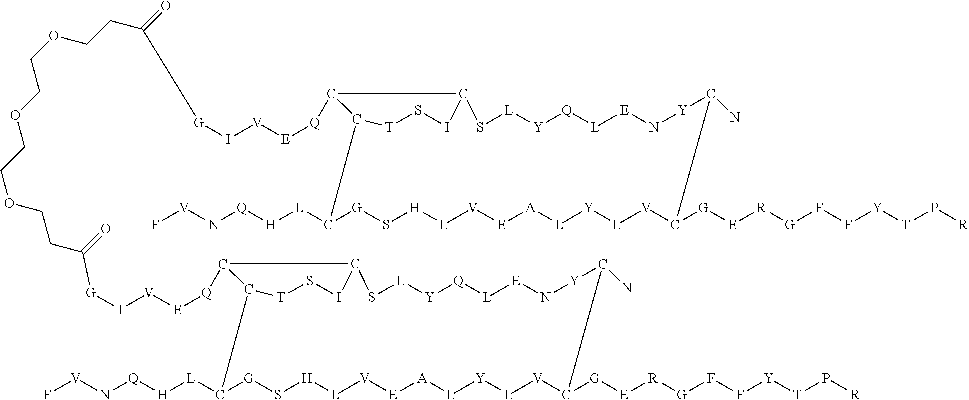 |
| 8 | 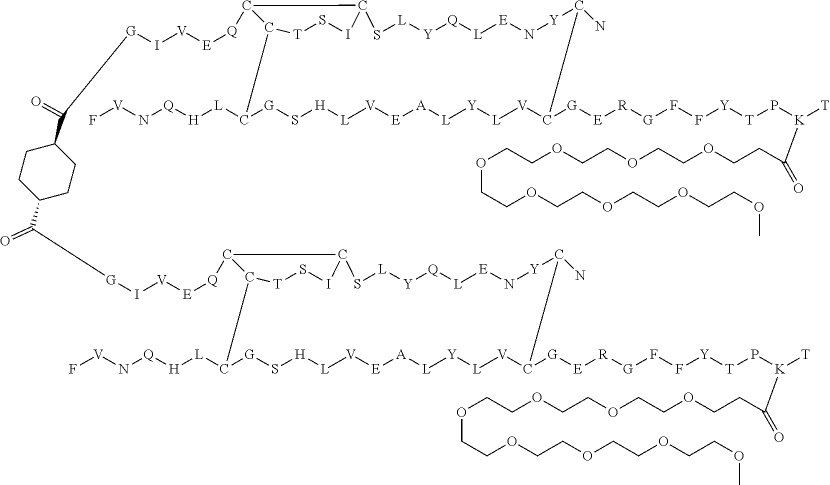 |
| 9 | 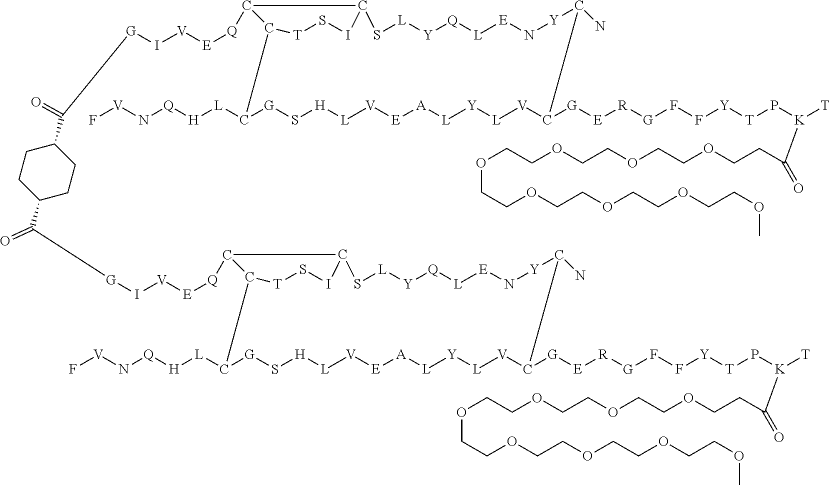 |
| 10 | 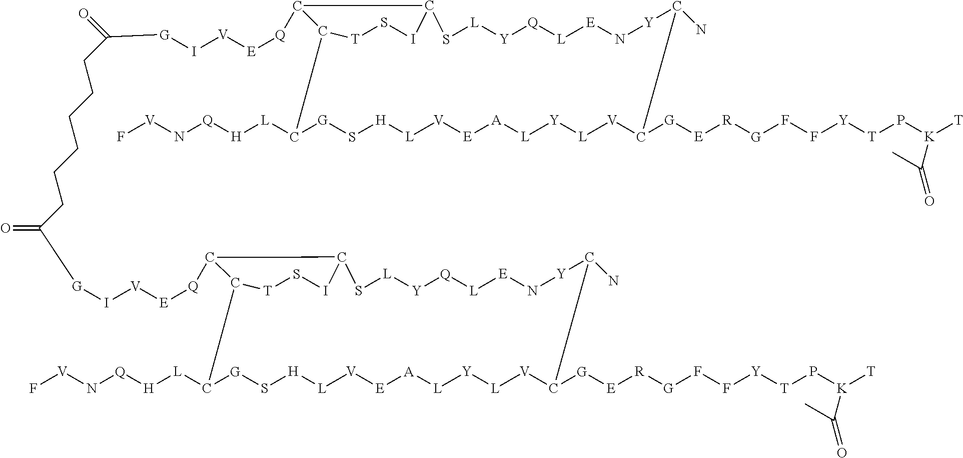 |
| 11 | 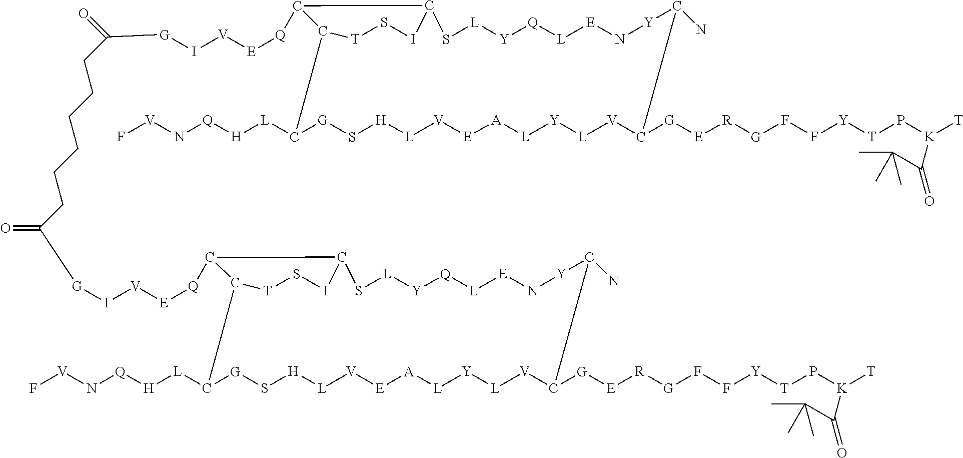 |
| 12 | 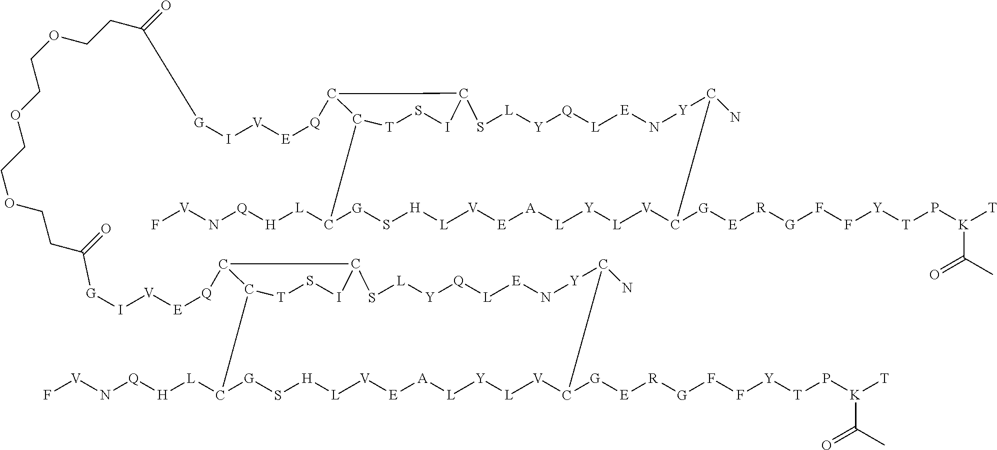 |
| 13 | 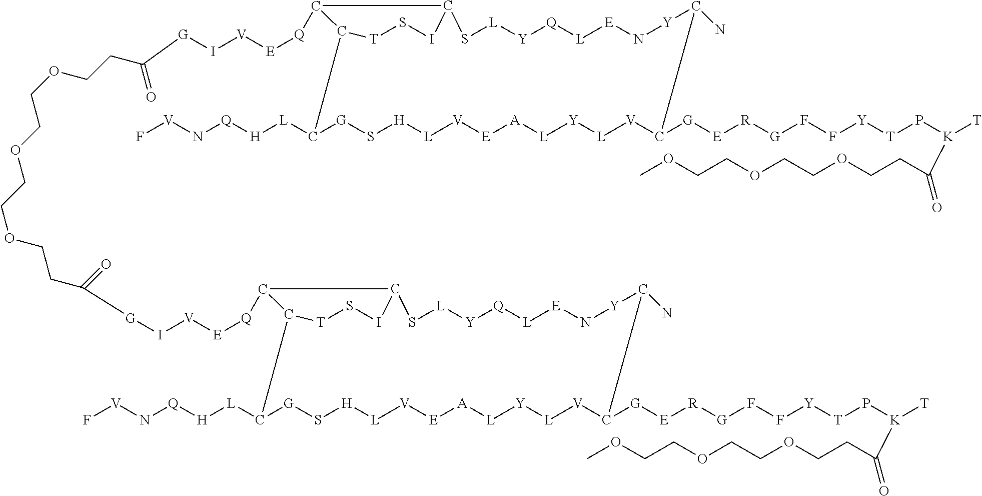 |
| 14 | 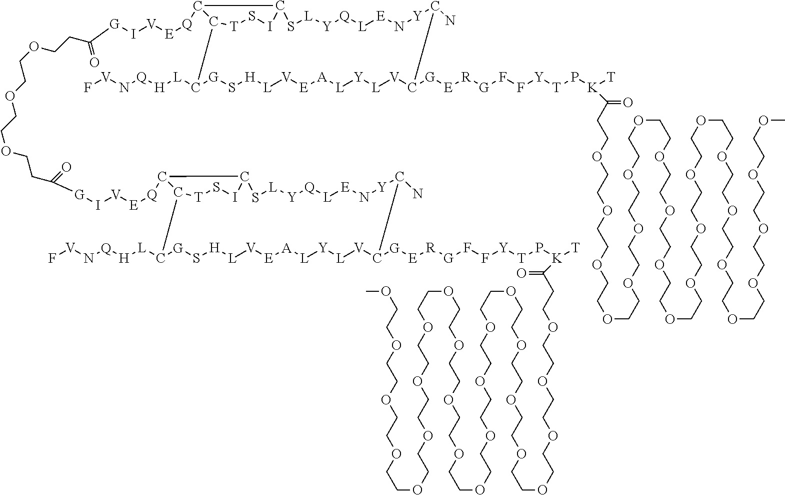 |
| 15 | 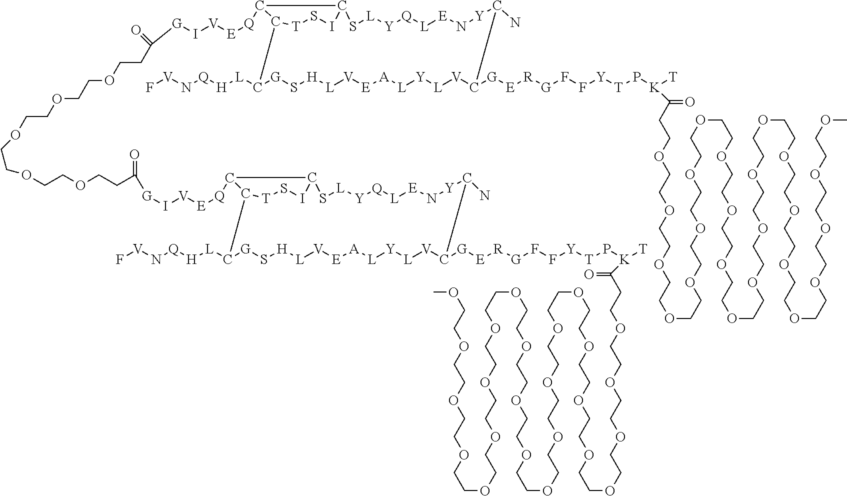 |
| 16 | 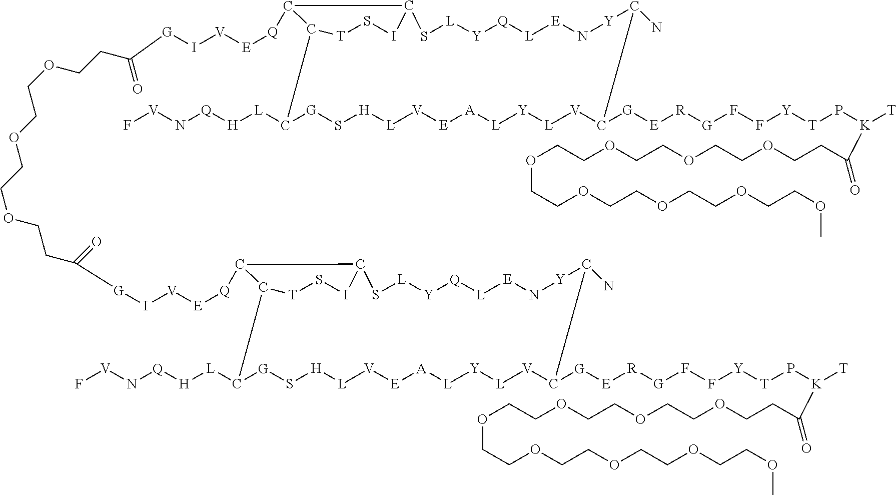 |
| 17 | 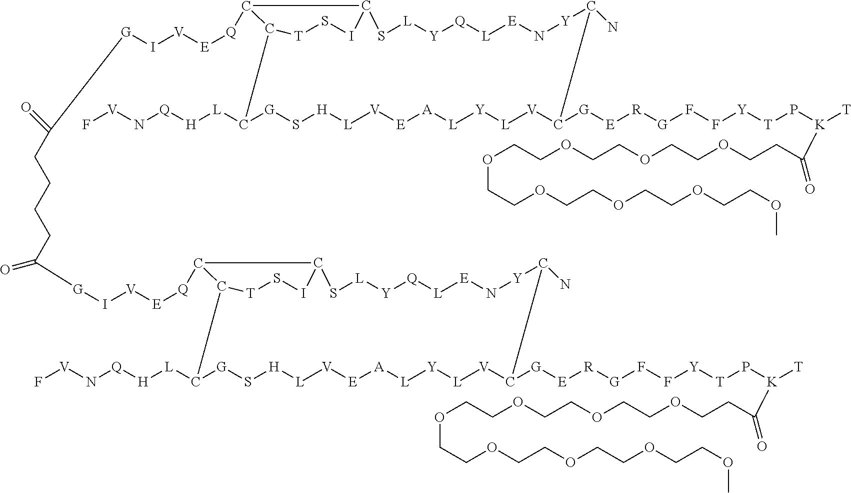 |
| 18 | 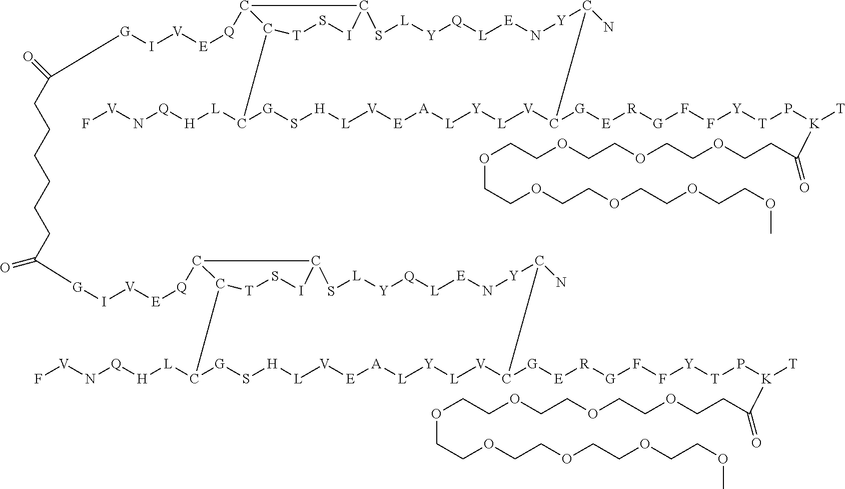 |
| 19 | 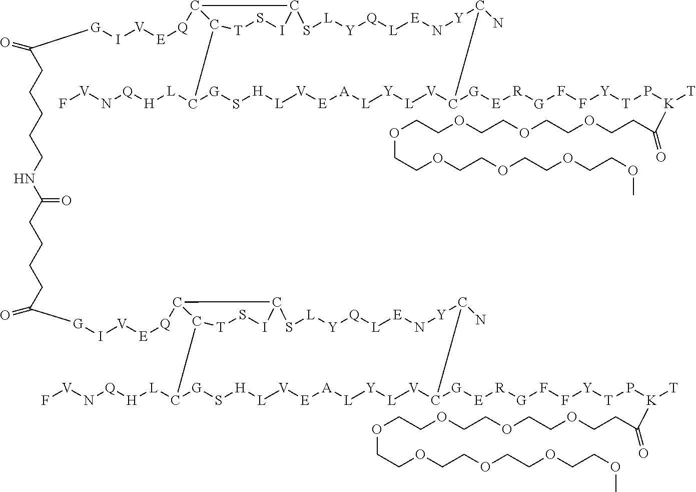 |
| 20 | 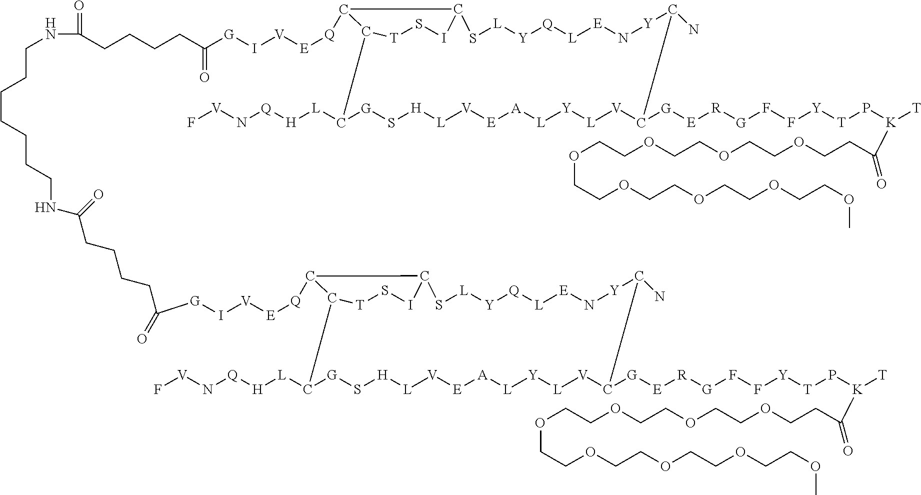 |
| 21 | 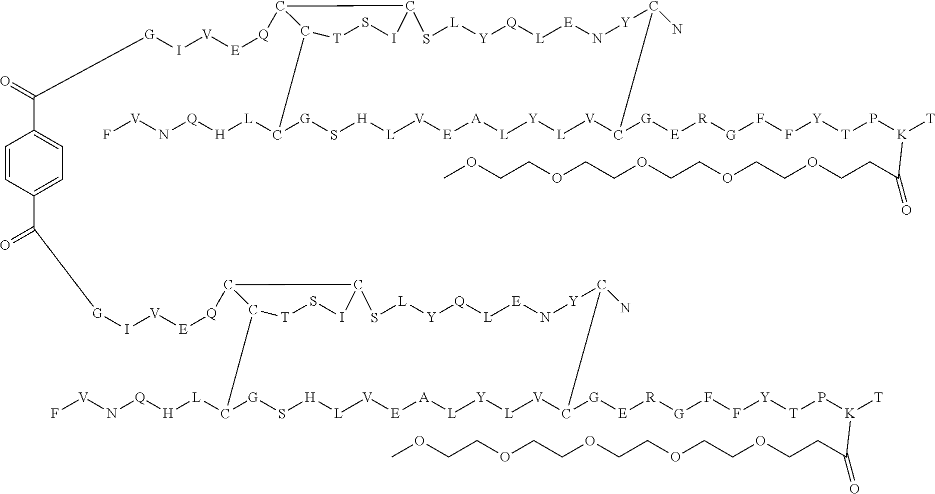 |
| 22 | 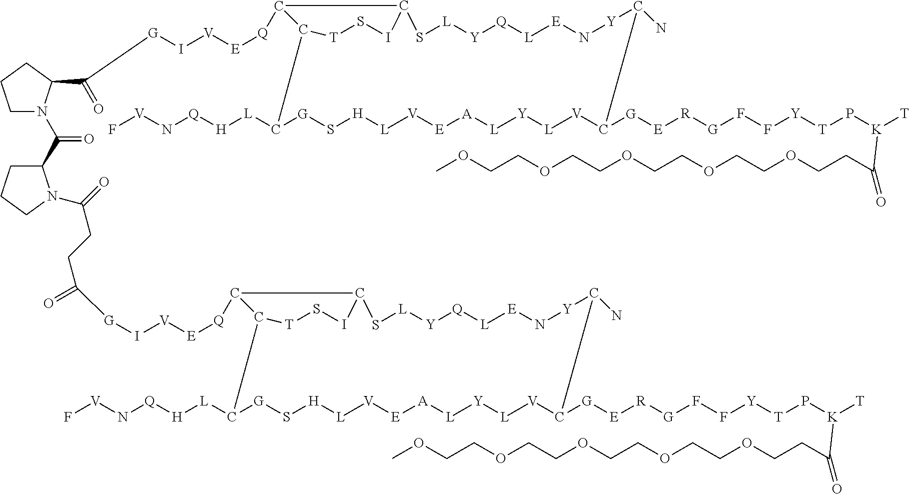 |
| 23 | 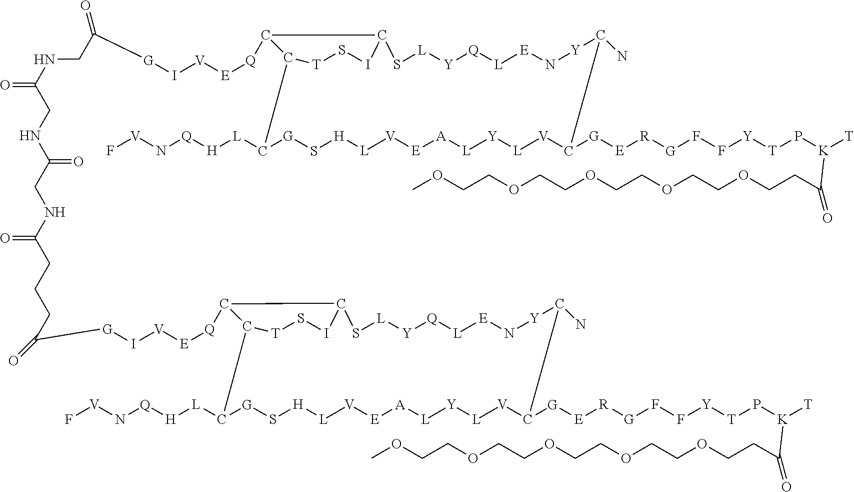 |
| 24 | 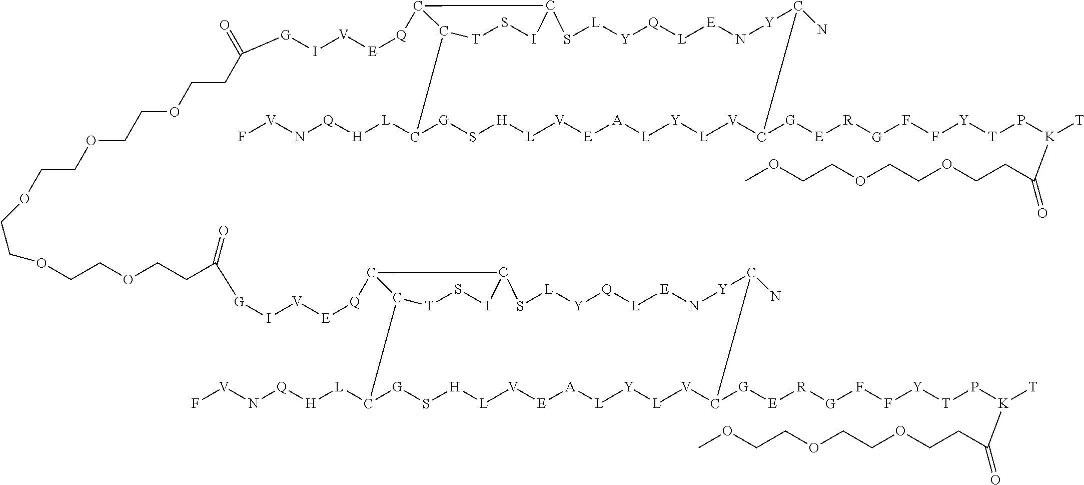 |
| 25 | 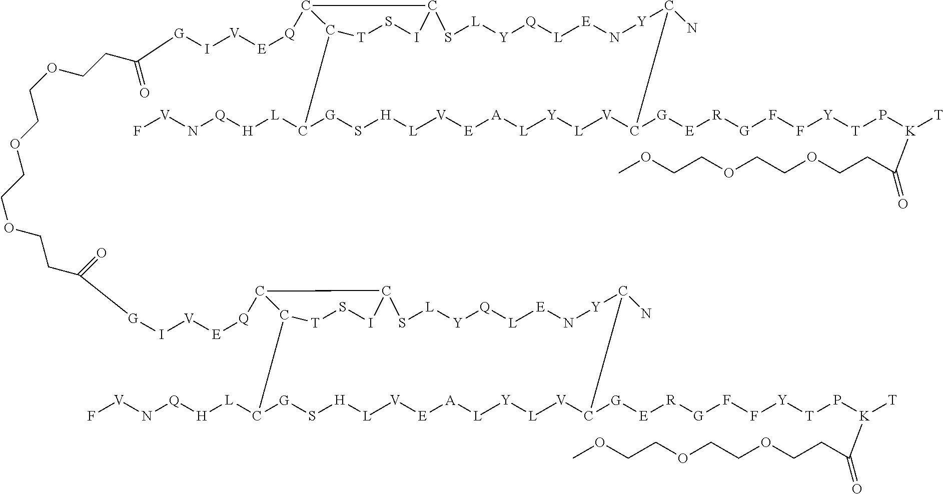 |
| 26 | 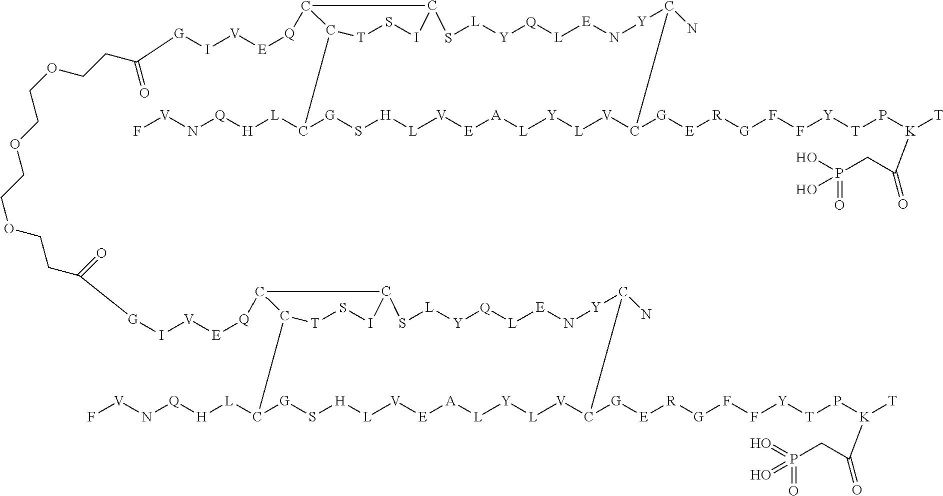 |
| 27 | 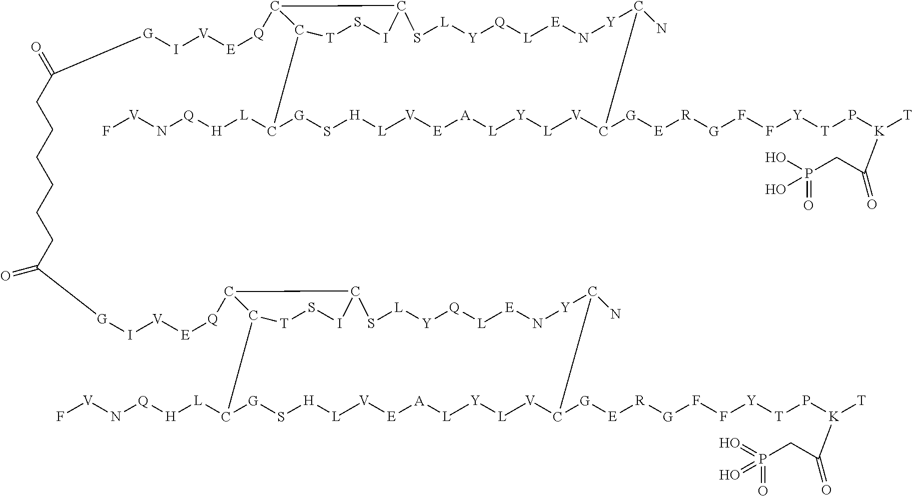 |
| 28 | 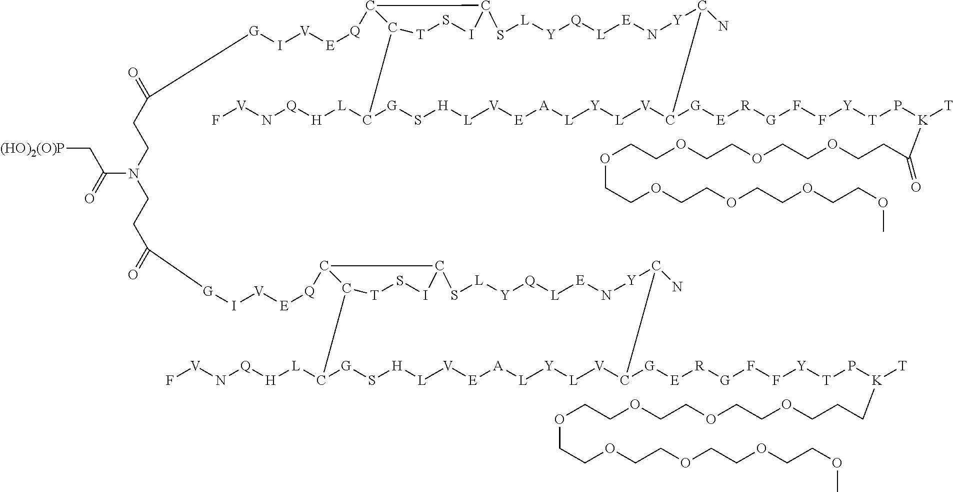 |
| 29 | 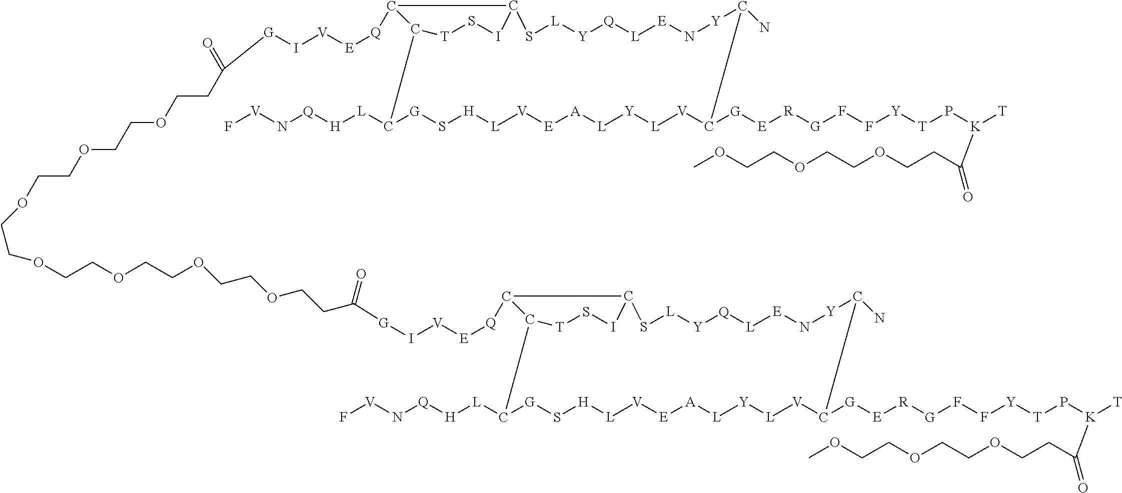 |
| 30 | 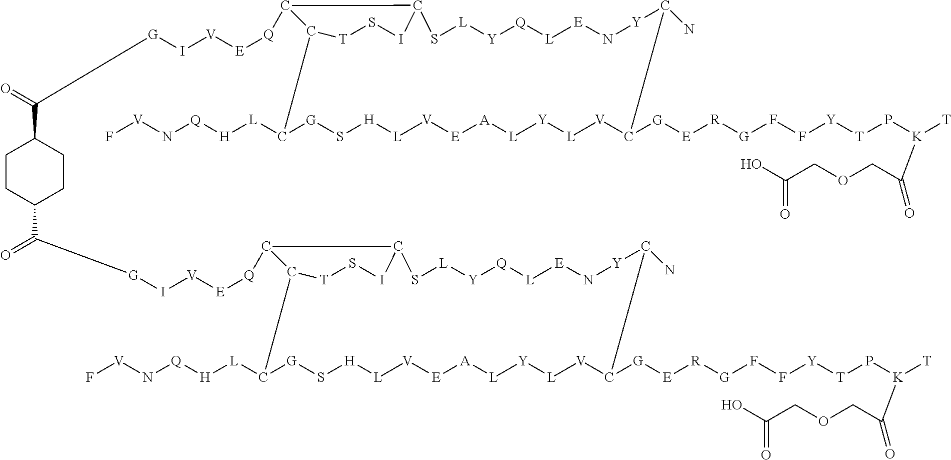 |
| 31 | 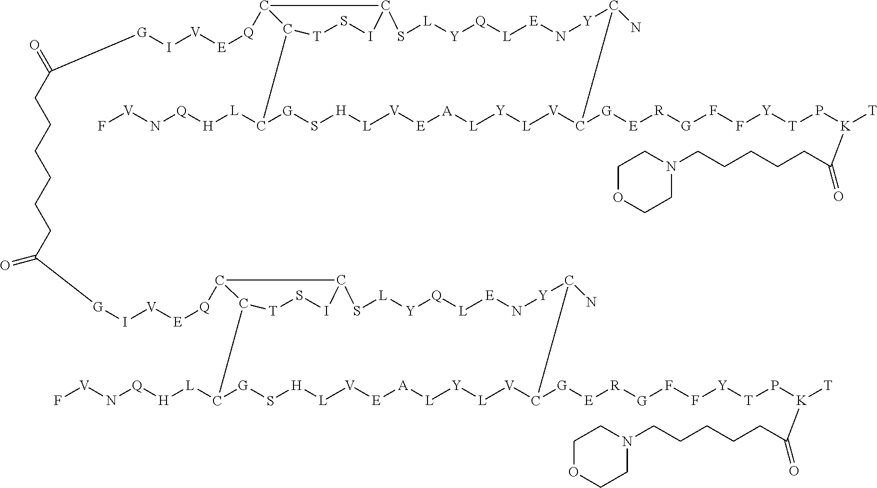 |
| 32 | 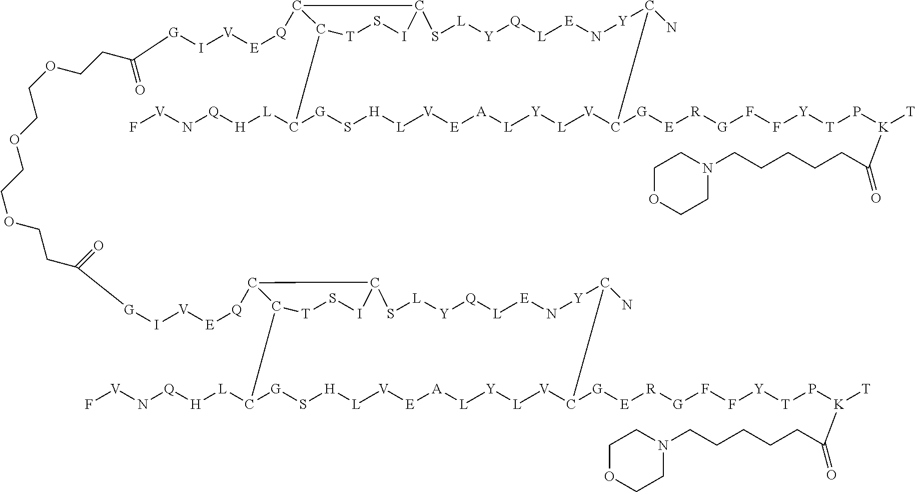 |
| 33 | 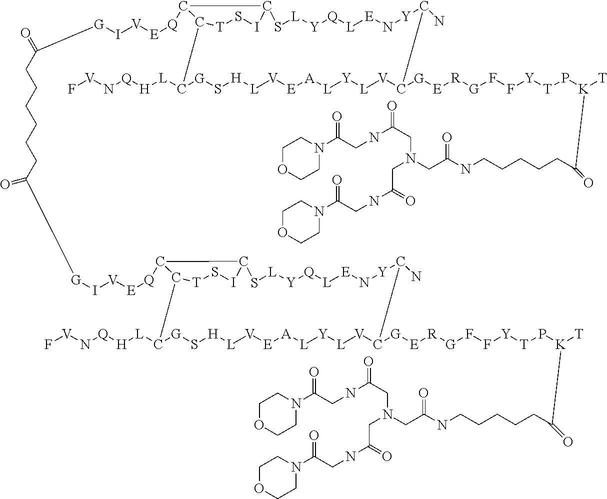 |
| 34 | 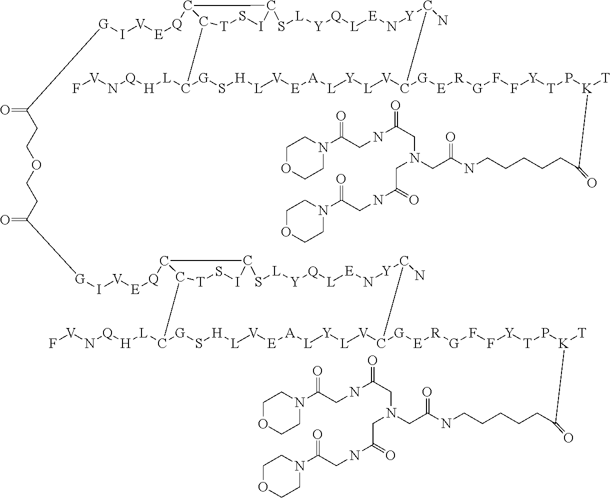 |
| 35 | 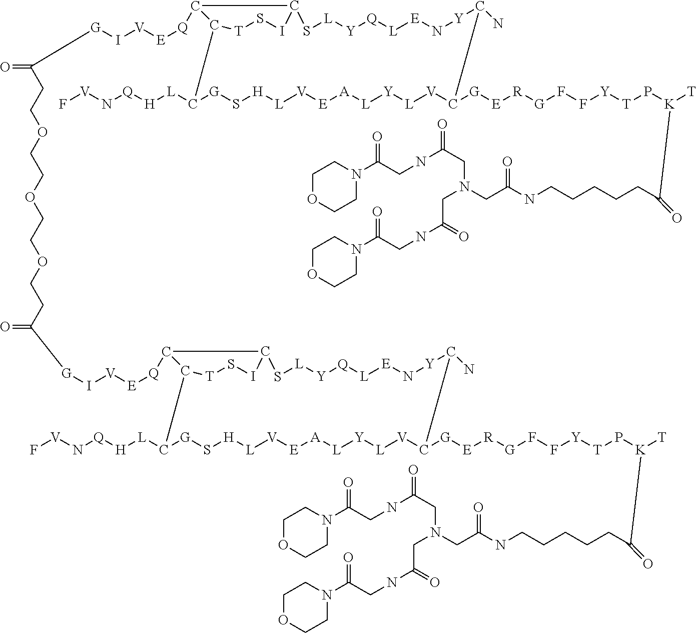 |
| 36 | 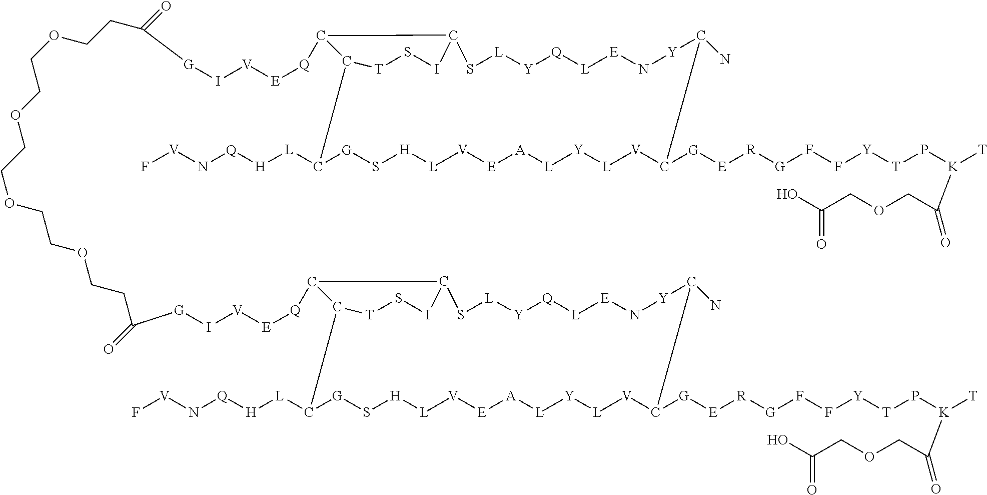 |
| 37 | 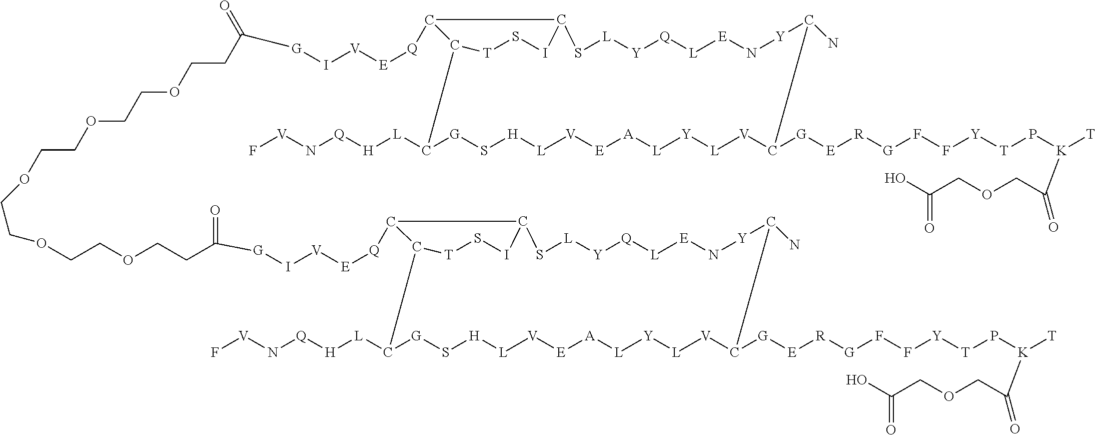 |
| 38 | 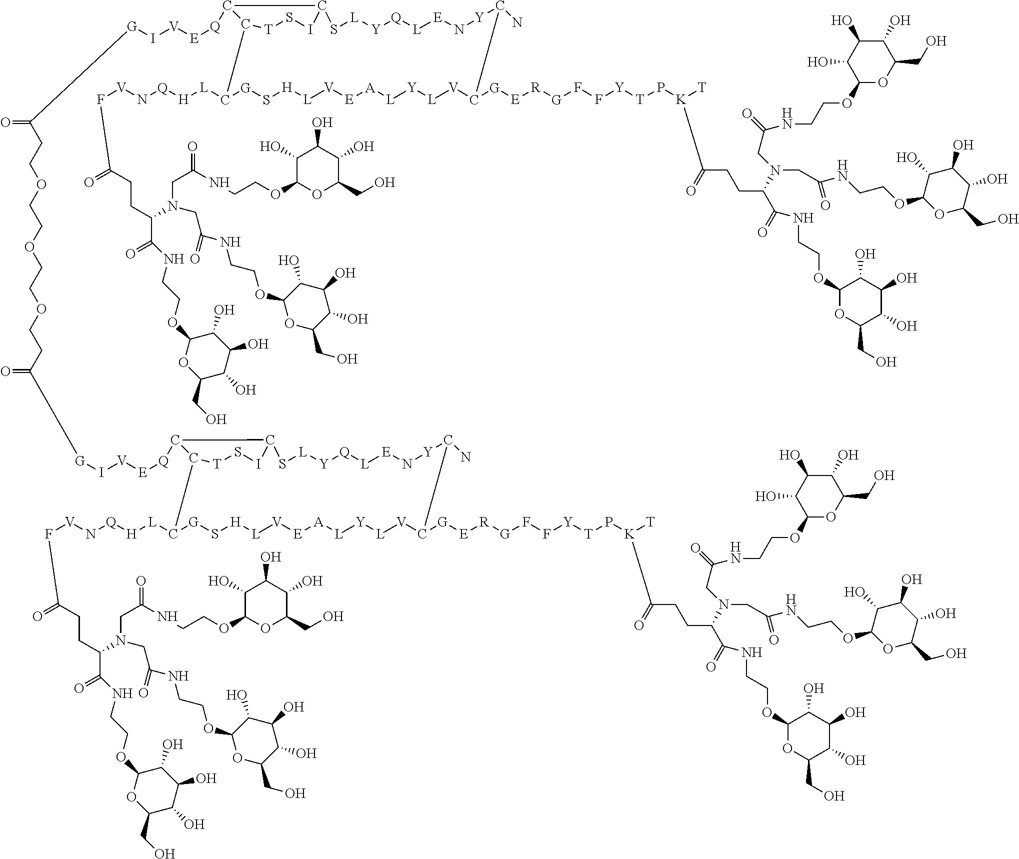 |
| 39 | 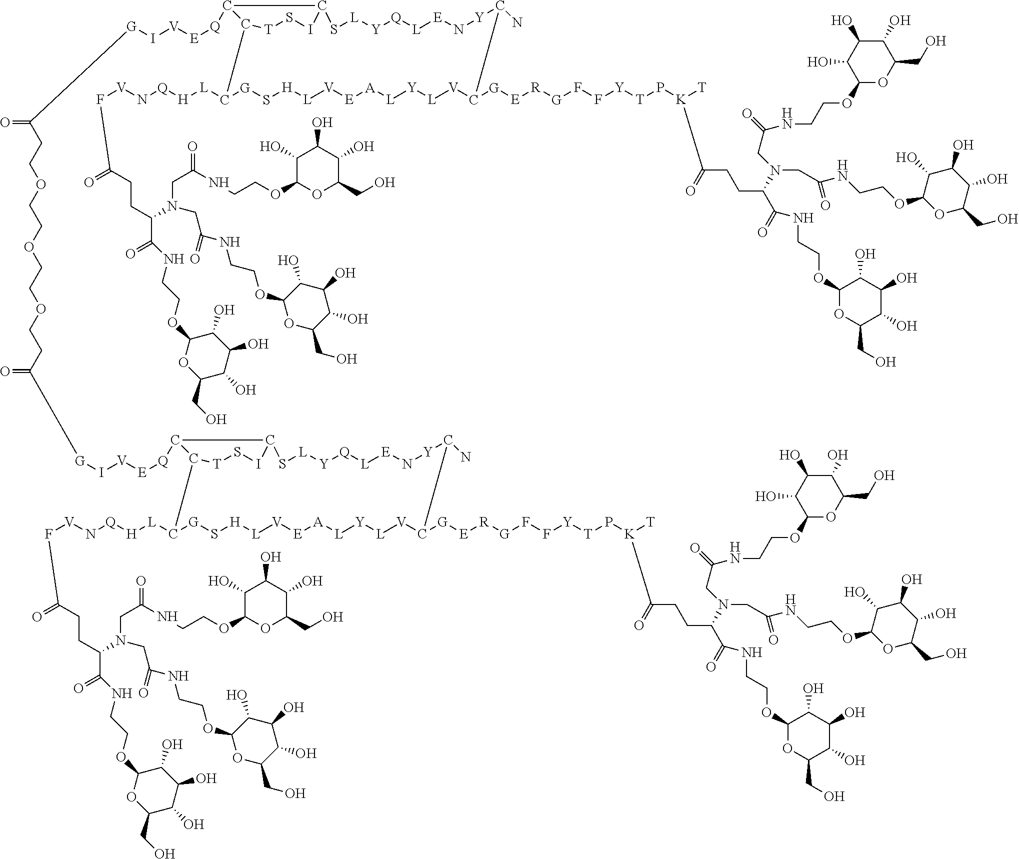 |
| 40 | 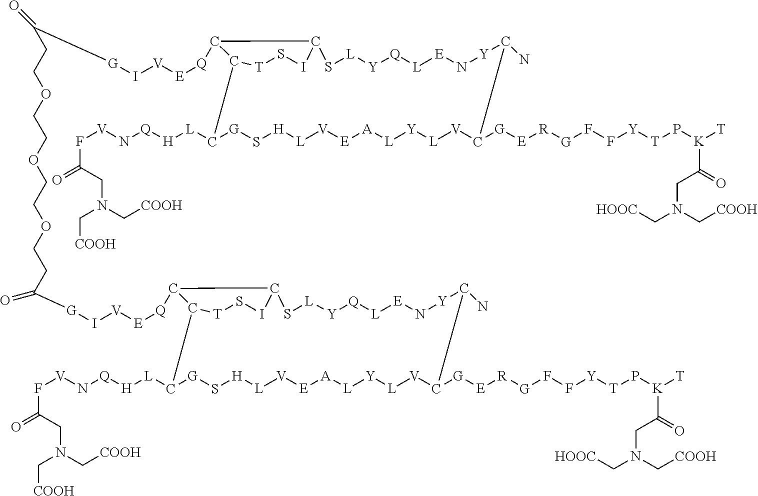 |
| 41 | 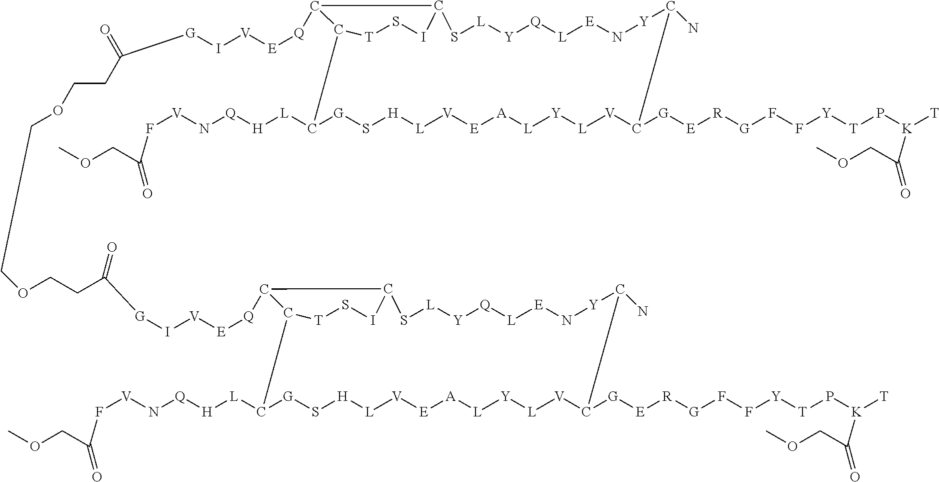 |
| 42 | 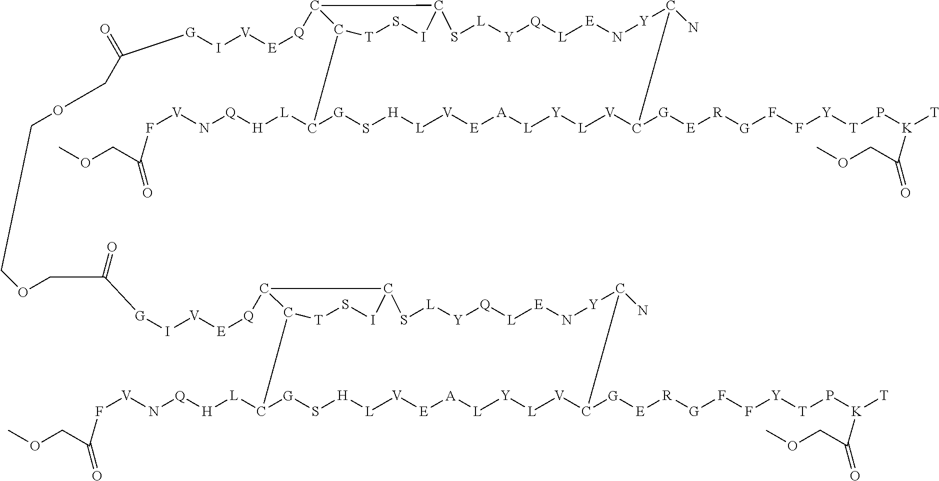 |
| 43 | 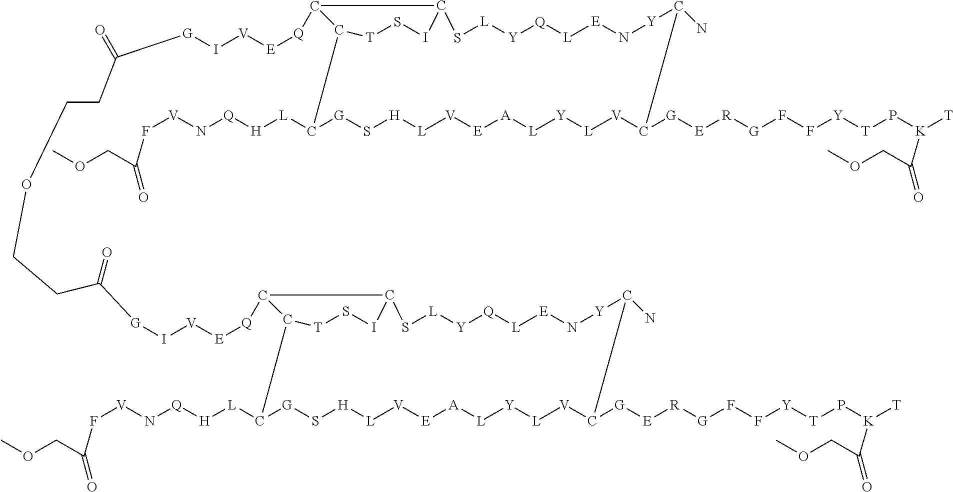 |
| 44 | 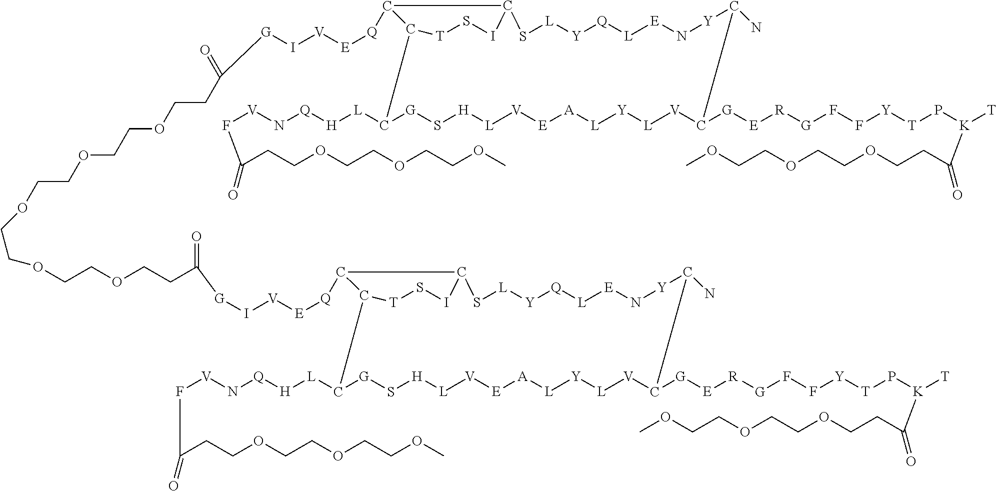 |
| 45 | 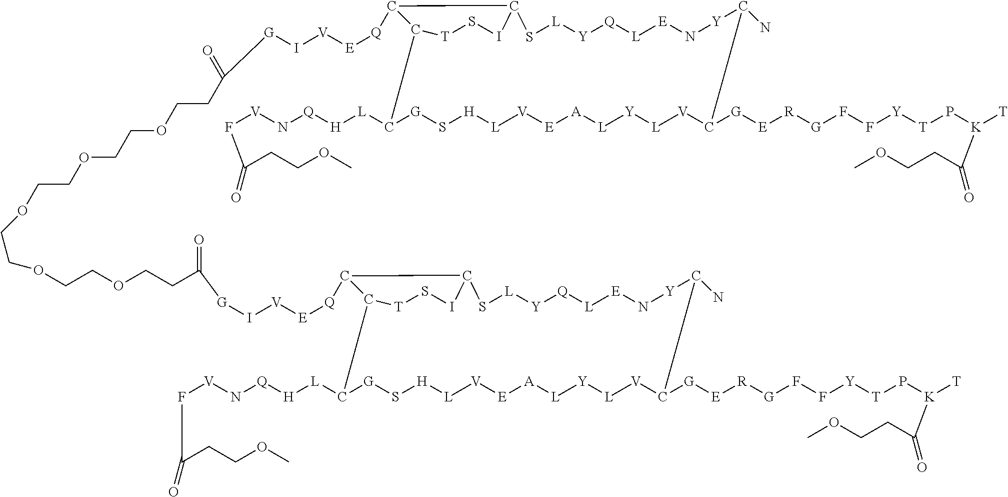 |
| 46 | 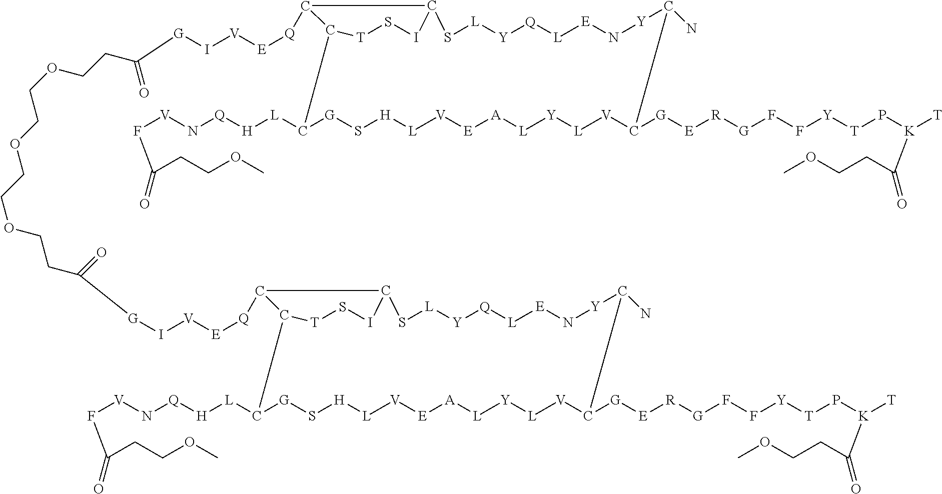 |
| 47 | 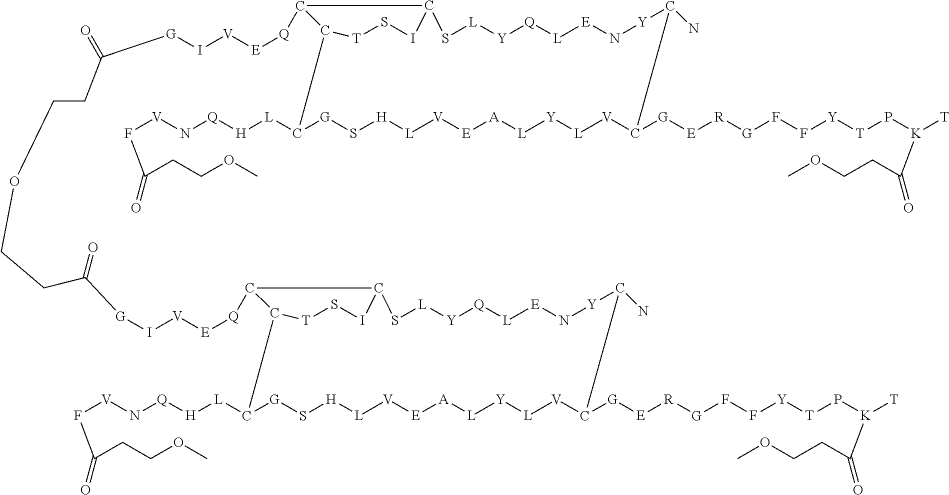 |
| 48 | 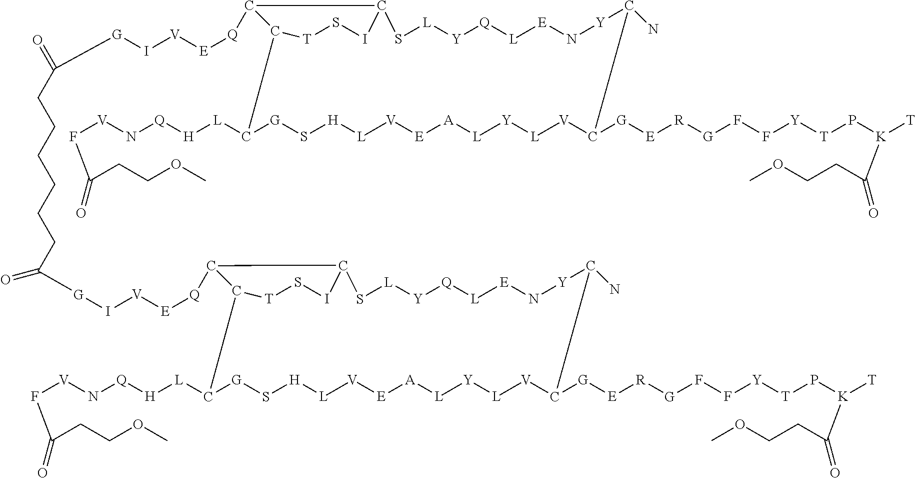 |
| 49 | 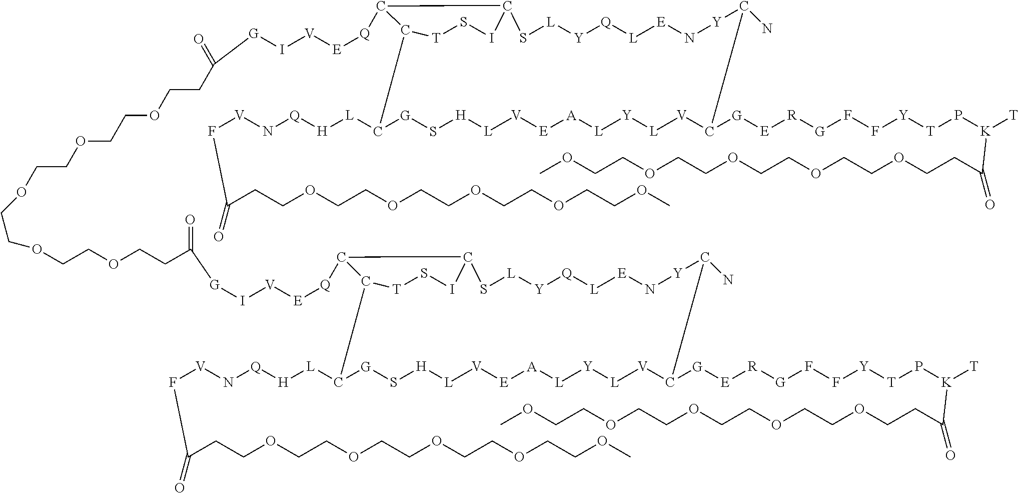 |
| 50 | 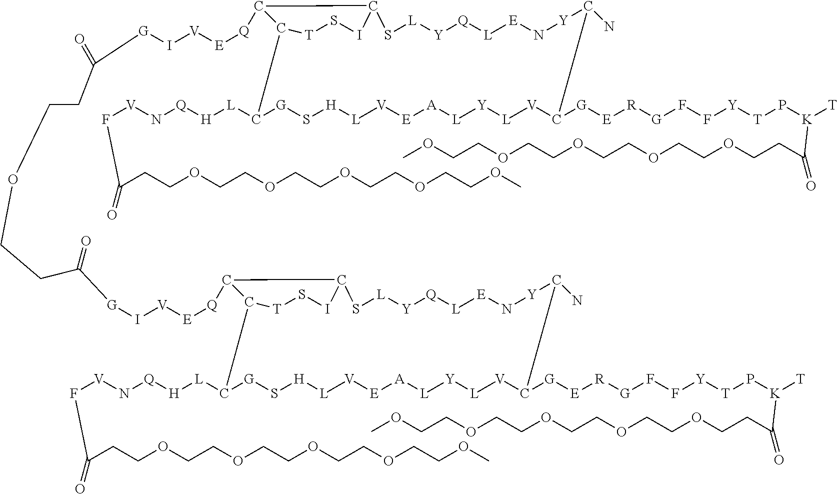 |
| 51 | 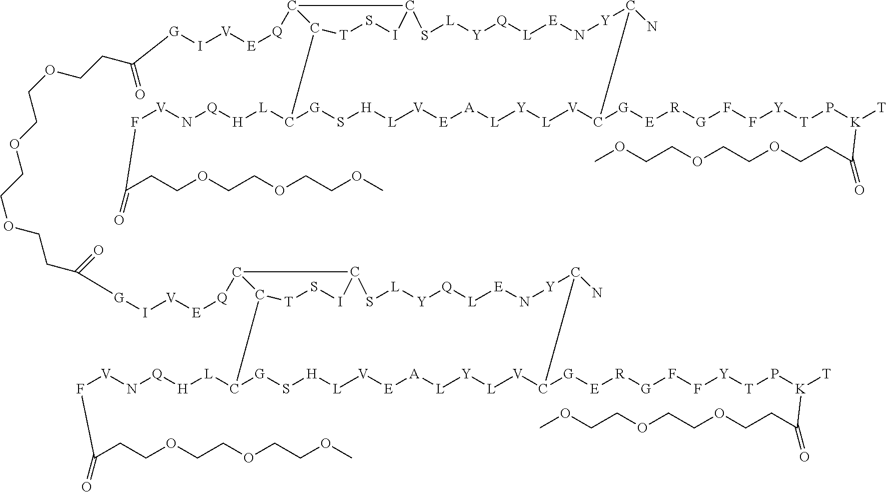 |
| 52 | 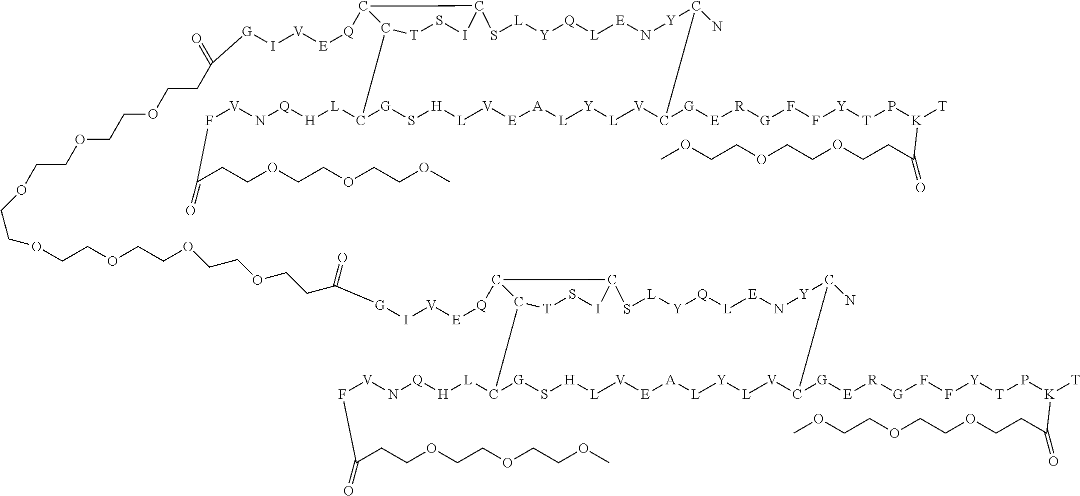 |
| 53 | 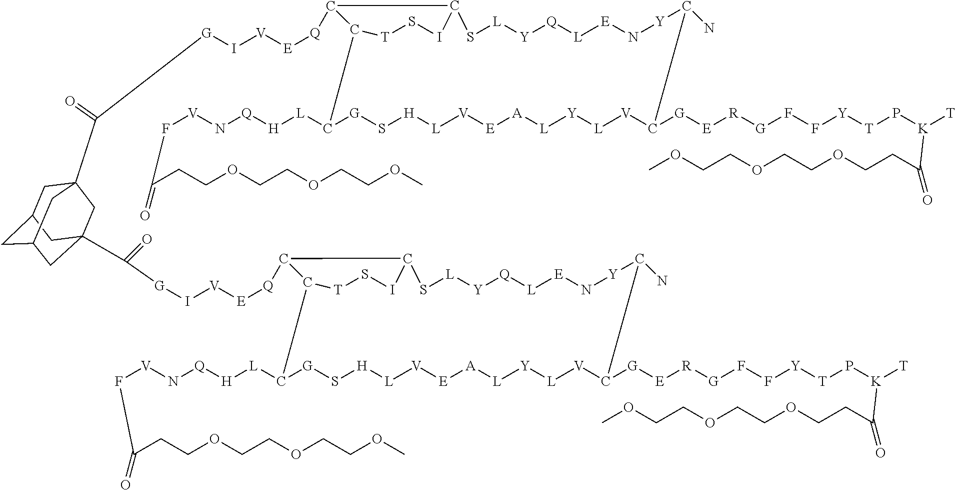 |
| 54 | 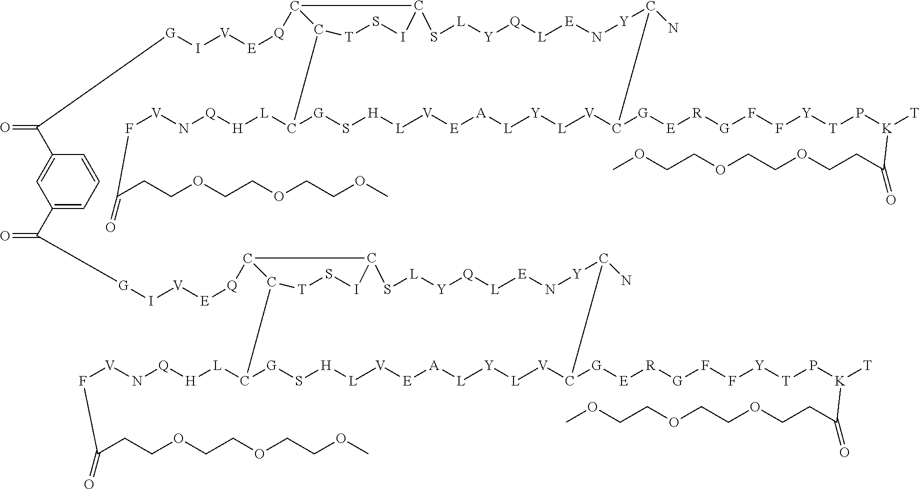 |
| 55 | 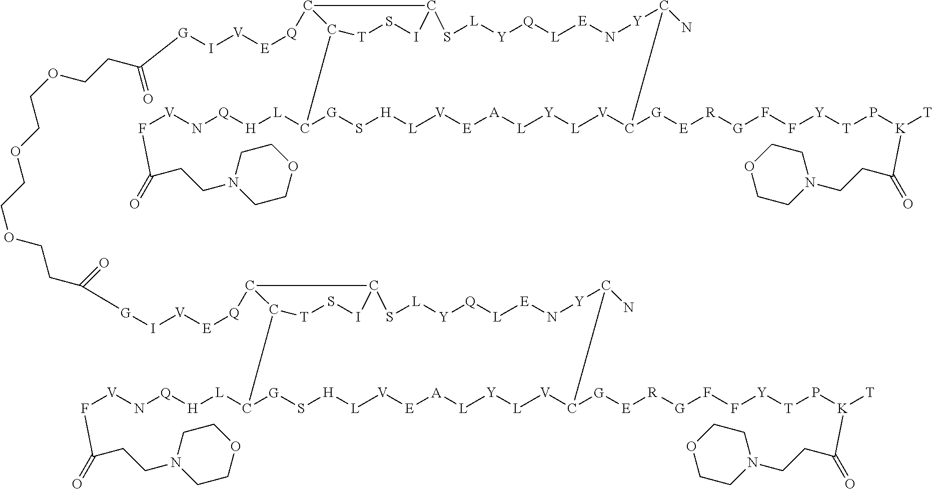 |
| 56 | 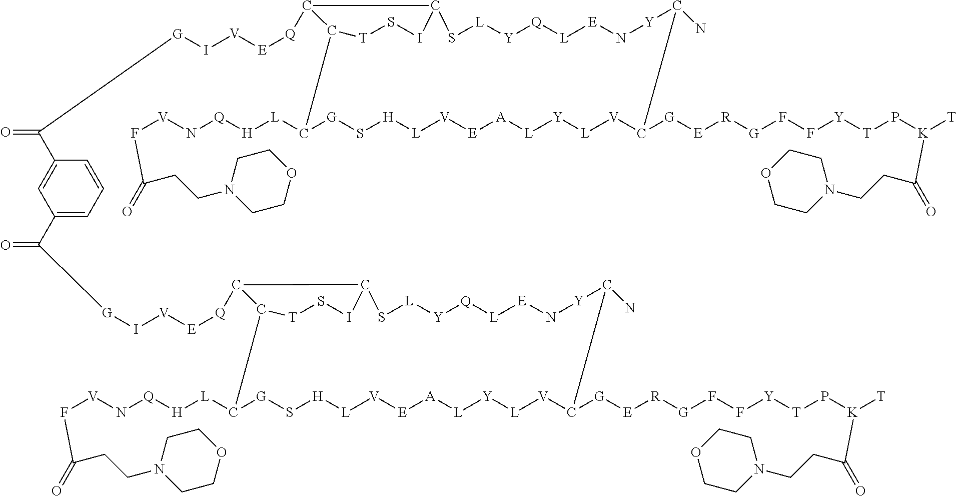 |
| 57 | 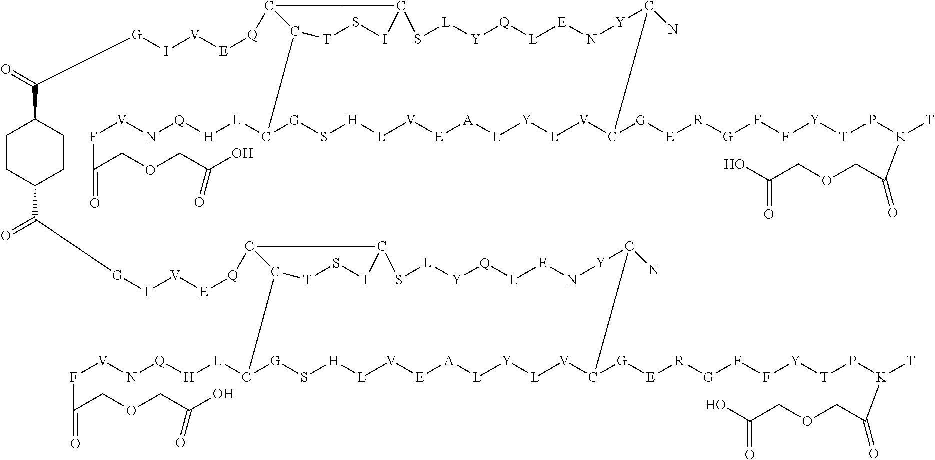 |
| 58 | 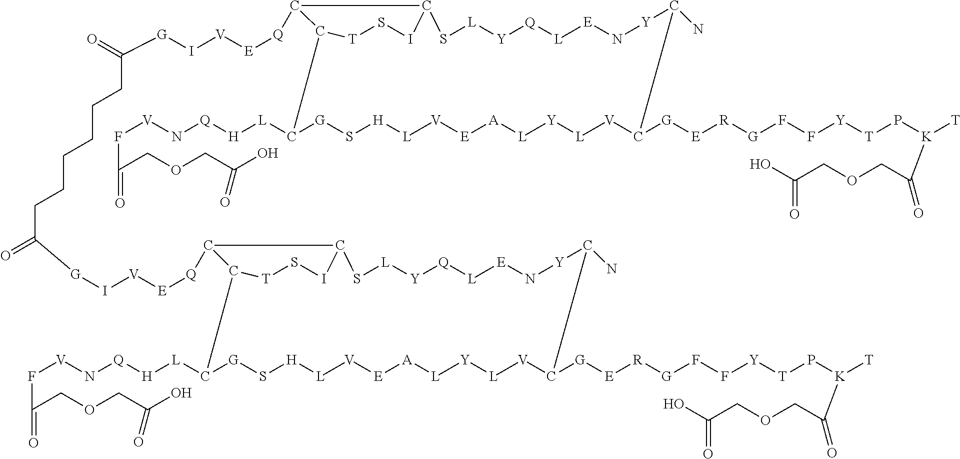 |
| 59 | 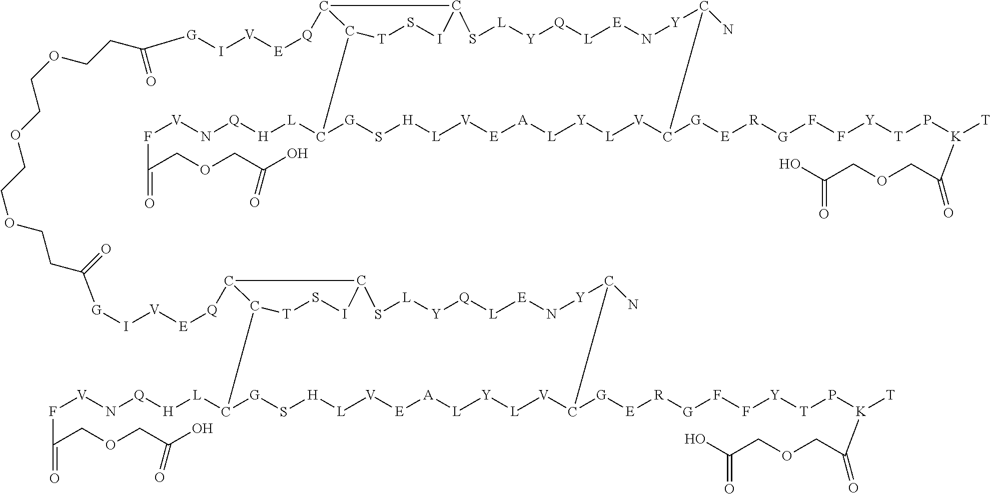 |
| 60 | 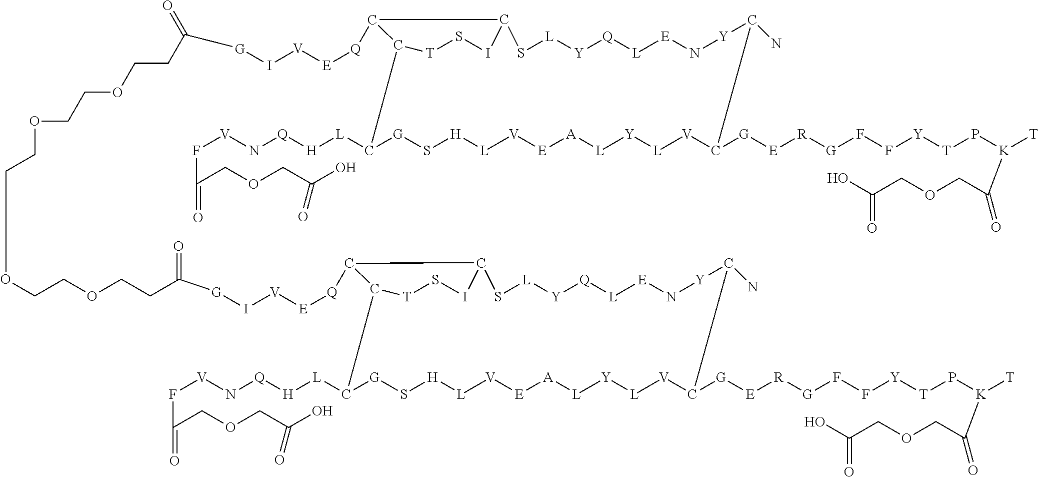 |
| 61 | 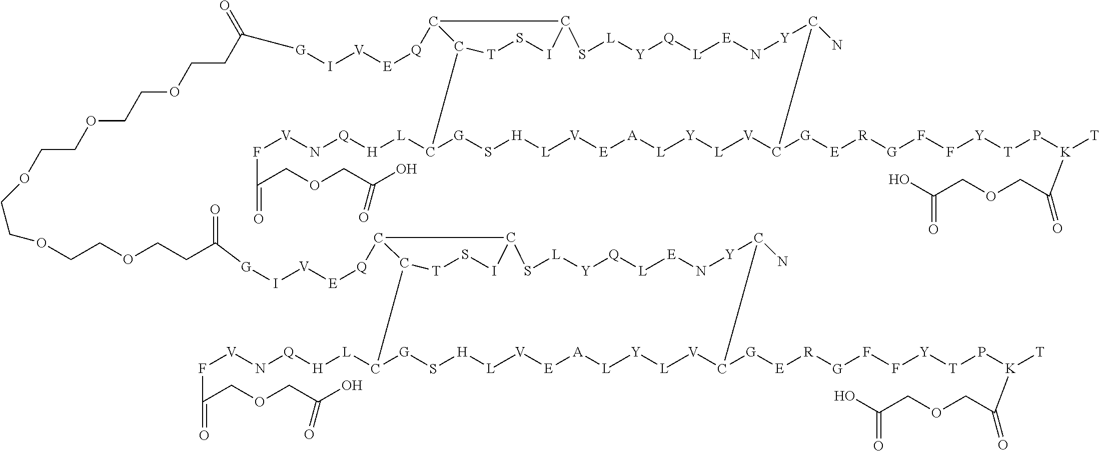 |
| 62 | 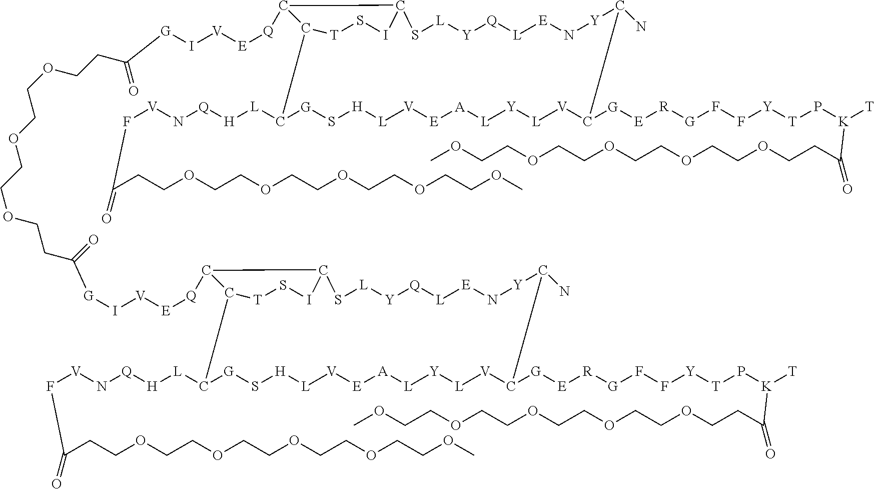 |
| 63 | 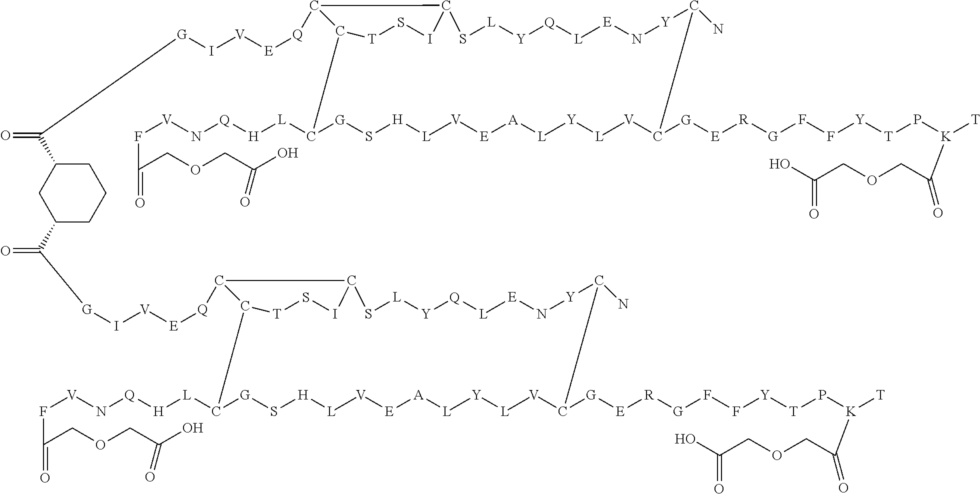 |
| 64 | 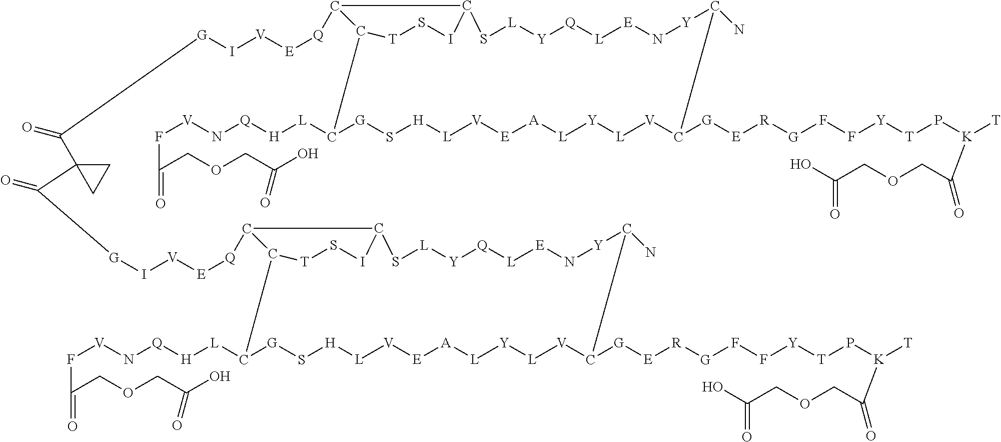 |
| 65 | 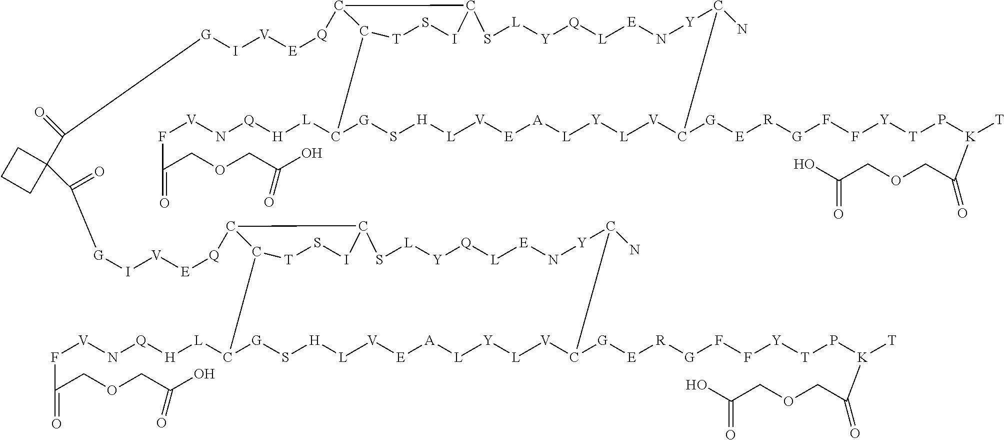 |
| 66 | 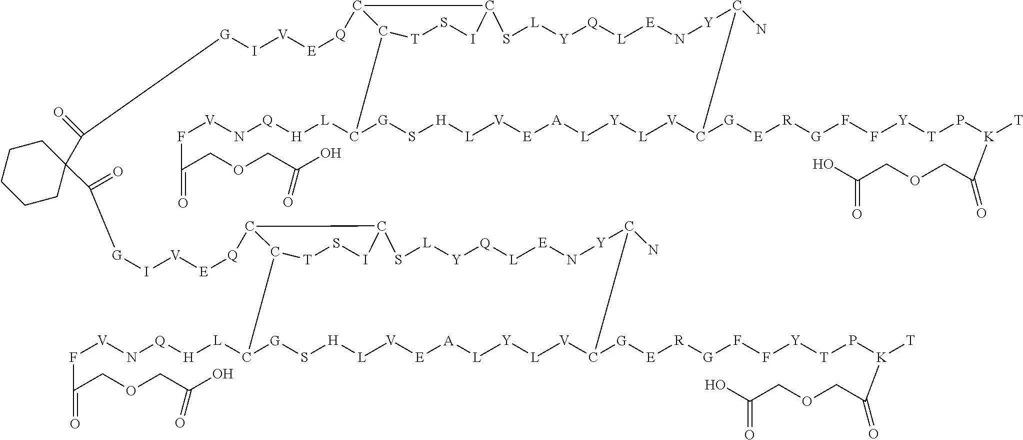 |
| 67 | 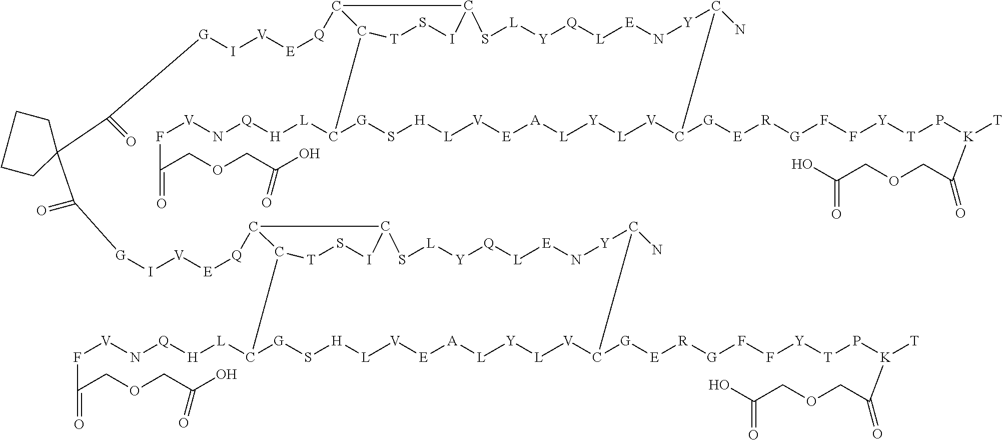 |
| 68 | 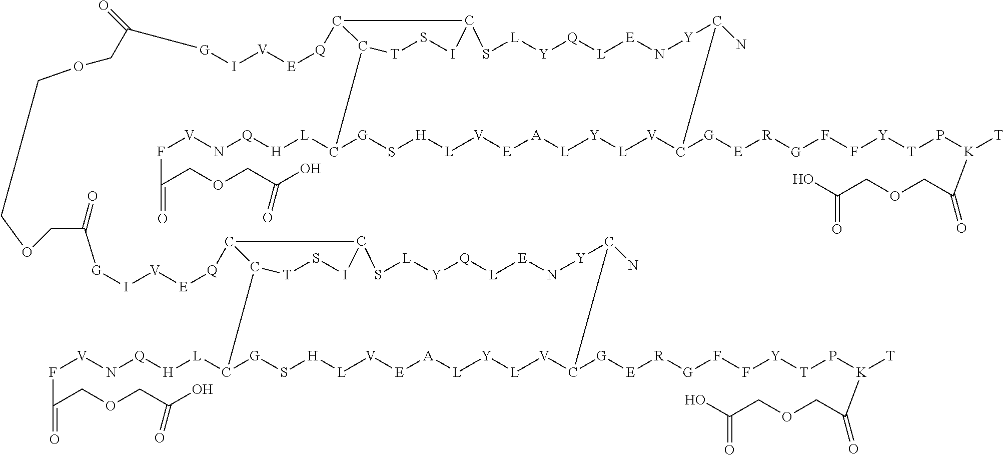 |
| 69 | 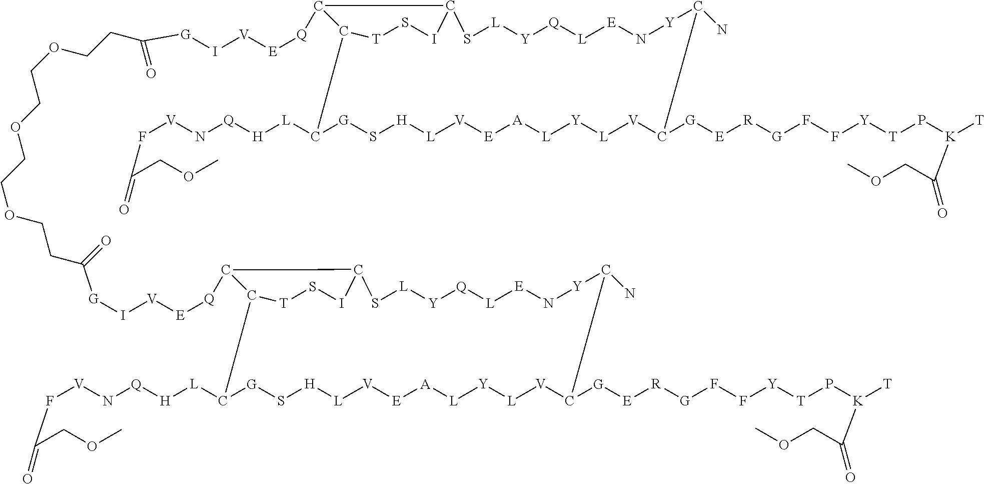 |
| 70 | 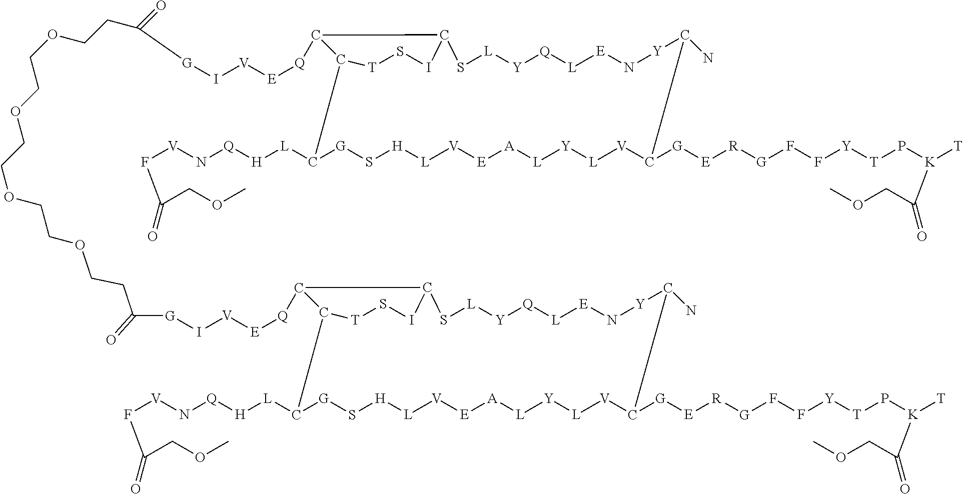 |
| 71 | 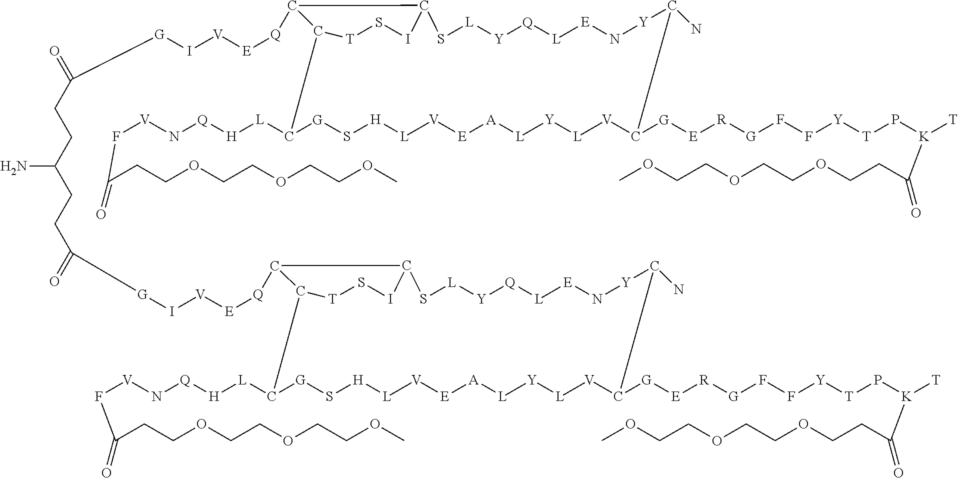 |
| 72 | 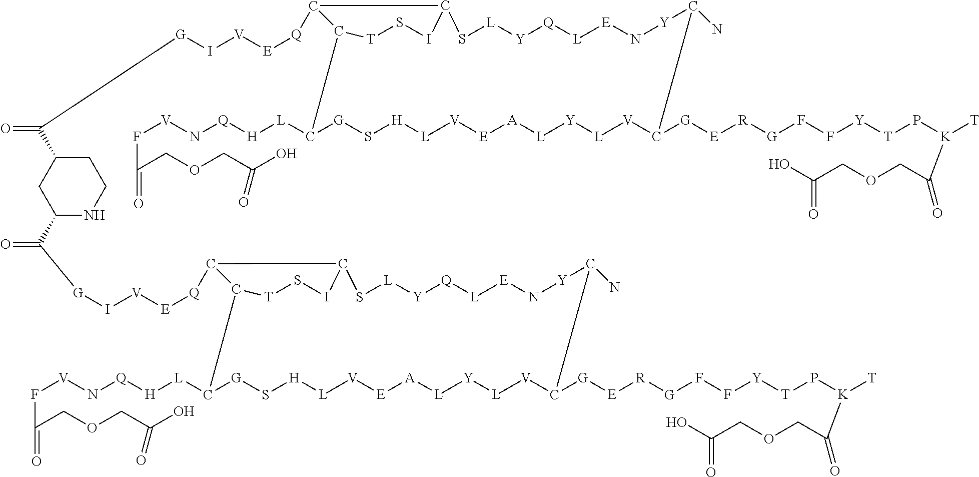 |
| 73 | 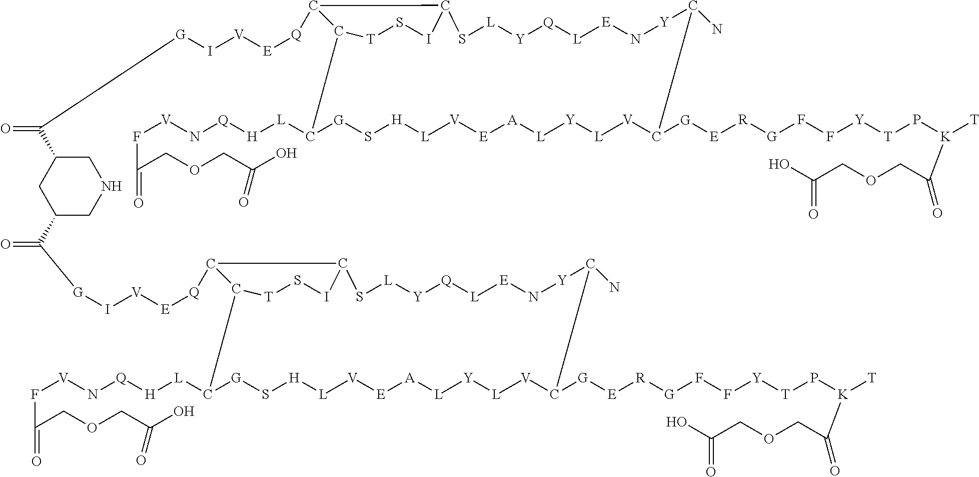 |
| 74 | 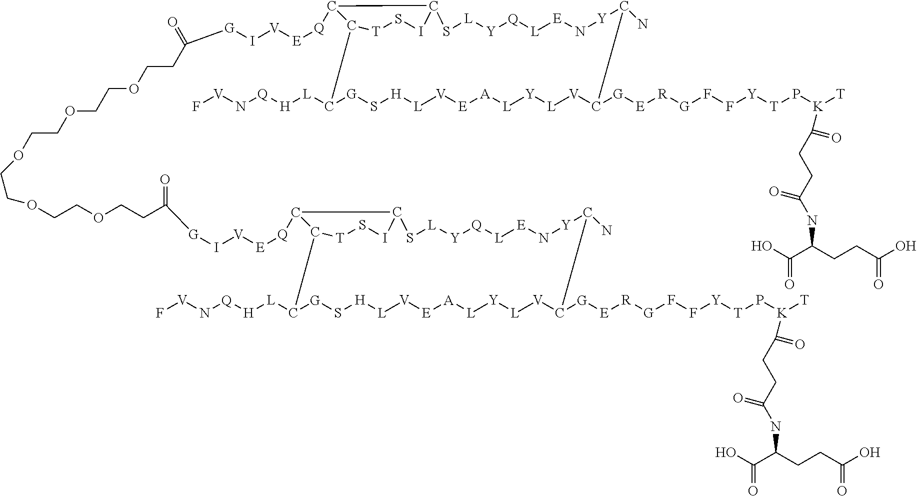 |
| 75 | 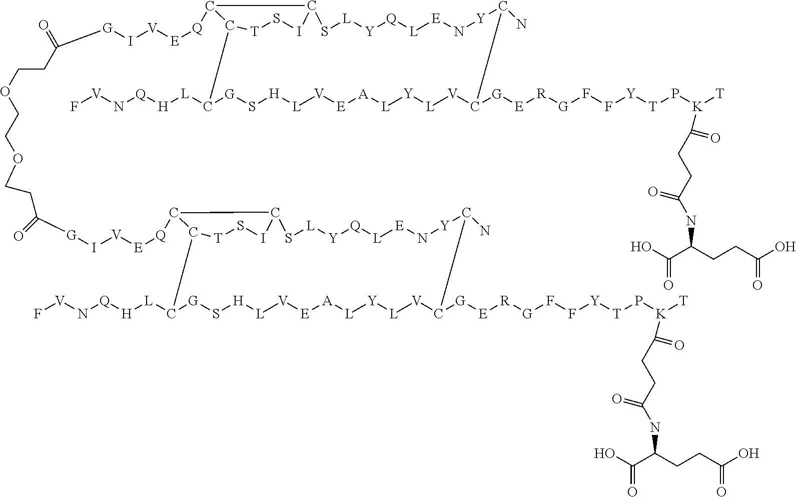 |
| 76 | 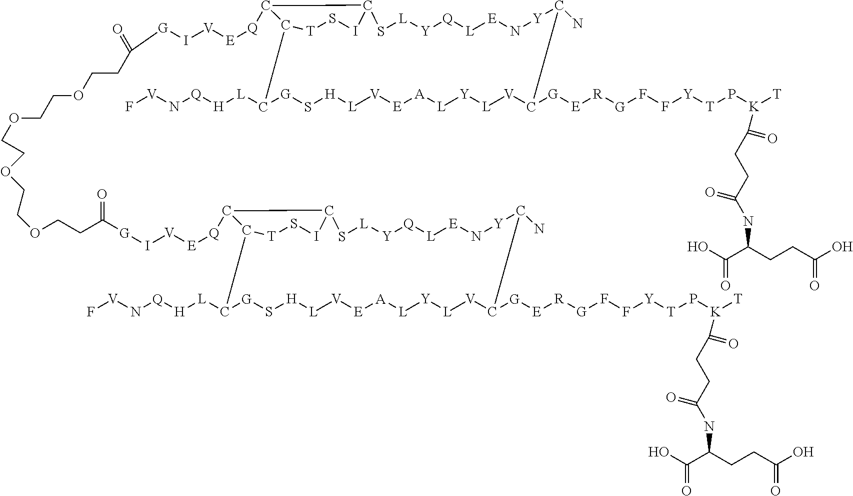 |
| 77 | 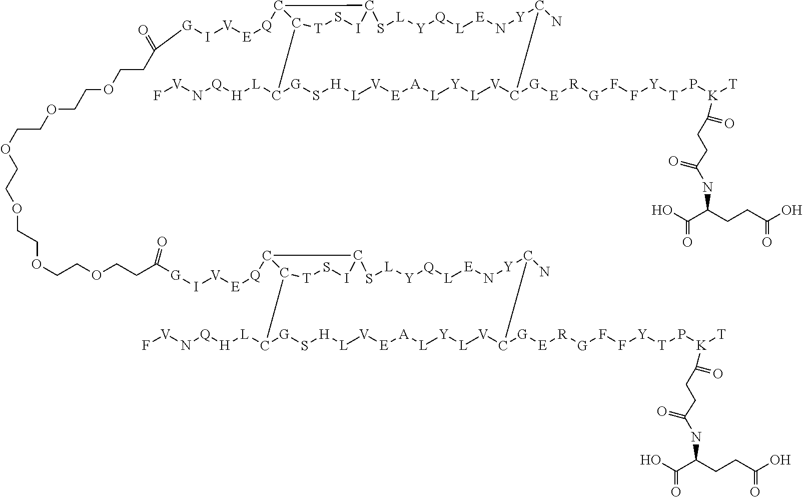 |
| 78 | 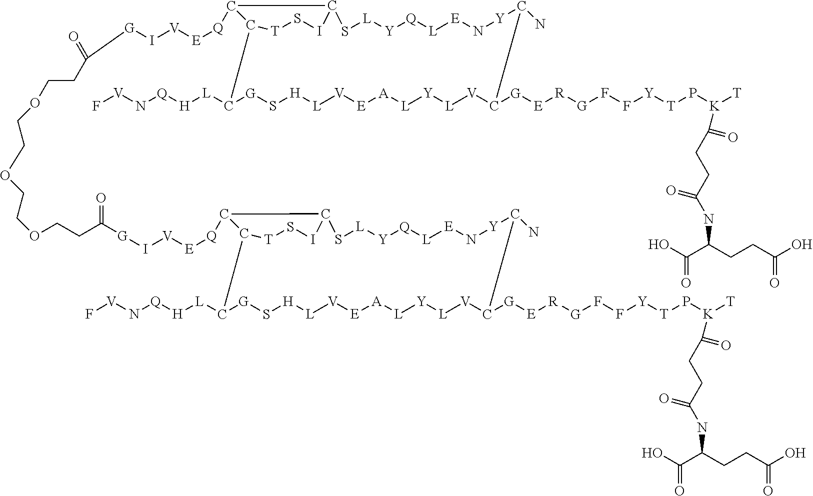 |
| 79 | 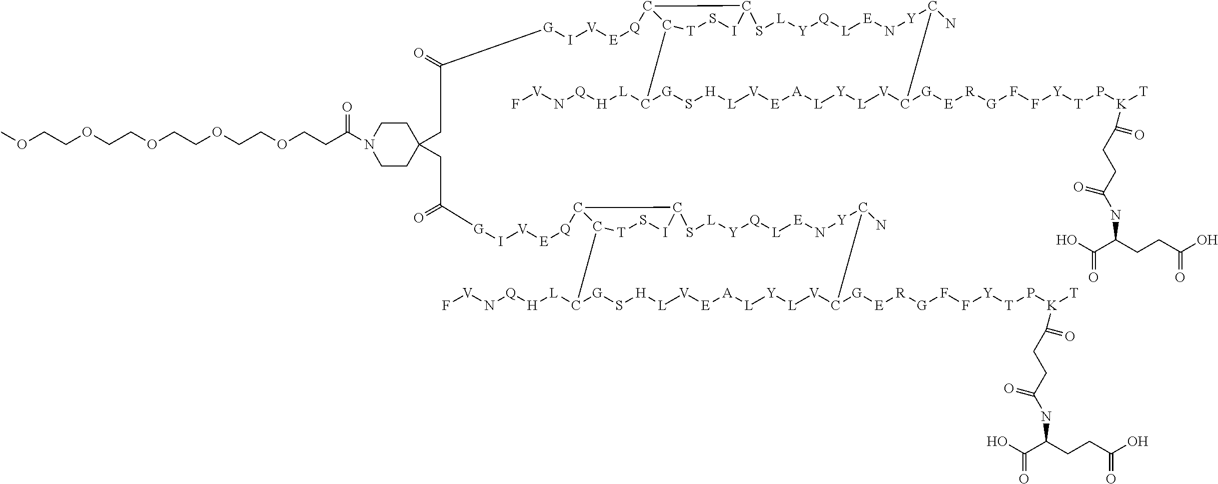 |
| 80 | 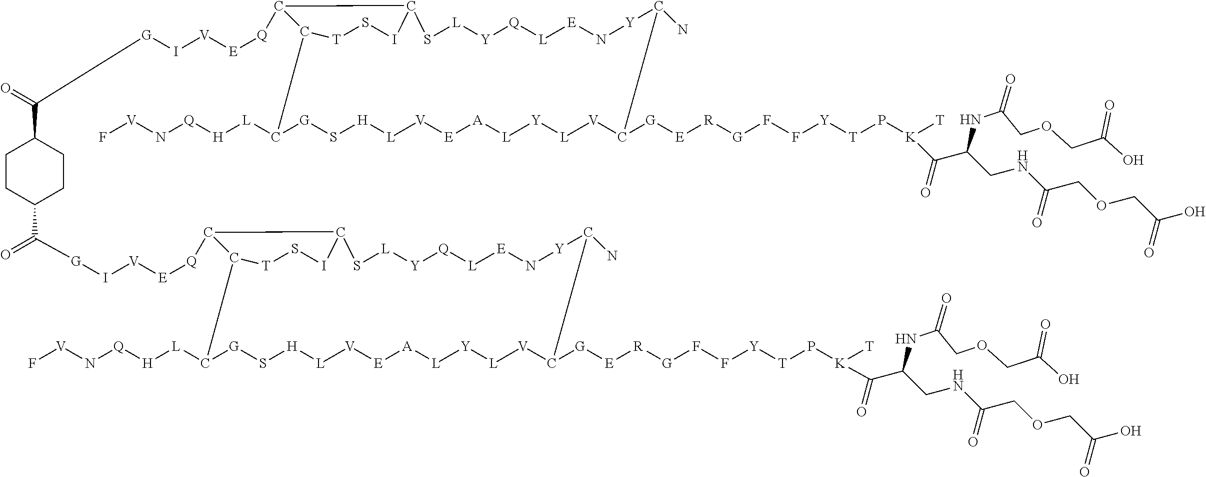 |
| 81 | 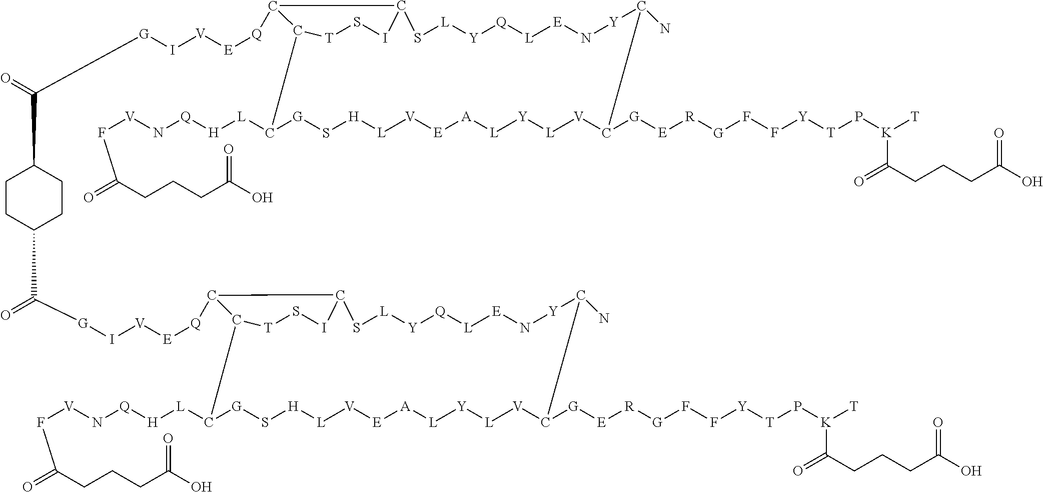 |
| 82 | 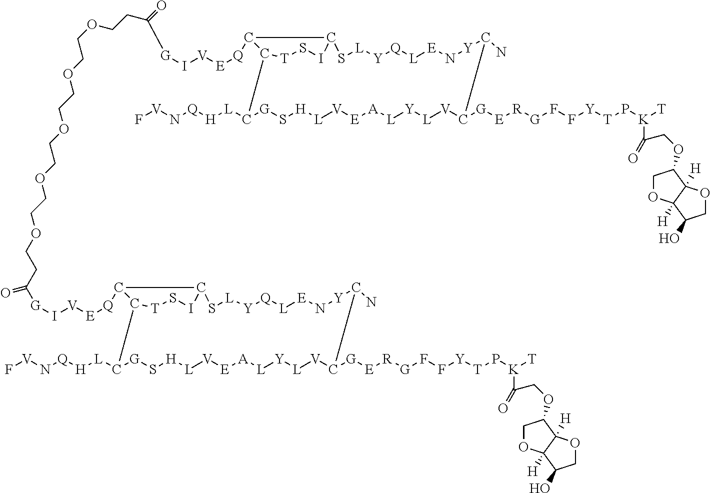 |
| 83 | 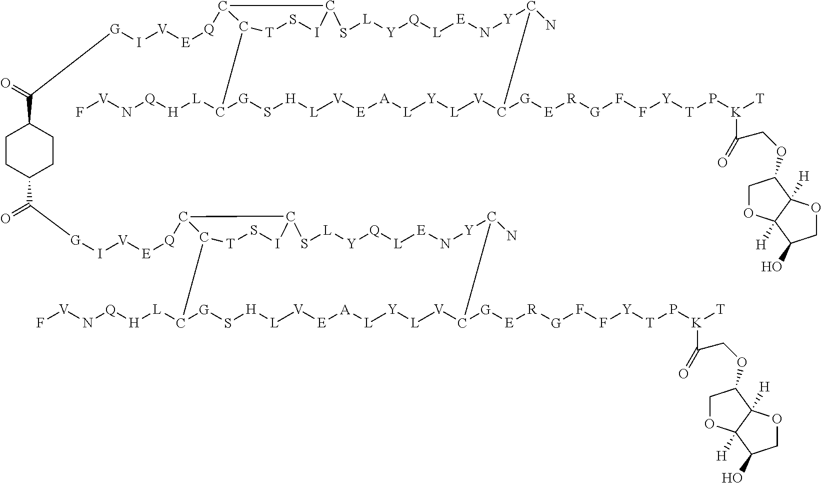 |
| 84 | 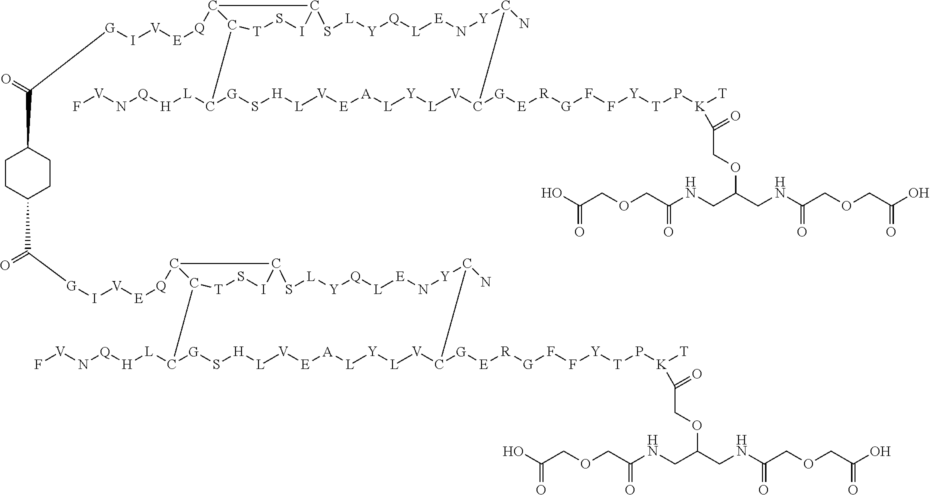 |
| 85 | 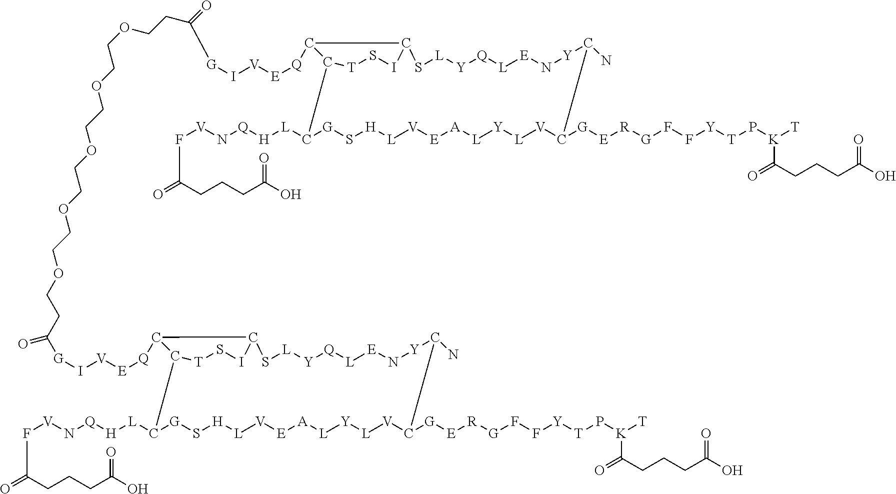 |
| 86 | 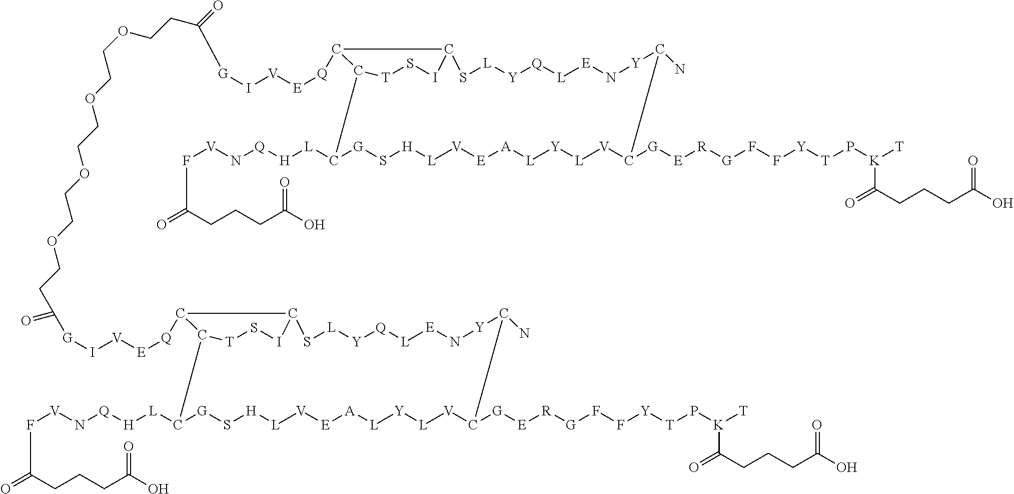 |
| 87 | 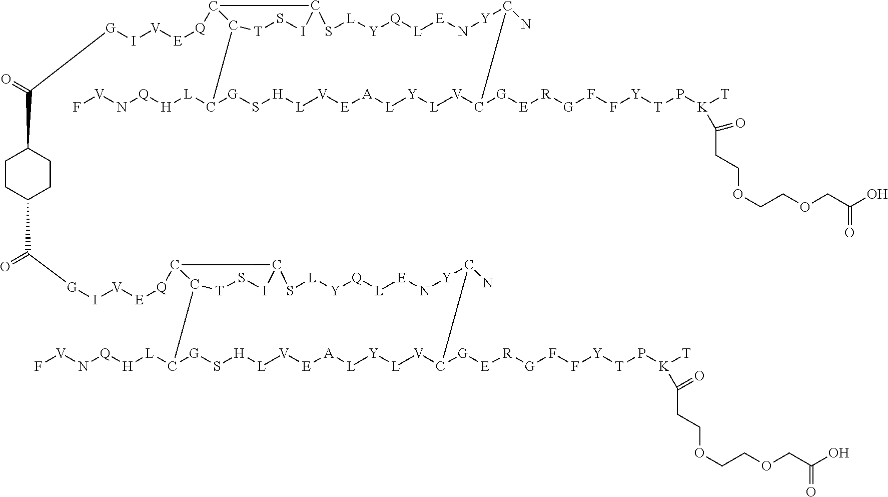 |
| 88 | 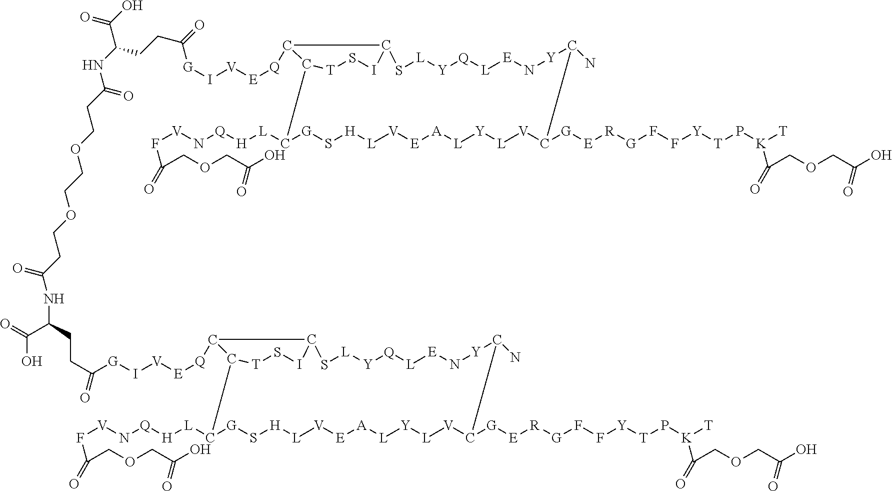 |
| 89 | 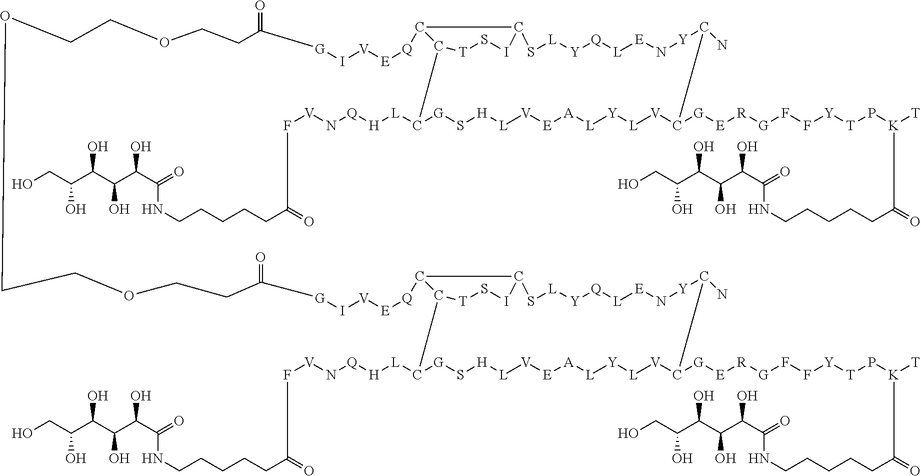 |
| 90 | 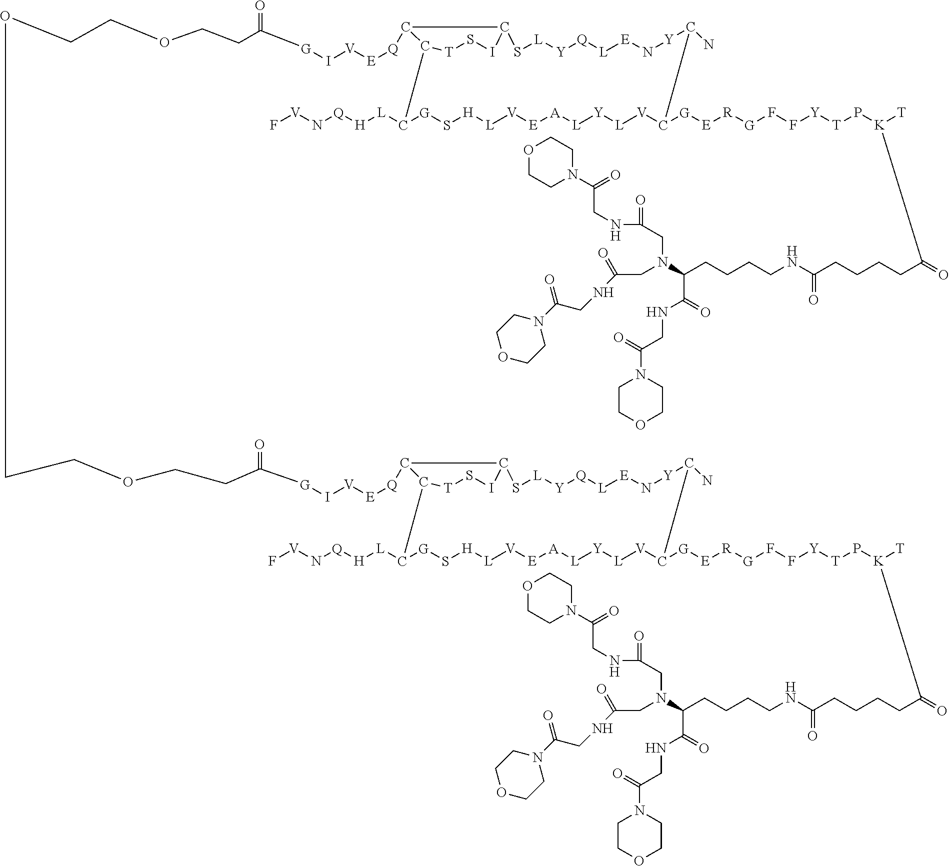 |
| 91 | 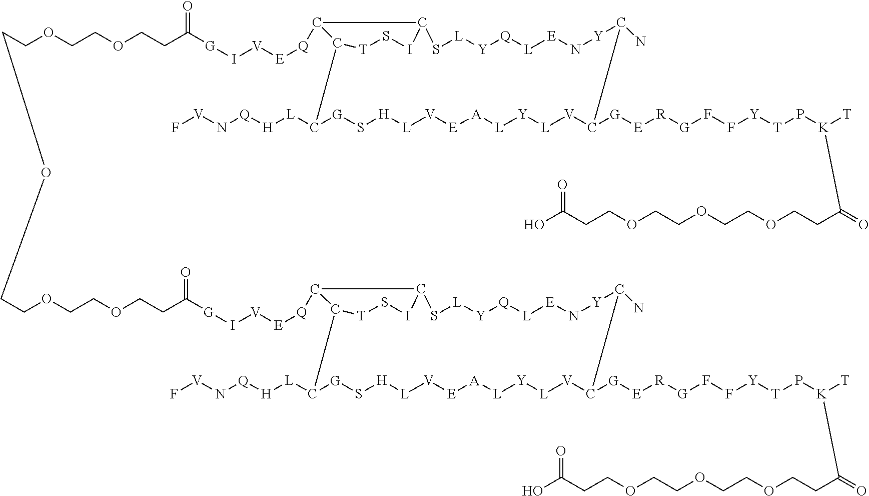 |
| 92 | 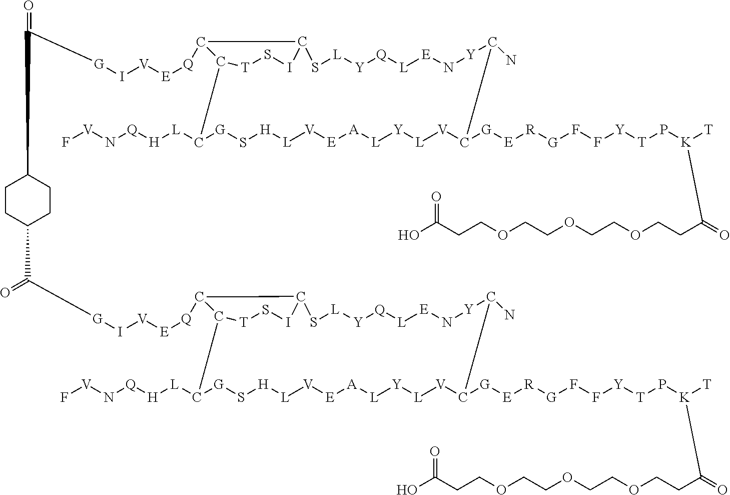 |
| 93 | 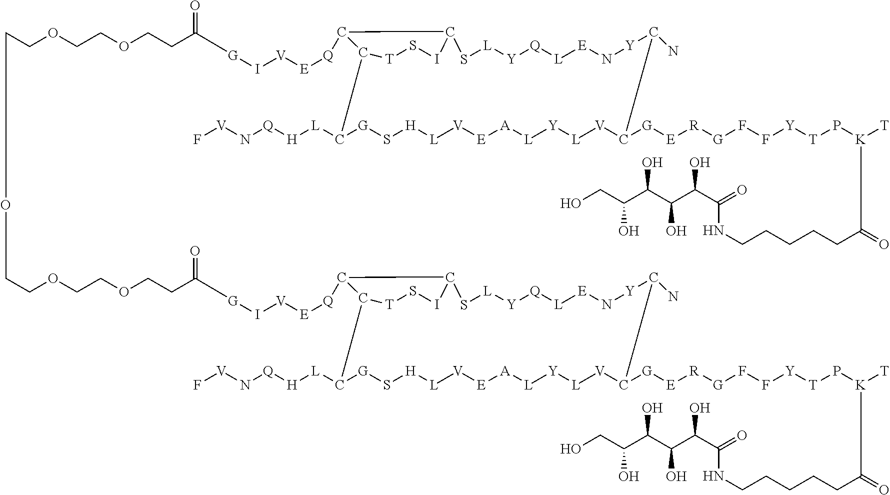 |
| 94 | 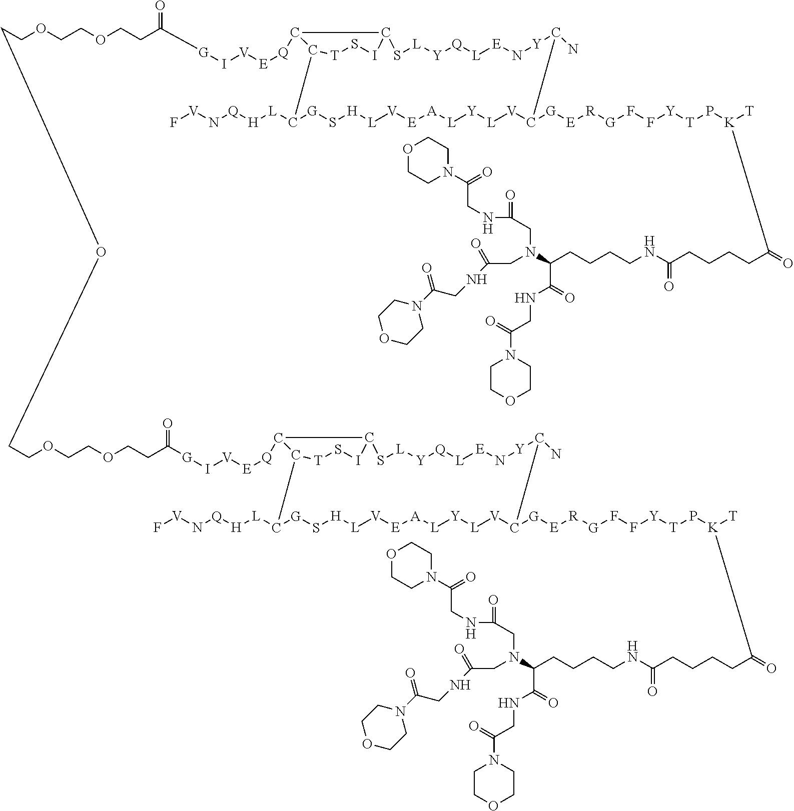 |
| 95 | 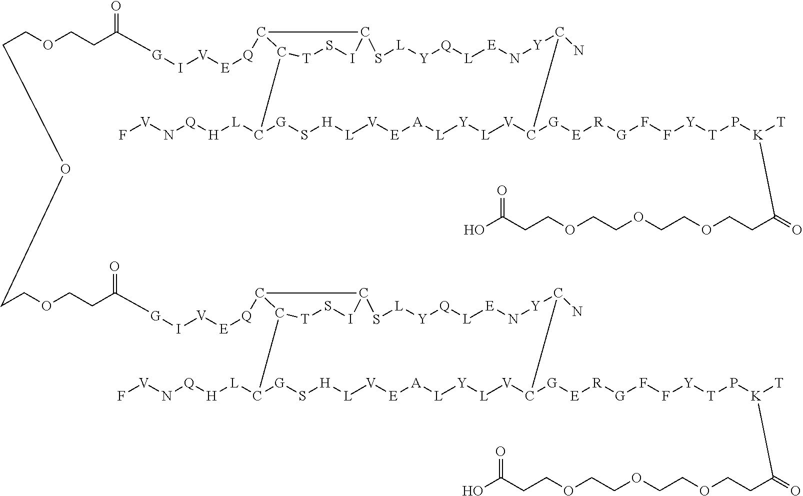 |
| 96 | 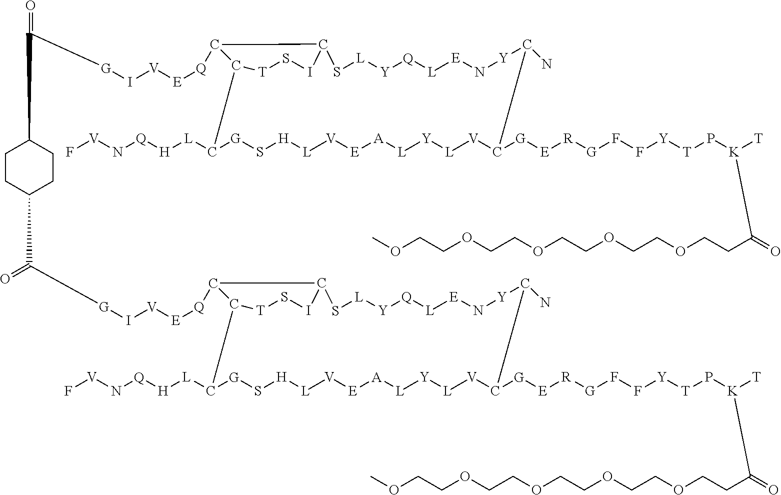 |
| 97 | 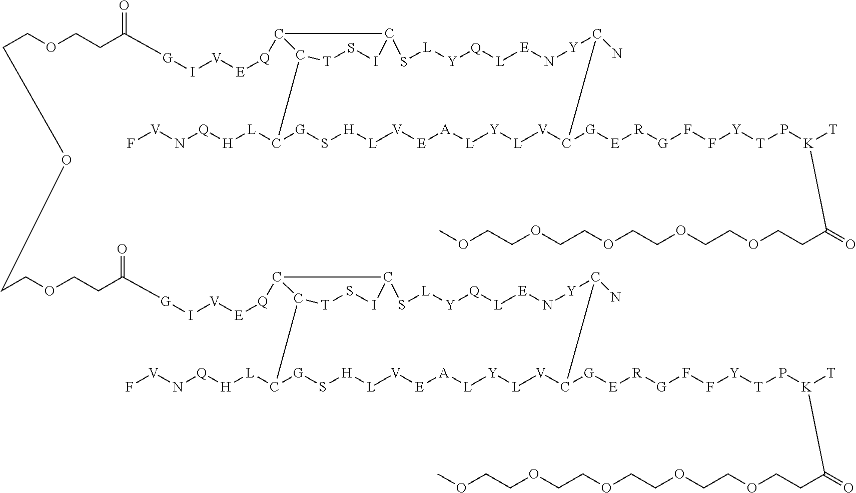 |
| 98 | 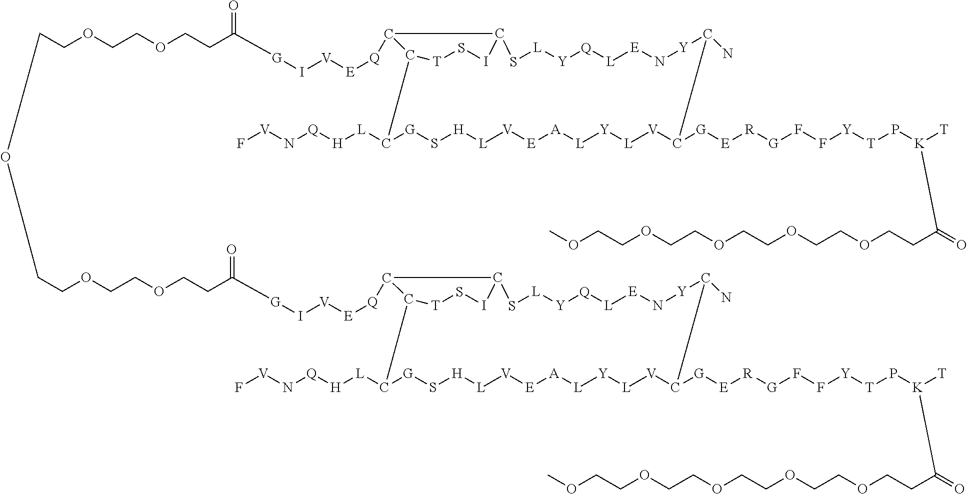 |
| 99 | 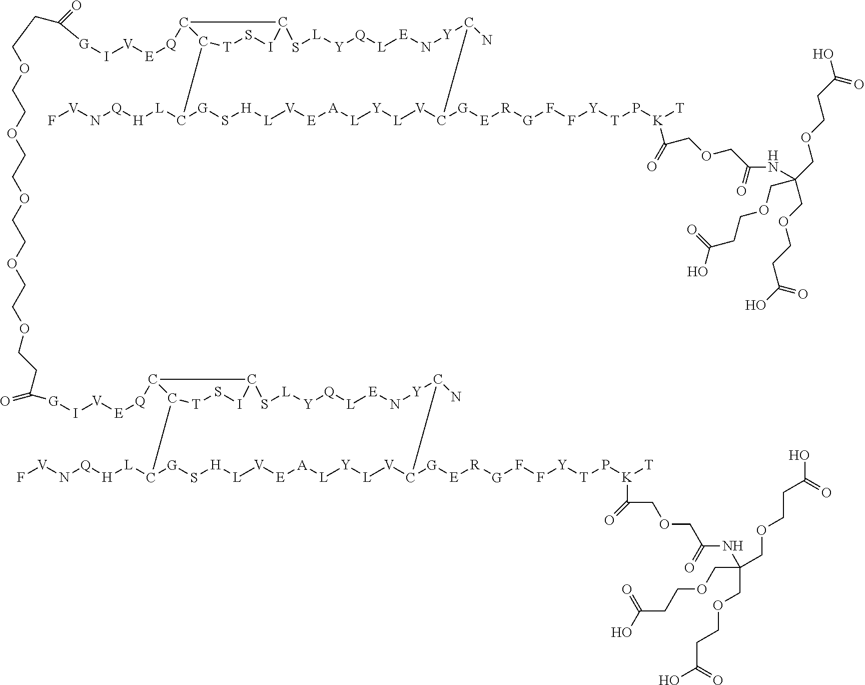 |
| 100 | 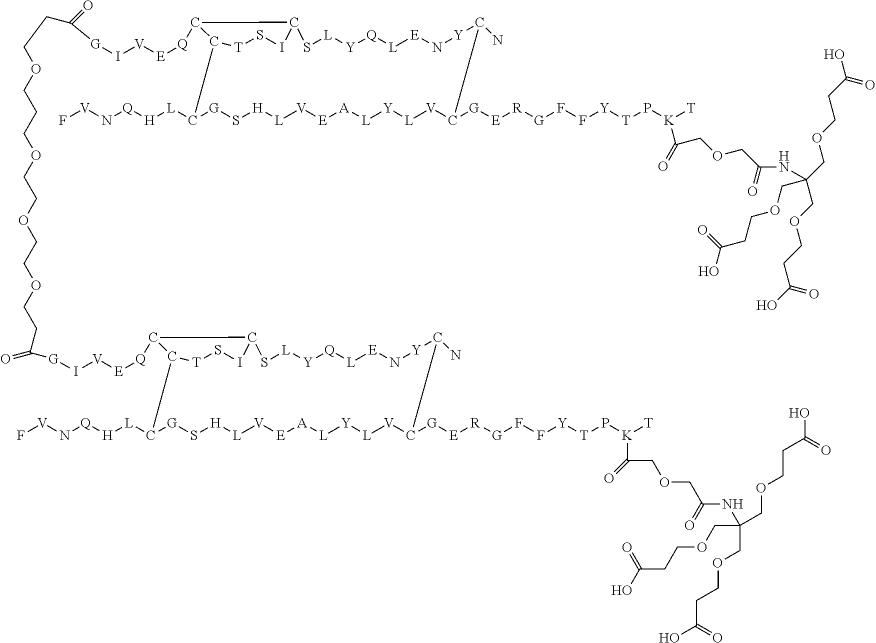 |
| 101 | 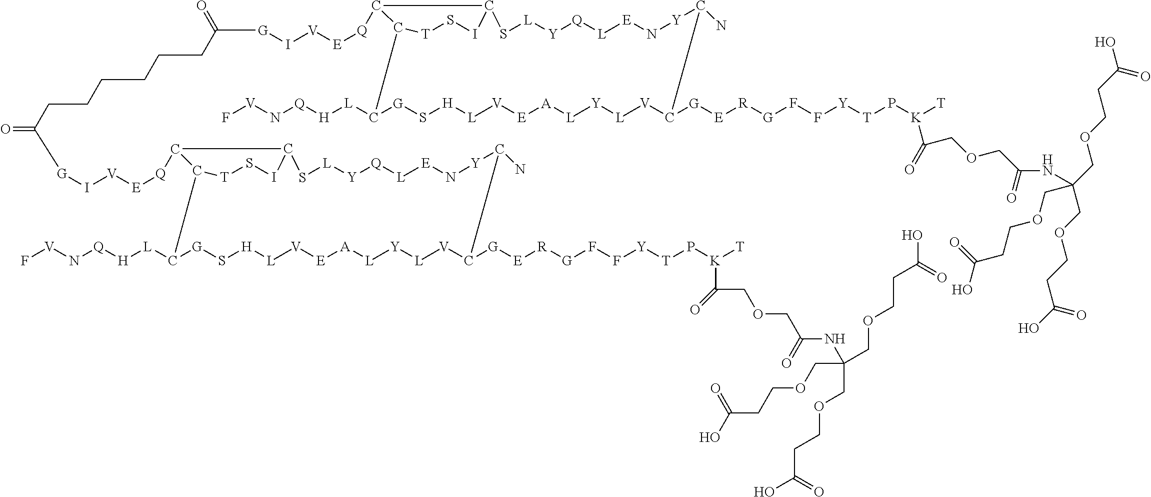 |
| 102 | 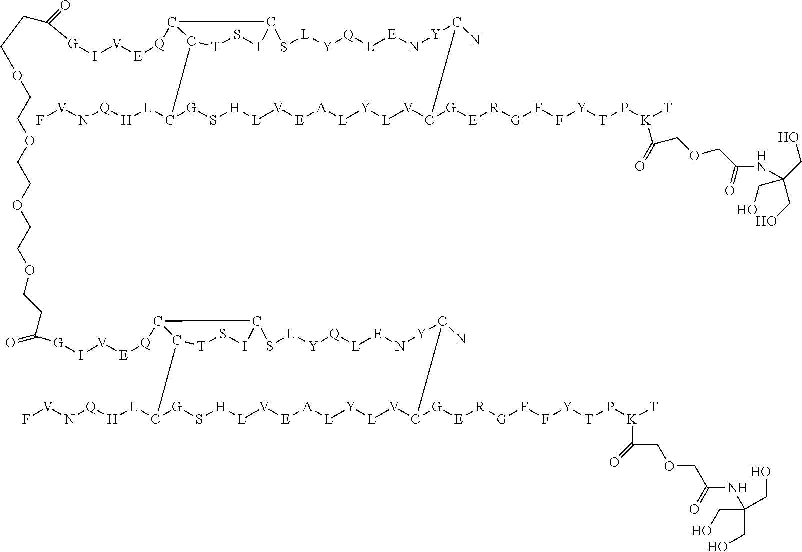 |
| 103 | 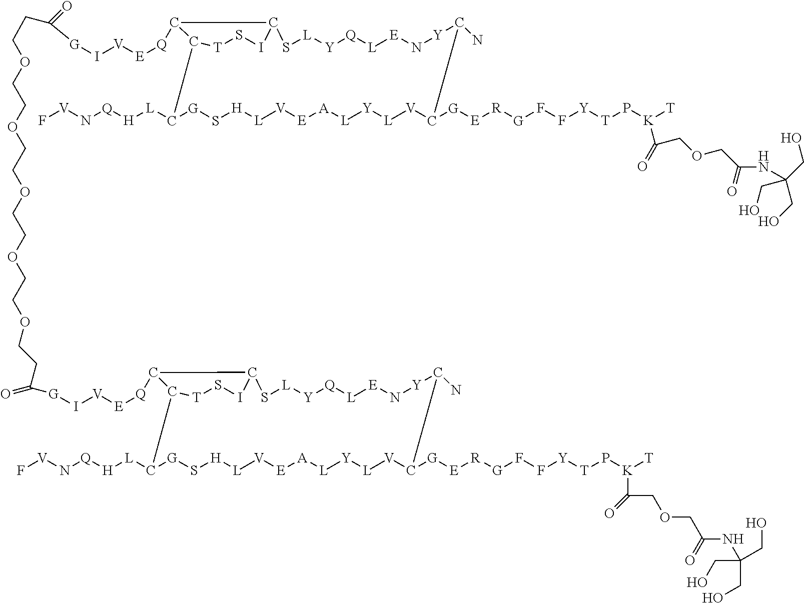 |
| 104 | 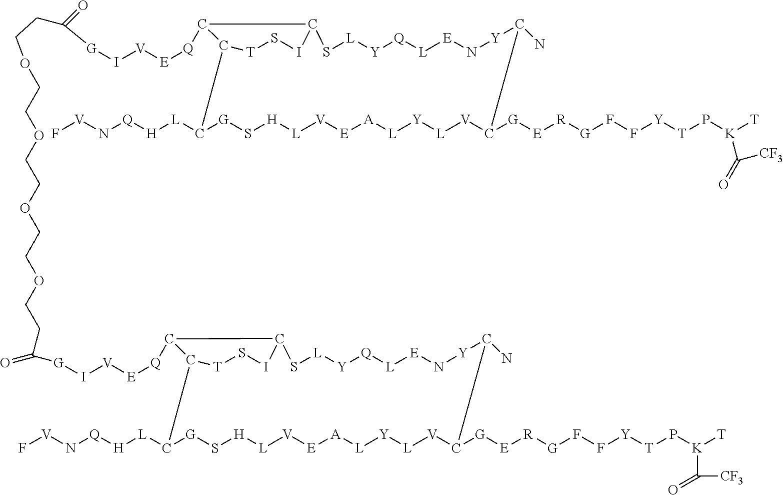 |
| 105 | 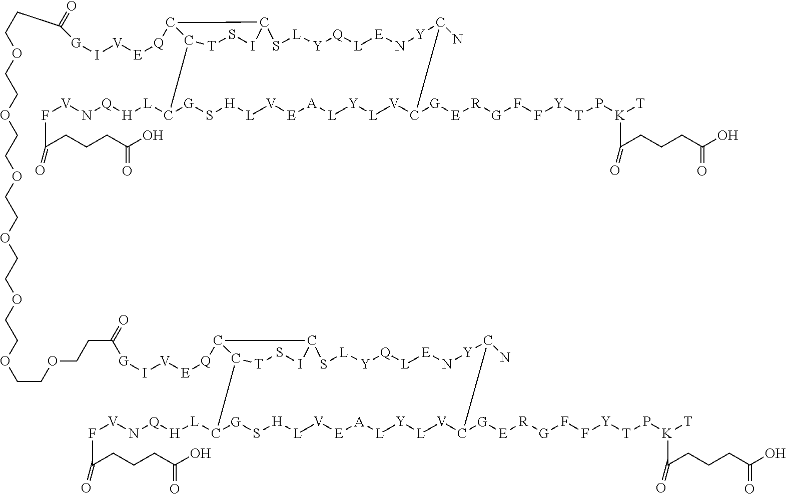 |
| 106 | 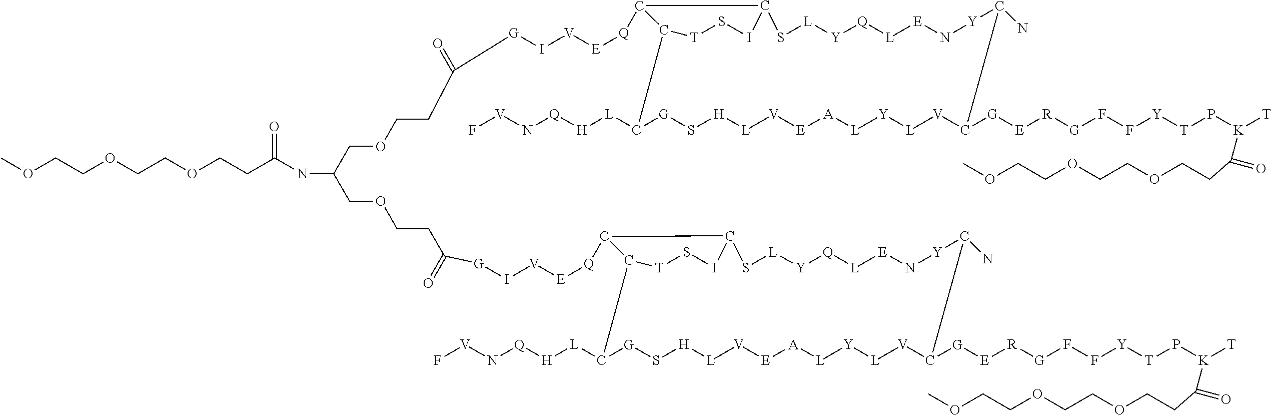 |
| 107 | 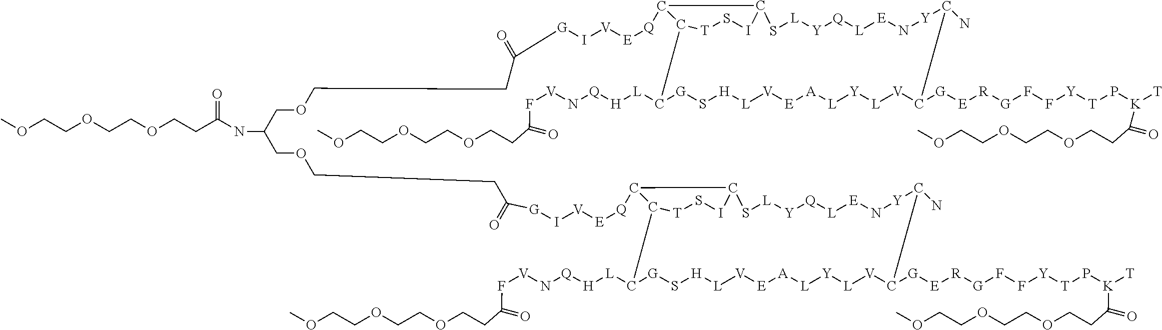 |
| 108 | 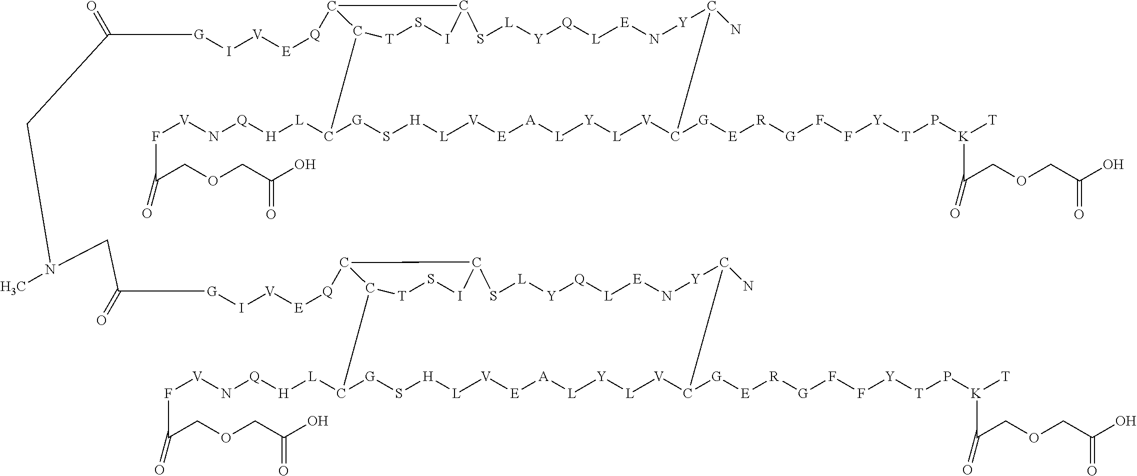 |
| 109 | 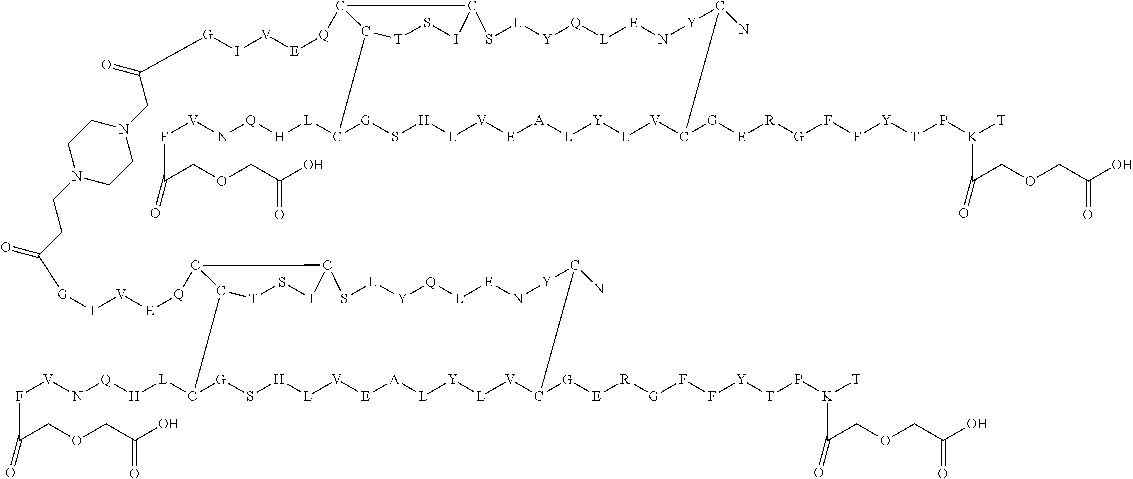 |
| 110 | 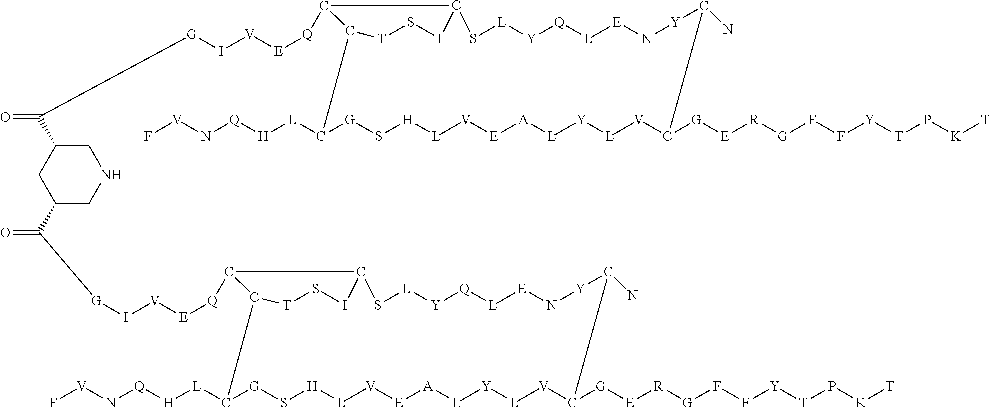 |
| 111 | 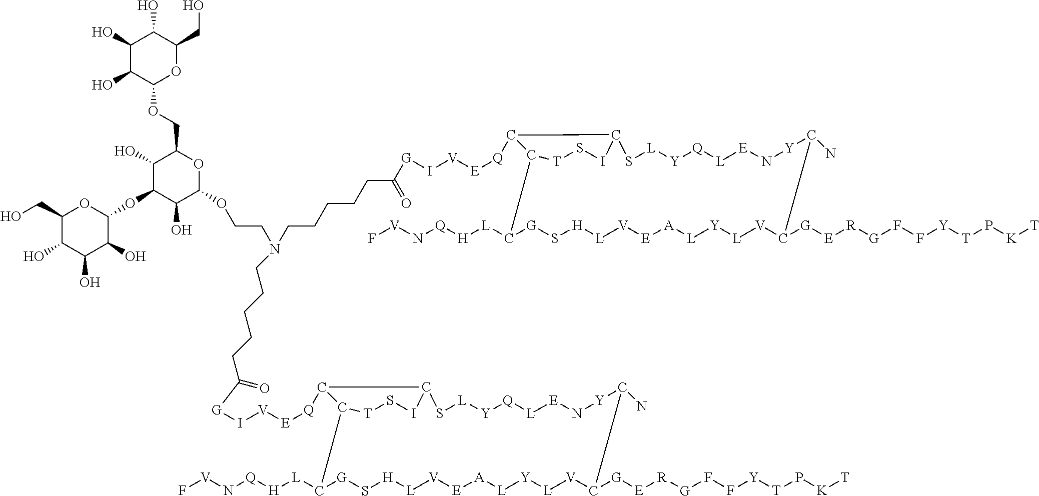 |
| 112 | 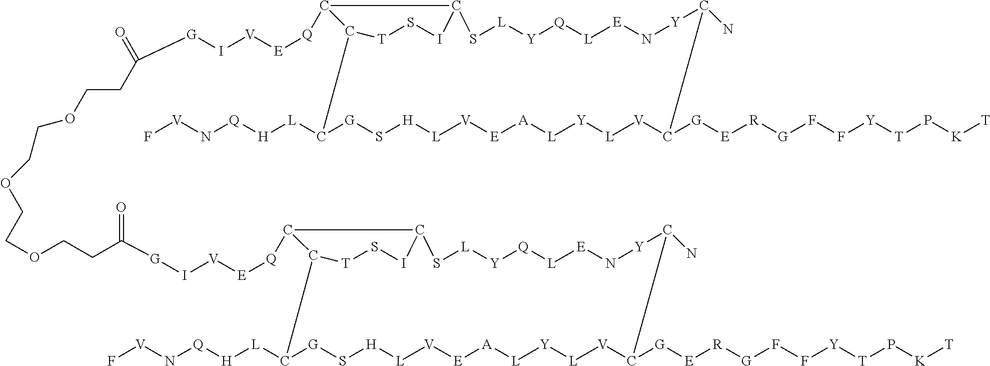 |
| 113 | 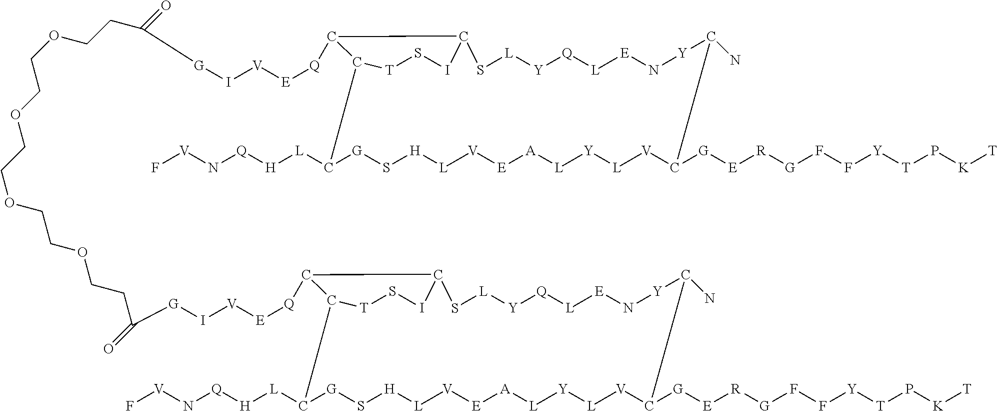 |
| 114 | 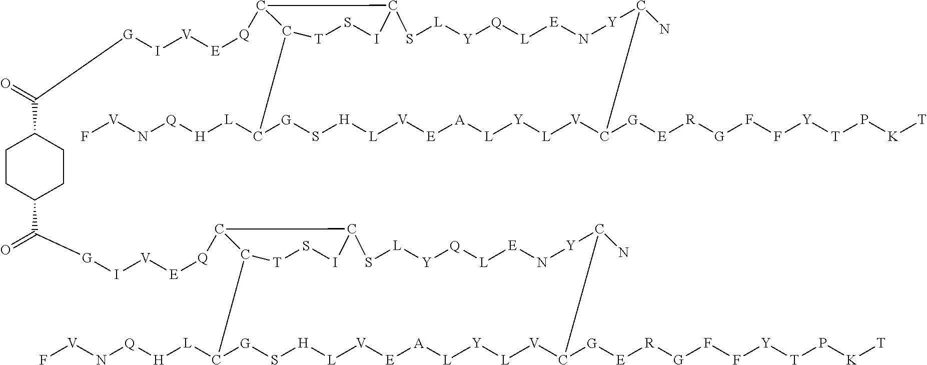 |
| 115 | 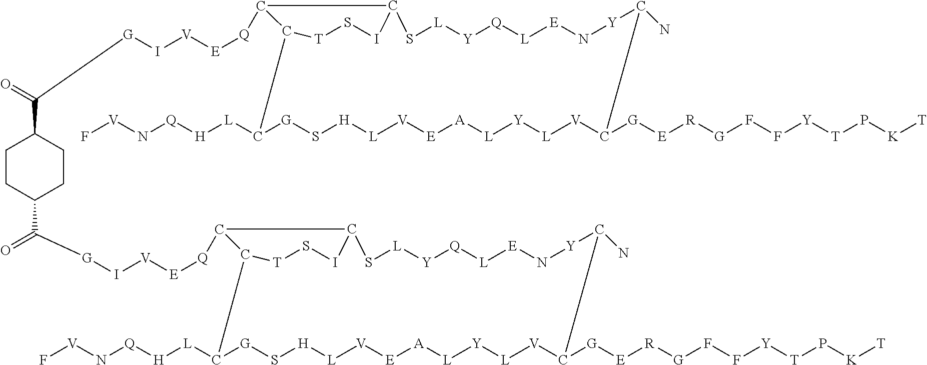 |
| 116 | 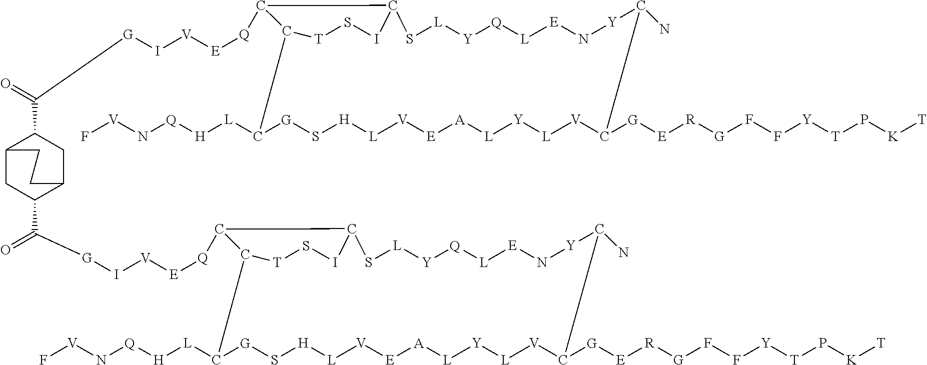 |
| 117 | 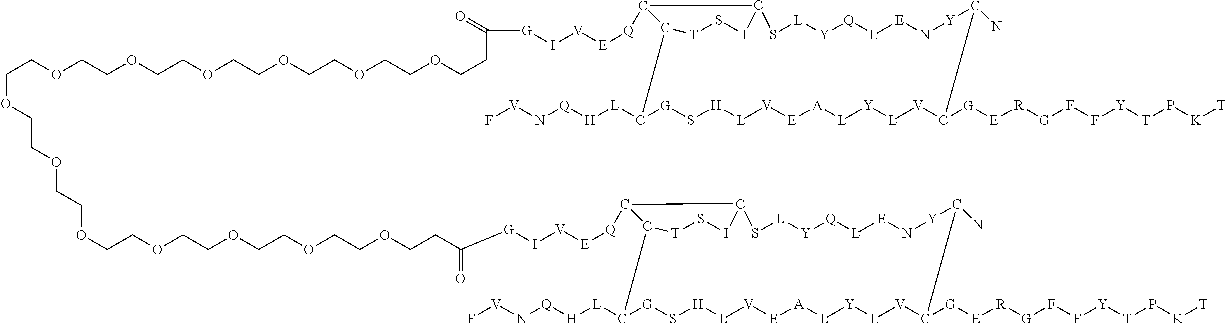 |
| 118 | 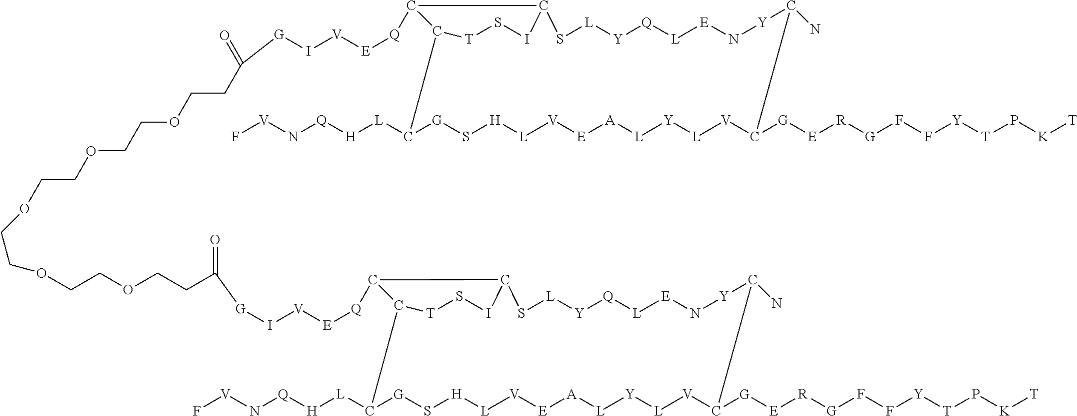 |
| 119 | 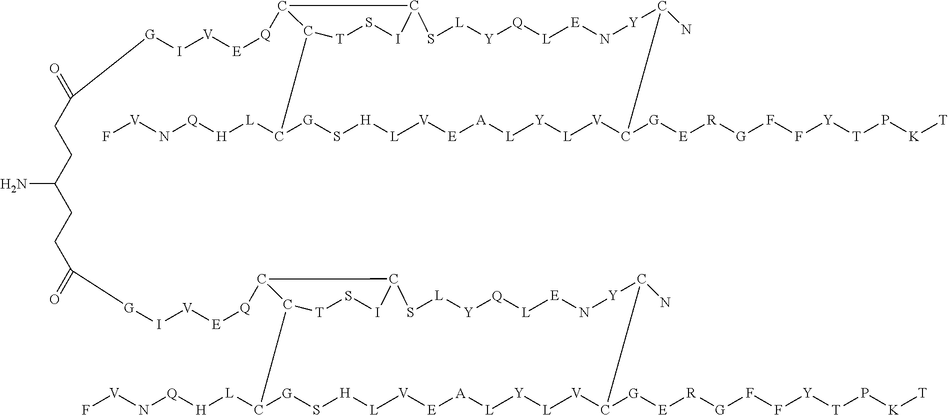 |
| 120 | 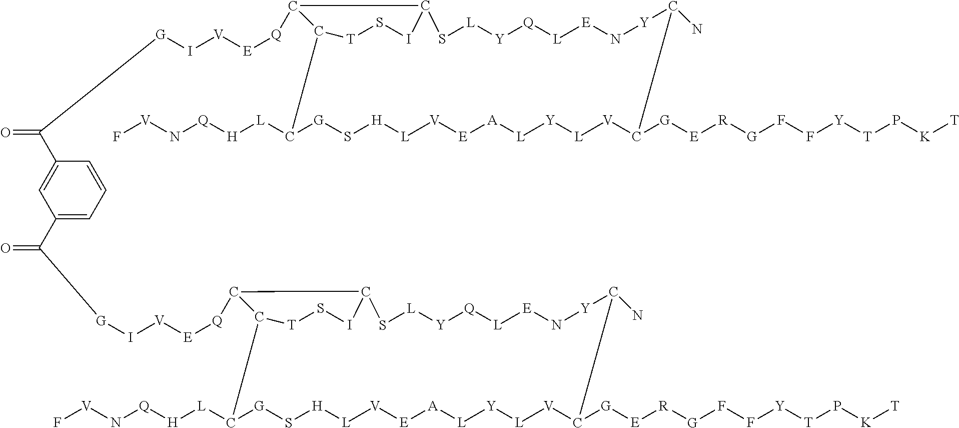 |
| 121 | 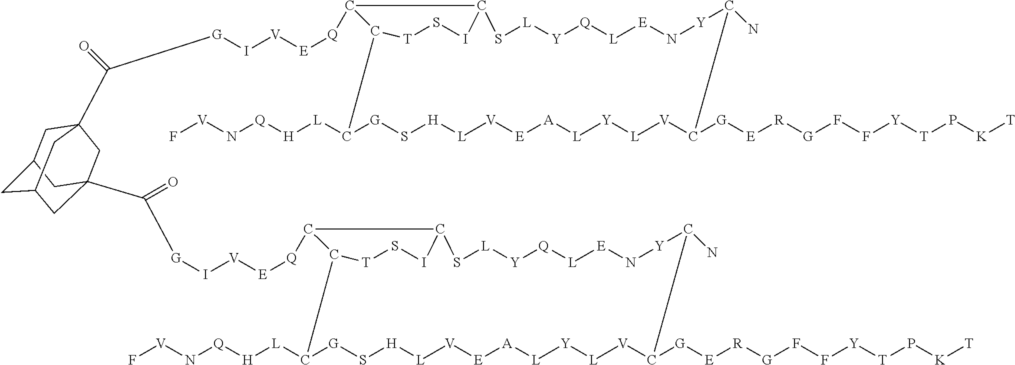 |
| 122 | 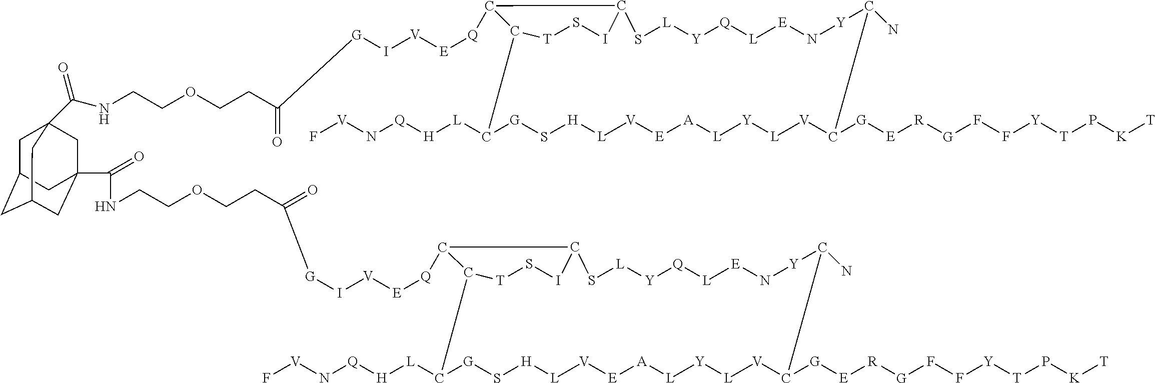 |
| 123 | 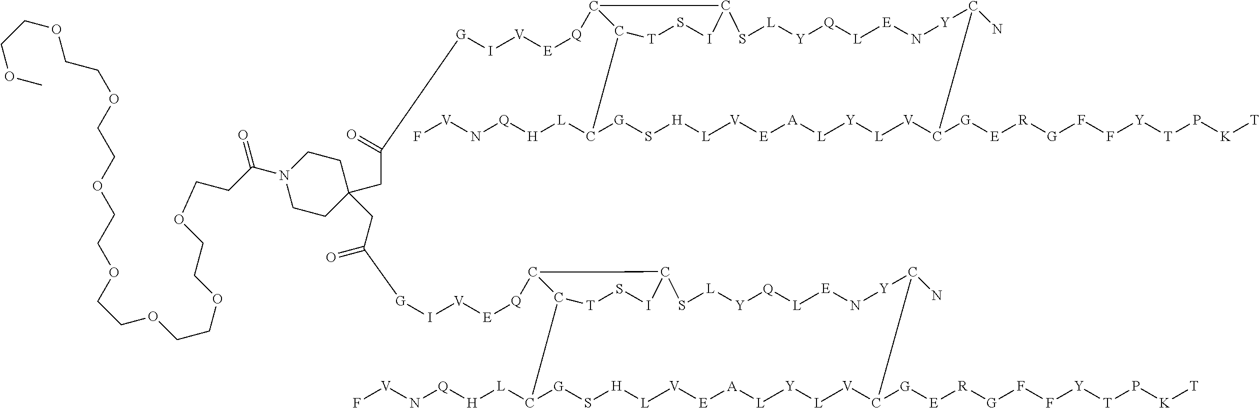 |
| 124 | 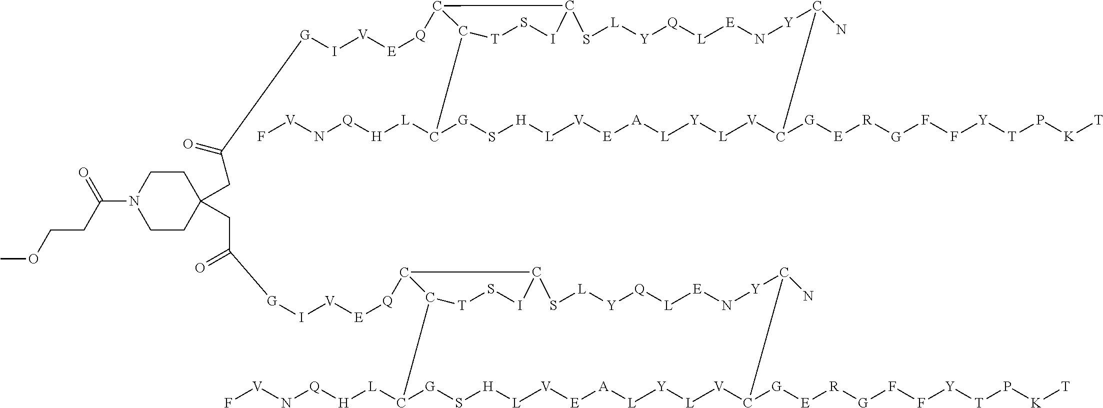 |
| 125 | 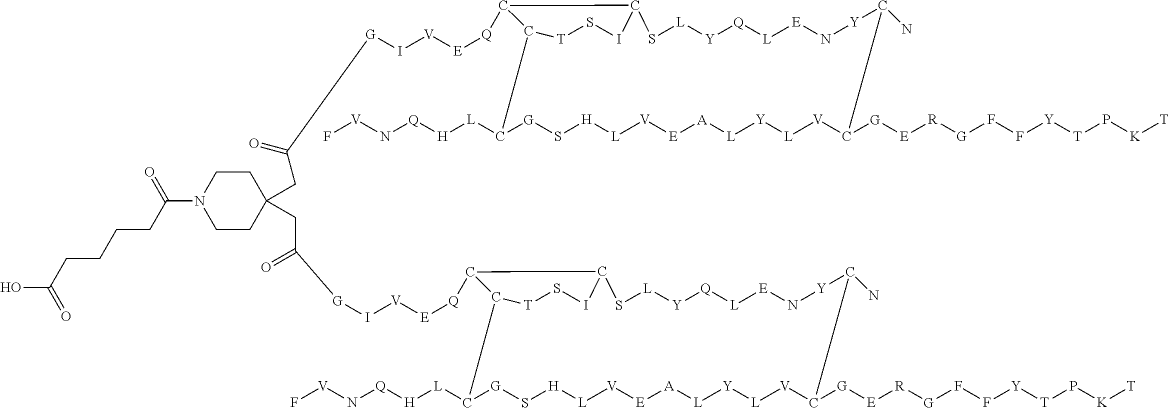 |
| 126 | 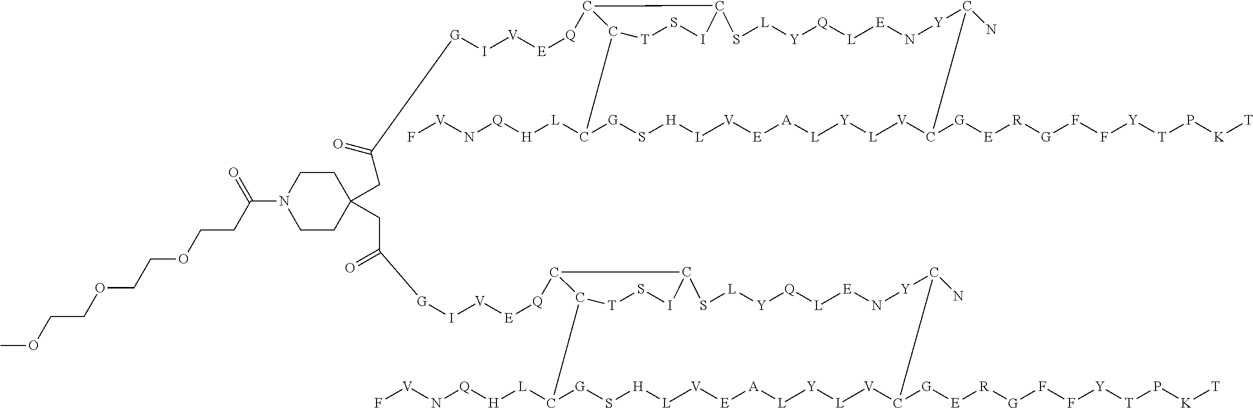 |
| 127 | 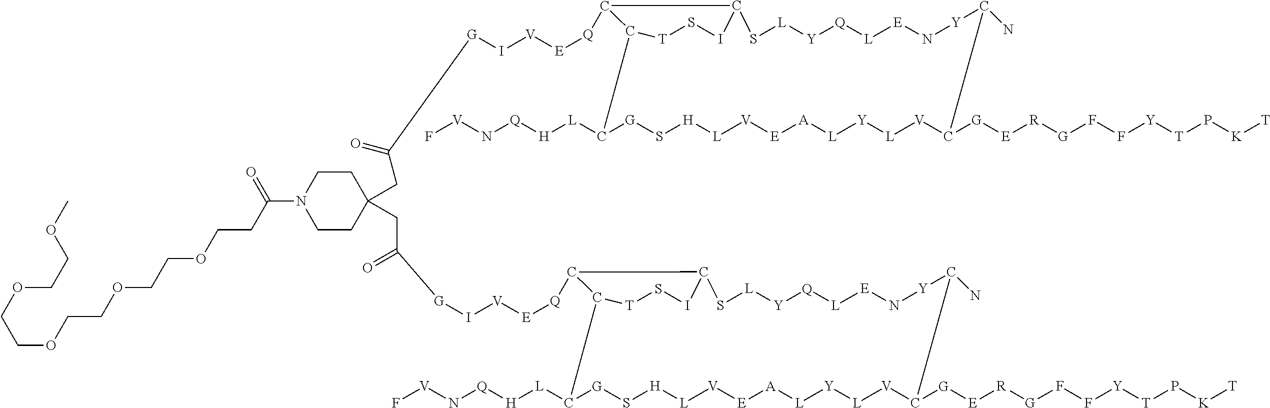 |
| 128 | 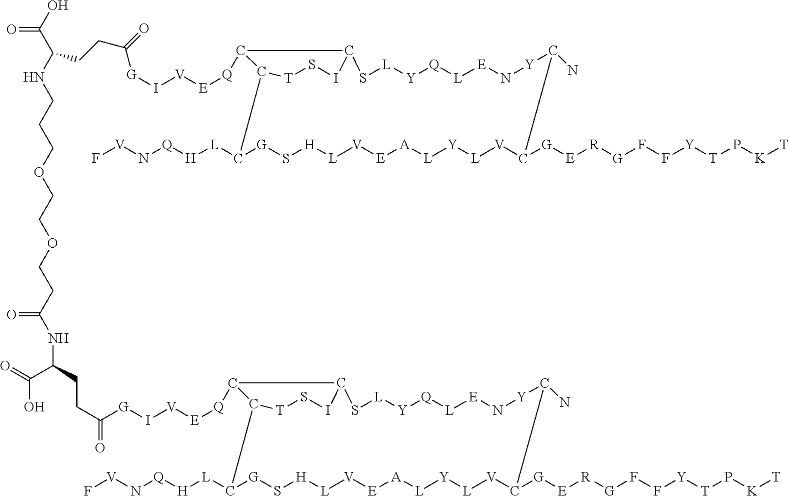 |
| 129 | 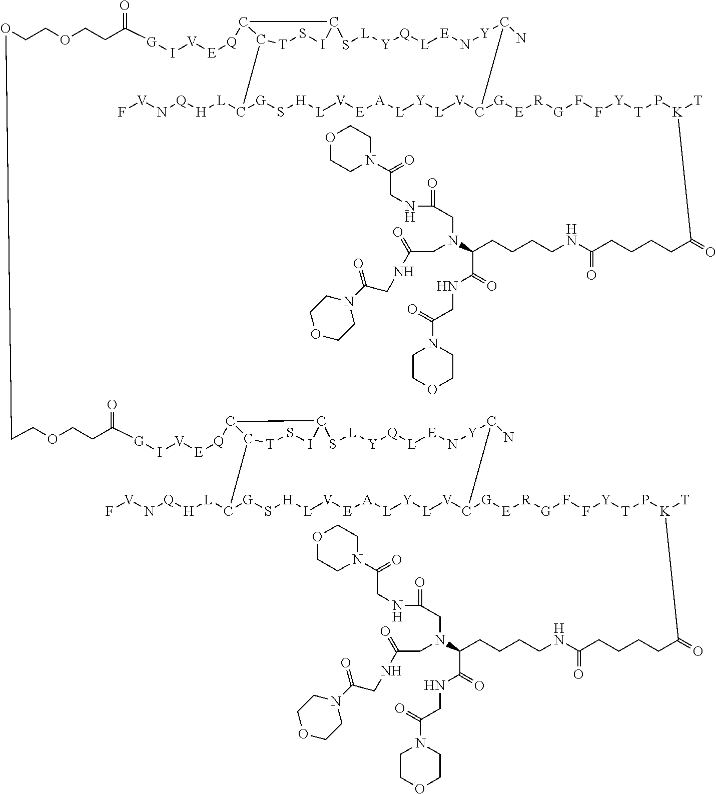 |
| 130 | 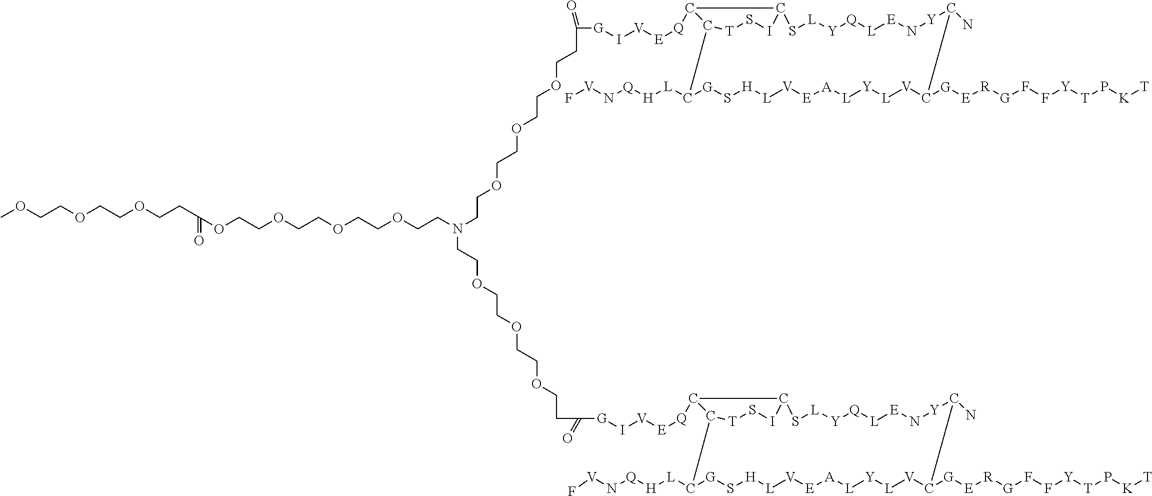 |
| 131 | 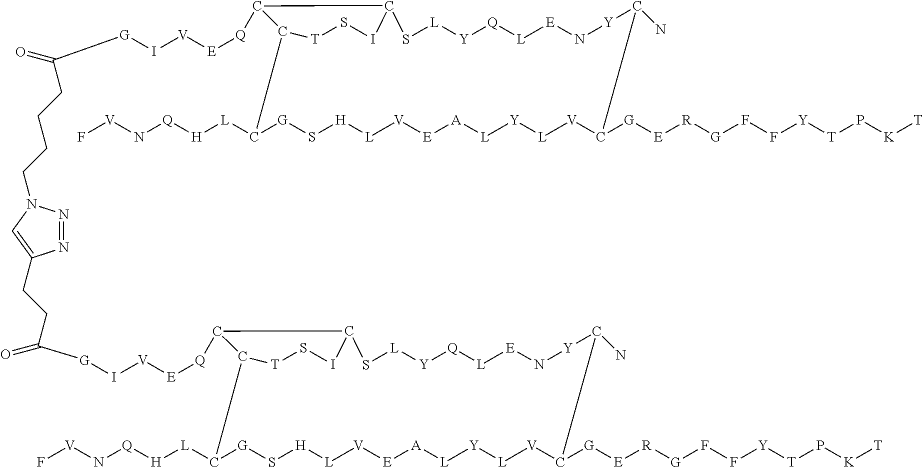 |
| 132 | 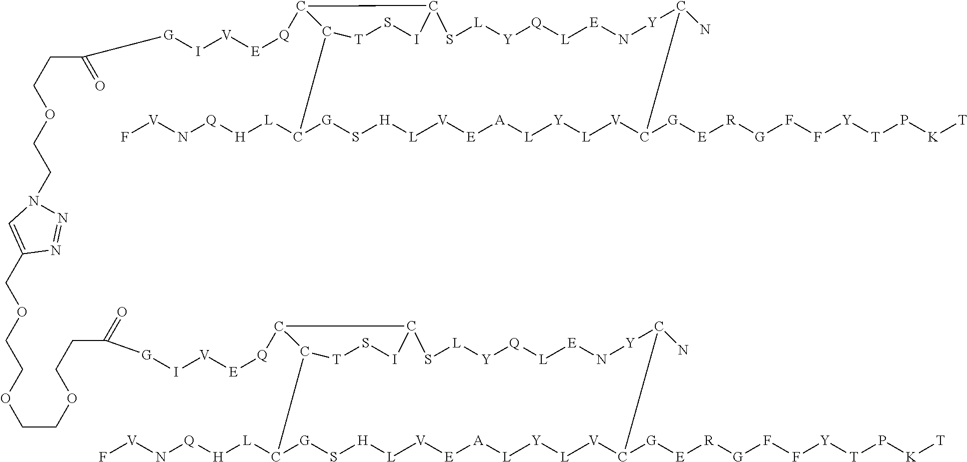 |
| 133 | 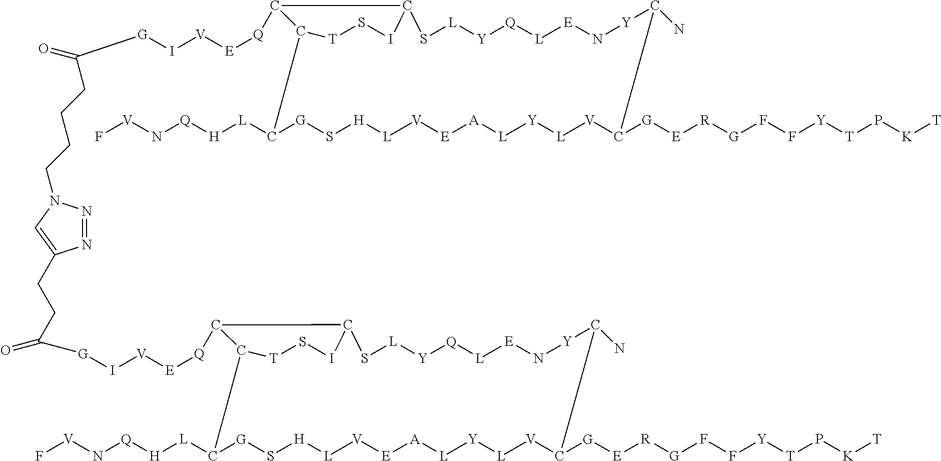 |
| 134 | 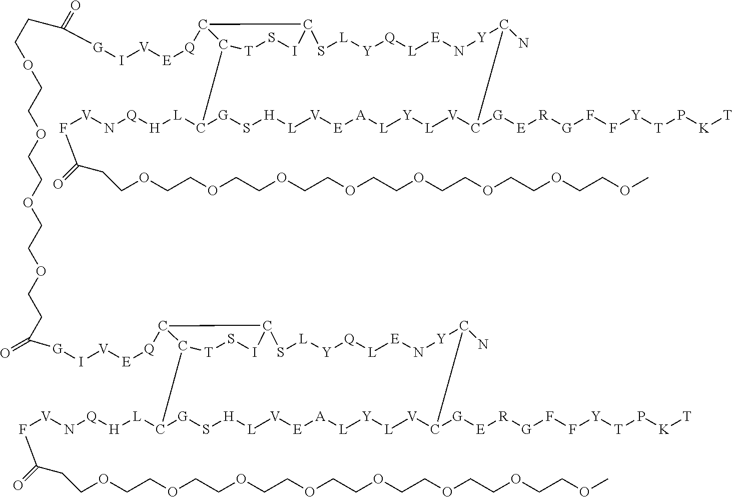 |
| 135 | 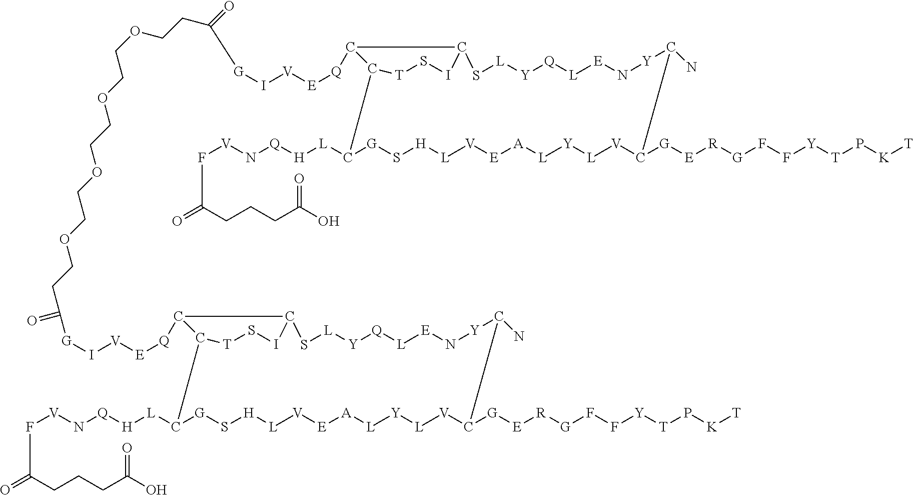 |
| 136 | 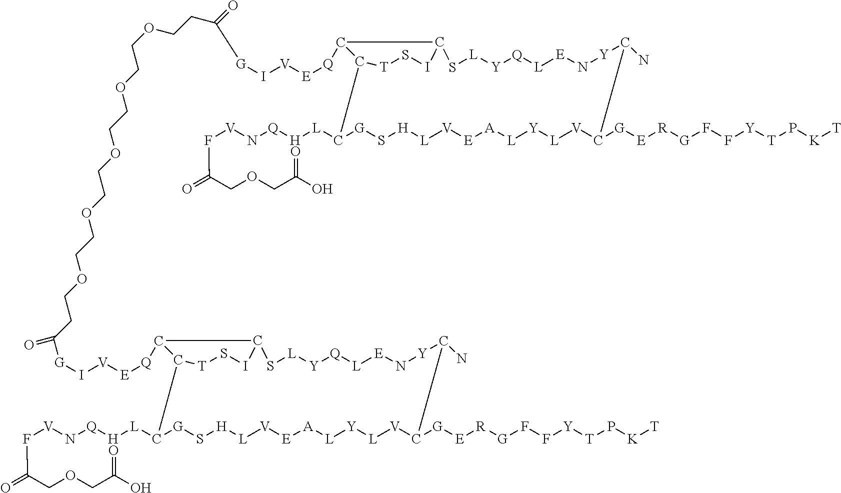 |
| 137 | 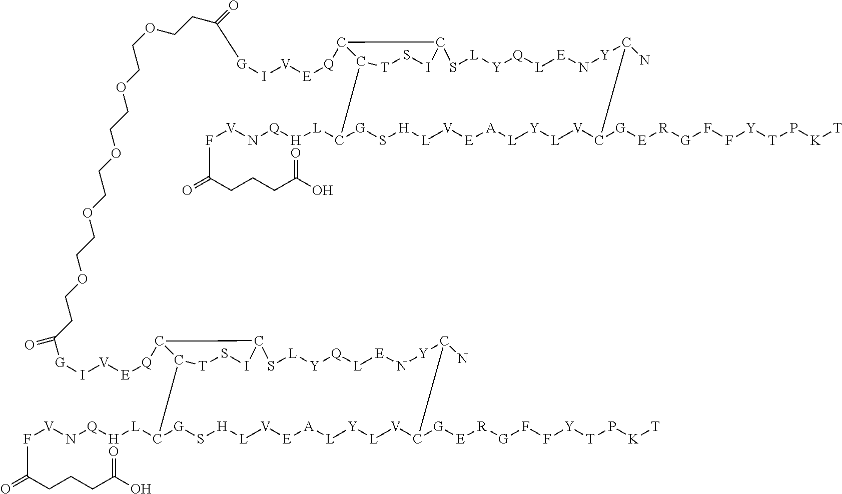 |
| 138 | 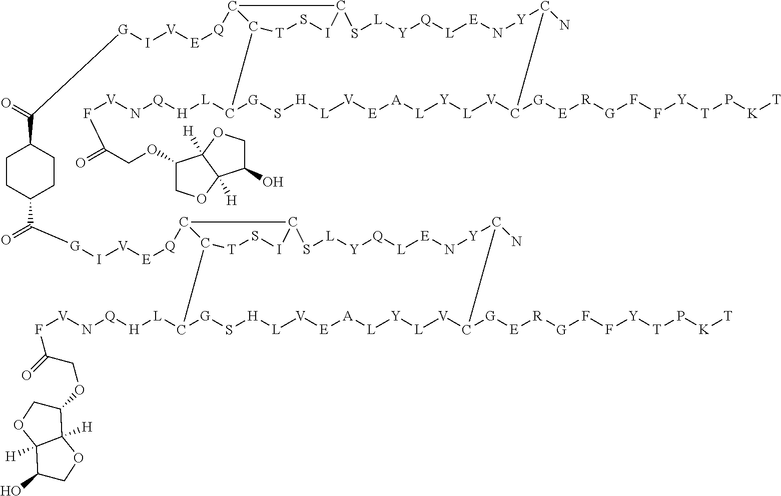 |
| 139 | 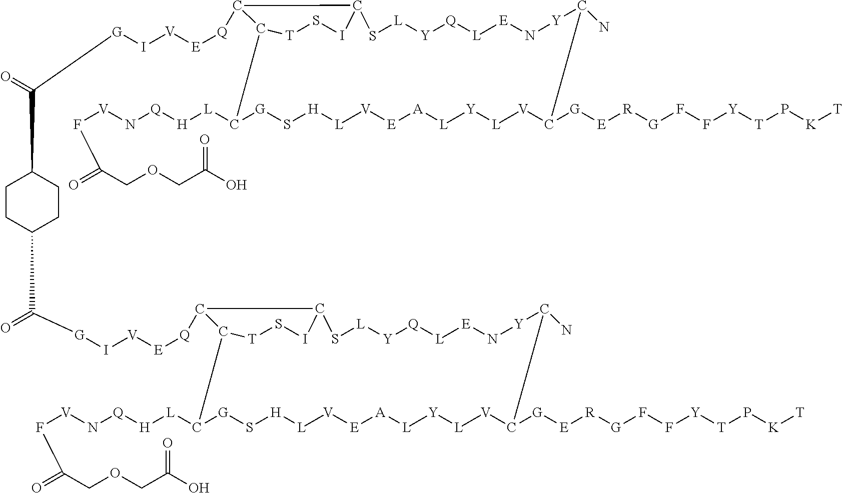 |
| 140 | 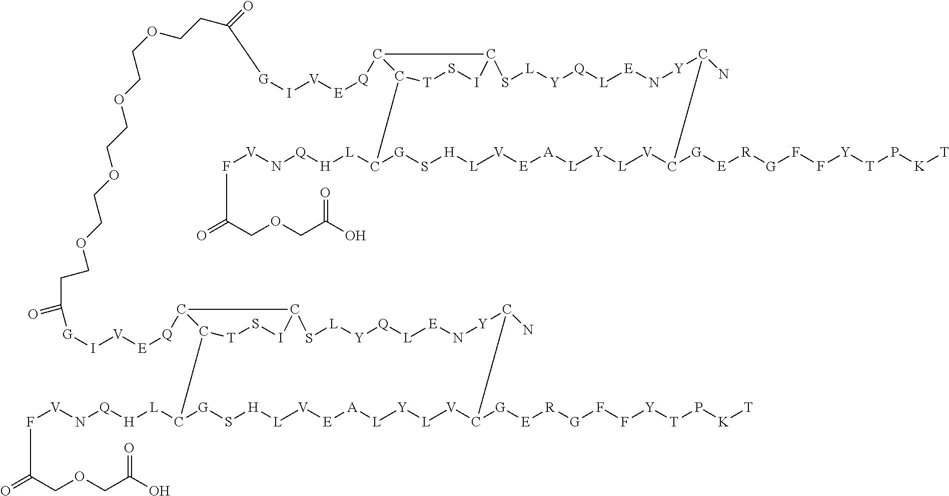 |
| 141 | 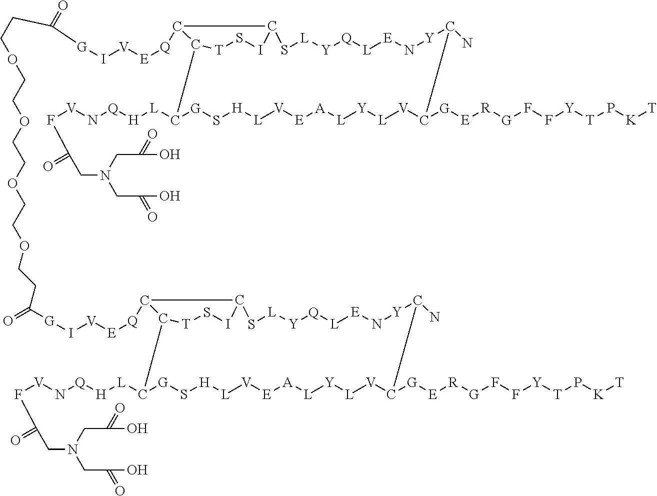 |
| 142 | 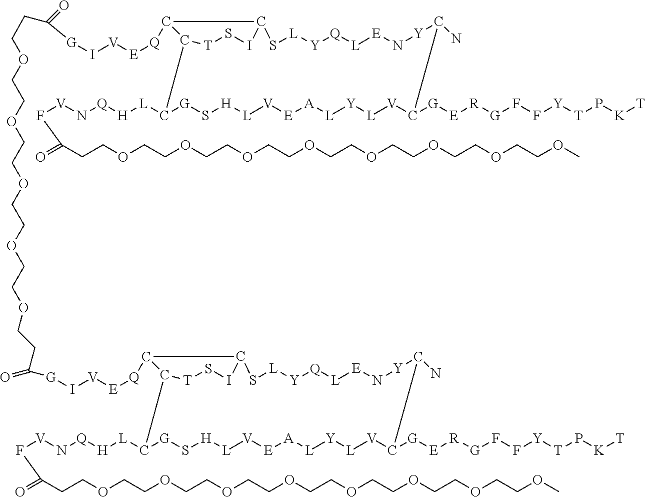 |
| 143 | 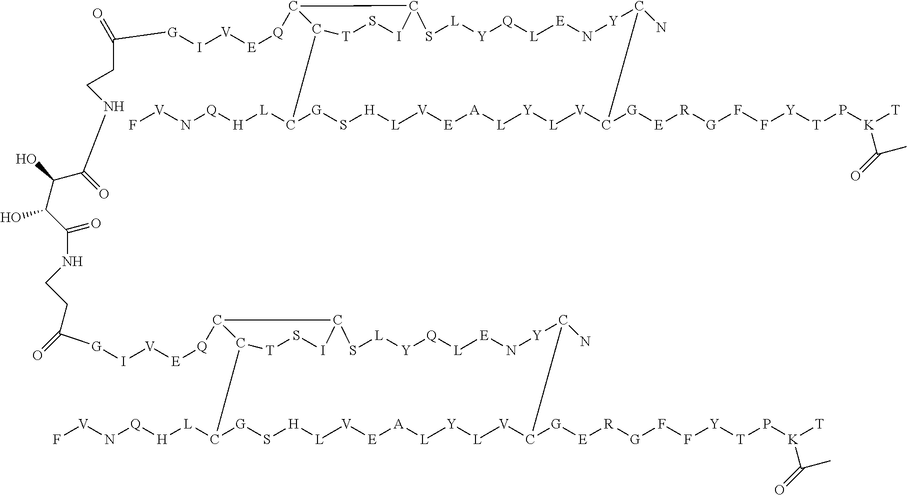 |
| 144 | 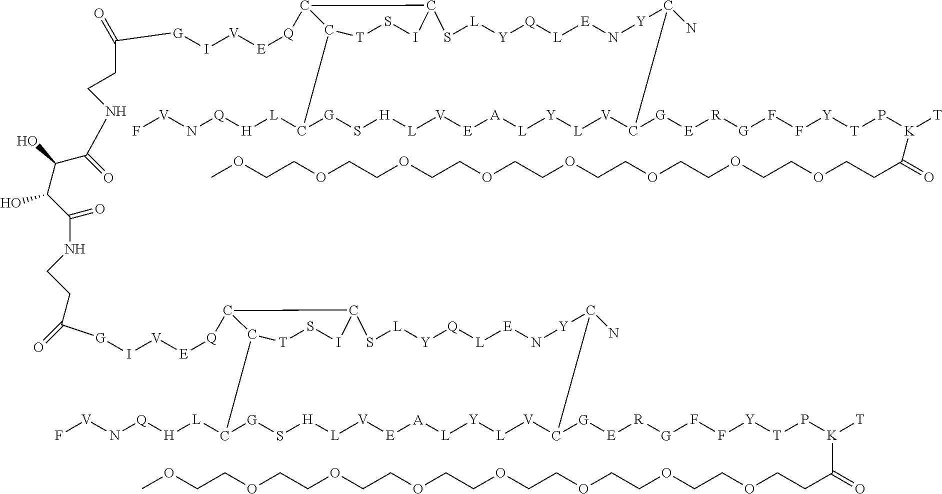 |

wherein the dimers comprise disulfide linkages between the Cys₆ and Cys₁₁ residues of the A-chain polypeptide and between the Cys₇ and Cys₂₀ of the A-chain to the Cys₇ and Cys₁₉ of the B-chain polypeptide, respectively; wherein the linking moieties Z are covalently linked to the α-amino groups of the A1 residue of each insulin, wherein each of the amines of the B-29 or B-28 lysine and the N-terminal amino acids of each of the B-chain of the two insulins, represented by X, U, X′ and U′ optionally and independently are conjugated with a capping group and wherein the A-chain polypeptide for Dimers 1, 3, 4, 6-132 and 135-144 has the amino acid sequence shown in SEQ ID NO:1; the B-chain polypeptide Dimers 1-144 has the amino acid sequence shown in SEQ ID NO:2, as depicted by Formula I herein, or wherein the A-chain polypeptide for Dimer s 2 and 5 has the amino acid sequence shown ins SEQ ID NO: 1 and the B-chain polypeptide Dimers 2 and 5 has the amino acid sequence shown in SEQ ID NO: 11, or wherein the A-chain polypeptide for Dimers 6 and 7 has the amino acid sequence shown ins SEQ ID NO: 1 and the B-chain polypeptide Dimers 6 and 7 has the amino acid sequence shown in SEQ ID NO:12, or wherein the A-chain polypeptide for Dimer 133 has the amino acid sequence shown ins SEQ ID NO: 10 and the B-chain polypeptide Dimer 133 has the amino acid sequence shown in SEQ ID NO:2.

### Pharmaceutical Compositions

In accordance with one embodiment a pharmaceutical composition is provided comprising any of the novel insulin dimers disclosed herein, preferably at a purity level of at least 90%, 91%, 92%, 93%, 94%, 95%, 96%, 97%, 98% or 99%, and a pharmaceutically acceptable diluent, carrier or excipient. Such compositions may contain an insulin dimer as disclosed herein at a concentration of at least 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or higher. In one embodiment the pharmaceutical compositions comprise aqueous solutions that are sterilized and optionally stored contained within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

The disclosed insulin dimers are believed to be suitable for any use that has previously been described for insulin peptides. Accordingly, the insulin dimers disclosed herein can be used to treat hyperglycemia, or treat other metabolic diseases that result from high blood glucose levels. Accordingly, the present invention encompasses pharmaceutical compositions comprising a insulin dimers as disclosed herein and a pharmaceutically acceptable carrier for use in treating a patient suffering from high blood glucose levels. In accordance with one embodiment the patient to be treated using a insulin dimer disclosed herein is a domesticated animal, and in another embodiment the patient to be treated is a human.

One method of treating hyperglycemia in accordance with the present disclosure comprises the steps of administering the presently disclosed insulin dimers to a patient using any standard route of administration, including parenterally, such as intravenously, intraperitoneally, subcutaneously or intramuscularly, intrathecally, transdermally, rectally, orally, nasally or by inhalation. In one embodiment the composition is administered subcutaneously or intramuscularly. In one embodiment, the composition is administered parenterally and the insulin polypeptide, or prodrug derivative thereof, is prepackaged in a syringe.

The insulin dimers disclosed herein may be administered alone or in combination with other anti-diabetic agents. Anti-diabetic agents known in the art or under investigation include native insulin, native glucagon and functional analogs thereof, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARγ inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; or FBPase (fructose 1,6-bisphosphatase) inhibitors.

Pharmaceutical compositions comprising the insulin dimers disclosed herein can be formulated and administered to patients using standard pharmaceutically acceptable carriers and routes of administration known to those skilled in the art. Accordingly, the present disclosure also encompasses pharmaceutical compositions comprising one or more of the insulin dimers disclosed herein, or a pharmaceutically acceptable salt thereof, in combination with a pharmaceutically acceptable carrier. For example, the pharmaceutical compositions comprising the insulin dimers disclosed herein may optionally contain zinc ions, preservatives (e.g., phenol, cresol, parabens), isotonicizing agents (e.g., mannitol, sorbitol, lactose, dextrose, trehalose, sodium chloride, glycerol), buffer substances, salts, acids and alkalis and also further excipients. These substances can in each case be present individually or alternatively as mixtures. Glycerol, dextrose, lactose, sorbitol and mannitol are customarily present in the pharmaceutical preparation in a concentration of 100-250 mM, NaCl in a concentration of up to 150 mM. Buffer substances, such as, for example, phosphate, acetate, citrate, arginine, glycylglycine or TRIS (i.e. 2-amino-2-hydroxymethyl-1,3-propanediol) buffer and corresponding salts, are present in a concentration of 5-250 mM, commonly from about 10-100 mM. Further excipients can be, inter alia, salts or arginine.

In one embodiment the pharmaceutical composition comprises a 1 mg/mL concentration of the insulin dimer at a pH of about 4.0 to about 7.0 in a phosphate buffer system.

The pharmaceutical compositions may comprise the insulin dimer as the sole pharmaceutically active component, or the insulin dimer can be combined with one or more additional active agents.

All therapeutic methods, pharmaceutical compositions, kits and other similar embodiments described herein contemplate that insulin dimers include all pharmaceutically acceptable salts thereof.

In one embodiment the kit is provided with a device for administering the insulin dimers composition to a patient. The kit may further include a variety of containers, e.g., vials, tubes, bottles, and the like. Preferably, the kits will also include instructions for use. In accordance with one embodiment the device of the kit is an aerosol dispensing device, wherein the composition is prepackaged within the aerosol device. In another embodiment the kit comprises a syringe and a needle, and in one embodiment the insulin dimer composition is prepackaged within the syringe.

The compounds of this invention may be prepared by standard synthetic methods, recombinant DNA techniques, or any other methods of preparing peptides and fusion proteins. Although certain non-natural amino acids cannot be expressed by standard recombinant DNA techniques, techniques for their preparation are known in the art. Compounds of this invention that encompass non-peptide portions may be synthesized by standard organic chemistry reactions, in addition to standard peptide chemistry reactions when applicable.

The following examples are intended to promote a further understanding of the present invention.

### EXAMPLES

### General Procedures

All chemicals were purchased from commercial sources, unless otherwise noted. Reactions were usually carried out at ambient temperature or at room temperature unless otherwise noted. Reactions sensitive to moisture or air were performed under nitrogen or argon using anhydrous solvents and reagents. The progress of reactions was monitored by analytical thin layer chromatography (TLC) reagents, and ultra performance liquid chromatography-mass spectrometry (UPLC-MS). TLC was performed on E. Merck TLC plates precoated with silica gel 60F-254, layer thickness 0.25 mm. The plates were visualized using 254 nm UV and/or by exposure to cerium ammonium molybdate (CAM) or p-anisaldehyde staining solutions followed by charring. Ultra performance liquid chromatography (UPLC) was performed on a Waters Acquity™ UPLC® system.

UPLC-MS Method A: Waters Acquity™ UPLC® BEH C18 1.7 μm 1.0×50 mm column with gradient 10:90-95:5 v/v CH₃CN/H₂O+v 0.05% TFA over 2.0 min; flow rate 0.3 mL/min, UV wavelength 215 nm; UPLC-MS;

Method B: Waters Acquity™ UPLC® BEH C18 1.7 μm 2.1×100 mm column with gradient 60:40-100:0 v/v CH₃CN/H₂O+v 0.05% TFA over 4.0 min and 100:0-95:5 v/v CH₃CN/H₂O+v 0.05% TFA over 40 sec; flow rate 0.3 mL/min, UV wavelength 200-300 nm; UPLC-MS;

Method C: Waters Acquity™ UPLC® BEH C18 1.7 μm 2.1×100 mm column with gradient 20:80-90:10 v/v CH₃CN/H₂O+v 0.05% TFA over 4.0 min and 90:10-95:5 v/v CH₃CN/H₂O+v 0.05% TFA over 0.5 min; flow rate 0.3 mL/min, UV wavelength 200-300 nm; UPLC-MS;

Method D: Waters Acquity™ UPLC® BEH C8 1.7 μm 2.1×100 mm column with gradient 10:90-55:45 v/v CH₃CN/H₂O+v 0.05% TFA over 4.0 min and 55:45-95:5 v/v CH₃CN/H₂O+v 0.05% TFA over 40 sec; flow rate 0.3 mL/min, UV wavelength 200-300 nm; UPLC-MS;

Method E: Waters Acquity™ UPLC® BEH300 C4 1.7 μm 2.1×100 mm column with gradient 10:90-50:50 v/v CH₃CN/H₂O+v 0.05% TFA over 4.3 min and 50:50-70:30 v/v CH₃CN/H₂O+v 0.05% TFA over 0.5 min; flow rate 0.3 mL/min, UV wavelength 200-300 nm; UPLC-MS;

Method F: Waters Acquity™ UPLC® BEH C8 1.7 μm 2.1×100 mm column with gradient 20:80-72.5:27.5 v/v CH₃CN/H₂O+v 0.05% TFA over 4.3 min and 72.5:27.5-95:5 v/v CH₃CN/H₂O+v 0.05% TFA over 0.5 min; flow rate 0.3 mL/min, UV wavelength 200-300 nm, and UPLC-MS;

Method G: Waters Acquity™ UPLC® BEH C8 1.7 μm 2.1×100 mm column with gradient 20:80-90:10 v/v CH₃CN/H₂O+v 0.1% TFA over 4.0 min and 90:10-95:5 v/v CH₃CN/H₂O+v 0.1% TFA over 0.4 min; flow rate 0.3 mL/min, UV wavelength 200-300 nm.

Mass analysis was performed on a Waters SQ Detector with electrospray ionization in positive ion detection mode and the scan range of the mass-to-charge ratio was 170-900 or a Waters Micromass® LCT Premier™ XE with electrospray ionization in positive ion detection mode and the scan range of the mass-to-charge ratio was 300-2000. The identification of the produced insulin conjugates or IRPA was confirmed by comparing the theoretical molecular weight to the experimental value that was measured using UPLC-MS. For the determination of the linkage positions, specifically, insulin dimers were subjected to DTT treatment (for a/b chain) or Glu-C digestion (with or without reduction and alkylation), and then the resulting peptides were analyzed by LC-MS. Based on the measured masses, the linkage positions were deduced.

Flash chromatography was performed using either a Biotage Flash Chromatography apparatus (Dyax Corp.) or a CombiFlash® Rf instrument (Teledyne Isco). Normal-phase chromatography was carried out on silica gel (20-70 μm, 60 Å pore size) in pre-packed cartridges of the size noted. Ion exchange chromatography was carried out on a silica-based material with a bonded coating of a hydrophilic, anionic poly(2-sulfoethyl aspartamide) (PolySULFOETHYL A column, PolyLC Inc., 250×21 mm, 5 □m, 1000 Å pore size). Reverse-phase chromatography was carried out on C18-bonded silica gel (20-60 μm, 60-100 Å pore size) in pre-packed cartridges of the size noted. Preparative scale HPLC was performed on Gilson 333-334 binary system using Waters DELTA PAK C4 15 μm, 300 Å, 50×250 mm column or KROMASIL® C8 10 μm, 100 Å, 50×250 mm column, flow rate 85 mL/min, with gradient noted. Concentration of solutions was carried out on a rotary evaporator under reduced pressure or freeze-dried on a VirTis Freezemobile Freeze Dryer (SP Scientific).

Abbreviations: acetonitrile (AcCN), aqueous (aq), 1-[bis(dimethylamino)methylene]-1H-1,2,3-triazolo[4,5-b]pyridinium 3-oxid hexafluorophosphate (HATU), dichloromethane (DCM), 4-dimethylaminopyridine (DMAP), N,N-diisopropylethylamine or Hünig's base (DIPEA), N,N-dimethylacetamide (DMA), N,N-dimethylformamide (DMF), dimethyl sulfoxide (DMSO), ethyl acetate (EtOAc), N-(3-dimethylaminopropyl)-N′-ethylcarbodiimide hydrochloride (EDC), gram(s) (g), 1-hydroxybenzotriazole hydrate (HOBt), hour(s) (h or hr), isopropyl acetate (IPAc), mass spectrum (ms or MS), methyl tert-butyl ether (MTBE), microgram(s) (μg), microliter(s) (μL), micromole (μmol), milligram(s) (mg), milliliter(s) (mL), millimole (mmol), minute(s) (min), retention time (t_{R} or Rt), room temperature (rt), saturated (sat. or sat′d), saturated aq sodium chloride solution (brine), 1,1,3,3-tetramethylguanidine (TMG), 2,2,6,6-tetramethylpiperidine (TMP), triethylamine (TEA), trifluoroacetic acid (TFA), and N,N,N′,N′-tetramethyl-O—(N-succinimidyl)uronium tetrafluoroborate (TSTU).

The term “RHI” refers to recombinant human insulin and is used to indicate that the insulin has the amino acid sequence characteristic of native, wild-type human insulin. As used herein in the tables, the term indicates that the amino acid sequence of the insulin comprising the dimer is that of native, wild-type human insulin.

Linking Reagents 1 through 9 below are commercially available and can be obtained, for example, from Quanta Biodesign LTD (Plain City, Ohio).

### Preparative Example 1

### Synthesis of 2,5-dioxopyrrolidin-1-yl 6-((6-((2,5-dioxopyrrolidin-1-yl)oxy)-6-oxohexyl)amino)-6-oxohexanoate (Linking Reagent 1 is Described

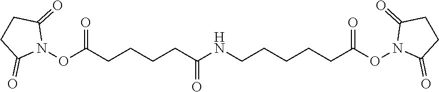 [SMILES: O=C(CCCCC(=O)ON1C(=O)CCC1=O)NCCCCCC(=O)ON1C(=O)CCC1=O]

### Step 1 Benzyl 6-((6-(benzyloxy)-6-oxohexyl)amino)-6-oxohexanoate

To a mixture of adipic acid monobenzyl ester (600 mg, 2.54 mmol) and 6-(benzyloxy)-6-oxohexan-1-aminium 4-methylbenzenesulfonate (1.0 g, 2.54 mmol) in DMF (12.71 mL) was added HOBt (584 mg, 3.81 mmol), Hunig's base (888 μL, 5.08 mmol), and EDC (731 mg, 3.81 mmol). After stirring overnight, the reaction mixture was partitioned between sat. NaHCO₃ and EtOAc. The organic phase was separated, washed with 1.0 M HCl and brine, dried over Na₂SO₄, and concentrated to give the title compound as a semi-solid and used in the next step without further purification. UPLC-MS Method A: Rt=1.26 min, m/z=440.3 [M+1].

### Step 2 6-((5-Carboxypentyl)amino)-6-oxohexanoic acid

A suspension of the product of Step 1 (1.08 g, 2.457 mmol) and Pearlman's catalyst (20% wt on carbon, 173 mg, 0.246 mmol) in MeOH (50 mL) was stirred under 50 psi H₂ overnight. The catalyst was filtered off and the filtrate was subjected to reverse-phase chromatography on C8 phase (Kromasil, C8 101□m 100 Å, 250×50 mm; solvent A=water/0.05% TFA, solvent B=AcCN/0.05% TFA), flow rate=85 mL/min, gradient B in A 5-30% in 30 min. UPLC-MS Method A: Rt=0.40 min, m/z=260.15 [M+1].

### Step 3 2,5-dioxopyrrolidin-1-yl 6-((6-((2,5-dioxopyrrolidin-1-yl)oxy)-6-oxohexyl)amino)-6-oxohexanoate

To a solution of the product of Step 2 (50 mg, 0.193 mmol) in DMF (964 μL) was added TSTU (116 mg, 0.386 mmol). After cooling down to 0° C., to the mixture was added triethylamine (53.8 μL, 0.386 mmol). After stirring for 45 minutes, formation of the desired compound was observed: UPLC-MS Method A: Rt=0.71 min, m/z=453.4 [M+1]. The resulting 2,5-dioxopyrrolidin-1-yl 6-((6-((2,5-dioxopyrrolidin-1-yl)oxy)-6-oxohexyl)amino)-6-oxohexanoate was used as 0.2 M solution in DMF without purification.

### Preparative Example 2

Synthesis of bis(2,5-dioxopyrrolidin-1-yl) (1S,4S)-cyclohexane-1,4-dicarboxylate (Linking reagent 2) a solution of trans-1,2-cyclohexane-1,4-dicarboxylic acid (200 mg, 1.162 mmol) in DCM (I1 mL) at 0° C. was added TSTU (734 mg, 2.439 mmol) and DIPEA (0.5 mL, 2.86 mmol). The resulting reaction mixture was stirred at room temperature for 1 hour. The product was crushed out in reaction solution as white solid; filtered and washed with DCM (2×5 ml); and dried in vacuo to obtain the title compound. UPLC-MS Method C: Rt=3.20 min, m/z=367.16 [M+1]. ¹H NMR (500 MHz, DMSO): δ 2.81-2.89 (m; 2H); 2.80 (s; 8H); 2.02-2.10 (m; 4H); 1.57-1.63 (m; 4H).

### Preparative Example 3

### Synthesis of bis(2,5-dioxopyrrolidin-1-yl) (1R,4R)-cyclohexane-1,4-dicarboxylate (Linking Reagent 3) is Described

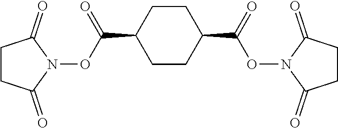 [SMILES: O=C1CCC(=O)N1OC(=O)[C@H]1CC[C@@H](C(=O)ON2C(=O)CCC2=O)CC1]

To a solution of (1R,4R)-cyclohexane-1,4-dicarboxylic acid (200 mg, 1.162 mmol) in DCM (11 mL) at 0° C. was added TSTU (734 mg, 2.439 mmol) and DIPEA (0.5 mL, 2.86 mmol). The resulting reaction mixture was stirred at room temperature for 1 hour. The residue was purified by silica gel chromatography (0-100% EtOAc/Hexanes) to provide the title compound. UPLC-MS Method C: Rt=3.17 min, m/z=366.11 [M+1]. ¹H NMR (500 MHz, DMSO): δ 3.02-3.08 (m; 2H); 2.80 (s; 8H); 1.80-1.90 (m; 8H).

### Preparative Example 4

### Synthesis of 1-(tert-butyl) 3,5-bis(2,5-dioxopyrrolidin-1-yl) (3R,5S)-piperidine-1,3,5-tricarboxylate (Linking Reagent 4) is Described

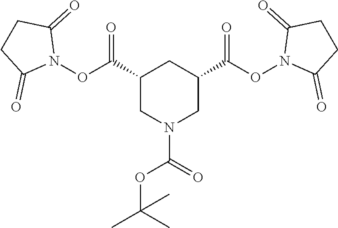 [SMILES: CC(C)(C)OC(=O)N1C[C@H](C(=O)ON2C(=O)CCC2=O)C[C@H](C(=O)ON2C(=O)CCC2=O)C1]

To a solution of (3R,5S)-1-(tert-butoxycarbonyl)piperidine-3,5-dicarboxylic acid (200 mg, 0.734 mmol) in DMF (7 mL) at 0° C. was added TSTU (485 mg, 1.611 mmol) and DIPEA (0.3 mL, 1.718 mmol). The resulting reaction mixture was stirred at room temperature for 2 hour. The residue was purified by silica chromatography (0-100% EtOAc/Hexanes) to provide the title compound. UPLC-MS Method A: Rt=0.98 min, m/z=468.30 [M+1].

### Preparative Example 5

### Synthesis of 2,5-dioxopyrrolidin-1-yl (4-((2,5-dioxopyrrolidin-1-yl)oxy)-4-oxobutanoyl)-L-prolyl-L-prolinate (Linking Reagent 5) is Described

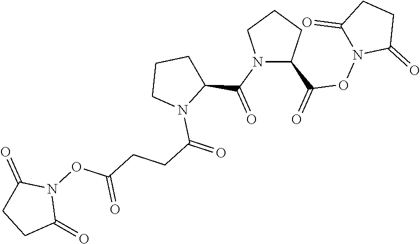 [SMILES: O=C(CCC(=O)N1CCC[C@H]1C(=O)N1CCC[C@H]1C(=O)ON1C(=O)CCC1=O)ON1C(=O)CCC1=O]

### Step 1 (S)-1-((S)-1-(4-(benzyloxy)-4-oxobutanoyl)pyrrolidine-2-carbonyl)pyrrolidine-2-carboxylic acid

To an ice cooled mixture of succinic acid monobenzyl ester (420 mg, 2.02 mmol) in DMF (5 mL), was quickly added a solution TSTU (607 mg, 2.02 mmol) in DMF (5 ml), and the reaction was stirred for 5 mins. DIPEA (0.42 ml, 2.421 mmol) was added and the reaction was stirred for 1 hour at this temperature. L-prolyl-L-proline hydrochloride (502 mg, 2.02 mmol) was added as a solid in one lot. The reaction mixture appeared as a suspension. Additional DIPEA (0.846 ml, 4.84 mmol) was added and the reaction was continued stirring overnight, over which period the mixture was allowed to warm up to room temp. At this point, all suspension dissolved. By LCMS the target product was observed. Solution was concentrated. Material was redissolved in small amount of water and purified by reverse-phase chromatography on Biotage C18 column, eluting with 0-70% AcN/H2O. UPLC-MS Method A: Rt=0.92 min, m/z=403.3 [M+1].

### Step 2 (3-carboxypropanoyl)-L-prolyl-L-proline

Dissolved the product of Step 1 (457 mg, 1.14 mmol) in acetone (5 mL) and added Pd/C (10% wt on carbon, 60.4 mg, 0.057 mmol) and attached a hydrogen balloon. Ambient air was evacuated and replaced by hydrogen and the reaction was stirred at rt for 3 hours. The catalyst was filtered off and the filtrate was concentrated. The obtained solid was redisolved in 30% AcN/H2O, 0.1% TFA (50 mL) and lyophilized overnight. Material was used without further purification. UPLC-MS Method A: Rt=0.32 min, m/z=313.10 [M+1].

### Step 3 2,5-dioxopyrrolidin-1-yl (4-((2,5-dioxopyrrolidin-1-yl)oxy)-4-oxobutanoyl)-L-prolyl-L-prolinate

To a solution of the product of Step 2 (310 mg, 0.993 mmol) in DMSO (2.0 mL) was added TSTU (747 mg, 2.48 mmol). After cooling to 0° C., to the mixture was added triethylamine (0.418 ml, 2.98 mmol). After stirring for 2 hours at rt, formation of the desired compound was observed. TFA (0.306 ml, 3.97 mmol) was added and the solution was diluted with water and added to C18 column, and elution with a gradient of 0-80% MeCN/H2O furnished the title compound. UPLC-MS Method A: Rt=0.72 min, m/z=507.16 [M+1].

### Preparative Example 6

### Synthesis of 2,5-dioxopyrrolidin-1-yl 5-((2-((2-((2-((2,5-dioxopyrrolidin-1-yl)oxy)-2-oxoethyl)amino)-2-oxoethyl)amino)-2-oxoethyl)amino)-5-oxopentanoate (Linking Reagent 6) is Described

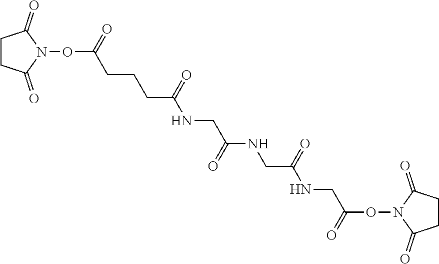 [SMILES: O=C(CCCC(=O)ON1C(=O)CCC1=O)NCC(=O)NCC(=O)NCC(=O)ON1C(=O)CCC1=O]

### Step 1 16,16-Dimethyl-4,7,10,14-tetraoxo-15-oxa-3,6,9-triazaheptadecan-1-oic acid

Using 5-tert-butoxy-5-oxypentanoic acid and 2-(2-(2-aminoacetamido)acetomido)acetic acid, the desired product was prepared in similar fashion to Step 1, Example 5. UPLC-MS Method D: Rt=3.20 min, m/z=360.20 [M+1].

### Step 2 5-((2-((2-((carboxymethyl)amino)-2-oxoethyl)amino)-2-oxoethyl)amino)-5-oxopentanoic acid

### Step 2 5-((2-((2-((Carboxymethyl)amino)-2-oxoethyl)amino)-2-oxoethyl)amino)-5-oxopentanoic acid

Dissolved the product of Step 1 (100 mg, 0.278 mmol) in premixed solution of 1:1 TFA:DCM (1 mL) and the reaction was stirred for 2 hours. The mixture was concentrated and the obtained solid was re-dissolved in 30% MeCN/H2O, 0.1% TFA (20 mL) and lyophilized overnight. Material was used without further purification. UPLC-MS Method (D): Rt=1.04 min, m/z=304.21 [M+1].

### Step 3 2,5-Dioxopyrrolidin-1-yl 5-((2-((2-((2-((2,5-dioxopyrrolidin-1-yl)oxy)-2-oxoethyl)amino)-2-oxoethyl)amino)-2-oxoethyl)amino)-5-oxopentanoate

Using the product of Step 2, the desired compound was prepared in similar fashion to Step 3, Example 5. UPLC-MS Method A: Rt=0.79 min, m/z=498.12 [M+1].

### Preparative Example 7

### Synthesis of bis(2,5-dioxopyrrolidin-1-yl) terephthalate (Linking reagent 7) is Described

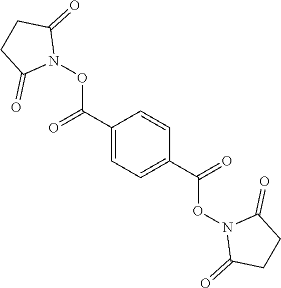 [SMILES: O=C(ON1C(=O)CCC1=O)c1ccc(C(=O)ON2C(=O)CCC2=O)cc1]

Using terephthalic acid, the title product was prepared in similar fashion to Step 3 of Example 5. UPLC-MS Method D: Rt=3.28 min, m/z=721.17 [2M+1].

### Preparative Example 8

### Synthesis of bis(2,5-dioxopyrrolidin-1-yl) isophthalate (bis(2,5-dioxopyrrolidin-1-yl) isophthalate (Linking reagent 8) is Described

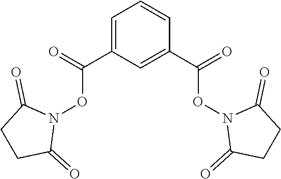 [SMILES: O=C(ON1C(=O)CCC1=O)c1cccc(C(=O)ON2C(=O)CCC2=O)c1]

Using isophthalic acid, the title product was prepared in similar fashion to Step 3 of Example 5. UPLC-MS Method A: Rt=0.73 min, m/z=721.21 [2M+1].

### Preparative Example 9

### Synthesis of (2-(bis(3-((2,5-dioxopyrrolidin-1-yl)oxy)-3-oxopropyl)amino)-2-oxoethyl)phosphonic acid (linking reagent 9) is Described

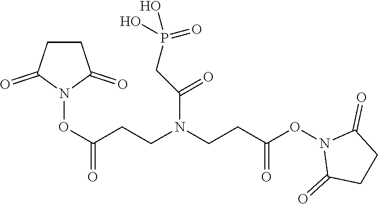 [SMILES: O=C(CCN(CCC(=O)ON1C(=O)CCC1=O)C(=O)CP(=O)(O)O)ON1C(=O)CCC1=O]

### Step 1 2,5-dioxopyrrolidin-1-yl 2-(bis(benzyloxy)phosphoryl)acetate

To a mixture of dibenzylphosphonoacetic acid (500 mg, 1.561 mmol) and TSTU (517 mg, 1.717 mmol) in acetonitrile (7.8 mL) at 0° C. was added dropwise triethylamine (261 μl, 1.873 mmol). Stirred the reaction 2 hrs at 0° C. Concentrated on rotovap using room temperature water bath. Re-dissolved the residue in 100 mL of EtOAc and washed with 50 mL of 1 M HCl, 50 mL of sat. sodium bicarbonate, and 50 mL of brine. Dried the organic phase over sodium sulfate and isolated the title product by chromatography on 40 g silica gel column, using gradient EtOAc/Hex of 0-80%. UPLC method C: Rt=3.50 min, m/z=417.953 [M+1].

### Step 2 3,3′-((2-(bis(benzyloxy)phosphoryl)acetyl)azanediyl)dipropanoic acid

To a solution of 3,3′-azanediyldipropanoic acid (102 mg, 0.630 mmol) in DMF (3.15 mL) was added 2,5-dioxopyrrolidin-1-yl 2-(bis(benzyloxy)phosphoryl)acetate (263 mg, 0.630 mmol) followed by triethylamine (351 μl, 2.52 mmol). Stirred the mixture for 1 hr. Removed most of DMF on rotovap. Isolated the target material by ISCO reverse phase C18 column (40 g, flow 40 mL/min, grad 0-60% in 30 min followed by hold). After lyophilization of fractions, obtained 3,3′-((2-(bis(benzyloxy)phosphoryl)acetyl)azanediyl)dipropanoic acid, UPLC method C: Rt=3.44 min, m/z=464.098 [M+1].

### Step 3 bis(2,5-dioxopyrrolidin-1-yl) 3,3′-((2-(bis(benzyloxy)phosphoryl)acetyl)azanediyl)dipropionate

To a solution of 3,3′-((2-(bis(benzyloxy)phosphoryl)acetyl)azanediyl)dipropanoic acid (187 mg, 0.404 mmol) in acetonitrile (3.8 mL) and DMF (1.0 mL) was added TSTU (255 mg, 0.847 mmol) followed by triethylamine (0.141 mL, 1.009 mmol). Stirred 1.5 hrs. As reaction was incomplete by UPLC, added more of TSTU (24.30 mg, 0.081 mmol), and stirred for 30 more min. Diluted the mixture with 100 mL of EtOAc and washed with 100 mL of sat. sodium bicarbonate, 100 mL of 1M HCl, 100 mL of brine. Dried the organic phase over sodium sulfate, removed volatiles in vacuo, and used in the next step without further purification. UPLC method C: Rt=4.04 min, m/z=658.205 [M+1].

### Step 4 (2-(bis(3-((2,5-dioxopyrrolidin-1-yl)oxy)-3-oxopropyl)amino)-2-oxoethyl)phosphonic acid

Dissolved bis(2,5-dioxopyrrolidin-1-yl) 3,3′-((2-(bis(benzyloxy)phosphoryl)acetyl)azanediyl)dipropionate (285 mg, 0.433 mmol) in THF (4.33 mL) add added Pearlman's catalyst (60.9 mg, 0.087 mmol). Hydrogenated under a balloon for 3 hrs. Added 1 mL of water to increase solubility of the forming product and continued hydrogenation for 3 more hrs. Filtered off the catalyst and concentrated the filtrate on rotovap using room temperature bath to remove most of THF, and removed the remaining aqueous solvent by lyophilization. The title product was obtained as a solid, UPLC method C: Rt=1.68 min, m/z=478.023 [M+1].

### Preparative Example 10

### Synthesis of bis(2,5-dioxopyrrolidin-1-yl) 2,2′-(1-(3-methoxypropanoyl)piperidine-4,4-diyl)diacetate (Linking reagent 10) is Described

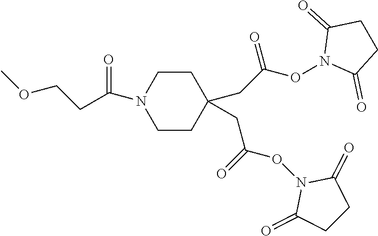 [SMILES: COCCC(=O)N1CCC(CC(=O)ON2C(=O)CCC2=O)(CC(=O)ON2C(=O)CCC2=O)CC1]

2,2′-(Piperidine-4,4-diyl)diacetic acid (250 mg, 1.242 mmol) was dissolved in anhydrous DMF (15 mL) at under nitrogen and the solution was cooled to 0° C. To the solution was added 2,5-dioxopyrrolidin-1-yl 3-methoxypropanoate (625 mg, 3.11 mmol) in DMF (4.8 mL) portionwise over a period of 15 min and then triethylamine (0.450 mL, 3.23 mmol) was added dropwise over a period of 10 min. The resulting mixture was stirred at r.t. under nitrogen overnight. The solvent was removed under reduced pressure. The residue was purified by column chromatography on 100 g C18 reverse phase column (ISCO), eluting with acetonitrile/water (gradient from 5% to 50% in 25 column volumes), furnishing, after lyophilization, 2,2′-(1-(3-methoxypropanoyl)piperidine-4,4-diyl)diacetic acid as a white solid. This material (287 mg, 0.99 mmol) was dissolved in DMF (15 mL) at 0° C. was treated with TSTU (752 mg, 2.497 mmol) and Hunig's base (0.480 mL, 2.75 mmol), and the mixture was stirred for 1 hour and at room temperature for 1 hour. The reaction mixture was concentrated down at 40° C. bath temperature under reduced pressure to remove most DMF. The residue was purified by column chromatography on 150 g C18 reverse phase column (ISCO), eluting with acetonitrile/water (gradient from 10% to 80% in 30 min). The desired fractions were lyophilized to give the title compound as white solid. UPLC method C: Rt=4.28 min, m/z=482.18 [M+1].

### Preparative Example 11

### Synthesis of bis(2,5-dioxopyrrolidin-1-yl) 4-((tert-butoxycarbonyl)amino)heptanedioate (Linking reagent 11) is Described

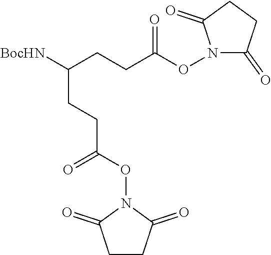 [SMILES: CC(C)(C)OC(=O)NC(CCC(=O)ON1C(=O)CCC1=O)CCC(=O)ON1C(=O)CCC1=O]

4-((Tert-butoxycarbonyl)amino)heptanedioic acid (17.8 mg, 0.065 mmol)) was dissolved in DMF (0.4 mL), treated with TSTU (42.8 mg, 0.142 mmol) and triethylamine (0.022 mL, 0.162 mmol)). Reaction was stirred at rt for 30 min and quenched with a drop of TFA. The product was purified by silica gel column (0-100% EtOAc/Hex in 16 column volumes). Fractions containing desired product were combined and concentrated to furnish the title product. UPLC method C: Rt=3.70 min, m/z=939.70 [2M+1].

Preparation of linking reagents 12-36, 38 and 39, modifying reagents as needed, were prepared in accordance with the procedures described herein.

### Preparative Example 12

### Synthesis of bis(2,5-dioxopyrrolidin-1-yl) 3,3′-(((2R,3R)-2,3-diacetoxysuccinyl)bis(azanediyl))dipropionate (Linking Reagent 37)

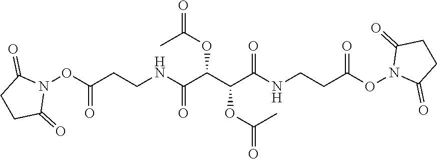 [SMILES: CC(=O)O[C@@H](C(=O)NCCC(=O)ON1C(=O)CCC1=O)[C@@H](OC(C)=O)C(=O)NCCC(=O)ON1C(=O)CCC1=O]

### Step 1 Dibenzyl 3,3′-(((2R,3R)-2,3-diacetoxysuccinyl)bis(azanediyl))dipropionate

Stirred overnight the mixture containing beta-alanine benzyl ester p-toluenesulfonate salt (3.00 g, 8.54 mmol), (−)-diacetyl-1-tartaric acid (1.00 g, 4.27 mmol), EDC (2.46 g, 12.8 mmol), HOBT (1.96 g, 12.8 mmol), DIPEA (2.98 mL, 17.08 mmol) and 21.3 mL of DMF as the solvent. Diluted the reaction mixture with 5×EtOAc and washed with equal volume of 1M HCl followed by sat NaHCO₃, followed by brine. Dried the organic phase over MgSO4, and concentrated.

The title material was purified by column chromatography on 80 g SiO₂, gradient 0-80% EtOAc/Hex over 30 min followed by hold. UPLC Method C: Rt=3.01 min, m/z=557.15 [M+1].

### Step 2 3,3′-(((2R,3R)-2,3-diacetoxysuccinyl)bis(azanediyl))dipropionic acid

Hydrogenated dibenzyl 3,3′-(((2R,3R)-2,3-diacetoxysuccinyl)bis(azanediyl))dipropionate (250 mg, 0.449 mmol) using Pearlman's Catalyst (31.5 mg, 0.045 mmol) and a mixture of THF (5.0 ml) and acetic acid (1.0 ml) as the solvent, 50 psi of hydrogen, over a period of 4 hrs. Removed catalyst by filtration, rotovaped solvent, pumped overnight on high vac (amorphous solid obtained). UPLC Method C: Rt=0.97 min, m/z=377.04 [M+1]

### Step 3 Bis(2,5-dioxopyrrolidin-1-yl) 3,3′-(((2R,3R)-2,3-diacetoxysuccinyl)bis(azanediyl))dipropionate

To a solution of 3,3′-(((2R,3R)-2,3-diacetoxysuccinyl)bis(azanediyl))dipropionic acid (29 mg, 0.077 mmol) in DMF (771 μl) was added TSTU (51.0 mg, 0.170 mmol), waited a few minutes to dissolve. Cooled the mixture to 0 C. Added 2,2,6,6-tetramethylpiperidine (23.95 mg, 0.170 mmol) and stirred 30 min UPLC Method C: Rt=2.32 min, m/z=571.44 [M+1]. The reagent was used as the solution in DMF with calculated. concentration 0.1 M.

### Preparative Example 13

### Synthesis of 16,3-di-tert-butyl 1,18-bis(2,5-dioxopyrrolidin-1-yl) (3S,16S)-5,14-dioxo-8,11-dioxa-4,15-diazaoctadecane-1,3,16,18-tetracarboxylate (Linking reagent 40)

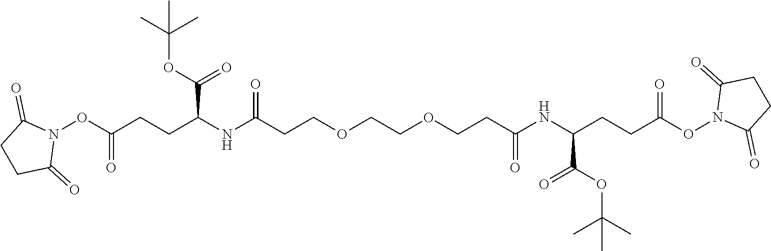 [SMILES: CC(C)(C)OC(=O)[C@H](CCC(=O)ON1C(=O)CCC1=O)NC(=O)CCOCCOCCC(=O)N[C@@H](CCC(=O)ON1C(=O)CCC1=O)C(=O)OC(C)(C)C]

### Step 1 (4S,17S)-4,17-bis(tert-butoxycarbonyl)-6,15-dioxo-9,12-dioxa-5,16-diazaicosanedioic acid

To a solution of L-Glu-OtBu (406 mg, 1.998 mmol) and sodium bicarbonate (369 mg, 4.40 mmol) in THF (6 mL) and Water (4 mL) at 0° C. was added bis(2,5-dioxopyrrolidin-1-yl) 3,3′-(ethane-1,2-diylbis(oxy))dipropionate (400 mg, 0.999 mmol) in THF (6 mL), and the mixture was allowed to warm to room temperature and stirred overnight. The mixture was concentrated at room temperature, partitioned between water and EtOAc, adjusted to pH ˜2-3 with 0.3M HCl, extracted with EtOAc, washed with brine, dried over Na₂SO₄, filtered and concentrated in vacuo. The crude was purified by column chromatography over C18 (Isco 100 g, eluting with water/acetonitrile 85:15 to 60:40; fractions analysed by LCMS and lyophilized, only the expected product was collected) to give the title compound as an oil, UPLC Method C: Rt=0.394 min, m/z=577.28 [M+1].

### Step 2 16,3-di-tert-butyl 1,18-bis(2,5-dioxopyrrolidin-1-yl) (3S,16S)-5,14-dioxo-8,11-dioxa-4,15-diazaoctadecane-1,3,16,18-tetracarboxylate

(4S,17S)-4,17-Bis(tert-butoxycarbonyl)-6,15-dioxo-9,12-dioxa-5,16-diazaicosanedioic (300 mg, 0.520 mmol) was dissolved in DMF (2 ml) and cooled to 0° C. and treated with Hunig's base (0.232 mL, 1.301 mmol) and TSTU (449 mg, 1.093 mmol). Reaction was stirred at 0° C. for 30 mins. TFA (0.120 ml, 1.561 mmol) was added and material was isolated on 150 g Biotage reverse phase C-18 column, eluting with 0-100% MeCN/H2O over 20 CV followed by 2 CV. UPLC Method C: Rt=3.16 min, m/z=771.27 [M+1].

### Preparative Example 14

### Synthesis of 2,5-dioxopyrrolidin-1-yl 17-((3-((2,5-dioxopyrrolidin-1-yl)oxy)-3-oxopropoxy)methyl)-4,8,15-trioxo-6-(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)-1-(((2R,3R,4S,5S,6R)-3,4,5-trihydroxy-6-(hydroxymethyl)tetrahydro-2H-pyran-2-yl)oxy)-19-oxa-3,6,9,16-tetraazadocosan-22-oate (Linking Reagent 41)

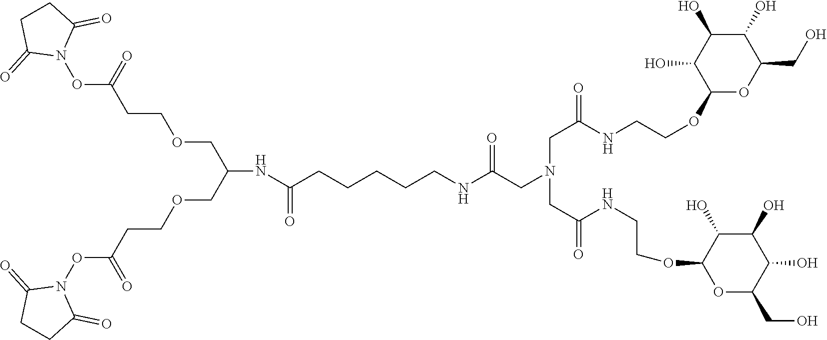 [SMILES: O=C(CN(CC(=O)NCCO[C@@H]1O[C@H](CO)[C@@H](O)[C@H](O)[C@H]1O)CC(=O)NCCO[C@@H]1O[C@H](CO)[C@@H](O)[C@H](O)[C@H]1O)NCCCCCC(=O)NC(COCCC(=O)ON1C(=O)CCC1=O)COCCC(=O)ON1C(=O)CCC1=O]

### Step 1 Benzyl 6-(2-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)acetamido)hexanoate

In a round bottom flask, 2,2′-((2-((6-(benzyloxy)-6-oxohexyl)amino)-2-oxoethyl)azanediyl)diacetic acid (800 mg, 2.028 mmol) was dissolved in DMF (8 mL). To above solution was added EDC (1166 mg, 6.08 mmol) and HOBT (155 mg, 1.014 mmol). The mixture was stirred at rt for 30 min. To above mixture was added (2R,3R,4S,5S,6R)-2-(2-aminoethoxy)-6-(hydroxymethyl)tetrahydro-2H-pyran-3,4,5-triol (996 mg, 4.46 mmol). The reaction was stirred at rt for 18 hr. DMF was removed under reduced pressure. The crude product was purified by C₁₈ reverse phase chromatography (eluted with 0-40% ACN/water in 16 CV). The title product was isolated as a powder after lyophilization. UPLC Method C: Rt=2.67 min, m/z=805.31 [M+1].

### Step 2 6-(2-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)acetamido)hexanoic acid

Benzyl 6-(2-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)acetamido)hexanoate (450 mg, 0.559 mmol) was dissolved in water (5.0 mL), treated with Pearlman's catalyst (108 mg, 0.101 mmol). The reaction was stirred under hydrogen balloon for 18 hr, filtered through a pad of celite, and the title product was isolated by lyophylization. UPLC Method C: Rt=1.25 min, m/z=715.3 [M+1].

Step 3 2,5-Dioxopyrrolidin-1-yl 6-(2-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)acetamido)hexanoate 6-(2-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)acetamido)hexanoic acid (410 mg, 0.574 mmol) was dissolved in DMF (2.0 mL), cooled to 0° C., treated with TSTU (259 mg, 0.860 mmol) followed by TEA (0.128 mL, 0.918 mmol). The reaction was stirred at 0° C. for 30 min then at rt overnight. DMF was removed under reduced pressure. The product was isolated by C₈ reverse phase chromatography (eluted with 5-25% ACN/water in 25 min) as a solid after lyophilization of fractions. UPLC Method C: Rt=1.75 min, m/z=812.3 [M+1].

### Step 4 17-((2-carboxyethoxy)methyl)-4,8,15-trioxo-6-(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)-1-(((2R,3R,4S,5S,6R)-3,4,5-trihydroxy-6-(hydroxymethyl)tetrahydro-2H-pyran-2-yl)oxy)-19-oxa-3,6,9,16-tetraazadocosan-22-oic acid

In a 20 ml vial, 3,3′-((2-aminopropane-1,3-diyl)bis(oxy))dipropanoic acid (23 mg, 0.098 mmol) was dissolved in DMF (1.0 mL), cooled to 0° C. To above solution was added 2,5-dioxopyrrolidin-1-yl 6-(2-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)acetamido)hexanoate (87 mg, 0.108 mmol) followed by TEA (10.67 μl, 0.077 mmol). The reaction was warmed to rt and stirred at rt for 18 hr. DMF was removed under reduced pressure. The crude was dissolved in the crude in 1.5 ml (30% ACN/water with 0.05% TFA). The product was isolated by C18 reverse phase chromatography as a solid after lyophilization of fractions. UPLC Method C: Rt=1.60 min, m/z=932.3 [M+1].

Step 5 2,5-Dioxopyrrolidin-1-yl 17-((3-((2,5-dioxopyrrolidin-1-yl)oxy)-3-oxopropoxy)methyl)-4,8,15-trioxo-6-(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)-1-(((2R,3R,4S,5S,6R)-3,4,5-trihydroxy-6-(hydroxymethyl)tetrahydro-2H-pyran-2-yl)oxy)-19-oxa-3,6,9,16-tetraazadocosan-22-oate 17-((2-carboxyethoxy)methyl)-4,8,15-trioxo-6-(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)-1-(((2R,3R,4S,5S,6R)-3,4,5-trihydroxy-6-(hydroxymethyl)tetrahydro-2H-pyran-2-yl)oxy)-19-oxa-3,6,9,16-tetraazadocosan-22-oic acid was converted to the bis-NHS ester using TSTU as previously described. UPLC Method C: Rt=2.14 min, m/z=563.2 [(M+2)/2].

### Preparative Example 15

### Synthesis of 2,5-dioxopyrrolidin-1-yl 13-((3-((2,5-dioxopyrrolidin-1-yl)oxy)-3-oxopropoxy)methyl)-11-oxo-2,5,8,15-tetraoxa-12-azaoctadecan-18-oate (Linking reagent 42)

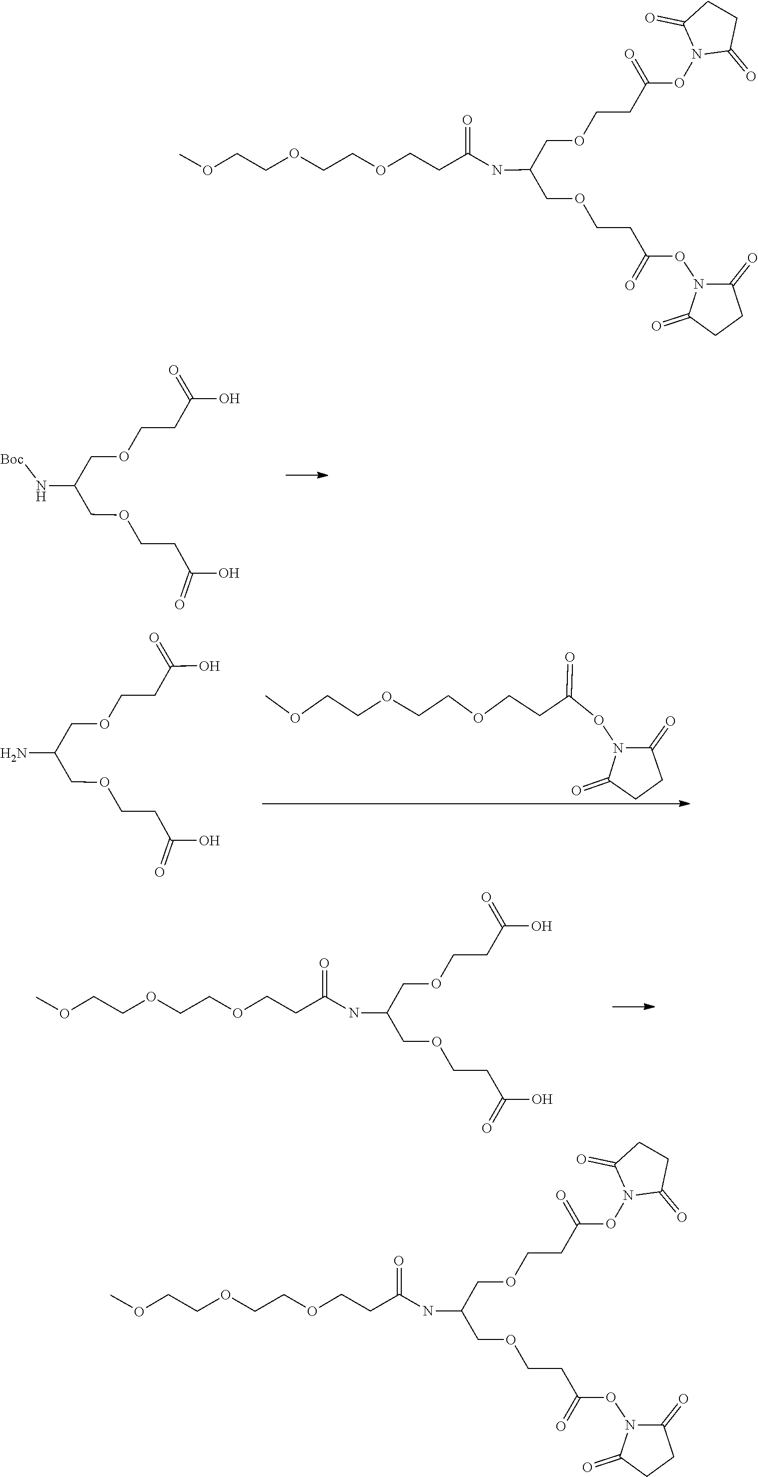

### Step 1 3,3′-((2-aminopropane-1,3-diyl)bis(oxy)dipropionic acid

In a 100 mL flask, 3,3′-((2-((tert-butoxycarbonyl)amino)propane-1,3-diyl)bis(oxy))dipropanoic acid (500 mg, 1.491 mmol) was coded 0° C. TFA (0.3 mL, 3.89 mmol) was added. The reaction mixture was stirred at 0° C. for 2 hr and added to 20 volumes of diethyl ether. After centrifugation, the supernatant was decanted and the title material was obtained as a syrup after pumping on high vacuum for 2 days. UPLC Method A: Rt=0.32 min, m/z=236.18 [M+1].

### Step 2: 13-((2-carboxyethoxy)methyl)-11-oxo-2,5,8,15-tetraoxa-12-azaoctadecan-18-oic acid acid

In a 20 mL vial, 3,3′-((2-aminopropane-1,3-diyl)bis(oxy)dipropionic acid (95 mg, 0.404 mmol) was dissolved in DMF (1.0 mL), cooled to 0° C., and treated with 2,5-dioxopyrrolidin-1-yl 3-(2-(2-methoxyethoxy)ethoxy)propanoate (128 mg, 0.444 mmol) in DMF (0.2 mL) followed by TEA (0.073 mL, 0.525 mmol). The reaction mixture was warmed to rt and stirred at rt for 18 hr. The solvent was removed under reduced pressure. The product was isolated by C₁₈ RP HPLC (eluted with 0-30% ACN/water in 16 CV). Fractions containing desired product were combined and lyophilized to furnish the title product as an oil. UPLC Method C: Rt=2.09 min, m/z=409.98 [M+1].

### Step 3: 2,5-Dioxopyrrolidin-1-yl 13-((3-((2,5-dioxopyrrolidin-1-yl)oxy)-3-oxopropoxy)methyl)-11-oxo-2,5,8,15-tetraoxa-12-azaoctadecan-18-oate

In a 20 mL vial, 13-((2-carboxyethoxy)methyl)-11-oxo-2,5,8,15-tetraoxa-12-azaoctadecan-18-oic acid acid (74 mg, 0.181 mmol) was dissolved in DMF (1.0 mL), cooled to zero deg, added TSTU (136 mg, 0.452 mmol) followed by addition of triethylamine (0.076 mL, 0.542 mmol). The reaction was stirred at 0° C. for 2 h. To above reaction mixture, added a drop of TFA, removed DMF, re-dissolved the residue in ACN/water (1/1 with 0.05% TFA), purified by C₁₈ reverse phase chromatography (eluted with 0-50% ACN/water in 10 CV). Fractions containing desired product were combined and lyophilized to furnish the title material as a powder. UPLC Method C: Rt=3.21 min, m/z=604.2 [M+1].

### Preparative Example 16

### Synthesis of bis(2,5-dioxopyrrolidin-1-yl) 13-(11-oxo-2,5,8,15,18,21-hexaoxa-12-azatricosan-23-yl)-4,7,10,16,19,22-hexaoxa-13-azapentacosanedioate (Linking reagent 43)

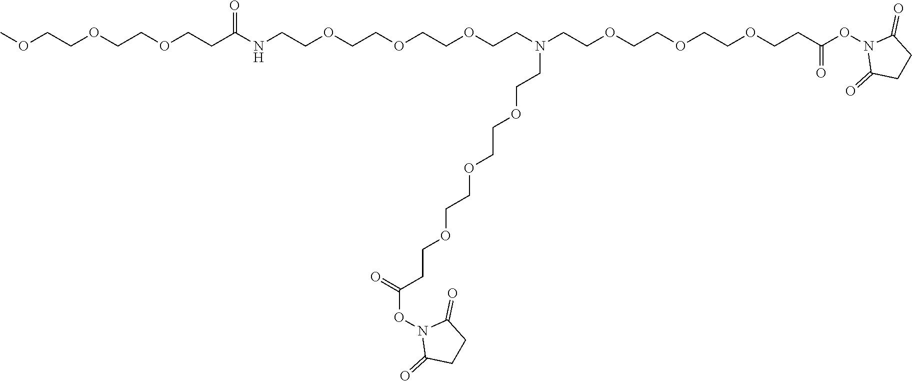 [SMILES: COCCOCCOCCC(=O)NCCOCCOCCOCCN(CCOCCOCCOCCC(=O)ON1C(=O)CCC1=O)CCOCCOCCOCCC(=O)ON1C(=O)CCC1=O]

### Step 1: 13-(11-oxo-2,5,8,15,18,21-hexaoxa-12-azatricosan-23-yl)-4,7,10,16,19,22-hexaoxa-13-azapentacosane-1,25-dioic acid

To the THF/water (4.0 mL/1.0 mL) solution of 13-(2-{2-[2-(2-aminoethoxy)ethoxy]ethoxy}ethyl)-4,7,10,16,19,22-hexaoxa-13-azapentacosane-1,25-dioic acid (105 mg, 0.175 mmol) was added 1-({3-[2-(2-methoxyethoxy)ethoxy]propanoyl}oxy)pyrrolidine-2,5-dione (50.6 mg, 0.175 mmol, in 1.0 ml THF) and TEA (0.029 ml, 0.210 mmol). The mixture was stirred at rt for 18 hr. The solvent was removed by rotavap. The product was isolated by C18 reverse phase column chromatography eluting with 0-50% ACN/water. The fractions containing desired product were combined and lyophilized to furnish a solid. UPLC Method B: Rt=2.32 min, m/z=775.35 [M+1].

### Step 2: Bis(2,5-dioxopyrrolidin-1-yl) 13-(11-oxo-2,5,8,15,18,21-hexaoxa-12-azatricosan-23-yl)-4,7,10,16,19,22-hexaoxa-13-azapentacosanedioate

13-(11-oxo-2,5,8,15,18,21-hexaoxa-12-azatricosan-23-yl)-4,7,10,16,19,22-hexaoxa-13-azapentacosane-1,25-dioic acid was converted to the title material using TSTU as previously described. UPLC Method B: Rt=2.90 min, m/z=969.1 [M+1].

### Preparative Example 17

### Synthesis of bis(2,5-dioxopyrrolidin-1-yl) 6,6′-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)azanediyl)dihexanoate (Linking Reagent 44)

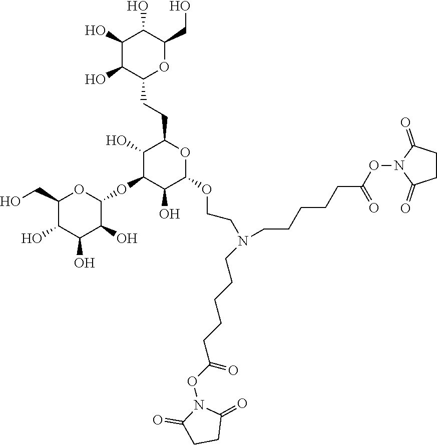 [SMILES: O=C(CCCCCN(CCCCCC(=O)ON1C(=O)CCC1=O)CCO[C@H]1O[C@H](CC[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)ON1C(=O)CCC1=O]

### Step 1: (2R,3R,4S,5S,6S)-2-((benzoyloxy)methyl)-6-(((2R,3R,4S,5S,6S)-3,5-bis(benzoyloxy)-6-(2-(bis(6-(benzyloxy)-6-oxohexyl)amino)ethoxy)-4-(((2R,3S,4S,5R,6R)-3,4,5-tris(benzoyloxy)-6-((benzoyloxy)methyl)tetrahydro-2H-pyran-2-yl)oxy)tetrahydro-2H-pyran-2-yl)methoxy)tetrahydro-2H-pyran-3,4,5-triyl tribenzoate

To a solution of 2-aminoethyl O-2,3,4,6-tetra-O-benzoyl-α-D-mannopyranosyl-(1→3)-O-[2,3,4,6-tetra-O-benzoyl-α-D-mannopyranosyl-(1→6)]-α-D-mannopyranoside-2,4-dibenzoate [WO 2015051052 Å2 20150409] (2.26 g, 1.42 mmol) and benzyl 6-oxohexanoate (0.69 g, 3.13 mmol) in DCM (15 mL) was added sodium triacetoxyborohydride (905 mg, 4.27 mmol) and the resulting mixture stirred at room temperature for 72 hours. The solvent was evaporated and the residue partitioned between EtOAc (50 mL) and sat.NaHCO₃ (100 mL); organic layer was washed with brine (50 mL), dried over Na2SO4, filtered and evaporated. The residue was purified by silica gel column chromatography (Teledyne Isco: SNAP 80 g GOLD): eluent: gradient 2-5% MeOH in DCM to give the title compound as an oil. ¹H NMR (CHCl3-d, 500 MHz): δ H 1.35-1.28 (5H, m), 2.32 (4H, t, J=7.5 Hz), 2.52 (4H, t, J=7.5 Hz), 2.79 (2H, t, J=6.2 Hz), 3.70-3.66 (1H, m), 3.81 (1H, d, J=10.7 Hz), 3.97-3.92 (1H, m), 4.19 (1H, dd, J=10.6, 4.9 Hz), 4.34 (3H, dd, J=11.9, 3.8 Hz), 4.56-4.49 (3H, m), 4.67-4.61 (2H, m), 5.07 (4H, s), 5.18-5.17 (2H, m), 5.38 (2H, s), 5.77-5.73 (2H, m), 5.83 (1H, dd, J=3.3, 1.8 Hz), 6.08-6.01 (3H, m), 6.16 (1H, t, J=10.1 Hz), 7.37-7.29 (20H, m), 7.39 (5H, d, J=7.6 Hz), 7.45-7.41 (7H, m), 7.51-7.46 (2H, m), 7.62-7.54 (7H, m), 7.74 (2H, dd, J=8.1, 1.4 Hz), 7.79-7.77 (2H, m), 7.89-7.86 (4H, m), 8.04 (2H, dd, J=8.0, 1.4 Hz), 8.13-8.07 (7H, m), 8.17-8.15 (2H, m), 8.36-8.34 (2H, m).

### Step 2: 6,6′-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((25,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)azanediyl)dihexanoic acid

To a solution of the product of step 1 (1.96 g, 0.981 mmol) in a mixture of MeOH (10 mL) and DCM (10 mL) was added NaOMe (0.182 mL of a 30 wt % soln in MeOH, 0.981 mmol) and the resulting mixture stirred at room temperature overnight. The mixture was evaporated to a volume ˜5 mL and added dropwise to stirred acetonitrile (180 mL) to form a white precipitate which was isolated by centrifugations. Solid pellet was re-suspended in acetonitrile (180 mL) and centrifuged was repeated. The pellet was dried in a stream of nitrogen. The resulting white solid was dissolved in water (10 mL) and 1N NaOH (2 mL, 2 mmol) was added. The mixture was stirred at room temperature for 1 hour and full hydrolysis was confirmed by UPLC-MS. The mixture was acidified by the addition of acetic acid and lyophilized. Taken up in DMSO and added dropwise to acetonitrile (100 mL) to form a white precipitate. Mixture centrifuged at 3500 rpm for 15 mins and the supernatent decanted. Solid pellet re-suspended in acetonitrile (180 mL) and centrifuged at 3500 rpm for 15 mins. Supernatent decanted and pellet dried under a stream of dry nitrogen to give the title compound. UPLC-MS Method C: Rt=0.872 min, m/z=776.4 [M+1].

### Step 3: bis(2,5-dioxopyrrolidin-1-yl) 6,6′-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)azanediyl)dihexanoate

To a suspension of the product of step 2 (50 mg, 0.064 mmol) in anhydrous DMF (1.0 mL) was added Hunig's base (0.045 mL, 0.26 mmol) and TSTU (80 mg, 0.193 mmol) and the resulting mixture was stirred at room temperature for 30 mins after which the mixture was added dropwise to ice bath cooled EtOAc (40 mL) to form a white precipitate. The precipitate was isolated by centrifugation, re-suspended in 40 mL of EtOAc, and centrifugation was repeated. The pellet was dried in a stream of nitrogen to give the title compound as a solid. UPLC-MS Method C: Rt=2.21 min, m/z=970.5 [M+1].

Preparation of capping reagent 5 is described in WO2010029159, incorporated herein by reference in its entirety. Capping reagents 6-11, 13, 15-19 and 28 were obtained from commercial sources such as Broadpharm.

### Preparative Example 18

### Synthesis of 2,5-dioxopyrrolidin-1-yl 2-(dimethylamino)acetate (Capping Reagent 1) is Described

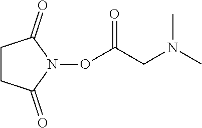

To solid N,N-dimethylglycine (50 mg, 0.485 mmol) was added a solution of TSTU (146 mg, 0.485 mmol) in DMF (4849 μl) and the mixture was sonicated for 5 min to achieve better solubility. To the resulting suspension was added triethylamine (101 μl, 0.727 mmol) and the mixture was stirred 30 min at room temp. LCMS indicates formation of the activated ester (Method A: Rt=0.20 min, m/z=201.08 [M+1]). Used this reagent as 0.1 M solution in DMF without purification.

### Preparative Example 19

### Synthesis of 16-((2,5-dioxopyrrolidin-1-yl)oxy)-16-oxohexadecanoic acid (Capping Reagent 2) is Described

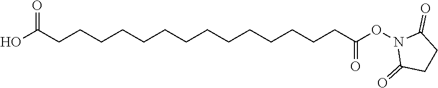 [SMILES: O=C(O)CCCCCCCCCCCCCCC(=O)ON1C(=O)CCC1=O]

### Step 1 16-(benzyloxy)-16-oxohexadecanoic acid

Hexadecanedioic acid (2.86 g, 10 mmol) and benzyl formate (3.13 ml, 25.00 mmol) were stirred overnight at 80° C. in n-octane (100 mL, 615 mmol) in the presence of DOWEX 50 WX2-200 ion exchange resin (10.0 g). The reaction mixture was filtered, the filtrate was concentrated under reduced pressure, and the residue was purified by column chromatography on silica gel, eluting with EtOAc/hexanes (gradient from 0 to 20%) to furnish the title compound as a solid. UPLC Method B: Rt=4.28 min, m/z=377.3 [M+1].

### Step 2 1-benzyl 16-(2,5-dioxopyrrolidin-1-yl) hexadecanedioate

To a solution of 16-(benzyloxy)-16-oxohexadecanoic acid (900 mg, 2.390 mmol) in anhydrous DMF (15 ml), at 0° C. was added TSTU (756 mg, 2.510 mmol), followed by Hunig's base (0.438 ml, 2.510 mmol). The reaction mixture was stirred for 1 hour and partitioned between Et2O and water, the organic phase was washed with brine, dried over MgSO4, filtered and concentrated. The title compound was obtained as an oil, UPLC Method B: Rt=4.65 min, m/z=474.32 [M+1].

### Step 3 16-((2,5-dioxopyrrolidin-1-yl)oxy)-16-oxohexadecanoic acid

1-benzyl 16-(2,5-dioxopyrrolidin-1-yl) hexadecanedioate (2.0 g, 4.22 mmol) acetone-0.1% TFA (42.2 mL) was hydrogenatd over palladium on carbon (10%) (0.449 g, 0.422 mmol), at 1 atm (balloon) overnight. The solvent was removed in vacuo and the residue was pumped overnight on high vacuum. The title compound was obtained as a solid, UPLC Method A: Rt=1.33 min, m/z=384.31 [M+1].

### Preparative Example 20

### Synthesis of 2,5-dioxopyrrolidin-1-yl (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoate (Capping Reagent 3) is Described

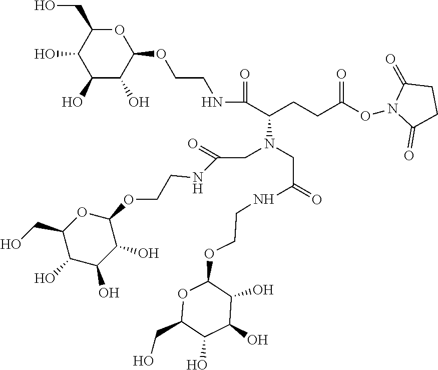 [SMILES: O=C(CN(CC(=O)NCCO[C@@H]1O[C@H](CO)[C@@H](O)[C@H](O)[C@H]1O)[C@@H](CCC(=O)ON1C(=O)CCC1=O)C(=O)NCCO[C@@H]1O[C@H](CO)[C@@H](O)[C@H](O)[C@H]1O)NCCO[C@@H]1O[C@H](CO)[C@@H](O)[C@H](O)[C@H]1O]

### Step 1 (S)-2,2′-((4-(benzyloxy)-1-carboxy-4-oxobutyl)azanediyl)diacetic acid

To a solution of 5-benzyl 1-(tert-butyl) L-glutamate hydrochloride (5 g, 15.17 mmol) in DMF (120 mL) was added tert-butyl-2-bromoacetate (11.85 g, 60.77 mmol) and DIEA (9.8 g, 76 mmol). The system was purged with N₂ and heated at 60° C. overnight. After the reaction was completed, DMF was removed under reduced pressure. The residue was purified by flash chromatography to afford 5-benzyl 1-(tert-butyl) N,N-bis(2-(tert-butoxy)-2-oxoethyl)-L-glutamate, which was dissolved in TFA (150 mL) and DCM (150 mL), purged with N₂ and heated at 40° C. for 4 hours. TFA and DCM were removed under reduced pressure. The oily residue was washed with cold Et20 twice. The suspension was filtered and the solid was dried to give (S)-2,2′-((4-(benzyloxy)-1-carboxy-4-oxobutyl)azanediyl)diacetic acid, ¹H-NMR (300 MHz, DMSO-d6): δ7.30-7.41 (m, 5H), 5.08 (s, 2H), 3.47 (s, 4H), 3.41-3.44 (t, 1H), 2.52-2.59 (m, 2H), 1.76-1.96 (m, 2H).

### Step 2 benzyl (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoate

Stirred a mixture of 2-aminoethyl-β-D-glucopyranoside, [CAS 70337-78-9] (790 mg, 3.54 mmol) and (S)-2,2′-((4-(benzyloxy)-1-carboxy-4-oxobutyl)azanediyl)diacetic acid (125 mg, 0.354 mmol) with HOBT (542 mg, 3.54 mmol), Hunig's base (618 μl, 3.54 mmol), EDC (678 mg, 3.54 mmol), using DMF (7.1 mL) as the solvent for a total period of 24 hours. Concentrated the reaction mixture on rotovap, re-dissolved in 20 mL of water and purified by reverse-phase chromatography (KROMASIL C8 250×50 mm, 10 μm, 100 Å column; Buffer A: 0.05% TFA in deionized water; Buffer B: 0.05% TFA in AcCN). After lyophilization of fractions, benzyl (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoate was obtained as a solid, UPLC-MS Method C, Rt=1.56 min, m/z=969.51 [M+1].

### Step 3 (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoic acid

Hydrogenated a mixture of benzyl (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoate (236 mg, 0.244 mmol) and Pearlman's Catalyst (17.1 mg, 0.024 mmol) in water (12.0 mL) at 50 psi overnight. Filtered out the catalyst and lyophilized the product. Obtained (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoic acid as a foam, UPLC-MS Method C: Rt=0.74 min, m/z=879.44 [M+1].

### Step 4 2,5-dioxopyrrolidin-1-yl (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoate

To a solution of (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoic acid (204 mg, 0.232 mmol) in DMF (2.3 mL) was added TSTU (77 mg, 0.255 mmol). Chilled to 0° C. and added triethylamine (48.5 μl, 0.348 mmol). Stirred 30 minutes and quenched the reaction by pouring into 20×volumes of acetone. Collected the precipitate by centrifugation. Re-suspended the precipitate in 20 mL of dry acetone and repeated the centrifugation. Lyophilized the precipitate from 5.0 mL of water and 1.0 mL AcN. Obtained 2,5-dioxopyrrolidin-1-yl (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoate as a powder, UPLC-MS Method C: Rt=1.11 min, m/z=976.44 [M+1].

### Preparative Example 21

### Synthesis of (2-((2,5-dioxopyrrolidin-1-yl)oxy)-2-oxoethyl)phosphonic acid (Capping Reagent 4) is Described

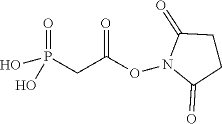 [SMILES: O=C(CP(=O)(O)O)ON1C(=O)CCC1=O]

### Step 1 2,5-dioxopyrrolidin-1-yl 2-(bis(benzyloxy)phosphoryl)acetate

To a mixture of dibenzylphosphonoacetic acid (500 mg, 1.561 mmol) and TSTU (517 mg, 1.717 mmol) in acetonitrile (7.8 mL) at 0° C. was added dropwise triethylamine (261 μl, 1.873 mmol). Stirred the reaction 2 hrs at 0° C. Concentrated on rotovap using room temperature water bath. Re-dissolved the residue in 100 mL of EtOAc and washed with 50 mL of 1 M HCl, 50 mL of sat. sodium bicarbonate, and 50 mL of brine. Dried the organic phase over sodium sulfate and isolated the title product by chromatography on 40 g silica gel column, using gradient EtOAc/Hex of 0-80%. UPLC method C: Rt=3.50 min, m/z=417.953 [M+1].

### Step 2 2-((2,5-dioxopyrrolidin-1-yl)oxy)-2-oxoethyl)phosphonic acid

Hydrogenated overnight a mixture of 2,5-dioxopyrrolidin-1-yl 2-(bis(benzyloxy)phosphoryl)acetate (305 mg, 0.731 mmol) and Pearlman's Catalyst (103 mg, 0.146 mmol) using THF (6 ml) as a solvent, under hydrogen balloon. Filtered out the catalyst and removed THF under vacuum. The title compound was obtained as an oil, UPLC-MS Method C: Rt=0.86 min, m/z=474.93 [2M+1].

### Preparative Example 22

### Synthesis of Tert-butyl 2-(2-((2,5-dioxopyrrolidin-1-yl)oxy)-2-oxoethoxy)acetate (Capping Reagent 12) is Described

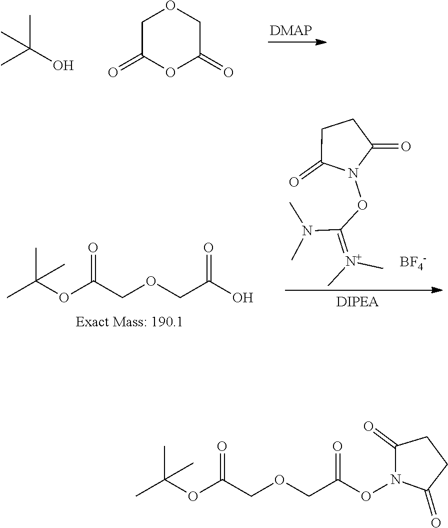

### Step 1. 2-(2-(Tert-butoxy)-2-oxoethoxy)acetic acid

To a solution of diglycolic anhydride (1.1 g, 9.48 mmol) in tert-butanol (5 mL), was added DMAP (0.116 g, 0.948 mmol) and the resulting mixture was stirred at 40° C. for 24 h. The reaction mixture was cooled down to room temperature and concentrated under reduce pressure to dryness, and re-dissolved in 100 mL of 0.1 N HCl. The product was then extracted into DCM (3×40 mL). Combined organic phase was washed with water, brine, dried over Na₂SO₄, filtered and concentrated. The residue was purified by column chromatography on 24 g ISCO silica gel column eluting with a gradient of hexanes-ethyl acetate to furnish 2-(2-(tert-butoxy)-2-oxoethoxy)acetic acid. ¹H NMR (500 MHz, CDCl₃): δ 4.23 (s; 2H); 4.14 (s; 2H); 1.50 (s; 9H).

### Step 2. Tert-butyl 2-(2-((2,5-dioxopyrrolidin-1-yl)oxy)-2-oxoethoxy)acetate

To a solution of 2-(2-(tert-butoxy)-2-oxoethoxy)acetic acid (760 mg, 4.00 mmol) in DCM (25 ml) at 0° C. was added TSTU (1.3 g, 4.32 mmol) and DIPEA (0.85 ml, 4.87 mmol). The reaction mixture were stirred at room temp for 2 h and quenched with TFA (0.45 mL, 5.84 mmol). The residue was purified by column chromatography on 40 g silica gel Column, eluting with a gradient of hexanes-ethyl acetate to furnish the title material as a solid. ¹H NMR (500 MHz, CDCl₃): δ 4.60 (s; 2H); 4.14 (s; 2H); 2.86 (s; 4H); 1.49 (s; 9H).

### Preparative Example 23

### Synthesis of 3,3′-((2-((2-carboxyethoxy)methyl)-2-(2-(2-((2,5-dioxopyrrolidin-1-yl)oxy)-2-oxoethoxy)acetamido)propane-1,3-diyl)bis(oxy))dipropionic acid

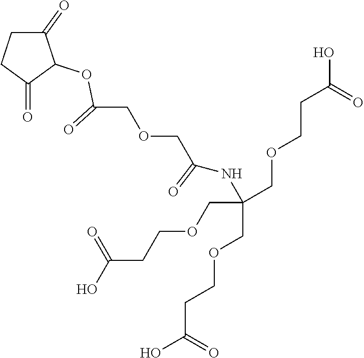 [SMILES: O=C(O)CCOCC(COCCC(=O)O)(COCCC(=O)O)NC(=O)COCC(=O)OC1C(=O)CCC1=O]

Capping reagent (Capping Reagent 20) is described

### Step 1 7,7-Bis((3-(tert-butoxy)-3-oxopropoxy)methyl)-14,14-dimethyl-5,12-dioxo-3,9,13-trioxa-6-azapentadecanoic acid

To a solution of tert-butyl 3,3′-(2-amino-2-((3-tert-butoxy-3-oxopropoxy)methyl)propane-1,3-diyl)bis(oxy)dipropanoate (500 mg, 0.989 mmol) in DMF (4944 μl) was added triethylamine (207 μl, 1.483 mmol) followed by diglycolic anhydride (172 mg, 1.483 mmol). After 30 min, the product was isolated by reverse-phase chromatography on C-8 phase (Column Kromasil, C8 10 uM 100 Å, size 250×50 mm; solvent A=water/0.05% TFA, solvent B=AcN/0.05% TFA), Flow=85 mL/min, gradient B in A 20-100% in 30 min. The product was obtained as oil after evaporation of fractions. UPLC Method B: Rt=2.04 min, m/z=622.28 [M+1].

### Step 2 3,3′-((2-((2-carboxyethoxy)methyl)-2-(2-(2-((2,5-dioxopyrrolidin-1-yl)oxy)-2-oxoethoxy)acetamido)propane-1,3-diyl)bis(oxy))dipropionic acid

To a solution of 1 7,7-Bis((3-(tert-butoxy)-3-oxopropoxy)methyl)-14,14-dimethyl-5,12-dioxo-3,9,13-trioxa-6-azapentadecanoic acid (134 mg, 0.216 mmol) in DCM (2.15 mL) was added TSTU (71.4 mg, 0.237 mmol) followed by triethylamine (0.039 mL, 0.280 mmol). Stirred 30 min. Added to the same reaction flask TFA (3 mL, 38.9 mmol) and stirred 4 hrs. Rotovaped at room temp bath and pumped on high vac overnight. Material obtained as oil and used without further purification. UPLC Method C: Rt=2.16 min, m/z=551.13 [M+1].

### Preparative Example 24

### Synthesis of 2,5-dioxopyrrolidin-1-yl 2-(2-((1,3-dihydroxy-2-(hydroxymethyl)propan-2-yl)amino)-2-oxoethoxy)acetate (Capping Reagent 21) is Described

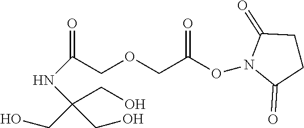 [SMILES: O=C(COCC(=O)ON1C(=O)CCC1=O)NC(CO)(CO)CO]

### Step 1 Benzyl 2-(2-((1,3-dihydroxy-2-(hydroxymethyl)propan-2-yl)amino)-2-oxoethoxy)acetate

To a solution of benzyl alcohol (466 mg, 4.31 mmol) in DCM (4308 μl) was added diglycolic anhydride (500 mg, 4.31 mmol) and DMAP (52.6 mg, 0.431 mmol) and the reaction mixture was stirred for 3 hr. To this mixture was added TSTU (1037 mg, 3.45 mmol) as solid followed by triethylamine (600 μl, 4.31 mmol) (additional DCM, ˜4 mL was added as needed to provide a well-stirred solution). To this mixture was added tris hydrochloride (543 mg, 3.45 mmol) and triethylamine (1201 μl, 8.62 mmol) and the mixture was stirred overnight. The title material was isolated by reverse-phase chromatography on C-8 phase (Column Kromasil, C8 10 uM 100 Å, size 250×50 mm; solvent A=water/0.05% TFA, solvent B=AcN/0.05% TFA), Flow=85 mL/min, gradient B in A 10-50% in 30 min. UPLC Method C: Rt=2.75 min, m/z=327.7 [M+1].

### Step 2 2,5-dioxopyrrolidin-1-yl 2-(2-((1,3-dihydroxy-2-(hydroxymethyl)propan-2-yl)amino)-2-oxoethoxy)acetate

Hydrogenated benzyl 2-(2-((1,3-dihydroxy-2-(hydroxymethyl)propan-2-yl)amino)-2-oxoethoxy)acetate (300 mg, 0.917 mmol) in water over Pearlman's Catalyst (161 mg, 0.229 mmol) at 50 psi over a period of 4 hrs. Removed catalyst by filtration and water by lyophylization. The oily residue was dissolved in DMF (2810 μl) and treated with TSTU (254 mg, 0.843 mmol) and triethylamine (118 μl, 0.843 mmol). After 30 min of reaction, formation of the title material was confirmed by UPLC Method C: Rt=2.75 min, m/z=334.72 [M+1]. The reagent was used as is (ca 0.1M in DMF).

### Preparative Example 25

### Synthesis of (4-((2,5-dioxopyrrolidin-1-yl)oxy)-4-oxobutanoyl)-L-glutamate (Capping Reagent 22) is Described

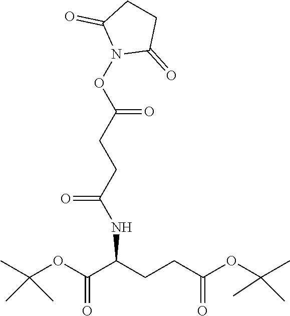 [SMILES: CC(C)(C)OC(=O)CC[C@H](NC(=O)CCC(=O)ON1C(=O)CCC1=O)C(=O)OC(C)(C)C]

### Step 1 (S)-4-((1, 5-di-tert-butoxy-1,5-dioxopentan-2-yl)amino)-4-oxobutanoic acid

To a solution of commercially available (S)-di-tert-butyl 2-aminopentanedioate (493 mg, 1.901 mmol) in THF (8.0 mL) and acetonitrile (8.0 mL) at 0° C. was added succinic anhydride (190 mg, 1.901 mmol) and Et3N (0.318 ml, 2.281 mmol). The solution was allowed to stir at 0° C. for 2 hours. The solution was concentrated under reduced pressure; the residue was purified by column chromatography on ISCO C18 column, eluting with acetonitrile in water (from 10% to 70% in 27 min.) to give the title compound as oil. UPLC Method C: Rt=4.0 min, m/z=359.88 [M+1].

### Step 2 (S)-4-((1, 5-di-tert-butoxy-1,5-dioxopentan-2-yl)amino)-4-oxobutanoic acid

To a solution of (S)-4-((1, 5-di-tert-butoxy-1, 5-dioxopentan-2-yl) amino)-4-oxobutanoic acid (404 mg, 1.124 mmol) in THF (5 ml) and CH2Cl2 (10 ml) at 0° C. was added TSTU (406 mg, 1.349 mmol) and Et3N (0.204 ml, 1.461 mmol). The solution was allowed to stir at 0° C. for 1 hour. The solution was concentrated under reduced pressure, the residue was purified by column chromatography on C18 Gold 100 g Column, eluting with acetonitrile in water (from 10% to 70% in 27 min.) to give the title compound (390 mg, 0.854 mmol, 76% yield) as a solid. UPLC Method C: Rt=4.50 min, m/z=457.08 [M+1].

### Preparative Example 26

### Synthesis of 1,12-di-tert-butyl 6-(2,5-dioxopyrrolidin-1-yl) (S)-4,9-dioxo-2,11-dioxa-5,8-diazadodecane-1,6,12-tricarboxylate (Capping Reagent 23) is Described

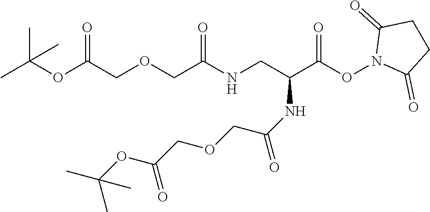 [SMILES: CC(C)(C)OC(=O)COCC(=O)NC[C@H](NC(=O)COCC(=O)OC(C)(C)C)C(=O)ON1C(=O)CCC1=O]

### Step 1 tert-Butyl 2-(2-((2,5-dioxopyrrolidin-1-yl)oxy)-2-oxoethoxy)acetate

To a solution of 2-(2-(tert-butoxy)-2-oxoethoxy)acetic acid (760 mg, 4.0 mmol) in DCM (25.0 mL) at 0° C. was added TSTU (1.3 g, 4.32 mmol) and DIPEA (0.85 mL, 4.87 mmol). After 2 h stirring at room temperature, crude mixture was quenched with TFA (0.45 ml, 5.84 mmol). The residue was purified by flash chromatography on silica gel (40 g), eluting with 0-100% EtOAc in hexanes to give the title compound. ¹H NMR (500 MHz, CDCl₃): δ 4.60 (s; 2H); 4.14 (s; 2H); 2.86 (s; 4H); 1.49 (s; 9H).

### Step 2 (S)-2,3-bis(2-(2-(tert-butoxy)-2-oxoethoxy)acetamido)propanoic acid

To a suspension of (S)-2,3-diaminopropanoic acid (63.5 mg, 0.61 mmol) in DMF (4.0 mL) was added DIPEA (0.35 mL, 2.0 mmol) and tert-butyl 2-(2-((2,5-dioxopyrrolidin-1-yl)oxy)-2-oxoethoxy)acetate (376 mg, 1.31 mmol). After stirring for 16 h, crude mixture was concentrated under reduce pressure and the residue was purified by column chromatography on 50 g C18 column eluting with 5-60% ACN in H2O to give the title compound. UPLC Method C: Rt=1.4 min, m/z=449.06 [M+1].

### Step 3: 1,12-di-tert-butyl 6-(2,5-dioxopyrrolidin-1-yl) (S)-4,9-dioxo-2,11-dioxa-5,8-diazadodecane-1,6,12-tricarboxylate

To a solution of (S)-2,3-bis(2-(2-(tert-butoxy)-2-oxoethoxy)acetamido)propanoic acid (30 mg, 0.067 mmol) in DMF (400 μL) at 0° C. were added TSTU (25 mg, 0.083 mmol) and DIPEA (20 μL, 0.115 mmol). After 1 h of stirring at room temperature, crude mixture was used in the following reaction without any purification. UPLC Method C: Rt=1.57 min, m/z=546.18 [M+1].

### Preparative Example 27

### Synthesis of di-tert-butyl 8-(2-((2,5-dioxopyrrolidin-1-yl)oxy)-2-oxoethoxy)-5,11-dioxo-3,13-dioxa-6,10-diazapentadecanedioate (Capping Reagent 24) is Described

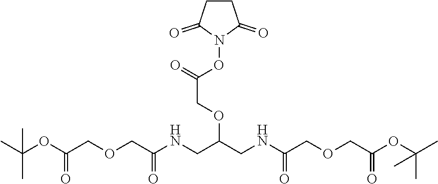 [SMILES: CC(C)(C)OC(=O)COCC(=O)NCC(CNC(=O)COCC(=O)OC(C)(C)C)OCC(=O)ON1C(=O)CCC1=O]

### Step 1 4-((2-(2-(tert-butoxy)-2-oxoethoxy)acetamido)methyl)-13,13-dimethyl-7,11-dioxo-3,9,12-trioxa-6-azatetradecanoic acid

To a solution of 2-((1,3-diaminopropan-2-yl)oxy)acetic acid (123 mg, 0.556 mmol) in 1N NaHCO₃ (3.0 mL) was added dropwise solution of tert-butyl 2-(2-((2,5-dioxopyrrolidin-1-yl)oxy)-2-oxoethoxy)acetate (339 mg, 1.180 mmol) in ACN (1.0 mL). After stirring for 1 h, the residue was directly purified by column chromatography on 50 g C18 column eluting with 5-80% ACN in H2O to give the title compound. UPLC Method C: Rt=1.38 min, m/z=493.13 [M+1].

### Step 2 di-tert-butyl 8-(2-((2,5-dioxopyrrolidin-1-yl)oxy)-2-oxoethoxy)-5,11-dioxo-3,13-dioxa-6,10-diazapentadecanedioate

To a solution of 4-((2-(2-(tert-butoxy)-2-oxoethoxy)acetamido)methyl)-13,13-dimethyl-7,11-dioxo-3,9,12-trioxa-6-azatetradecanoic acid (120 mg, 0.244 mmol) in DMF (1.5 mL) at 0° C. were added TSTU (88 mg, 0.292 mmol) and DIPEA (85 μL, 0.487 mmol). After 1 h stirring at room temperature, crude mixture was quenched with TFA (55 μL, 0.714 mmol) and the residue was purified by column chromatography on 50 g C18 column eluting with 5-80% ACN in H2O to give the title compound. UPLC Method C: Rt=1.48 min, m/z=590.21 [M+1].

### Preparative Example 28

### Synthesis of 2-(2-(2-((2,5-dioxopyrrolidin-1-yl)oxy)-2-oxoethoxy)ethoxy)acetic acid (Capping Reagent 25) is Described

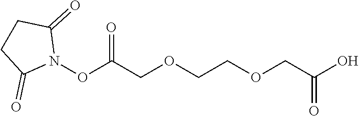 [SMILES: O=C(O)COCCOCC(=O)ON1C(=O)CCC1=O]

### Step 1 benzyl 2-(2-(2-((2,5-dioxopyrrolidin-1-yl)oxy)-2-oxoethoxy)ethoxy)acetate

To a solution of 2-(2-(2-(benzyloxy)-2-oxoethoxy)ethoxy)acetic acid (285 mg, 1.062 mmol, organic letters 2002, 4(14), 2293-2296) in DCM (10.0 mL) at 0° C. was added TSTU (352 mg, 1.169 mmol) and DIPEA (0.3 mL, 1.718 mmol). After 2 h stirring at room temperature, crude mixture was quenched with TFA (0.164 mL, 2.125 mmol). The residue was purified by flash chromatography on silica gel (40 g), eluting with 0-100% EtOAc in hexanes to give the title compound. UPLC Method C: Rt=0.91 min, m/z=366.3 [M+1].

### Step 2 2-(2-(2-((2,5-dioxopyrrolidin-1-yl)oxy)-2-oxoethoxy)ethoxy)acetic acid

A mixture of benzyl 2-(2-(2-((2,5-dioxopyrrolidin-1-yl)oxy)-2-oxoethoxy)ethoxy)acetate (325 mg, 0.890 mmol) and Pearlman's Catalyst (30 mg, 0.043 mmol) in acetone (8.0 mL) was allowed to stir under a balloon of hydrogen at rt for 90 min. The catalyst was filtered off and washed with acetone:H2O (6:1) (3×2 mL). The filtrate was concentrated to give the title compound. UPLC Method C: Rt=1.45 min, m/z=298.04 [M+1].

### Preparative Example 29

### Synthesis of 2,5-dioxopyrrolidin-1-yl 2-(((3S,3aR,6R,6aR)-6-hydroxyhexahydrofuro[3,2-b]furan-3-yl)oxy)acetate (Capping Reagent 26) is described

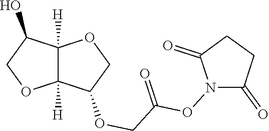 [SMILES: O=C(CO[C@H]1CO[C@H]2[C@@H]1OC[C@H]2O)ON1C(=O)CCC1=O]

### Step 1 (3S,3aR,6R,6aR)-3-(allyloxy)-6-(benzyloxy)hexahydrofuro[3,2-b]furan

To a solution of (3S,3aR,6R,6aR)-6-(benzyloxy)hexahydrofuro[3,2-b]furan-3-ol (780 mg, 3.30 mmol, European Journal of Organic Chemistry 2013 (10), 1937-1949) in allyl bromide (4.6 mL, 53.2 mmol) was added silver oxide (985 mg, 4.25 mmol), and calcium chloride (2.32 g, 17.04 mmol). The resulting suspension was stirred at room temperature for 6 days. Upon completion, crude mixture was diluted with ether (100 mL) and filtered through celite and concentrated under reduce pressure. The residue was purified by flash chromatography on silica gel (40 g), eluting with 0-100% EtOAc in hexanes to give the title compound. UPLC Method C: Rt=1.04 min, m/z=277.3 [M+1].

### Step 2: 2-(((3S,3aR,6R,6aR)-6-(benzyloxy)hexahydrofuro[3,2-b]furan-3-yl)oxy)acetic acid

To a solution of (3S,3aR,6R,6aR)-3-(allyloxy)-6-(benzyloxy)hexahydrofuro[3,2-b]furan (549 mg, 1.987 mmol) in 21 mL of ACN:CCl4:Water (2:2:3) at 0 C ° was added sodium periodate (2.2 g, 10.29 mmol), and then ruthenium (III) chloride trihydrate (30 mg, 0.115 mmol). The resulting suspension was stirred at room temperature for 72 h. Upon completion, crude mixture was diluted with water (50 mL) and extracted with DCM (3×50 mL). Combined organic layers were washed with brine, dried over Na2SO4, filtered and concentrated under reduce pressure. The residue was purified by reverse phase chromatography on C₁₈ column (50 g), eluting with 0-50% ACN in water to give the title compound. UPLC Method C: Rt=0.75 min, m/z=295.19 [M+1].

### Step 3: 2,5-dioxopyrrolidin-1-yl 2-(((3S,3aR,6R,6aR)-6-(benzyloxy)hexahydrofuro[3,2-b]furan-3-yl)oxy)acetate

To a solution of 2-(((3S,3aR,6R,6aR)-6-(benzyloxy)hexahydrofuro[3,2-b]furan-3-yl)oxy)acetic acid (120 mg, 0.408 mmol) in DMF (2.7 mL) was added TSTU (131 mg, 0.435 mmol) and DIPEA (0.085 mL, 0.489 mmol). After 2 h stirring at room temperature, crude mixture was quenched with TFA (0.070 mL, 0.909 mmol). The residue was purified by column chromatography on silica gel (40 g), eluting with 0-100% Acetone in DCM to give the title compound. UPLC Method C: Rt=0.84 min, m/z=392.32 [M+1].

### Step 4: 2,5-dioxopyrrolidin-1-yl 2-(((3S,3aR,6R,6aR)-6-hydroxyhexahydrofuro[3,2-b]furan-3-yl)oxy)acetate

A mixture of 2,5-dioxopyrrolidin-1-yl 2-(((3S,3aR,6R,6aR)-6-(benzyloxy)hexahydrofuro[3,2-b]furan-3-yl)oxy)acetate (160 mg, 0.409 mmol) and Pearlman's Catalyst (12 mg, 0.023 mmol) in acetone (4.0 mL) was allowed to stir under a balloon of hydrogen at rt for 4 h. The catalyst was filtered off and washed with acetone (3×2 mL). The filtrate was concentrated to give the title compound. UPLC Method C: Rt=0.19 min, m/z=302.23 [M+H].

### Preparative Example 30

### (2R,3S,4R,5R)—N-{6-[(2,5-dioxopyrrolidin-1-yl)oxy]-6-oxohexyl}-2,3,4,5,6-pentahydroxyhexanamide (Capping Reagent 27) is Described

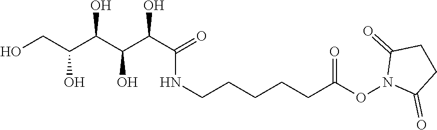 [SMILES: O=C(CCCCCNC(=O)[C@H](O)[C@@H](O)[C@H](O)[C@H](O)CO)ON1C(=O)CCC1=O]

### Step 1: Benzyl 6-{[(2R,3S,4R,5R)-2,3,4,5,6-pentahydroxyhexanoyl]amino}hexanoate

To the solution of (2R,3S,4R,5R)-2,3,4,5,6-pentahydroxyhexanoic acid (1.0 g, 5.10 mmol) in DMF (5.0 mL) at room temperature was added 6-amino-hexanoic acid benzyl ester, compound with toluene-4-sulfonic acid (2.508 g, 6.37 mmol), followed by Et3N (1.776 mL, 12.75 mmol), HOBT (0.976 g, 6.37 mmol) and EDC (1.222 g, 6.37 mmol), the mixture was stirred at rt over night. Concentrated down and purified by Biotage snap on 120 g C₁₈ column, eluting with 0-25% AcCN in water, 20 CV. Combined desired product fractions and lyohpilized to provide the title compound as a powder. UPLC Method C: Rt=3.04 min, m/z=399.96 [M+H].

### Step 2: 6-{[(2R,3S,4R,5R)-2,3,4,5,6-pentahydroxyhexanoyl]amino}hexanoic acid

To the solution of benzyl 6-((2R,3S,4R,5R)-2,3,4,5,6-pentahydroxyhexanamido)hexanoate (2.0 g, 5.01 mmol) in water (20 mL) was added 20% Pd/C (0.533 g, 5.01 mmol) and the reaction mixture was hydrogenated overnight. The catalyst was filtered off by celite pad, the filtrate was lyophilized to provide the title compound as a powder. UPLC Method C: Rt=1.20 min, m/z=309.31 [M+H].

### Step 3: (2R,3S,4R,5R)—N-{6-[(2,5-dioxopyrrolidin-1-yl)oxy]-6-oxohexyl}-2,3,4,5,6-pentahydroxyhexanamide

To the solution of 6-((2R,3S,4R,5R)-2,3,4,5,6-pentahydroxyhexanamido)hexanoic acid (1.52 g, 4.91 mmol) in DMF (8 mL) at room temperature was added TSTU (1.553 g, 5.16 mmol), followed by Et3N (0.719 mL, 5.16 mmol), the mixture was stirred at room temperature overnight. Concentrated down and purified by Biotage snap on 120 g C18 column, elute with 0-25% AcCN in water, 20 CV, collected the product fractions and lyophilized to provide the title compound as a white powder. UPLC Method C: Rt=1.83 min, m/z=406.95 [M+H].

### Preparative Example 31

### Synthesis of 2,5-dioxopyrrolidin-1-yl (S)-6-((5-(bis(2-((2-morpholino-2-oxoethyl)amino)-2-oxoethyl)amino)-6-((2-morpholino-2-oxoethyl)amino)-6-oxohexyl)amino)-6-oxohexanoate (Capping Reagent 29) is Described

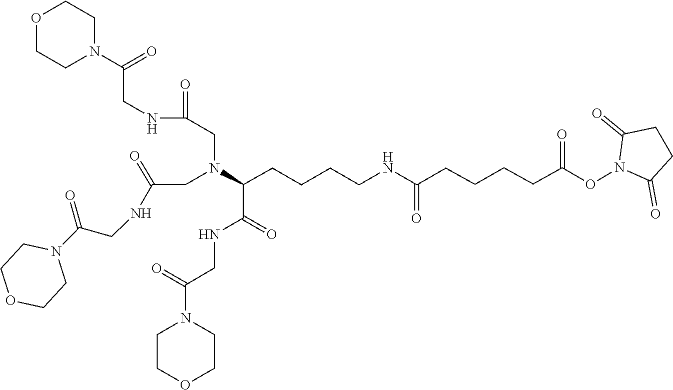 [SMILES: O=C(CCCCC(=O)ON1C(=O)CCC1=O)NCCCC[C@@H](C(=O)NCC(=O)N1CCOCC1)N(CC(=O)NCC(=O)N1CCOCC1)CC(=O)NCC(=O)N1CCOCC1]

### Step 1: (S)-benzyl (5-(bis(2-((2-morpholino-2-oxoethyl)amino)-2-oxoethyl)amino)-6-((2-morpholino-2-oxoethyl)amino)-6-oxohexyl)carbamate

To the solution of lysine (S)-2,2′-((5-(((benzyloxy)carbonyl)amino)-1-carboxypentyl)azanediyl)diacetic acid (600 mg, 1.514 mmol) in dry DMF (20 mL) was added HOBT (1.16 g, 7.57 mmol), EDC (1451 mg, 7.57 mmol) at 0° C. under nitrogen, the mixture was stirred at 0° C. for 30 minutes, then 2-amino-1-morpholin-4-yl-ethanone HCL (1367 mg, 7.57 mmol) and Et3N (2.110 ml, 15.14 mmol) was added, the mixture was gradually warmed up to rt and stirred over night. Concentrated down at rt bath temperature to dryness. loaded onto a biotage snap on 120 g C18 column, eluted with 0-25% AcCN in water in 20 CV, collected fractions and lyophilized to furnish the title material as a powder. UPLC Method C: Rt=3.103 min, m/z=775.34 [M+H].

### Step 2: (S)-2,2′-((6-amino-1-((2-morpholino-2-oxoethyl)amino)-1-oxohexan-2-yl)azanediyl)bis(N-(2-morpholino-2-oxoethyl)acetamide)

To the solution of (S)-benzyl (5-(bis(2-((2-morpholino-2-oxoethyl)amino)-2-oxoethyl)amino)-6-((2-morpholino-2-oxoethyl)amino)-6-oxohexyl)carbamate (1.15 g, 1.484 mmol) in Water (20 mL), was added 20% Pd/C (0.158 g, 1.484 mmol), set up hydrogen balloon, stirred at rt over night, filtered off the catalyst, lyophilized to powder. UPLC Method C: Rt=1.70 min, m/z=641.33 [M+H].

### Step 3: (S)-benzyl 6-((5-(bis(2-((2-morpholino-2-oxoethyl)amino)-2-oxoethyl)amino)-6-((2-morpholino-2-oxoethyl)amino)-6-oxohexyl)amino)-6-oxohexanoate

To the solution of (S)-2,2′-((6-amino-1-((2-morpholino-2-oxoethyl)amino)-1-oxohexan-2-yl)azanediyl)bis(N-(2-morpholino-2-oxoethyl)acetamide) (633 mg, 0.988 mmol) in DMF (3.5 mL) was added benzyl (2,5-dioxopyrrolidin-1-yl) adipate (412 mg, 1.235 mmol), followed by Et3N (0.172 mL, 1.235 mmol). The mixture was stirred at rt for 2 hours. Concentrated down, purified by Biotage snap on 120 g C₁₈ column, elute with 0-25% AcCN in water, 20 CV, combined fractions and lyophilized to powder. UPLC Method C: Rt=3.21 min, m/z=859.4 [M+H].

### Step 4: of (S)-6-((5-(bis(2-((2-morpholino-2-oxoethyl)amino)-2-oxoethyl)amino)-6-((2-morpholino-2-oxoethyl)amino)-6-oxohexyl)amino)-6-oxohexanoic acid

To the solution of (S)-benzyl 6-((5-(bis(2-((2-morpholino-2-oxoethyl)amino)-2-oxoethyl)amino)-6-((2-morpholino-2-oxoethyl)amino)-6-oxohexyl)amino)-6-oxohexanoate (690 mg, 0.803 mmol) in Water (50 mL), added 20% Pd/C (85 mg, 0.080 mmol), set up hydrogen balloon, stirred at rt over night. Filtered off the catalyst and lyophilized the filtrate to furnish the title compound as a powder. UPLC Method C: Rt=2.01 min, m/z=769.37 [M+H].

### Step 5: 2,5-dioxopyrrolidin-1-yl (S)-6-((5-(bis(2-((2-morpholino-2-oxoethyl)amino)-2-oxoethyl)amino)-6-((2-morpholino-2-oxoethyl)amino)-6-oxohexyl)amino)-6-oxohexanoate

To the solution of (S)-6-((5-(bis(2-((2-morpholino-2-oxoethyl)amino)-2-oxoethyl)amino)-6-((2-morpholino-2-oxoethyl)amino)-6-oxohexyl)amino)-6-oxohexanoic acid (700 mg, 0.910 mmol) in DMF (5.0 mL) was added TSTU (288 mg, 0.956 mmol), followed by Et3N (0.133 mL, 0.956 mmol), the mixture was stirred at room temperature for 2 hr, concentrated down and purified by Bioatage snap on 120 g C18 column, elute with 0-25% AcCN in water, collected fractions and lyophilized to powder to furnish the title compound. UPLC Method C: Rt=2.45 min, m/z=866.44 [M+H].

### Preparative Example 32

### Synthesis of 2,5-dioxopyrrolidin-1-yl 6-(2-(bis(2-((2-morpholino-2-oxoethyl)amino)-2-oxoethyl)amino)acetamido)hexanoate (Capping Reagent 14)

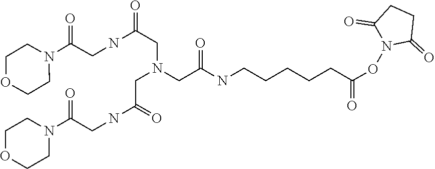

### Step 1 Benzyl 6-(2-(bis(2-((2-morpholino-2-oxoethyl)amino)-2-oxoethyl)amino)acetamido)hexanoate

To the solution of lysine 2,2′-((2-((6-(benzyloxy)-6-oxohexyl)amino)-2-oxoethyl)azanediyl)diacetic acid (1.0 g, 2.54 mmol) in dry DMF (7.0 ml) was added 2-amino-1-morpholin-4-yl-ethanone HCL (1.374 g, 7.61 mmol), followed by Et3N (2.120 mL, 15.21 mmol) and HOBT (1.165 g, 7.61 mmol), and to this mixture was added EDC (1.458 g, 7.61 mmol). The mixture was stirred at rt over night. The product was isolated by chromatography on 180 g C18 column, eluting with 0-30% AcCN in water (20 CV). UPLC Method A: Rt=0.83 min, m/z=647.82 [M+H].

### Step 2 6-(2-(bis(2-((2-morpholino-2-oxoethyl)amino)-2-oxoethyl)amino)acetamido)hexanoic acid

To the solution of benzyl 6-(2-(bis(2-((2-morpholino-2-oxoethyl)amino)-2-oxoethyl)amino)acetamido)hexanoate (1.6 g, 2.474 mmol) in 20 mL of water was added 20% Pd/C (0.263 g, 2.474 mmol), set up hydrogenation balloon, and stirred at rt over night. Filtered off the catalyst through glassfiber filter paper, and the filtrate was lyophilized to powder. UPLC Method A: Rt=0.45 min, m/z=557.37 [M+H].

### Step 3 2,5-dioxopyrrolidin-1-yl 6-(2-(bis(2-((2-morpholino-2-oxoethyl)amino)-2-oxoethyl)amino)acetamido)hexanoate

To the solution of 6-(2-(bis(2-((2-morpholino-2-oxoethyl)amino)-2-oxoethyl)amino)acetamido)hexanoic acid (1.33 g, 2.389 mmol) in DMF (5.0 mL) was added TSTU (0.755 g, 2.509 mmol), followed by Hunig's Base (0.438 mL, 2.509 mmol). The mixture was stirred at rt for 3 hour, concentrated, and purified on Biotage 180 g C₁₈ column, eluting with 0-20% AcCN in water, 20 CV, combined the product fractions and lyophilized to powder. UPLC Method C: Rt=2.34 min, m/z=654.29 [M+H].

General Method A: Synthesis of N^{6,29B} Acylated Human Insulins (Analogs) is described.

### Preparative Example 33

In an appropriately sized container, insulin or insulin analog was dissolved, with gentle stirring, at room temperature in a mixed solvent: 2:3 v/v 0.1 M Na₂CO₃:AcCN. After the mixture cleared, the pH was adjusted to the value of 10.5-10.8 using alkaline solution, e.g., 0.1 N NaOH. In a separate vial, an activated ester intermediate (Capping Reagent) was dissolved in an organic solvent, e.g., DMSO, at room temperature. Aliquots of the solution of the activated ester (Capping Reagent) were added over a period of time to the solution containing insulin until UPLC chromatogram showed that most of the unmodified insulin had been reacted and that a substantial portion of the reaction mixture had been converted into B29-conjugated insulin. The reaction was quenched by the addition of an amine nucleophile, e.g., 2-aminoethanol. The reaction solution was stirred at room temperature for 30 minutes. The resulting solution was carefully diluted with cold H₂O (20×) at 0° C. and its pH was adjusted to a final pH of 2.5 using 1 N HCl (and 0.1 N NaOH if needed). The solution was first concentrated by ultrafiltration, either through a tangential flow filtration (TFF) system or using Amicon Ultra-15 Centrifugal Units, with 1K, 3K or 10K MWCO membrane. The resulting solution was then further purified by reverse phase HPLC (Kromasil C8 250×50 mm, 10 □m, 100 Å column; Buffer A: 0.05-0.1% TFA in water; Buffer B: 0.05-0.1% TFA in AcCN). Fractions containing the title conjugate were combined and freeze-dried or buffer exchanged using TFF system and/or Amicon Ultra-15 to give the title product. Optionally, the material can also be subjected to ion exchange chromatography (PolySULFOETHYL A column, PolyLC Inc., 250×21 mm, 5 □m, 1000 Å; Buffer A: 0.1%(v/v)H₃PO₄/25% AcCN; Buffer B: 0.1%(v/v)H₃PO₄/25% AcCN/0.5 M NaCl). Fractions containing B29-conjugate with desired purity are combined, concentrated using TFF system or Amicon Ultra-15, and de-salted by reverse phase HPLC as described earlier.

As an alternative to the acylation in aqueous media with Na₂CO₃ as the base described above, RHI may be dissolved in an a polar organic solvent (e.g. DMSO) and treated with 20-40 eq. of a strong organic base, such as TMG or TMP followed by a drowise addition of a soluiton of the acylating agent in DMSO. The resulting mixture is stirred for 20-40 min, and then added dropwise to 50-100 vol. of a stirred mixture of 5:1 IPAC:MTBE. After stirring for 15 minutes, the suspended solids are collected via filtration, washed with 5:1 IPAC:MTBE, and the cake is dried. The product is purified as described above.

As another alternative for the synthesis of B29-conjugates with improved selectivity of the reaction, A1-trifluoroacetyl-protected RHI (F. Liu et. al., Journal of Peptide Sci., 2012, 18, 336-341) can be used as a starting material. Following conjugation, the trifluoroacetamide protective group is removed by aqueous ammonium hydroxide.

### Preparative Example 34

General Method A: Synthesis of N^{6,29B} Dimethylaminoacetyl RHI, (Analog 1).

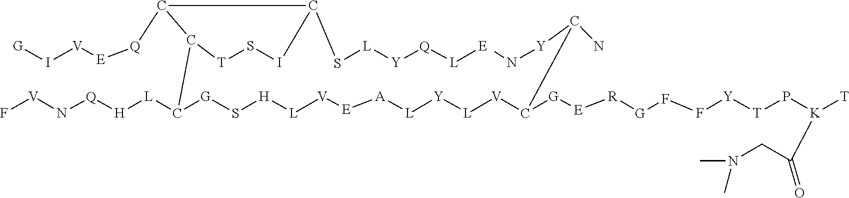

In a vial, dissolved RHI (300 mg, 0.052 mmol) in a mixture of 0.1M Na₂CO₃ (1.8 mL) and Acetonitrile (1.2 mL). Adjusted pH to 10.8. Added a solution of 2,5-dioxopyrrolidin-1-yl 2-(dimethylamino)acetate (Capping Reagent 1) (0.1M/DMF), (517 μl, 0.052 mmol) and stirred 30 min. Diluted the mixture with 15 mL of water and acidified with 1M HCl to pH 2.5. The product was purified by ion-exchange chromatography (IEC) (PolySULFOETHYL A column, PolyLC Inc., 250×21 mm, 5 □m, 1000 Å; Buffer A: 0.1%(v/v)H₃PO₄/25% AcCN; Buffer B: 0.1%(v/v) H₃PO₄/25% AcCN/0.5 M NaCl), gradient 10-60% of Solvent B in 24 min. Fractions containing the desired material were combined, and the product was re-purified by reverse-phase chromatography (KROMASIL C8 250×50 mm, 10 μm, 100 Å column; Buffer A: 0.05% TFA in deionized water; Buffer B: 0.05% TFA in AcCN). Fractions containing the desired material were lyophilized to furnish N^{6,29B} acylated RHI (Analog 1), UPLC Method D: Rt=3.15 min, m/z=1474.22 [(M+4)/4]).

### Preparative Example 35

### Synthesis of N^{6,29B} Acylated RHI (Analog 2) is Described

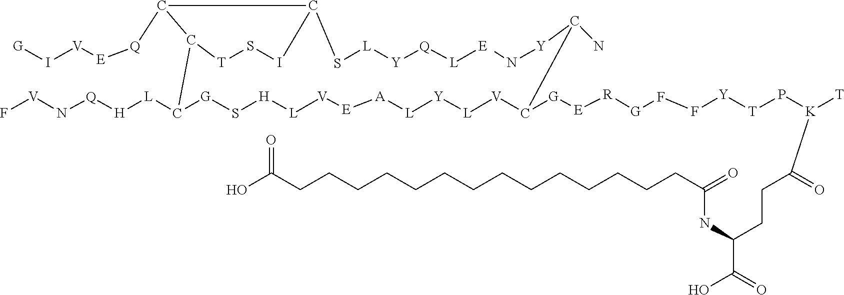

Starting from RHI and using (S)-16-((1-carboxy-4-((2,5-dioxopyrrolidin-1-yl)oxy)-4-oxobutyl)amino)-16-oxohexadecanoic acid (Capping Reagent 5) as the B29 capping reagent (previously described for insulin degludec, WO2010/029159 A1), Analog 2 was synthesized according to General Method A. UPLC Method D: Rt=2.99 min, m/z=1552.37 [(M+4)/4].

### Preparative Example 36

### Synthesis of N^{6,29B} Acylated RHI (Analog 3) is described

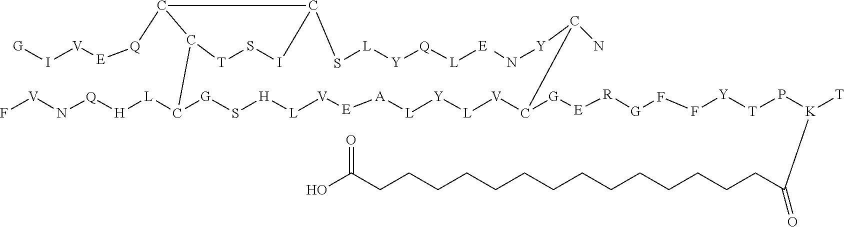

Starting from RHI and 16-((2,5-dioxopyrrolidin-1-yl)oxy)-16-oxohexadecanoic acid (Capping Reagent 2), Analog 3 was obtained according to General Method A. UPLC Method G: Rt=3.90 min, m/z=1519.22 [(M+4)/4]).

### Preparative Example 36

### Synthesis of N^{6,29B} Acylated des-B30-RHI (Analog 3a) is Described

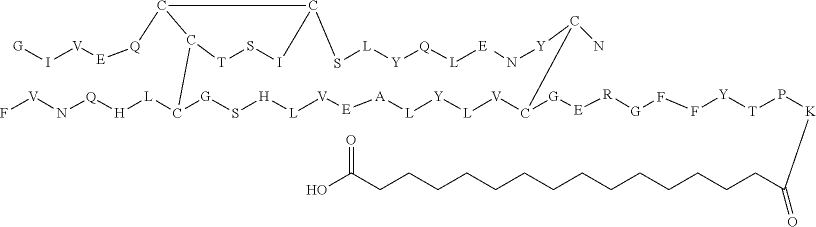

Starting from des-B30-RHI and 16-((2,5-dioxopyrrolidin-1-yl)oxy)-16-oxohexadecanoic acid (Capping Reagent 2), Analog 3a was obtained according to General Method A. UPLC Method G: Rt=3.94 min, m/z=1992.43 [(M+4)/4]).

### Preparative Example 37

### Synthesis of N^{6,29B}-Boc RHI (Analog 4) is Described

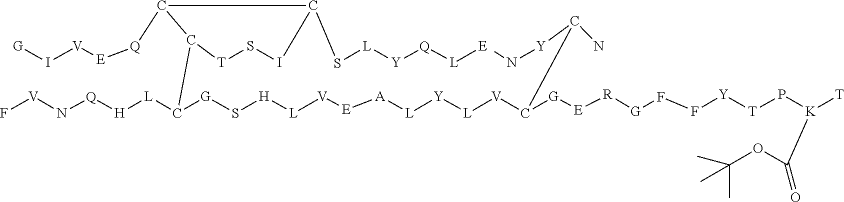

To a solution of RHI (20 g, 3.44 mmol) in DMSO (180 mL) was added 1,1,3,3-tetramethylguanidine (8.64 mL, 68.9 mmol) followed by a dropwise addition of a solution of tert-butyl (2,5-dioxopyrrolidin-1-yl) carbonate (0.741 g, 3.44 mmol) in DMSO (20.00 mL). The resulting mixture was stirred for 40 min, and was then added dropwise to a stirred mixture of 5:1 IPAC:MTBE (2000 ml). After stirring for 15 minutes, the suspended solid was collected via filtration and washed with 3×200 mL 5:1 IPAC:MTBE. The cake was dried by pulling vacuum through it with a stream of nitrogen above it overnight. The desired material was purified by HPLC (KROMASIL C8 250×50 mm, 10 μm, 100 Å column; Buffer A: 0.05% TFA in deionized water; Buffer B: 0.05% TFA in AcCN). UPLC-MS Method G: Rt=2.88 min, m/z=1477.54 [(M+4)/4

### Preparative Example 38

### Synthesis of N^{6,29B} (2,5,8,11,14,17,20,23-octaoxahexacosan-26-oyl) RHI (Analog 5)

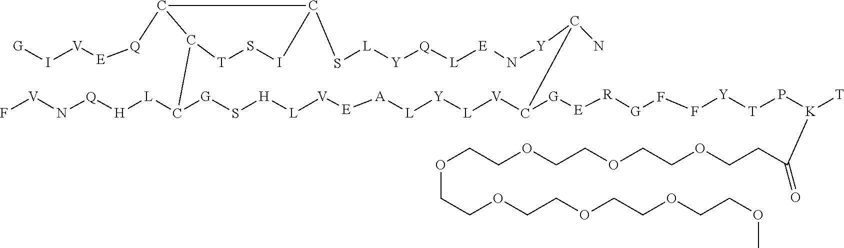

is described.

Using the procedures of General Method A and 2,5-dioxopyrrolidin-1-yl 2,5,8,11,14,17,20,23-octaoxahexacosan-26-oate (Capping Reagent 6), the title compound was isolated as a white solid after reverse-phase chromatography on C8 phase (Column KROMASIL, C8 10 μm 100 Å, 250×50 mm; solvent A=water/0.05% TFA, solvent B=AcCN/0.05% TFA), flow rate=85 mL/min, gradient B in A 26-43% over 30 min). UPLC Method D, Rt=3.64 min, m/z=1551.29 [(M+4)/4].

### Preparative Example 39

### Synthesis of Analog 6 is Described

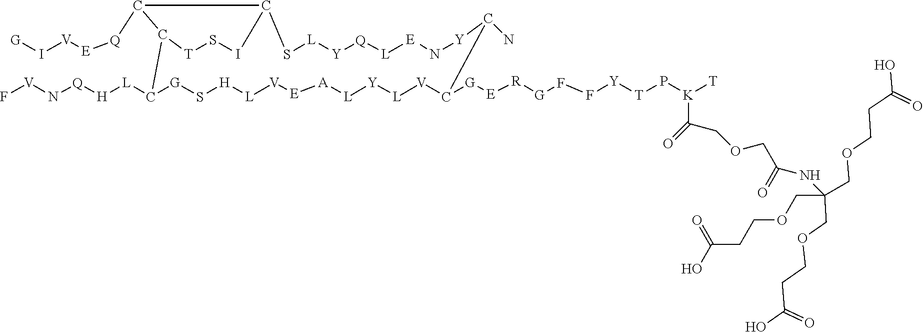

To a mixture of RHI (500 mg, 0.086 mmol) and 2,2,6,6-tetramethylpiperidine (581 μl, 3.44 mmol) in DMSO (5064 μl) was added a solution of 3,3′-((2-((2-carboxyethoxy)methyl)-2-(2-(2-((2,5-dioxopyrrolidin-1-yl)oxy)-2-oxoethoxy)acetamido)propane-1,3-diyl)bis(oxy))dipropionic acid (Capping Reagent 20) (47.4 mg, 0.086 mmol) in 200 μL of dmso. Stirred 1 hr. Added the reaction mixture to 20 vol of IPAC-20% Et2O. Aged in fridge overnight. Filtered the resulting solid product and dried on sucction in stream of nitrogen for 2 hr. The product was purified by reverse-phase chromatography on C-8 phase (Column KROMASIL, C8 10 μm 100 Å, 250×50 mm; solvent A=water/0.05% TFA, solvent B=AcCN/0.05% TFA), Flow=85 mL/min, gradient B in A 26-34% in 30 min. UPLC Method D: Rt=2.04 min, m/z=1562.38 [(M+4)/4]).

### Preparative Example 40

### Synthesis of Analog 7 is Described

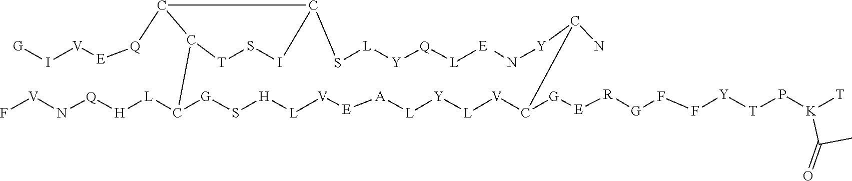

Dissolved RHI (3.0 g, 0.517 mmol) in a mixture of Na2CO3 (0.1M) (18.23 mL) and acetonitrile (12.15 mL) and adjusted pH ot 10.5. Added a solution of N-acetoxysuccinimide (Capping Reagent 7) (0.106 g, 0.672 mmol) in a small (600 μL) volume of acetonitrile. The reaction mixture was pH-adjusted to pH=2.5 with 1M HCl and the product of mono-conjugation was separated from the product of di-conjugation by reverse-phase chromatography on C-8 phase (Column KROMASIL, C8 10 μm 100 Å, 250×50 mm; solvent A=water/0.05% TFA, solvent B=AcCN/0.05% TFA), Flow=85 mL/min, gradient B in A 26-35% in 30 min. UPLC-MS Method C: Rt=3.58 min, m/z=1463.17[(M+4/4)].

### Preparative Example 41

### Synthesis of Analog 8 Starting from A1-Trifluoroacetyl-RHI (A1-TFA-RHI) is Described

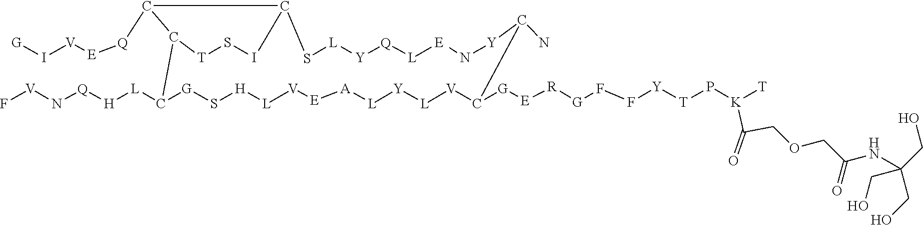

To a solution of A1-TFA-RHI (F. Liu et. al., Journal of Peptide Sci., 2012, 18, 336-341) (1000 mg, 0.169 mmol) in DMSO (9964 μl) was added triethylamine (708 μl, 5.08 mmol) followed by 2,5-dioxopyrrolidin-1-yl 2-(2-((1,3-dihydroxy-2-(hydroxymethyl)propan-2-yl)amino)-2-oxoethoxy)acetate (Capping Reagent 21) (0.1M/DMF) (1694 μl, 0.169 mmol). Stirred reaction 30 min. Isolated the product of the acylation by reverse-phase chromatography on C-8 phase (Column Kromasil, C8 10 uM 100 Å, size 250×50 mm; solvent A=water/0.05% TFA, solvent B=AcN/0.05% TFA), Flow=85 mL/min, gradient B in A 26-35% in 30 min. UPLC-MS Method D: Rt=3.66 min, m/z=1531.46 [(M+4/4)]. After lyophilization of the fractions, the product of acylation was taken in 5 mL of commercial conc NH4OH (˜28%) at 0 C for a period of 1 hr. This resulted in removal of TFA group. Removed most of NH4OH by diafiltration in 10K Amicon tubes. Adjusted pH to 3, and isolated the product by reverse-phase chromatography on C-8 phase (Column Kromasil, C8 10 uM 100 Å, size 250×50 mm; solvent A=water/0.05% TFA, solvent B=AcN/0.05% TFA), Flow=85 mL/min, gradient B in A 26-33% in 30 min. UPLC-MS Method D: Rt=3.53 min, m/z=1507.20[(M+4/4)].

### Preparative Example 42

### Analog 9 B29-Trifluoroacetyl-RHI

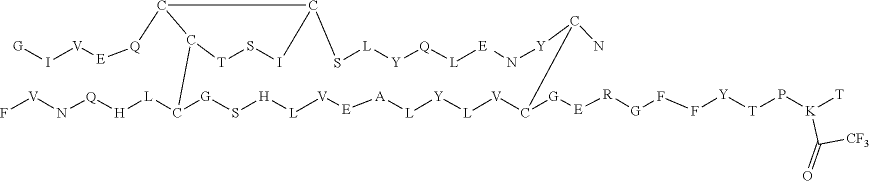

The title material was prepared using Capping Reagent 30 according to F. Liu et. al., Journal of Peptide Sci., 2012, 18, 336-341. UPLC-MS Method D: Rt=3.36 min, m/z=1476.48 [(M+4/4)].

The following examples of Analogs (monomeric insulins) in Table IV were prepared using the procedure analogous to General Method A but substituting appropriate starting materials that are either commercially available or prepared using procedure analogous to those described herein.

### Preparative Example 43

TABLE IV

| | | UPLC | | |
|---|---|---|---|---|
| Ana- | | Meth- | Rt | (M + |
| log # | Structure | od | (min) | 4)/4 |
| 10 | 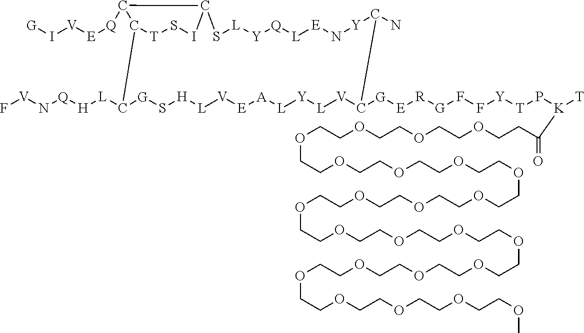 | D | 3.36 | 172 7.70 |
| 11 | 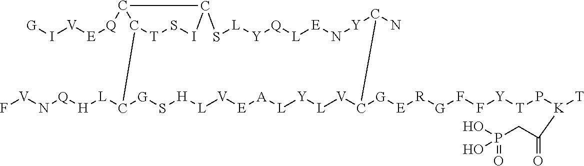 | D | 3.54 | 148 2.86 |
| 12 | 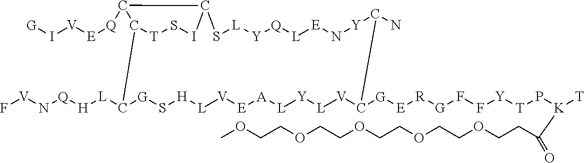 | D | 3.64 | 151 8.13 |
| 13 | 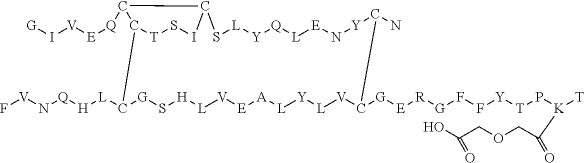 | D | 3.68 | 148 1.99 |
| 14 | 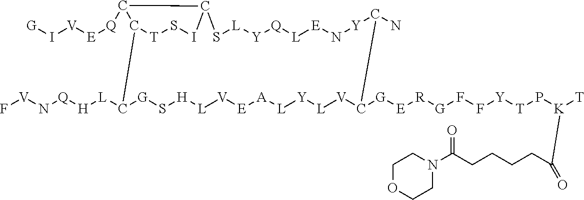 | D | 3.66 | 150 2.03 |
| 15 | 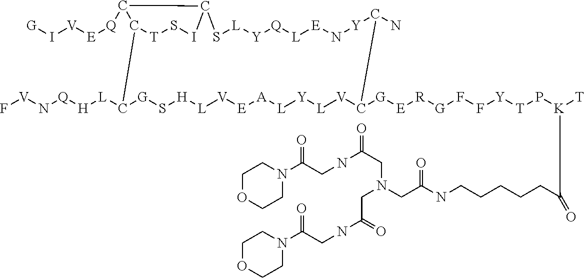 | D | 3.57 | 158 6.8 |
| 16 | 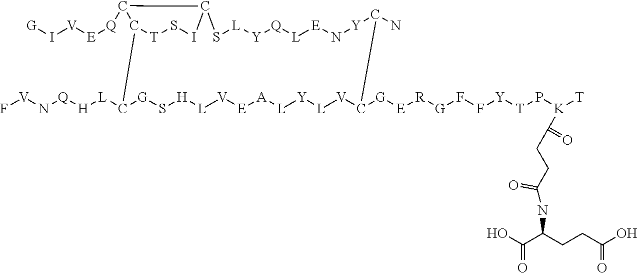 | D | 3.57 | 150 9.5 |
| 17 | 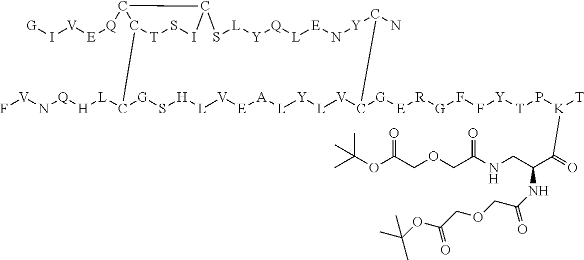 | D | 4.03 | 156 0.32 |
| 18 | 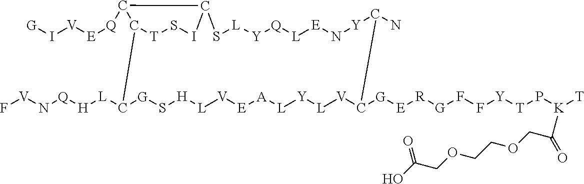 | D | 3.59 | 149 2.68 |
| 19 | 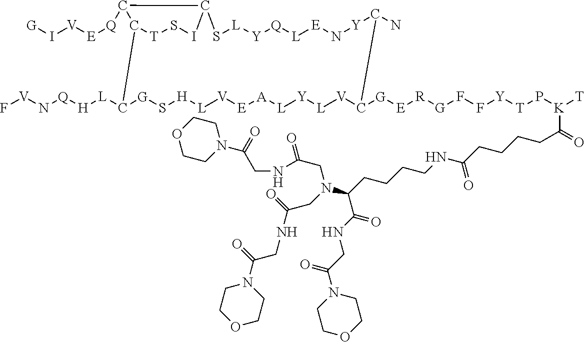 | A | 0.81 | 164 1.23 |
| 20 | 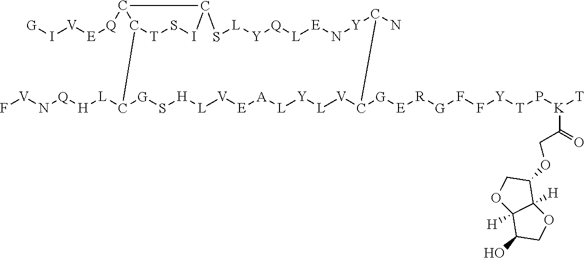 | D | 3.55 | 149 9.57 |
| 21 | 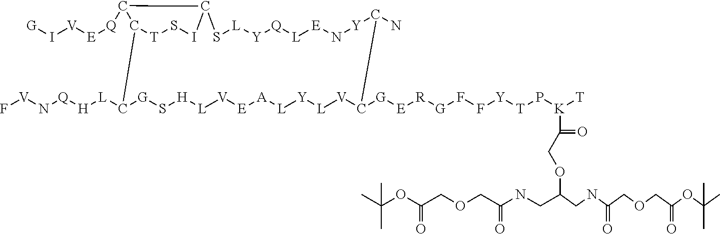 | D | 3.95 | 157 0.98 |
| 22 | 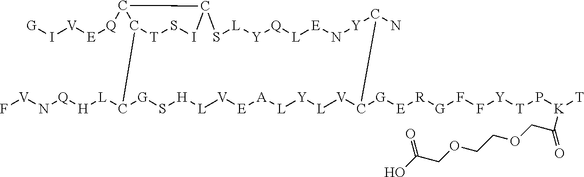 | D | 3.59 | 149 2.25 |
| 23 | 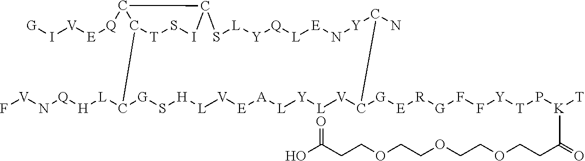 | D | 3.58 | 151 0.54 |
| 24 | 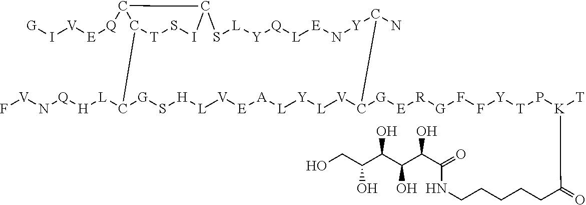 | D | 3.53 | 152 5.38 |
| 25 | 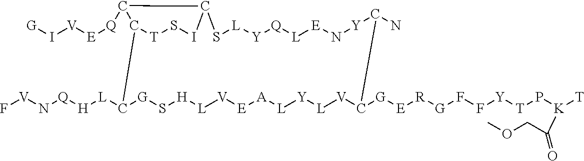 | D | 3.21 | 147 0.29 |

### Preparative Example 44

### General Method B: Synthesis of B1,B29-Bis-Capped Insulins (Analogs) is Described

To a solution A1-TFA-RHI (F. Liu et. al., Journal of Peptide Sci., 2012, 18, 336-341) in DMSO (optionally generated in situ from RHI and ethyl trifluoroacetate) a DMSO solution of an activated ester intermediate (Capping Reagent) is added at room temperature. The mixture is stirred until UPLC chromatogram shows that most of the A1-TFA-RHI has been reacted and that a substantial portion of the reaction mixture had been converted into A1-TFA, B1,B29-capped insulin. The reaction mixture is then mixed with commercial concentrated ammonium hydroxide solution. Optionally, before treatment with ammonium hydroxide, the DMSO solvent of the reaction mixture can be removed either by exchange for water using a tangential flow filtration (TFF) system or using Amicon Ultra-15 Centrifugal Units, with 1K, 3K or 10K MWCO membrane, or by precipitation of the product by addition of the reaction mixture into 50-100 volumes of weak organic solvent, such as ether, MTBE, IPAC, or a mixture of thereof. After confirmation by UPLC-MS that most of A1-TFA group has been cleaved by ammonium hydroxide, the solution is diluted with water and neutralized by addition of 3N HCl to pH=7.5. The solution is concentrated using a tangential flow filtration (TFF) system or using Amicon Ultra-15 Centrifugal Units, with 1K, 3K or 10K MWCO membrane, and the product is purified by reverse phase HPLC (Kromasil C8 250×50 mm, 10 □m, 100 Å column; Buffer A: 0.05% TFA in water; Buffer B: 0.05% TFA in AcCN). Fractions containing the title conjugate were combined and freeze-dried or buffer exchanged using TFF system and/or Amicon Ultra-15 to give insulin acylated with a capping group at B1 and B29 sites.

### Preparative Example 45

### General Method B. Synthesis of Analog 26

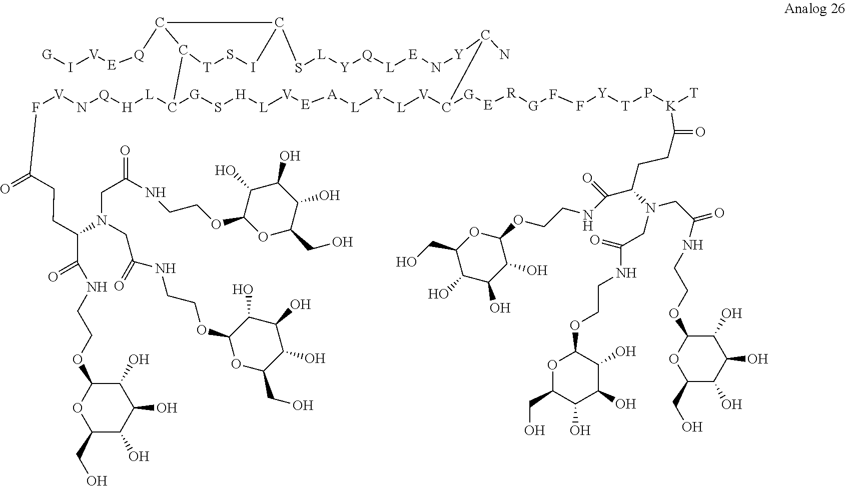

To a solution of A1-TFA-RHI (F. Liu et. al., Journal of Peptide Sci., 2012, 18, 336-341) (200 mg, 0.034 mmol) in DMSO (1.99 mL) was added Hunig's base (89 μl, 0.508 mmol) followed by 2,5-dioxopyrrolidin-1-yl (S)-4-(bis(2-oxo-2-((2-(((2R,3R,4S,5S,6R)-3,4,5-trihydroxy-6-(hydroxymethyl)tetrahydro-2H-pyran-2-yl)oxy)ethyl)amino)ethyl)amino)-5-oxo-5-((2-(((2R,3R,4S,5S,6R)-3,4,5-trihydroxy-6-(hydroxymethyl)tetrahydro-2H-pyran-2-yl)oxy)ethyl)amino)pentanoate (Capping Reagent 3) (99 mg, 0.102 mmol). Stirred the reaction mixture for 4 hours. Diluted the reaction mixture by 15 mL of water and removed most of DMSO by repeated diafiltration in Amicon tubes (10 K membrane), eventually concentrating to a volume of 3.0 mL. Added to this solution 3.0 mL of concentrated commercial ammonium hydroxide solution and allowed to stand for 4 hours. Diluted the mixture to the total volume of 15 mL and diafiltrated in 3 cycles in Amicon tubes (10 K membrane), from volume of 15 mL to 5 mL, adding fresh water after each cycle. Adjusted pH to 2.5 with 1M HCl and purified the product by reverse-phase chromatography (KROMASIL C8 250×50 mm, 10 μm, 100 Å column; Buffer A: 0.05% TFA in deionized water; Buffer B: 0.05% TFA in AcCN). After lyophilization of fractions, obtained B1,B29-blocked RHI as white solid, UPLC method D: Rt=3.04 min, m/z=1883.15 [(M+4)/4].

### Preparative Example 46

### General Method B. Synthesis of Analog 27

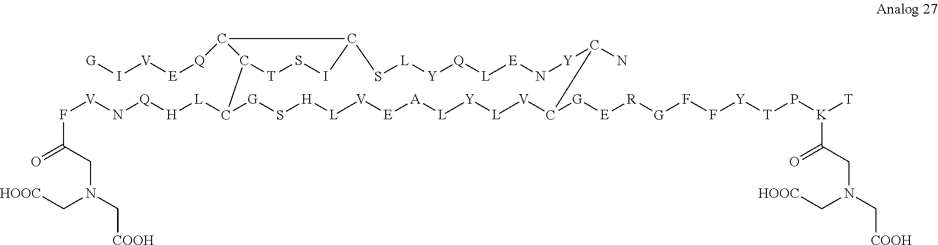

To a mixture of A1-TFA-insulin (F. Liu et. al., Journal of Peptide Sci., 2012, 18, 336-341) (100 mg, 0.017 mmol) in DMF (1.88 mL) was added 2-(2,6-dioxomorpholino)acetic acid (Capping Reagent 15) (8.80 mg, 0.051 mmol) as solid followed by triethylamine (47.2 μl, 0.339 mmol). Stirred at room temp for 3 hrs, removed most of DMF by diafiltration in Amicon tubes (10K MWCO membrane) using 3 cycles, adding fresh water after each cycle, reducing the volume from 15 mL to 3 mL per each cycle. After the last cycle (3 mL volume remaining) added 3 mL of concentrated ammonium hydroxide (commercial) and let the mixture stand for 3 hr. Removed most of the ammonium hydroxide by diafiltration (Amicon tube, 10K MWCO, 2 cycles). The material was pH-adjusted to 2.5 and purified by reverse-phase chromatography (KROMASIL C8 250×50 mm, 10 μm, 100 Å column; Buffer A: 0.05% TFA in deionized water; Buffer B: 0.05% TFA in AcCN). UPLC method D: Rt=3.27 min, m/z=1539.06 [(M+4)/4].

### Preparative Example 47

### Synthesis of N^{2,1B},N^{6,29B}-bis(3-(2-(2-methoxyethoxy)ethoxy)propanoyl) Human Insulin (Analog 28) is Described

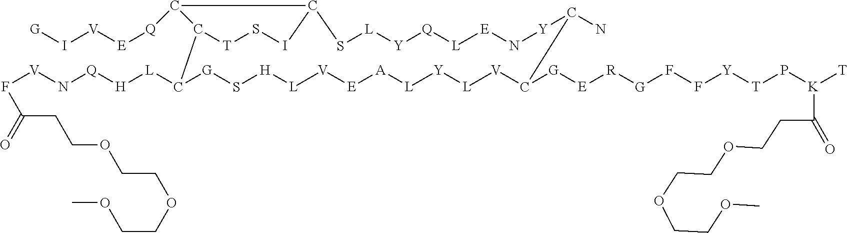

Starting from A1-TFA-RHI (F. Liu et. al., Journal of Peptide Sci., 2012, 18, 336-341), using 2,5-dioxopyrrolidin-1-yl 3-(2-(2-methoxyethoxy)ethoxy)propanoate (Capping Reagent 9) as the capping reagent and procedures of General method B, the title compound was obtained as a solid after reverse-phase chromatography on C8 phase (Column KROMASIL, C8 10 μm 100 Å, 250×50 mm; solvent A=water/0.05% TFA, solvent B=AcCN/0.05% TFA), flow rate=85 mL/min, gradient B in A 26-33% over 30 min). UPLC Method D, Rt=4.18 min, m/z=1939.67 [(M+4)/4].

### Preparative Example 48

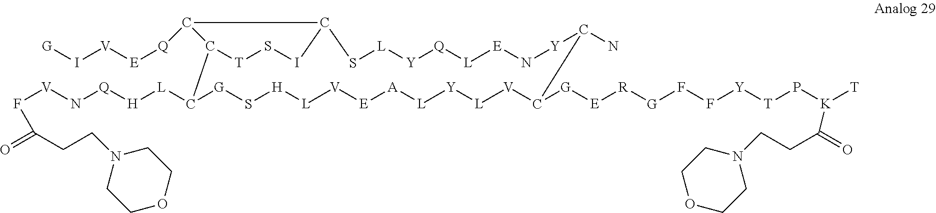

Using 2,5-dioxopyrrolidin-1-yl 3-(2-(2-methoxyethoxy)ethoxy)propanoate (Capping Reagent 18) as the capping reagent, the title compound was synthesized using procedures of General Method B. The product was purified by reverse-phase chromatography (KROMASIL C8 250×50 mm, 10 μm, 100 Å column; Buffer A: 0.05% TFA in deionized water; Buffer B: 0.05% TFA in AcCN, B in A 29-35% in 30 min.). UPLC Method D, Rt=4.08 min, m/z=1523.41 [(M+4)/4].

### Preparative Example 49

This example illustrates synthesis of N^{2,1B},N^{6,29B}-bis oxyacetic acid RHI (Analog 30) according to general method B.

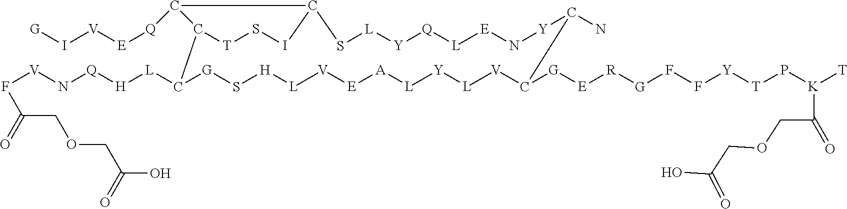

### Step 1 Synthesis of N²,1 Å-Trifluoroacetyl-RHI

To a solution of RHI (1.0 g, 0.172 mmol) in DMSO (10.0 mL) was added DIPEA 400 μl, 2.290 mmol) and the resulting mixture was stirred at room temperature for 5 minutes. Isopropyl 2,2,2-trifluoroacetate (150 μl, 1.065 mmol) was then added dropwise to the reaction mixture and the resulting mixture was stirred at room temperature for 2 h. UPLC-MS Method D: Rt=3.65 min, m/z=1476.42 [(M+4)/4]. Crude reaction mixture was used in the following step without any purification.

### Step 2 B1/B29 Bis oxyacetylation on N^{2,1A}-Trifluoroacetyl-RHI

To the reaction mixture from step 1 was added a solution of 1,4-dioxane-2,6-dione (Capping Reagent 12) (40 mg, 0.345 mmol) in 100 μL DMSO. and the resulting mixture was stirred at rom temperature for 2 h. Crude reaction mixture was added dropwise to a round-bottom flask containing 150 mL of IPAc/MTBE (4:1). The resulting white suspension was filtered and rinsed with (3×50 mL of IPAc). The material was dried under high vacuum for 1 h and used in the following step without any further purification. UPLC-MS Method D: Rt=3.86 min, m/z=1534.95 [(M+4)/4].

### Step 3 Deprotection of trifluoroacetyl

The crude product of step 2 was dissolved in 5.0 mL of 10% CH3CN in H2O, then 5.0 mL of commercial NH4OH (28% m/v) was added dropwise at 0° C., and the mixture was stirred at same temperature for 2 hours. Upon completion, the crude reaction mixture was concentrated to 5.0 mL using spin-dialysis on a 10K MWCO membrane Amicon tube, and diafiltration was continued using 100 mL water (pH=3.00) to a final volume about 20 mL and purified by HPLC. (Kromasil C8 250×50 mm, 10 μm, 100 Å column; Buffer A:0.1% TFA in water; Buffer B: 0.1% TFA in AcCN). Fractions containing the title conjugate were combined and lyophilized to give the title product as a white solid. UPLC-MS D: Rt=3.68 min, m/z=1510.64 [(M+4)/4].

### Preparative Example 50

### Synthesis of Analog 31 is Described B1,B29=Phenylacetyl RHI

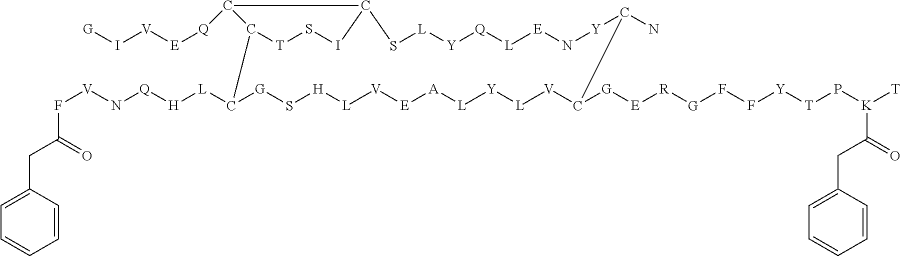

A1-TFA protected Insulin (F. Liu et. al., Journal of Peptide Sci., 2012, 18, 336-341, 400 mg, 0.068 mmol) was dissolved in DMSO (3 mL) and treated with Hunig's Base (0.355 mL, 2.033 mmol) and 2,5-dioxopyrrolidin-1-yl 2-phenylacetate (Capping Reagent 31) (63 mg, 0.271 mmol). After 2 hr of stirring, the reaction mixture was diluted with ice-cold water (30 mL) and treated with 6 mL of concentrated commercial ammonium hydroxide. After 4 hr of stirring, the reaction mixture was diafiltrated with addition of fresh water so that total of 50 mL of liquid was removed. The remaining solution was acidified to pH ˜2.5 (the initially formed a precipitate was re-dissolved by addition of acetonitrile so that the final volume was ˜80 mL of 1:1 water:AcN). The product was isolated by reverse phase prep HPLC (KROMASIL C8 250×50 mm, 10 μm, 100 Å column; Buffer A: 0.05% TFA in deionized water; Buffer B: 0.05% TFA in AcCN), Flow=85 mL/min, gradient B in A 30-45% in 30 min. UPLC-MS Method B: Rt=2.23 min, m/z=1511.51[(M+4/4)].

### Preparative Example 51

The following examples of Analogs (monomeric insulins) in Table V were prepared using the procedure analogous to General Method B but substituting appropriate starting materials that are either commercially available or prepared using procedure analogous to those described herein.

TABLE V

| | | UPLC | Rt | |
|---|---|---|---|---|
| Analog # | Structure | Method | (min) | (M + 4)/4 |
| 32 | 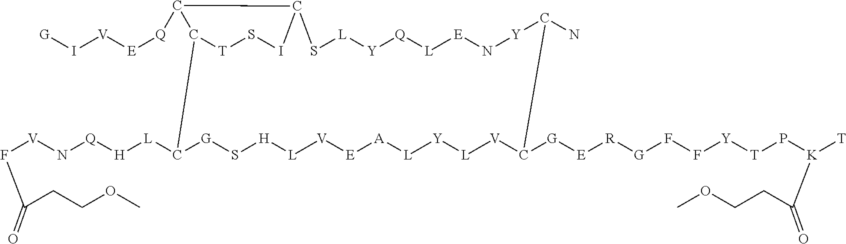 | D | 3.40 | 1495.33 |
| 33 | 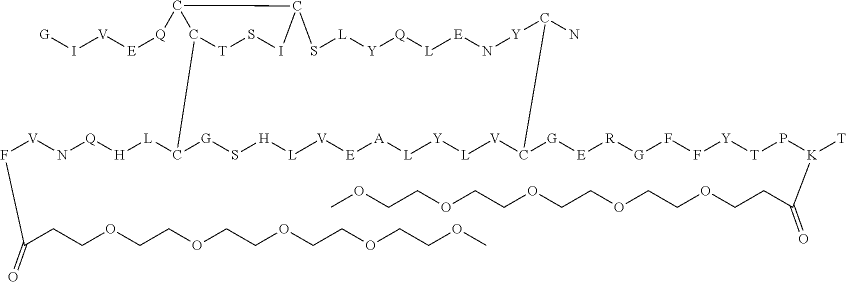 | D | 3.60 | 1583.69 |
| 34 | 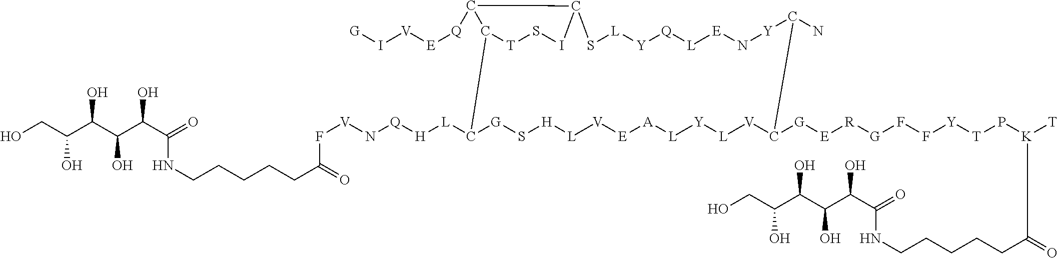 | D | 3.55 | 1598.22 |
| 35 | 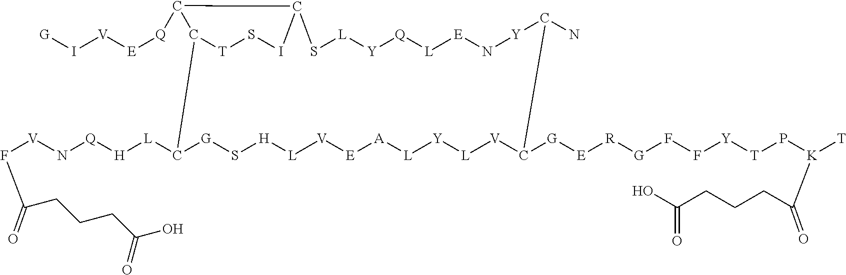 | A | 0.84 | 1509.9 |

### General Method C: Synthesis of N^{2,1A} Insulin Conjugates (Analogs)

### Preparative Example 52

In an appropriately sized container, insulin was dissolved, with gentle stirring, at rt in an aqueous pH=3.0 solvent. After the mixture cleared, the pH was adjusted to the value of 8.0-8.5 using alkaline solution, e.g., 0.1 N NaOH. In a separate vial, an activated ester intermediate was dissolved in an organic solvent, e.g., DMSO, at rt. Aliquots of the solution of the activated ester was added over a period of time to the solution containing insulin until UPLC chromatogram showed that most of the unmodified insulin had been reacted and that a substantial portion of the reaction mixture had been converted into A1-conjugated insulin. The reaction was quenched by the addition of an amine nucleophile, e.g., 2-aminoethanol. The reaction solution was stirred at rt for 30 min. The resulting solution was carefully diluted with cold H₂O (20×) at 0° C. and its pH was adjusted to a final pH of 2.5 using 1 N HCl (and 0.1 N NaOH if needed). The solution was first concentrated by ultrafiltration, either through a tangential flow filtration (TFF) system or using Amicorn Ultra-15 Centrifugal Units, with 1K, 3K or 10K MWCO membrane. The concentrated solution was usually first subjected to ion exchange chromatography (PolySULFOETHYL A column, PolyLC Inc., 250×21 mm, 5 μm, 1000 Å; Buffer A: 0.1%(v/v)H₃PO₄/25% AcCN; Buffer B: 0.1%(v/v)H₃PO₄/25% AcCN/0.5 M NaCl). Fractions containing B29-conjugate with desired purity were combined and concentrated using TFF system or Amicon Ultra-15. The resulting solution was then further purified by reverse phase HPLC (Waters C4 250×50 mm column, 10 μm, 1000 Å column or Kromasil C8 250×50 mm, 10 μm, 100 Å column; Buffer A: 0.05-0.1% TFA in water; Buffer B: 0.05-0.1% TFA in AcCN). Fractions containing the title conjugate were combined and freeze-dried or buffer exchaged using TFF system and/or Amicorn Ultra-15 to give the title product.

### Preparative Example 53

This example illustrates synthesis of RHI Analog 36 according to General Method C.

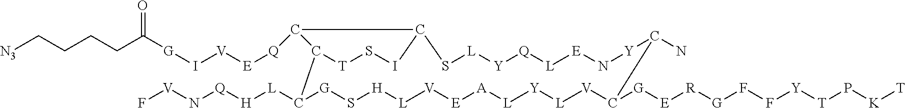

To a vial containing RHI (500 mg, 0.086 mmol) was added 20 mL of water, and the pH of the resulting solution was adjusted to 8.0-8.5 by Na2CO3 solution (0.1M). The mixture was allowed to stir at rt until all insulin was dissolved, and was treated with a solution of 2,5-dioxopyrrolidin-1-yl 5-azidopentanoate (41.4 mg, 0.172 mmol) in 430 μL of MeCN. After 2 h of stirring, the reaction mixture was analyzed by LCMS and additional portions of 2,5-dioxopyrrolidin-1-yl 5-azidopentanoate (41.4 mg, 0.172 mmol) were added as needed to complete the reaction. The product was isolated by ion-exchange chromatography (PolySULFOETHYL A column, PolyLC Inc., 250×21 mm, 5 μm, 1000 Å, flow rate 15 mL/min, 20-80% buffer B in buffer A; A: 0.1% (v/v) H3PO4/25% AcCN and B: 0.1% (v/v) H3PO4/25% AcCN/0.5 M NaCl). Fractions containing the desired mono-conjugated product were combined and re-purified by HPLC (KROMASIL C8 250×50 mm, 10 μm, 100 Å column; Buffer A: 0.05% TFA in deionized water; Buffer B: 0.05% TFA in AcCN). Fractions containing the desired products were combined and lyophilized to give a powder, UPLC method D: Rt=3.25 min, m/z=1484.13 [(M+4)/4].

### Preparative Example 54

This example illustrates synthesis of RHI Analog 37.

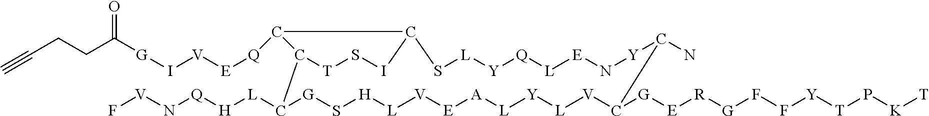

The title compound was synthesized according to General Method C. UPLC method D: Rt=4.08 min, m/z=1472.46 [(M+4)/4].

### Preparative Example 55

This example illustrates synthesis of RHI Analog 38.

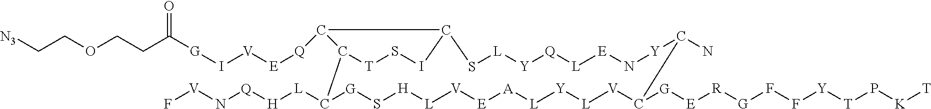

The title compound was synthesized according to General Method C. UPLC method D: Rt=3.04 min, m/z=1487.855 [(M+4)/4].

### Preparative Example 56

This example illustrates synthesis of RHI Analog 39.

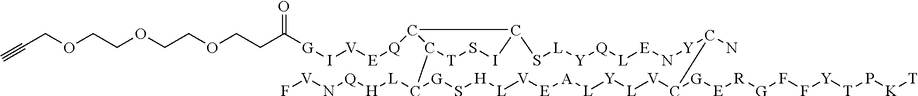

The title compound was synthesized according to General Method C. UPLC method D: Rt=3.22 min, m/z=1502.369 [(M+4)/4].

### Preparative Example 57

This example illustrates synthesis of RHI Analog 40.

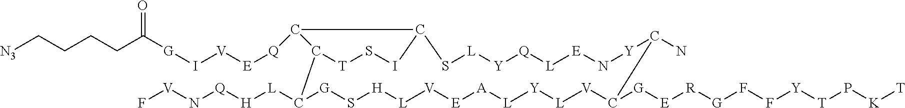

The title compound was synthesized according to General Method C from Y(19)A mutant of RHI. UPLC method D: Rt=3.87 min, m/z=1436.23 [(M+4)/4].

General Method D Synthesis of insulin dimers by linking at N^{2,1A},N^{2,1A′} positions, where B29 and B29′ are blocked with a capping group.

In an appropriately sized container, acylated insulin (Analog) is suspended at room temperature in an organic solvent, e.g., DMSO, or mixed aqueous (aq)/organic solvents, in the presence of a base, e.g., TEA, TMG, or TMP. The mixture is allowed to stir gently until insulin is completely dissolved. To the resulting solution is added a bis-functional activated ester intermediate (Linking Reagent) in solution of organic solvents, such as DMSO or DMF. After UPLC chromatogram shows that a substantial portion of the reaction mixture has converted into N^{2,1A},N^{2,1A′} insulin dimer, the reaction mixture may be subjected directly to reverse phase HPLC purification (KROMASIL C8 250×50 mm, 10 μm, 100 Å column; Buffer A: 0.05-0.1% TFA in deionized water; Buffer B: 0.05-0.1% TFA in AcCN), or the reaction may be quenched by careful dilution with cold acidic H2O (20×, pH about 3.0) at 0° C. and its pH is adjusted to a final pH of 2.5 using 1 N HCl (and 0.1 N NaOH if needed). The solution may first be concentrated by ultrafiltration, either through a tangential flow filtration (TFF) system or using Amicon Ultra-15 Centrifugal Units, with 1K, 3K or 10K MWCO membrane. The concentrated solution is usually first subjected to ion exchange chromatography (PolySULFOETHYL A column, PolyLC Inc., 250×21 mm, 5 μm, 1000 Å; Buffer A: 0.1%(v/v) H3PO4/25% AcCN; Buffer B: 0.1%(v/v)H3PO4/25% AcCN/0.5 M NaCl). Fractions containing A1-A1′-conjugate with desired purity are combined and concentrated using TFF system or Amicon Ultra-15. The concentrated solution is then subjected to reverse phase HPLC purification (KROMASIL C8 250×50 mm, 10 μm, 100 Å column; Buffer A: 0.05-0.1% TFA in deionized water; Buffer B: 0.05-0.1% TFA in AcCN). Fractions containing the desired insulin dimer are combined and freeze-dried or buffer exchanged using TFF system and/or Amicon Ultra-15 to give the N^{2,1A},N^{2,1A′} insulin dimers.

### Example 1

### Dimer 1 Illustration of General Method D: Synthesis of

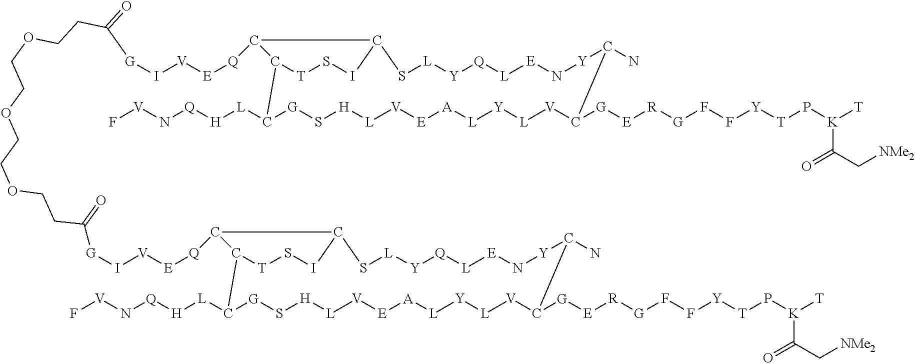

Dissolved Analog 1 (15 mg, 2.55 μmol) in DMSO (300 μl) and added triethylamine (7.10 μl, 0.051 mmol) followed by a solution of the linking reagent, diethylene glycol 1,2-bis(succinimidyl 3-oxypropionate) (Linking Reagent 31) (0.566 mg, 1.273 pmol) dissolved in 10 μL of DMSO. Stirred 1 hour, diluted with 20% AcN-water-0.05% TFA (15 mL) and diafiltrated in an Amicon tubes (10K MWCO membrane) to the volume of 1.5 mL in order to remove most of DMSO. The product was purified by ion-exchange chromatography (IEC) (PolySULFOETHYL A column, PolyLC Inc., 250×21 mm, 5 μm, 1000 Å; Buffer A: 0.1%(v/v)H₃PO₄/25% AcCN; Buffer B: 0.1%(v/v) H₃PO₄/25% AcCN/0.5 M NaCl), gradient 10-60% of Buffer B in 24 min. Fractions containing the desrired material were combined, and the product was re-purified on KROMASIL® C8 10 μm, 100 Å, 50×250 mm column, flow 85 mE/min, solvent A=water/0.05% TFA, solvent B=AcN/0.05% TFA, gradient B 26-35% in 30 min. The product was isolated as a solid after lyophilization of fractions. UPLC Method D: Rt=3.34 min, m/z=1714.88 [(M+7)/7]).

### Example 2

### Dimer 2 Synthesis of A1-A1′ Dimer of Degludec

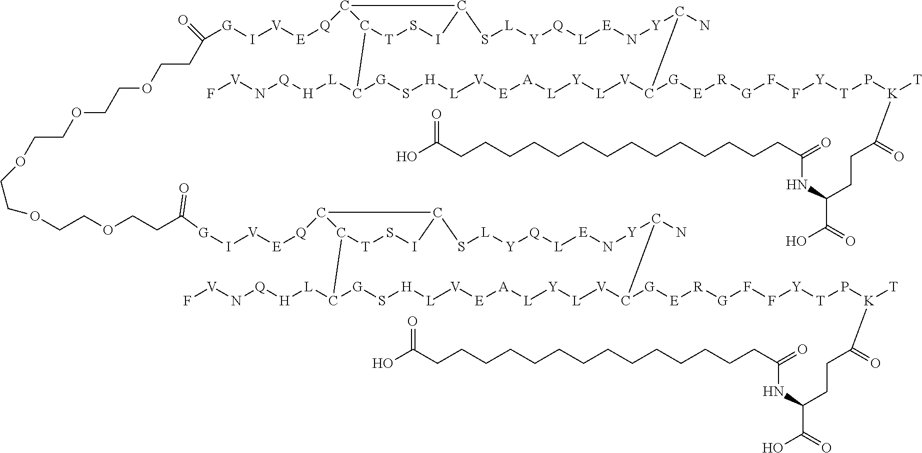

Starting from degludec (WO2010/029159 A1) and using bis(2,5-dioxopyrrolidin-1-yl) 4,7,10,13,16-pentaoxanonadecanedioate (Linking Reagent 33) as the linking reagent, the title compound was obtained according to General Method D. UPLC Method D: Rt=4.11 min, m/z=1788.28 [(M+7)/7].

### Example 3

### Dimer 3 Synthesis is Described

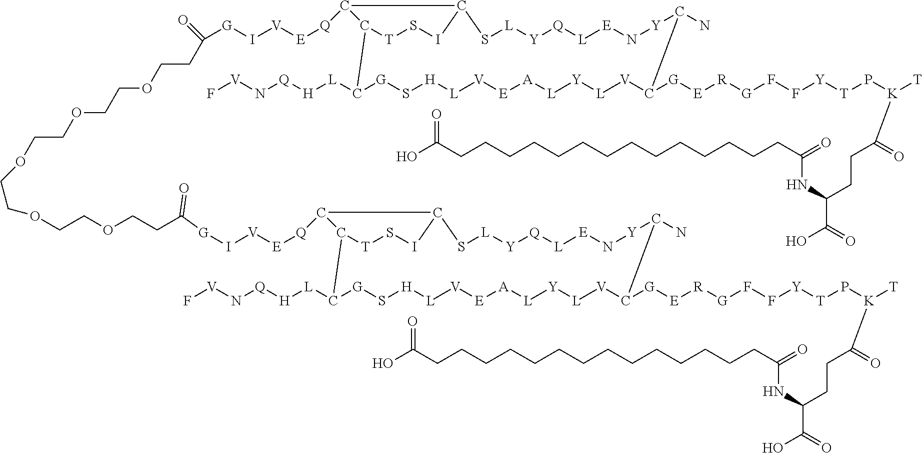

Starting from Analog 2 (Example 15) and Linking Reagent 33, using General Method D, the title compound was synthesized, UPLC Method D: Rt=4.06 min, m/z=1817.17 [(M+7)/7]).

### Example 4

### Dimer 4 Synthesis of

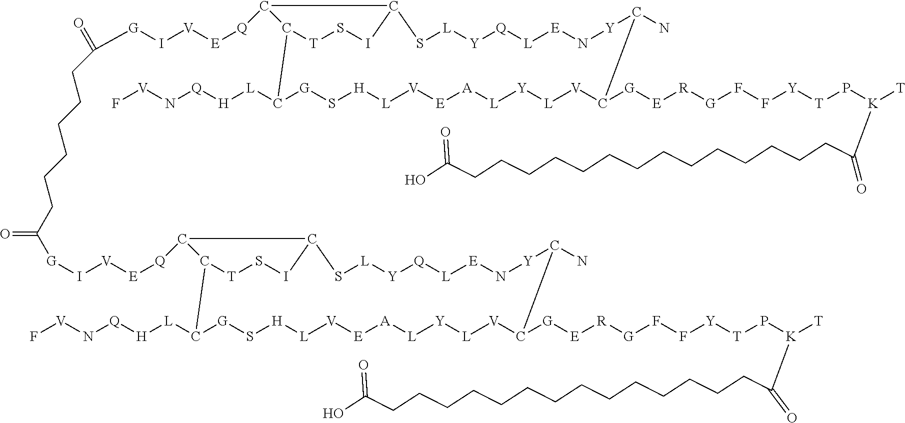

Starting from Analog 3 and using disuccinimidyl suberate (Linking reagent 35) as the linker and conditions of General Method D, the title compound was synthesized. UPLC Method D: Rt=3.96 min, m/z=1756.43 [(M+7)/7]).

### Example 5

### Dimer 5 Synthesis

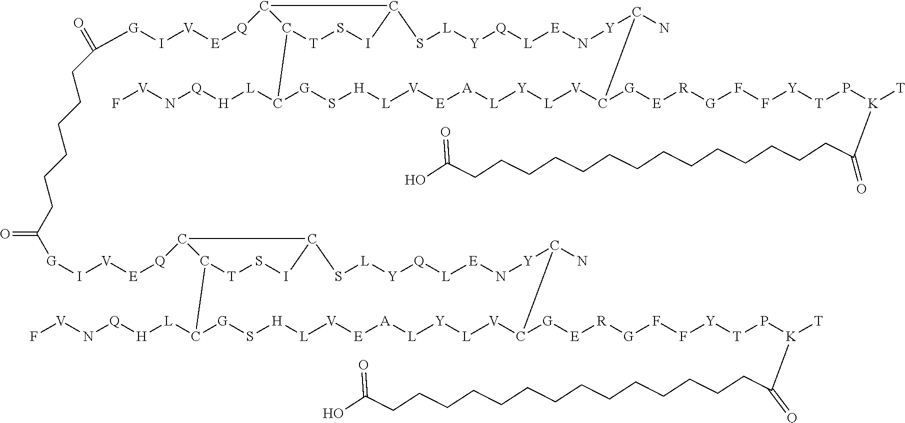

Starting from Analog 3a (acylated des-B30-RHI) and using disuccinimidyl suberate (Linking Reagent 35), Dimer 5 was obtained according to General Method D. UPLC Method D: Rt=3.81 min m/z=1728.04 [(M+7)/7]).

Dimers 1, 3, 4, 8 to 132 and 134 to 144 are represented by Formula I, wherein X and U are capping groups and Z is the linking moiety, including for dimers herein where the insulin backbone is not shown.

Dimers 2 and 5, which have Des-B30 backbone, are represented by Formula Ia, wherein X and U are capping groups and Z is the linking moiety, including dimers in dimer tables herein where the insulin backbone is not shown.

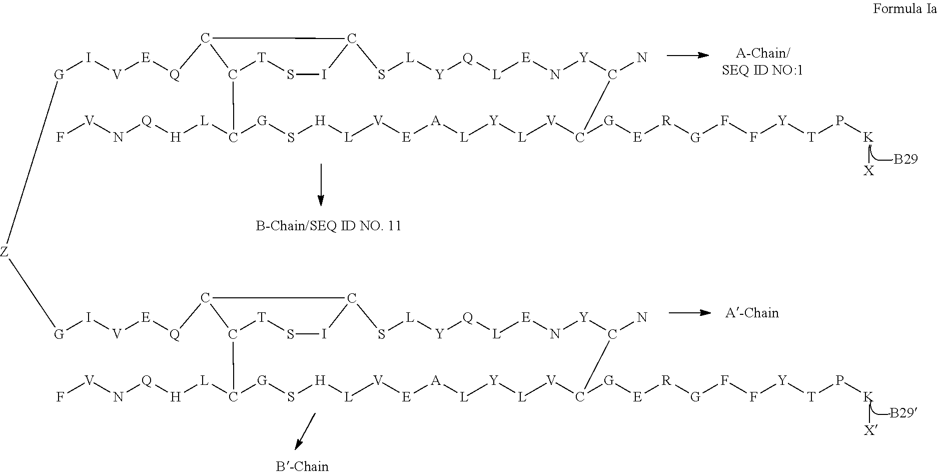

Dimers 6 and 7, which have Des-B30-B29R backbone, are represented by Formula Ib, wherein Z is the linking moiety, including dimers in dimer tables herein where the insulin backbone is not shown.

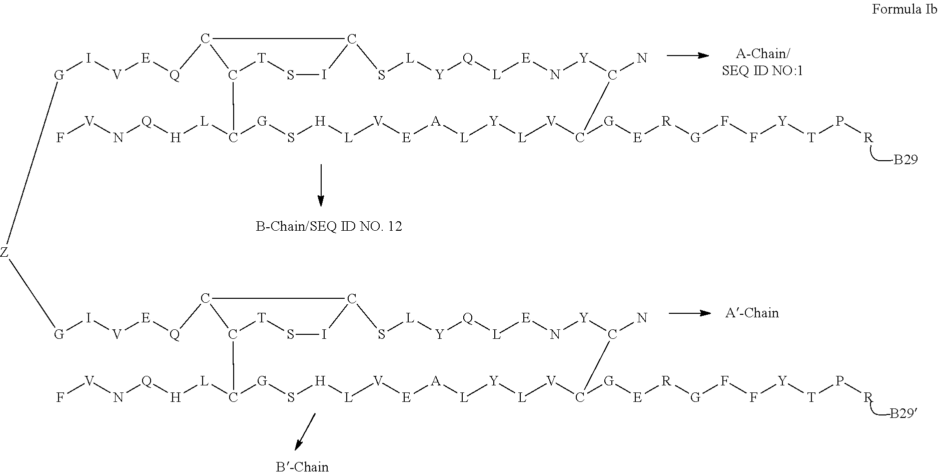

Dimer 133, which comprises a Y19A mutation, is represented by Formula Ic wherein Z is the linking moiety, including dimers in tables where the insulin backbone is not shown.

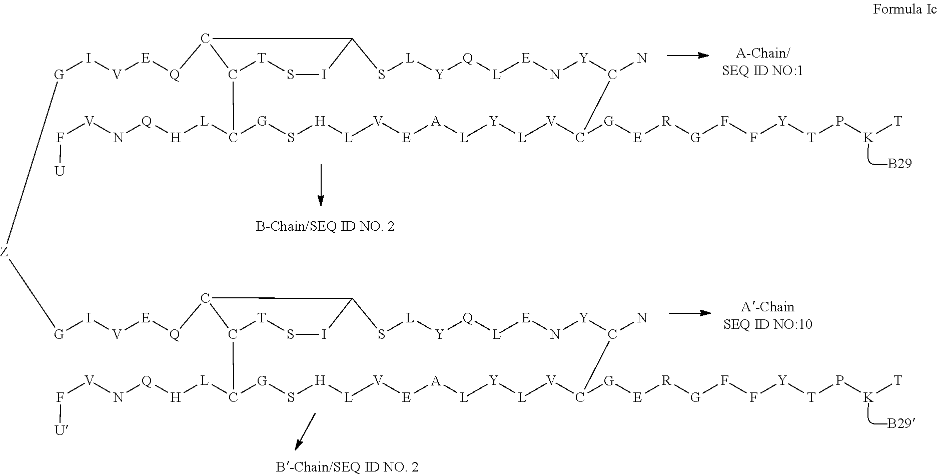

### Examples 6 and 7 (Dimers 6 and 7—the Backbone Depicted by Formula Ib)

Starting from an insulin containing B-chain SEQ ID NO:12, which, compared to RHI, contains a deletion at B30 position and replacement of B29 for Arginine, the following dimers listed in Table III and exemplified in Table VI below were obtained by reaction with appropriate Linking Reagent in conditions of General Method D.

TABLE VI
Dimers prepared with B29R-desB30-RHI using Method D, without capping groups.

| | | UPLC | Rt | (M + 6)/6 or |
|---|---|---|---|---|
| Dimer # | Linker Chemdraw | Method | (min) | ((M + 7)/7) |
| Dimer 6 | 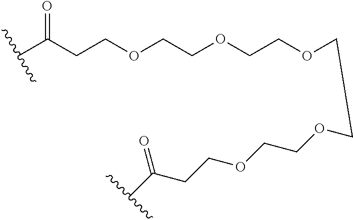 [SMILES: *C(=O)CCOCCOCCOCCOCCOCCC(*)=O] | D | 3.39 | 1962.73 |
| Dimer 7 | 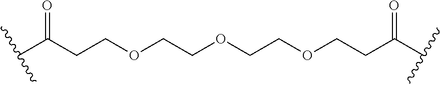 [SMILES: *C(=O)CCOCCOCCOCCC(*)=O] | D | 3.40 | 1947.77 |

### Example 8

Table III: Dimers 8 through 38 (backbone depicted by Formula I) in Table VII below were prepared from appropriate starting materials using General Method D: A1-A′ dimers with capping groups on B29,B29′ and uncapped positions at B1,B1′.

TABLE VII

| | | Capping group (wavy | | | |
|---|---|---|---|---|---|
| | Linker (wavy line | line indicates | | | (M + 6)/6 |
| Dimer | indicates attachment to A1 | attachment to B29 and | UPLC | Rt | or |
| # | and A1′ sites of insulins) | B29′ sites of insulins) | Method | (min) | ((M + 7)/7) |
| Dimer 8 | 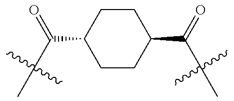 [SMILES: *C(=O)[C@H]1CC[C@H](C(*)=O)CC1] | 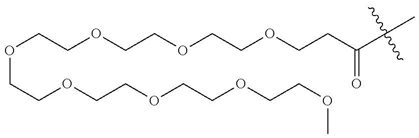 [SMILES: *C(=O)CCOCCOCCOCCOCCOCCOCCOCCOC] | D | 3.78 | 1792.52 |
| Dimer 9 | 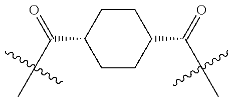 | 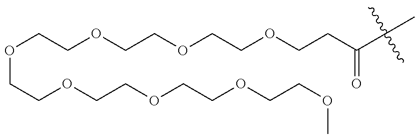 [SMILES: *C(=O)CCOCCOCCOCCOCCOCCOCCOCCOC] | D | 3.82 | 1792.32 |
| Dimer 10 | 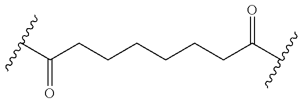 [SMILES: *C(=O)CCCCCCC(*)=O] | 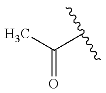 [SMILES: *C(C)=O] | D | 3.38 | 1973.61 |
| Dimer 11 | 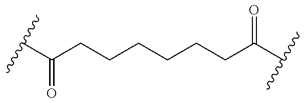 [SMILES: *C(=O)CCCCCCC(*)=O] | 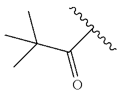 [SMILES: *C(=O)C(C)(C)C] | D | 3.80 | 1988.16 |
| Dimer 12 | 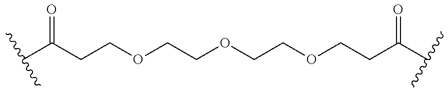 [SMILES: *C(=O)CCOCCOCCOCCC(*)=O] | 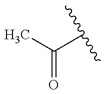 [SMILES: *C(C)=O] | D | 3.69 | 1702.76 |
| Dimer 13 | 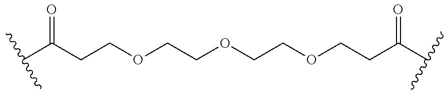 [SMILES: *C(=O)CCOCCOCCOCCC(*)=O] | 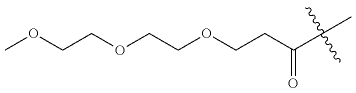 [SMILES: *C(=O)CCOCCOCCOC] | D | 4.4 | 1740.90 |
| Dimer 14 | 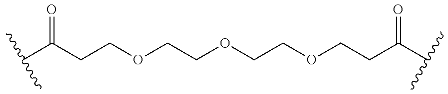 [SMILES: *C(=O)CCOCCOCCOCCC(*)=O] | 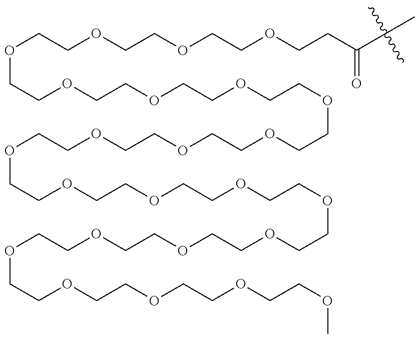 [SMILES: *C(=O)CCOCCOCCOCCOCCOCCOCCOCCOCCOCCOCCOCCOCCOCCOCCOCCOCCOCCOCCOCCOCCOCCOCCOCCOC] | D | 3.61 | 1754.54 (M + 8)/8 |
| Dimer 15 | 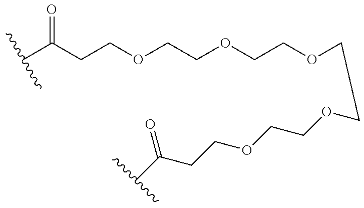 [SMILES: *C(=O)CCOCCOCCOCCOCCOCCC(*)=O] | 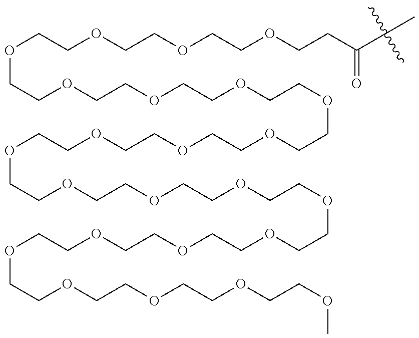 [SMILES: *C(=O)CCOCCOCCOCCOCCOCCOCCOCCOCCOCCOCCOCCOCCOCCOCCOCCOCCOCCOCCOCCOCCOCCOCCOCCOC] | D | 3.62 | 1765.31 |
| Dimer 16 | 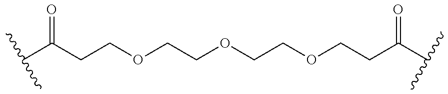 [SMILES: *C(=O)CCOCCOCCOCCC(*)=O] | 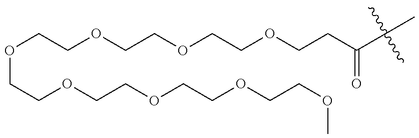 [SMILES: *C(=O)CCOCCOCCOCCOCCOCCOCCOCCOC] | D | 3.61 | 1803.63 |
| Dimer 17 | 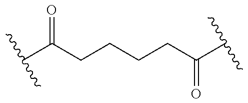 [SMILES: *C(=O)CCCCC(*)=O] | 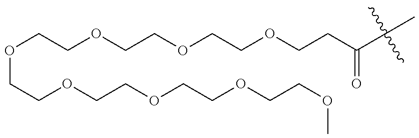 [SMILES: *C(=O)CCOCCOCCOCCOCCOCCOCCOCCOC] | D | 3.63 | 1788.70 |
| Dimer 18 | 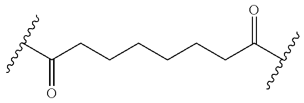 [SMILES: *C(=O)CCCCCCC(*)=O] | 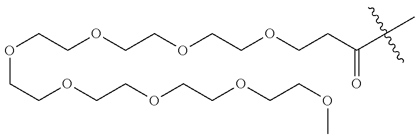 [SMILES: *C(=O)CCOCCOCCOCCOCCOCCOCCOCCOC] | D | 3.7 | 1792.75 |
| Dimer 19 | 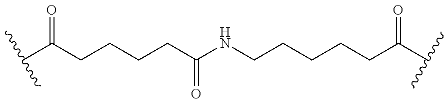 [SMILES: *C(=O)CCCCCNC(=O)CCCCC(*)=O] | 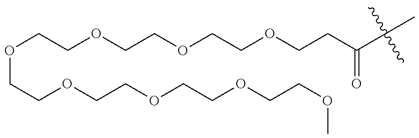 [SMILES: *C(=O)CCOCCOCCOCCOCCOCCOCCOCCOC] | D | 3.73 | 1805 |
| Dimer 20 | 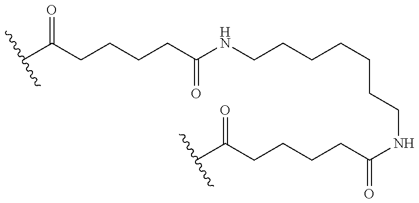 [SMILES: *C(=O)CCCCC(=O)NCCCCCCCNC(=O)CCCCC(*)=O] | 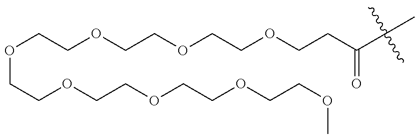 [SMILES: *C(=O)CCOCCOCCOCCOCCOCCOCCOCCOC] | D | 3.92 | 1820.8 |
| Dimer 21 | 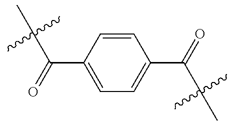 [SMILES: *C(=O)c1ccc(C(*)=O)cc1] | 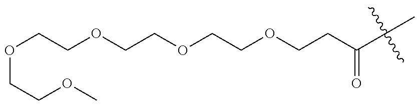 [SMILES: *C(=O)CCOCCOCCOCCOCCOC] | D | 3.72 | 1735.69 |
| Dimer 22 | 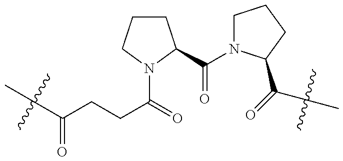 [SMILES: *C(=O)CCC(=O)N1CCC[C@H]1C(=O)N1CCC[C@H]1C(*)=O] | 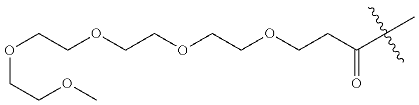 [SMILES: *C(=O)CCOCCOCCOCCOCCOC] | D | 3.74 | 1774.61 |
| Dimer 23 | 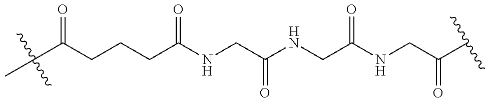 [SMILES: *C(=O)CCCC(=O)NCC(=O)NCC(=O)NCC(*)=O] | 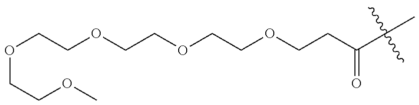 [SMILES: *C(=O)CCOCCOCCOCCOCCOC] | D | 3.74 | 1773.39 |
| Dimer 24 | 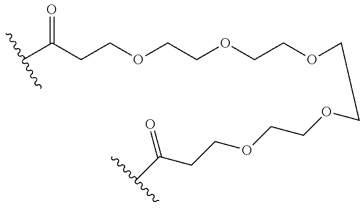 [SMILES: *C(=O)CCOCCOCCOCCOCCOCCC(*)=O] | 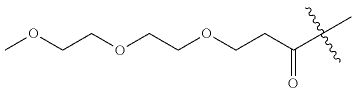 [SMILES: *C(=O)CCOCCOCCOC] | D | 3.62 | 1727.62 |
| Dimer 25 | 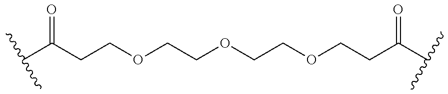 [SMILES: *C(=O)CCOCCOCCOCCC(*)=O] | 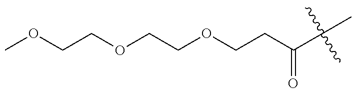 [SMILES: *C(=O)CCOCCOCCOC] | D | 3.72 | 1740.86 |
| Dimer 26 | 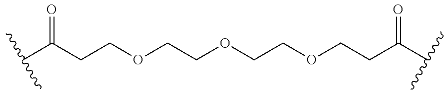 [SMILES: *C(=O)CCOCCOCCOCCC(*)=O] | 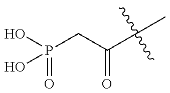 [SMILES: *C(=O)CP(=O)(O)O] | D | 3.39 | 1725.57 |
| Dimer 27 | 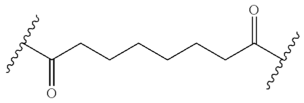 [SMILES: *C(=O)CCCCCCC(*)=O] | 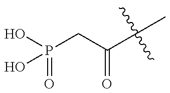 [SMILES: *C(=O)CP(=O)(O)O] | D | 3.49 | 1714.96 |
| Dimer 28 | 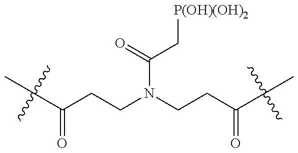 | 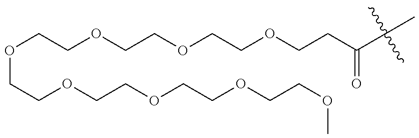 [SMILES: *C(=O)CCOCCOCCOCCOCCOCCOCCOCCOC] | D | 3.62 | 1808.48 |
| Dimer 29 | 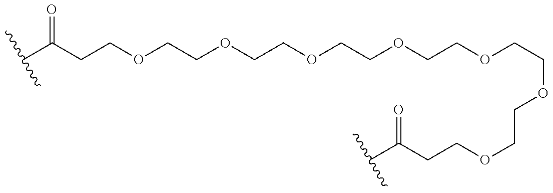 [SMILES: *C(=O)CCOCCOCCOCCOCCOCCOCCOCCC(*)=O] | 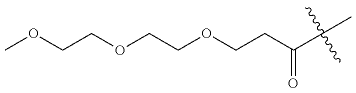 [SMILES: *C(=O)CCOCCOCCOC] | D | 3.69 | 1765.50 |
| Dimer 30 | 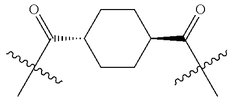 [SMILES: *C(=O)[C@H]1CC[C@H](C(*)=O)CC1] | 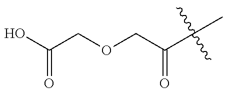 [SMILES: *C(=O)COCC(=O)O] | D | 3.64 | 1713.01 |
| Dimer 31 | 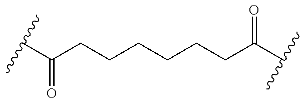 [SMILES: *C(=O)CCCCCCC(*)=O] | 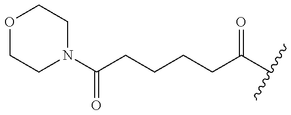 [SMILES: *C(=O)CCCCC(=O)N1CCOCC1] | D | 3.71 | 1735.96 |
| Dimer 32 | 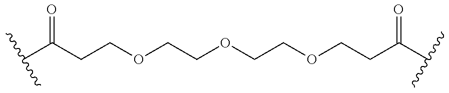 [SMILES: *C(=O)CCOCCOCCOCCC(*)=O] | 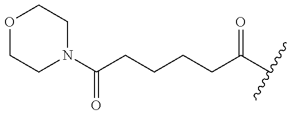 [SMILES: *C(=O)CCCCC(=O)N1CCOCC1] | D | 3.7 | 1747.34 |
| Dimer 33 | 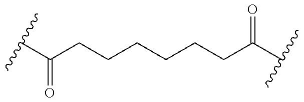 [SMILES: *C(=O)CCCCCCC(*)=O] | 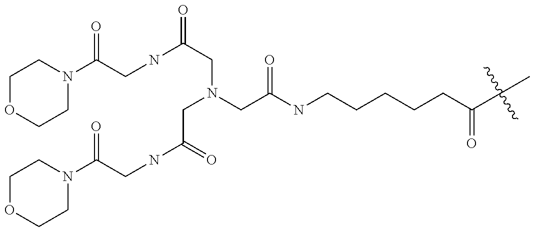 [SMILES: *C(=O)CCCCCNC(=O)CN(CC(=O)NCC(=O)N1CCOCC1)CC(=O)NCC(=O)N1CCOCC1] | D | 3.06 | 1833.7 |
| Dimer 34 | 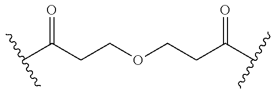 [SMILES: *C(=O)CCOCCC(*)=O] | 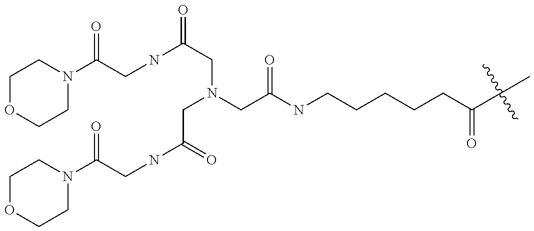 [SMILES: *C(=O)CCCCCNC(=O)CN(CC(=O)NCC(=O)N1CCOCC1)CC(=O)NCC(=O)N1CCOCC1] | D | 3.68 | 1831.94 |
| Dimer 35 | 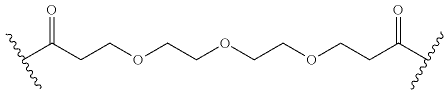 [SMILES: *C(=O)CCOCCOCCOCCC(*)=O] | 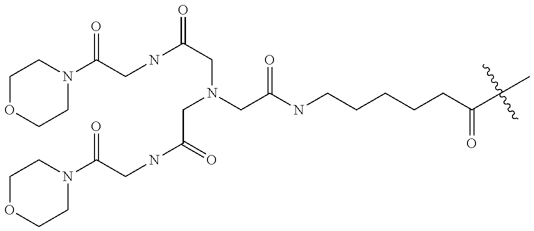 [SMILES: *C(=O)CCCCCNC(=O)CN(CC(=O)NCC(=O)N1CCOCC1)CC(=O)NCC(=O)N1CCOCC1] | D | 3.68 | 1844.34 |
| Dimer 36 | 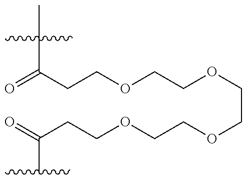 [SMILES: *C(=O)CCOCCOCCOCCOCCC(*)=O] | 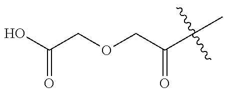 [SMILES: *C(=O)COCC(=O)O] | D | 3.66 | 1730.06 |
| Dimer 37 | 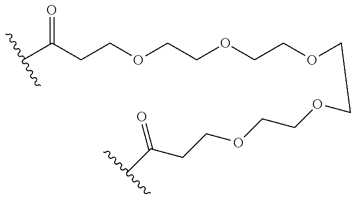 [SMILES: *C(=O)CCOCCOCCOCCOCCOCCC(*)=O] | 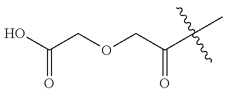 [SMILES: *C(=O)COCC(=O)O] | D | 3.67 | 1736.63 |
| Dimer 38 | 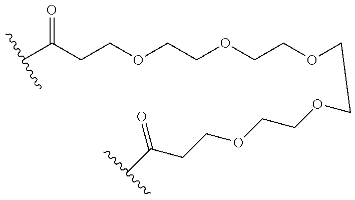 [SMILES: *C(=O)CCOCCOCCOCCOCCOCCC(*)=O] | 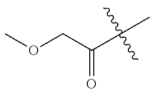 [SMILES: *C(=O)COC] | D | 3.42 | 1724.27 |

### Example 9

Method D is also applicable for the coupling step of analogs used containing capping groups on B1,B29, B1′, and B29′ positions

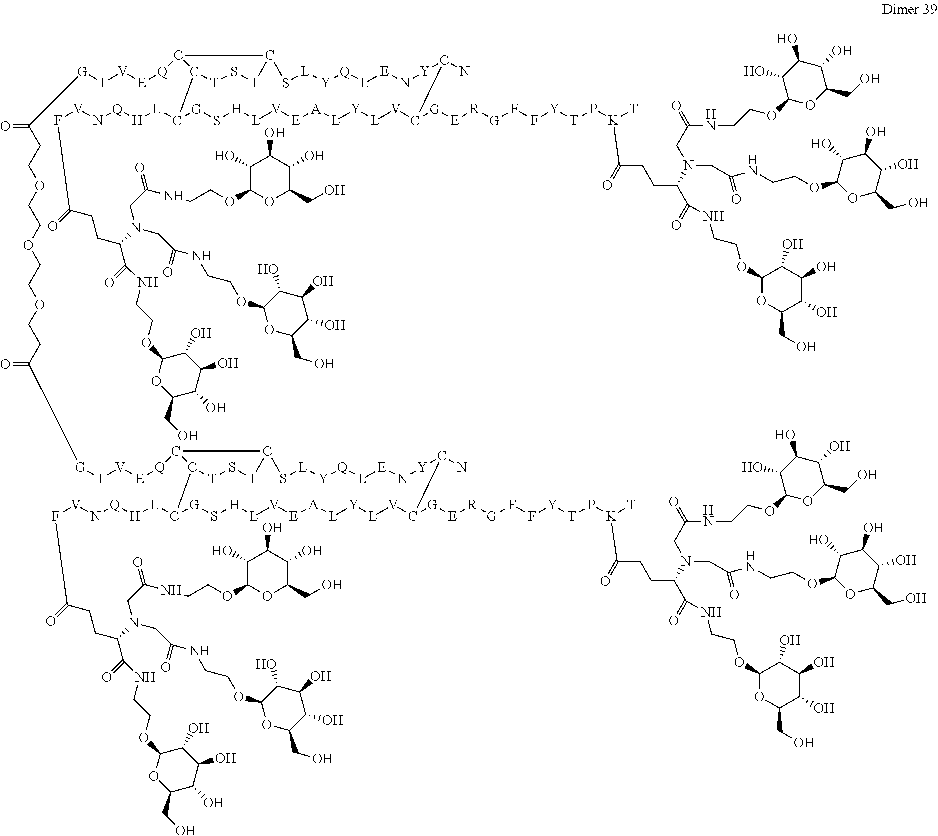

To a solution of Analog 26 (105 mg, 0.014 mmol) in DMSO (820 μl) was added triethylamine (78 μl, 0.558 mmol) followed by solution of the Linking Reagent 31, diethylene glycol 1,2-bis(succinimidyl 3-oxypropionate) (3.10 mg, 6.97 μmol) in 30 μL of DMSO. Stirred the reaction mixture for 1 hour. Diluted the reaction mixture with 15 mL of water and diafiltrated in Amicon tubes (10K MWCO membrane) to remove most of DMSO. The product was purified by ion exchange chromatography (PolySULFOETHYL A column, PolyLC Inc., 250×21 mm, 5 μm, 1000 Å; Buffer A: 0.1%(v/v)H3PO4/25% AcCN; Buffer B: 0.1%(v/v) H3PO4/25% AcCN/0.5 M NaCl), fractions containing the desired material were concentrated using Amicon tubes (10K MWCO membrane), and the product was re-purified by reverse phase HPLC (Kromasil C8 250×50 mm, 10 μm, 100 Å column; Buffer A: 0.05% TFA in water; Buffer B: 0.05% TFA in AcCN). The product was obtained as a solid after lyophilization of the HPLC fractions. UPLC-MS Method D: Rt=3.27 min, m/z=1909.86 [(M+8)/8].

### Example 10

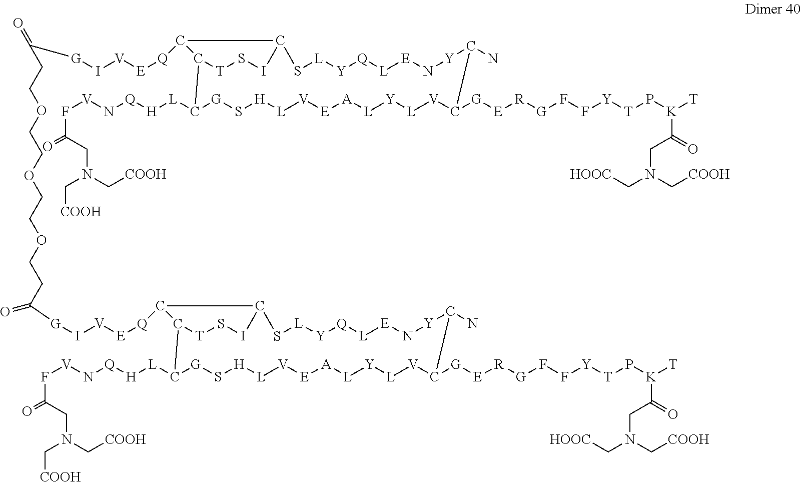

Starting from Analog 27 and Linking Reagent 31, using the procedures described in General Method D, the title compound was obtained. UPLC-MS Method D: Rt=3.25 min, m/z=1789.77 [(M+7)/7].

### Example 11

Table III insulin dimers 41 through 70 (backbone depicted by Formula I), in Table VIII below were synthesized from appropriate insulin Analogs and Linking Reagent using procedures of General Method D, which have capping group on B1, B29, B1′, and B29′.

TABLE VIII

| | | Capping group | | | |
|---|---|---|---|---|---|
| | | (wavy line | | | |
| | | indicates | | | |
| | Linker (wavy line | attachments to | | | (M + 6)/ |
| | indicates attachment to | B1, B29 and | | | 6 or |
| Dimer | A1 and A1′ sites of | B1′, B29′ sites | UPLC | | ((M + 7)/ |
| # | insulins) | of insulins) | Method | Rt(min) | 7) |
| Dimer 41 | 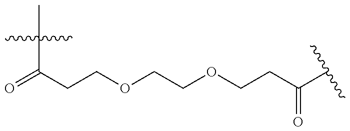 [SMILES: *C(=O)CCOCCOCCC(*)=O] | 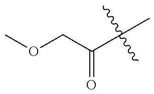 [SMILES: *C(=O)COC] | D | 3.66 | 1725.824 |
| Dimer 42 | 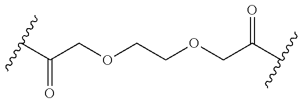 [SMILES: *C(=O)COCCOCC(*)=O] | 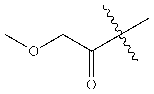 [SMILES: *C(=O)COC] | D | 3.67 | 1721.91 |
| Dimer 43 | 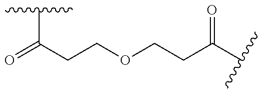 [SMILES: *C(=O)CCOCCC(*)=O] | 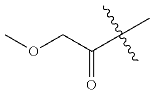 [SMILES: *C(=O)COC] | D | 3.66 | 1719.432 |
| Dimer 44 | 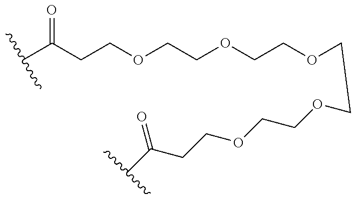 [SMILES: *C(=O)CCOCCOCCOCCOCCOCCC(*)=O] | 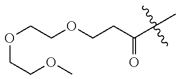 [SMILES: *C(=O)CCOCCOCCOC] | | 3.621 | 1803.526 |
| Dimer 45 | 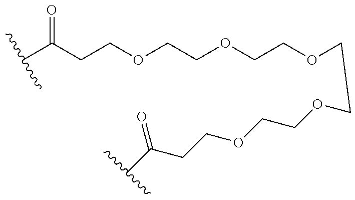 [SMILES: *C(=O)CCOCCOCCOCCOCCOCCC(*)=O] | 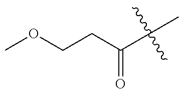 [SMILES: *C(=O)CCOC] | | 3.492 | 1752.340 |
| Dimer 46 | 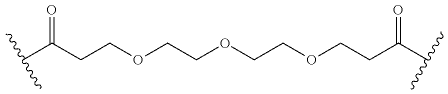 [SMILES: *C(=O)CCOCCOCCOCCC(*)=O] | 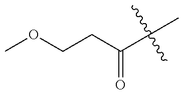 [SMILES: *C(=O)CCOC] | | 3.637 | 1739.761 |
| Dimer 47 | 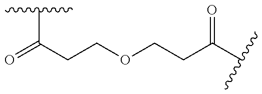 [SMILES: *C(=O)CCOCCC(*)=O] | 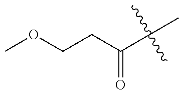 [SMILES: *C(=O)CCOC] | | 3.712 | 1727.081 |
| Dimer 48 | 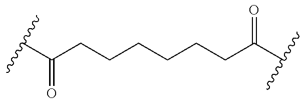 [SMILES: *C(=O)CCCCCCC(*)=O] | 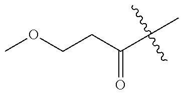 [SMILES: *C(=O)CCOC] | A | 3.710 | 1729.181 |
| Dimer 49 | 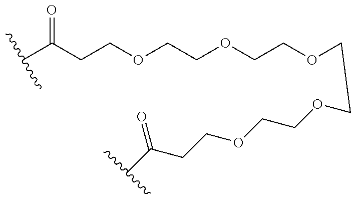 [SMILES: *C(=O)CCOCCOCCOCCOCCOCCC(*)=O] | 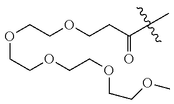 [SMILES: *C(=O)CCOCCOCCOCCOCCOC] | | 4.647 | 1853.390 |
| Dimer 50 | 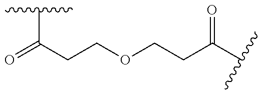 [SMILES: *C(=O)CCOCCC(*)=O] | 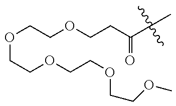 [SMILES: *C(=O)CCOCCOCCOCCOCCOC] | | 3.780 | 1802.856 |
| Dimer 51 | 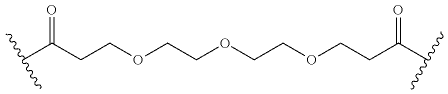 [SMILES: *C(=O)CCOCCOCCOCCC(*)=O] | 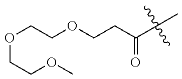 [SMILES: *C(=O)CCOCCOCCOC] | | 2.898 | 1790.356 |
| Dimer 52 | 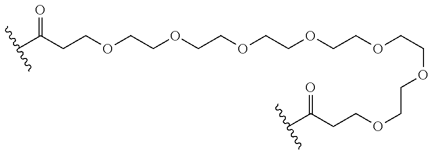 [SMILES: *C(=O)CCOCCOCCOCCOCCOCCOCCOCCC(*)=O] | 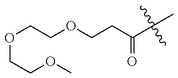 [SMILES: *C(=O)CCOCCOCCOC] | | 2.555 | 1815.786 |
| Dimer 53 | 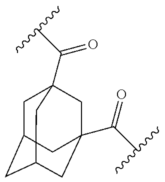 [SMILES: *C(=O)C12CC3CC(C1)CC(C(*)=O)(C3)C2] | 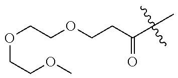 [SMILES: *C(=O)CCOCCOCCOC] | D | 4.58 | 1786.5 |
| Dimer 54 | 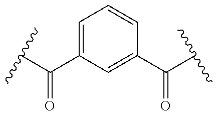 [SMILES: *C(=O)c1cccc(C(*)=O)c1] | 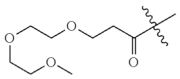 [SMILES: *C(=O)CCOCCOCCOC] | D | 4.50 | 1778.5 |
| Dimer 55 | 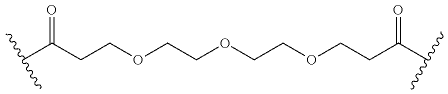 [SMILES: *C(=O)CCOCCOCCOCCC(*)=O] | 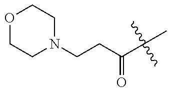 [SMILES: *C(=O)CCN1CCOCC1] | D | 3.66 | 1771.8 |
| Dimer 56 | 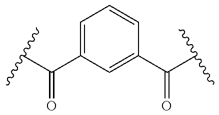 [SMILES: *C(=O)c1cccc(C(*)=O)c1] | 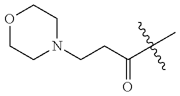 [SMILES: *C(=O)CCN1CCOCC1] | D | 3.65 | 1759.6 |
| Dimer 57 | 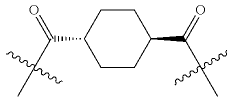 [SMILES: *C(=O)[C@H]1CC[C@H](C(*)=O)CC1] | 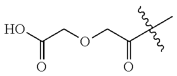 [SMILES: *C(=O)COCC(=O)O] | D | 3.82 | 1745.89 |
| Dimer 58 | 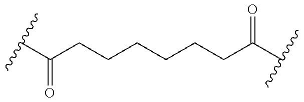 [SMILES: *C(=O)CCCCCCC(*)=O] | 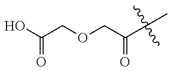 [SMILES: *C(=O)COCC(=O)O] | A | 0.88 | 1746.6 |
| Dimer 59 | 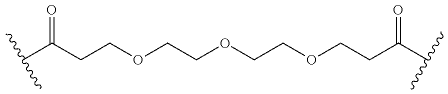 [SMILES: *C(=O)CCOCCOCCOCCC(*)=O] | 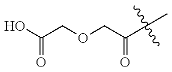 [SMILES: *C(=O)COCC(=O)O] | A | 0.88 | 1757.2 |
| Dimer 60 | 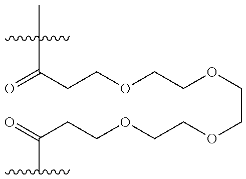 [SMILES: *C(=O)CCOCCOCCOCCOCCC(*)=O] | 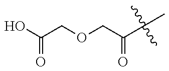 [SMILES: *C(=O)COCC(=O)O] | A | 0.88 | 1763.5 |
| Dimer 61 | 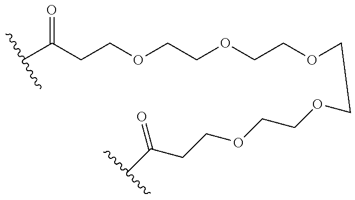 [SMILES: *C(=O)CCOCCOCCOCCOCCOCCC(*)=O] | 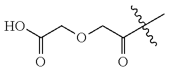 [SMILES: *C(=O)COCC(=O)O] | A | 0.88 | 1770.2 |
| Dimer 62 | 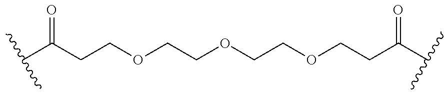 [SMILES: *C(=O)CCOCCOCCOCCC(*)=O] | 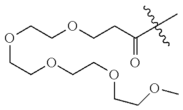 [SMILES: *C(=O)CCOCCOCCOCCOCCOC] | A | 0.91 | 1840.9 |
| Dimer 63 | 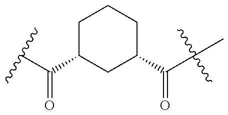 [SMILES: *C(=O)[C@@H]1CCC[C@H](C(*)=O)C1] | 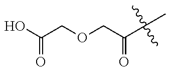 [SMILES: *C(=O)COCC(=O)O] | D | 3.84 | 1745.80 |
| Dimer 64 Ahmet | 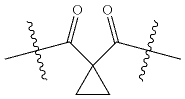 [SMILES: *C(=O)C1(C(*)=O)CC1] | 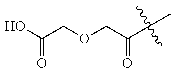 [SMILES: *C(=O)COCC(=O)O] | D | 3.86 | 1739.83 |
| Dimer 65 Ahmet | 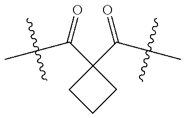 [SMILES: *C(=O)C1(C(*)=O)CCC1] | 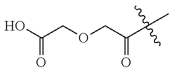 [SMILES: *C(=O)COCC(=O)O] | D | 3.83 | 1741.80 |
| Dimer 66 | 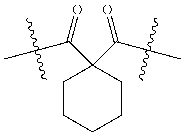 [SMILES: *C(=O)C1(C(*)=O)CCCCC1] | 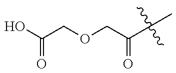 [SMILES: *C(=O)COCC(=O)O] | D | 3.87 | 1745.86 |
| Dimer 67 | 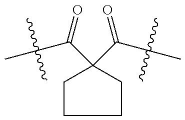 [SMILES: *C(=O)C1(C(*)=O)CCCC1] | 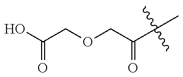 [SMILES: *C(=O)COCC(=O)O] | | 3.85 | 1743.81 |
| Dimer 68 | 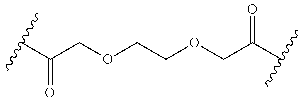 [SMILES: *C(=O)COCCOCC(*)=O] | 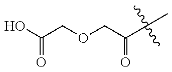 [SMILES: *C(=O)COCC(=O)O] | | 3.81 | 1746.51 |
| Dimer 69 | 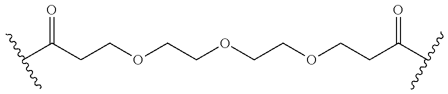 [SMILES: *C(=O)CCOCCOCCOCCC(*)=O] | 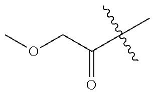 [SMILES: *C(=O)COC] | | 3.95 | 1731.749 |
| Dimer 70 | 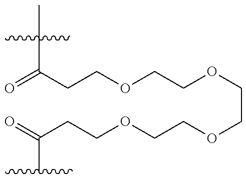 [SMILES: *C(=O)CCOCCOCCOCCOCCC(*)=O] | 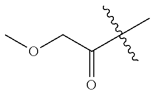 [SMILES: *C(=O)COC] | | 3.95 | 1738.097 |

### General Method D-1: Deprotection with TFA is Included to Remove Protective Groups from the Linker of from the Capping Group

### Example 12

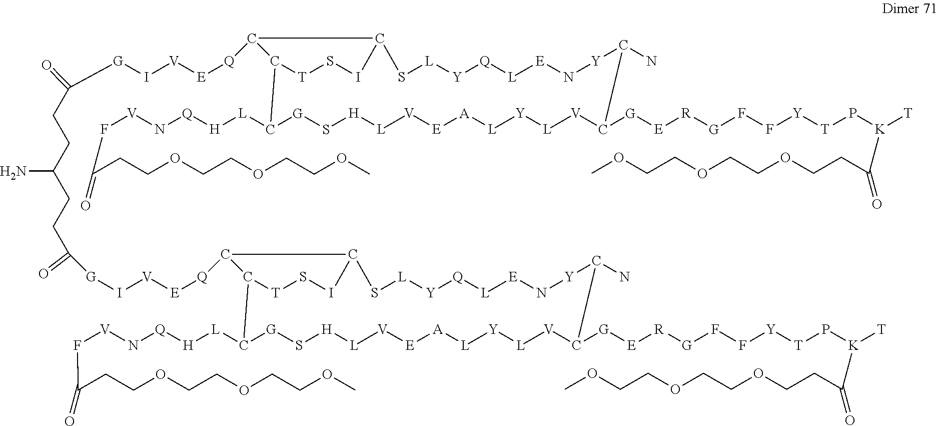

### Step 1 Coupling of Insulins

Using Linking Reagent 11 (3.89 mg) and Analog 28 (102 mg), dimeric insulin was prepared in the conditions of General Method D. UPLC Method G: Rt=3.49 min, m/z=1793.95 [(M+7)/7].

### Step 2 Deprotection of Boc

The product of Step 1 was dissolved at 0° C. in 0.5 mL of TFA and the reaction mixture was stirred for 30 min. The reaction mixture was added to 35 mL of ether and the formed precipitate was collected by centrifugation. The product was purified by reverse phase HPLC (KROMASIL C8 250×50 mm, 10 μm, 100 Å column; Buffer A: 0.05% TFA in deionized water; Buffer B: 0.05% TFA in AcCN). Fractions containing the desired insulin dimer are combined and freeze-dried to give the desired material. UPLC Method G: Rt=3.56 min, m/z=1779.70 [(M+7)/7].

### Example 13

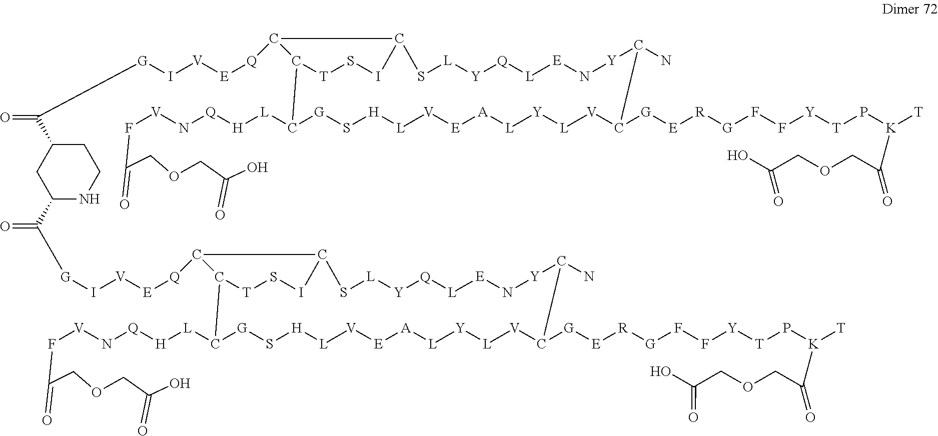

was prepared according to General Method D-1 from Analog 30 and Linking Reagent 22. UPLC Method D: Rt=3.75 min, m/z=1745.95 [(M+7)/7].

### Example 14

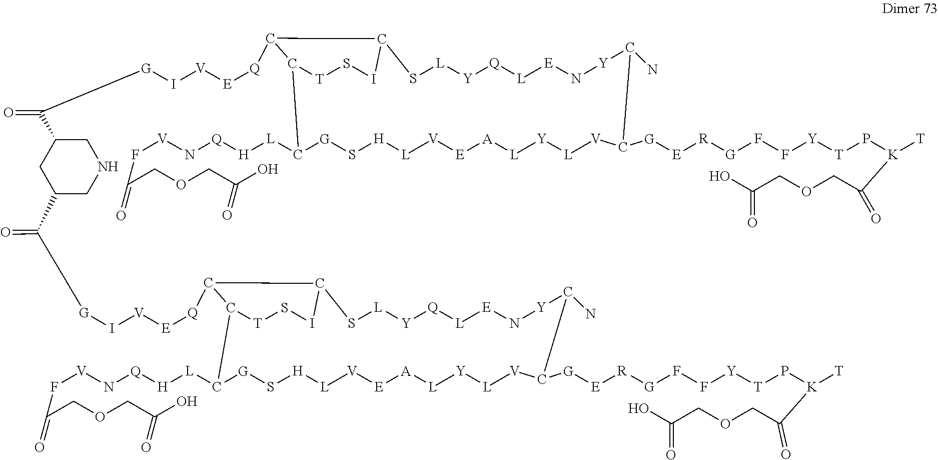

was prepared according to General Method D-1 from Analog 30 and Linking Reagent 4. UPLC Method D: Rt=3.83 min, m/z=1745.99 [(M+7)/7].

### Example 15

Table III insulin dimers 74 through 109, depicted in Table IX below, were synthesized from appropriate insulin Analogs and Linking Reagent using procedures of General Method D or D-1, which have capping groups on B29, and B29′.

TABLE IX

| Di- | | | | (M + 6)/ |
|---|---|---|---|---|
| mer | | UPLC | | 6 or |
| Num- | | Meth- | Rt | (M + 7)/ |
| ber | Composite structure Chemdraw Structues | od | (min) | 7 |
| Di- mer 74 | 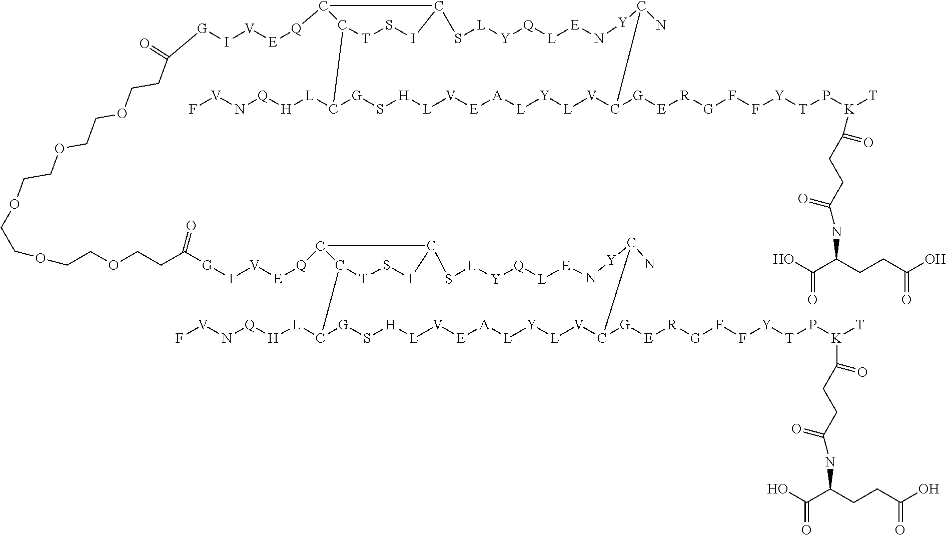 | D | 3.62 | 1768.705 |
| Di- mer 76 | 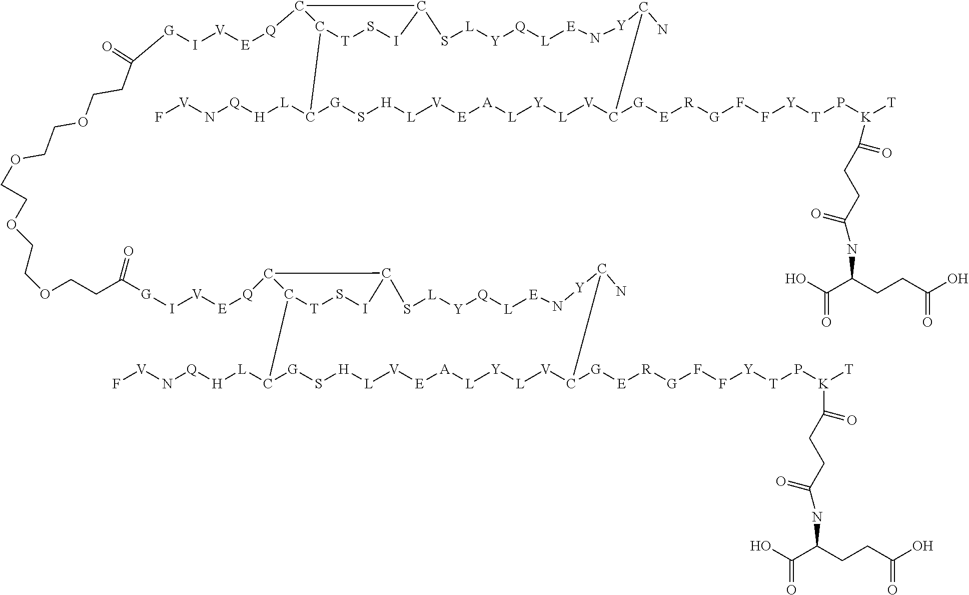 | D | 3.63 | 1762.404 |
| Di- mer 77 | 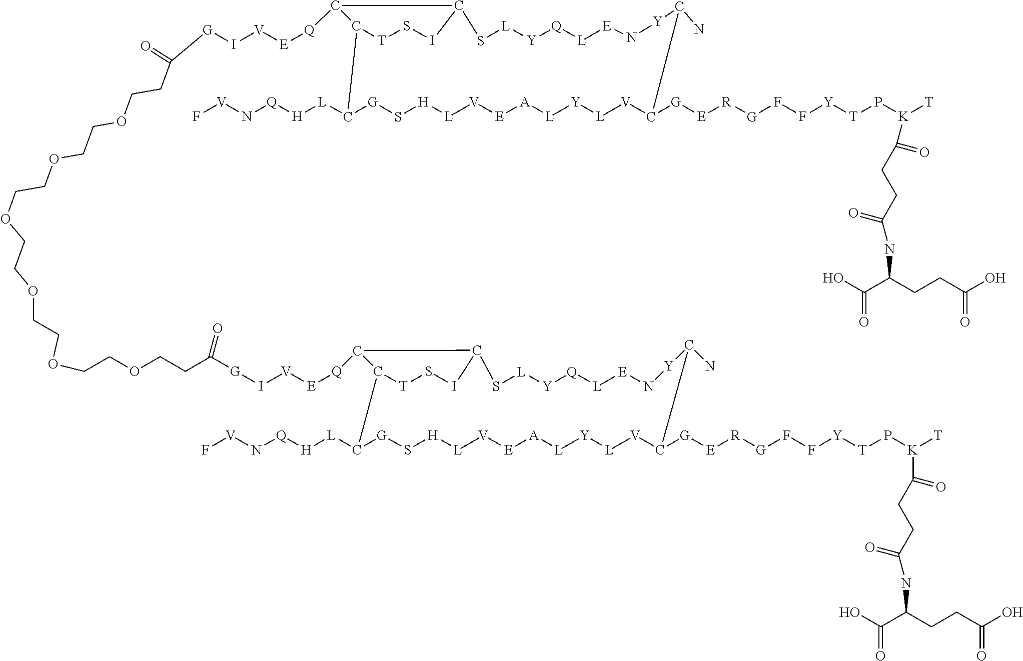 | D | 3.58 | 1775.727 |
| Di- mer 78 | 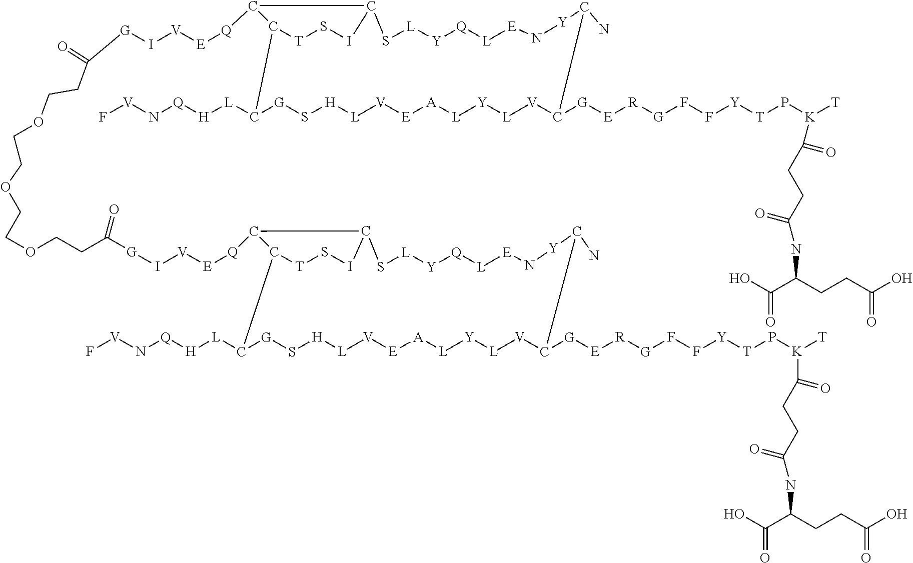 | D | 3.66 | 1756.135 |
| Di- mer 79 | 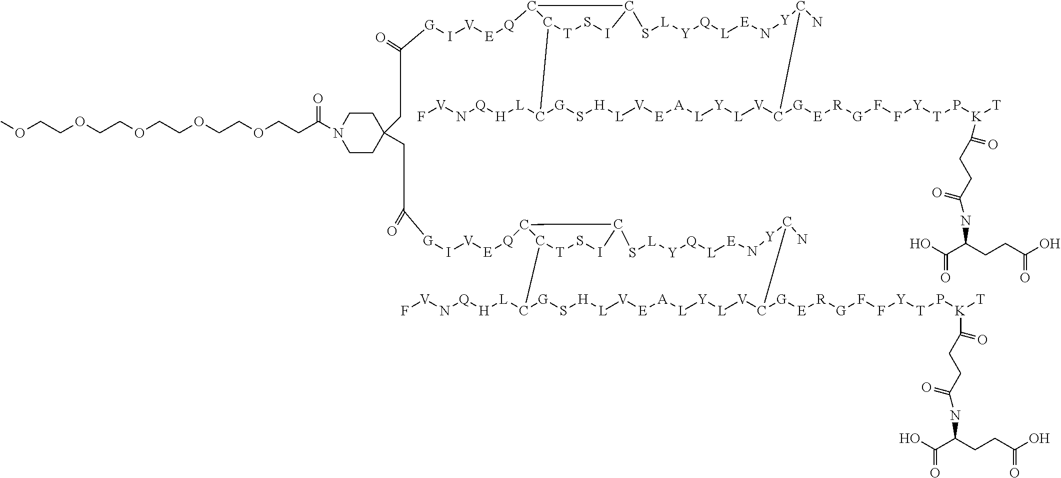 | D | 3.90 | 1786.794 |
| Di- mer 80 | 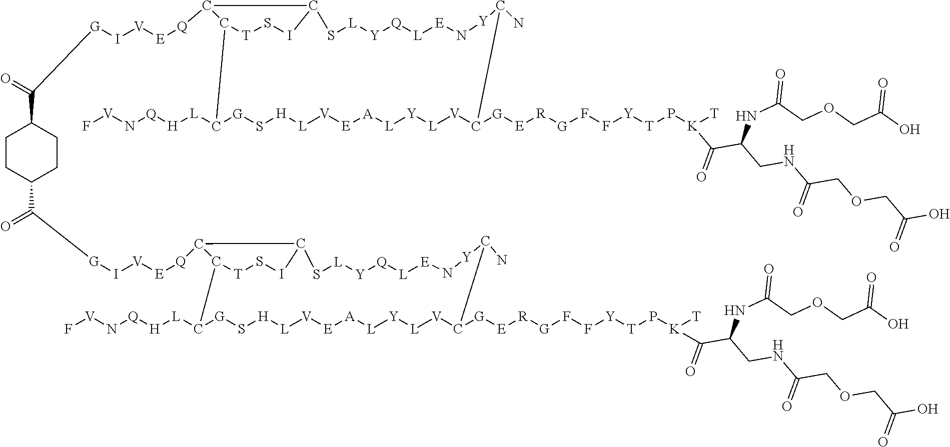 | D | 3.63 | 1770.45 |
| Di- mer 81 | 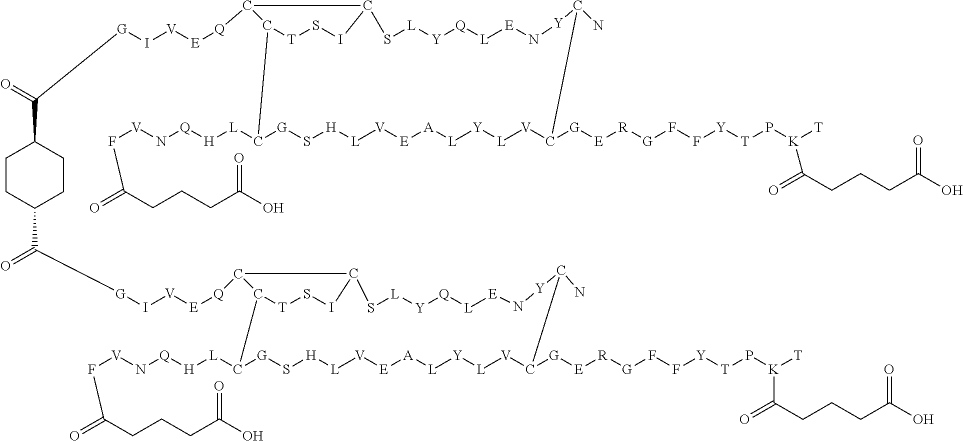 | D | 3.80 | 1744.46 |
| Di- mer 82 | 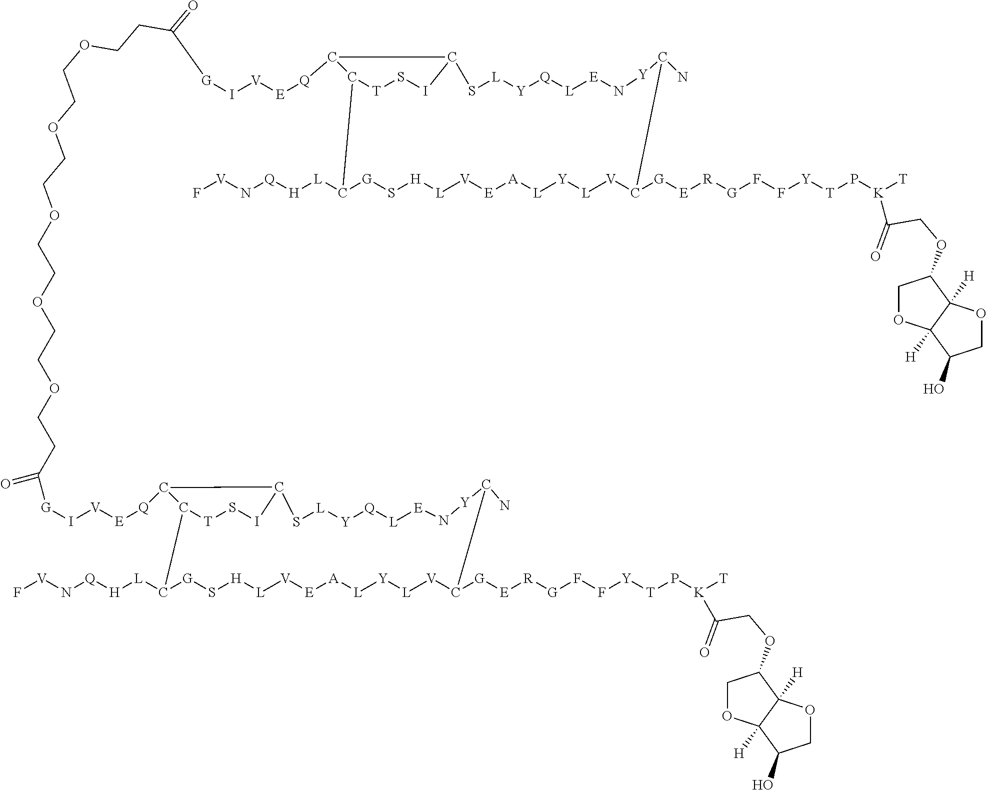 | D | 3.65 | 1757.40 |
| Di- mer 83 | 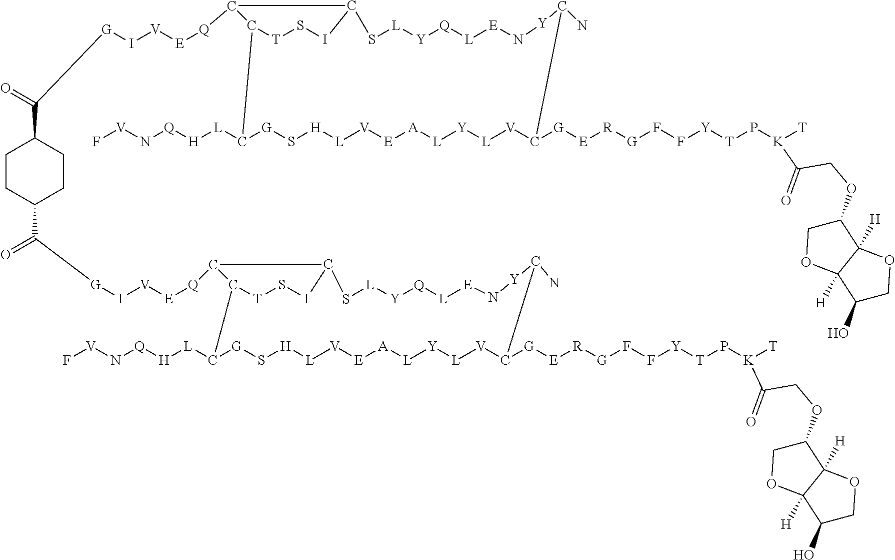 | D | 3.64 | 1733.65 |
| Di- mer 84 | 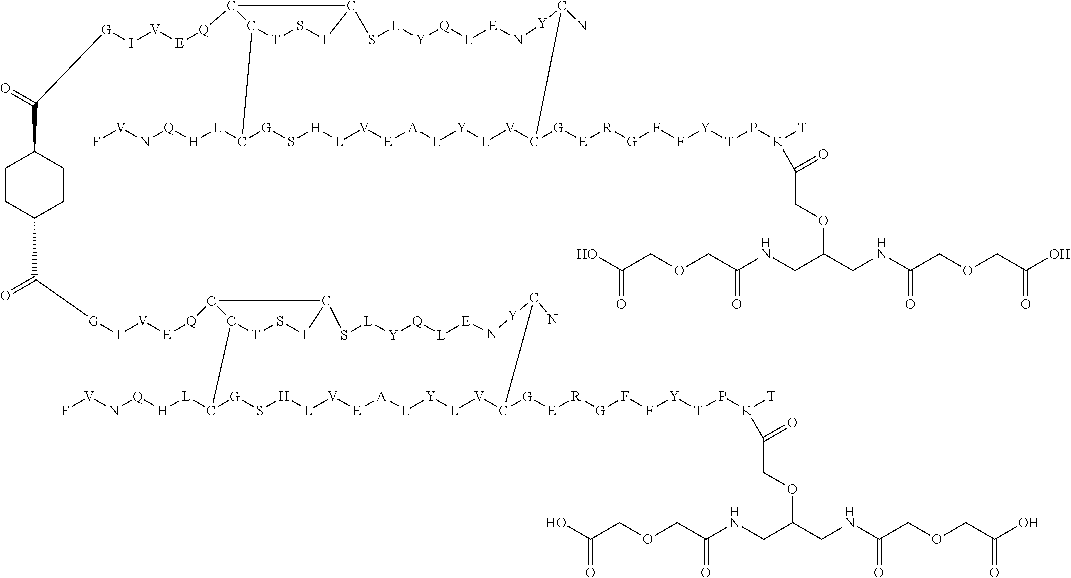 | D | 3.63 | 1782.90 |
| Di- mer 85 | 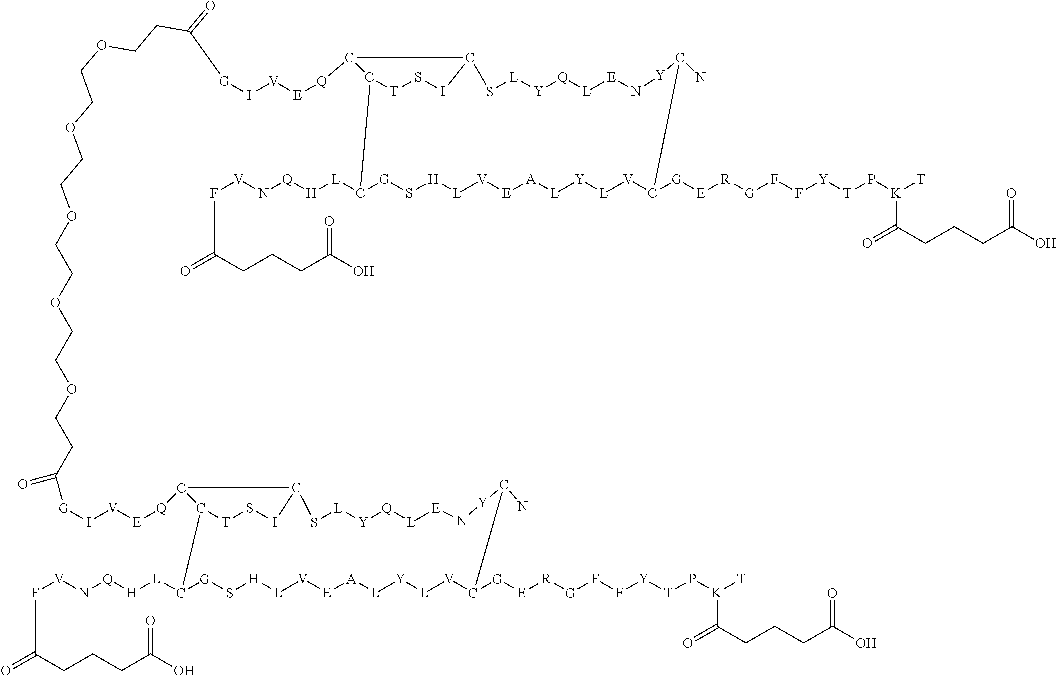 | D | 3.82 | 1769.61 |
| Di- mer 86 | 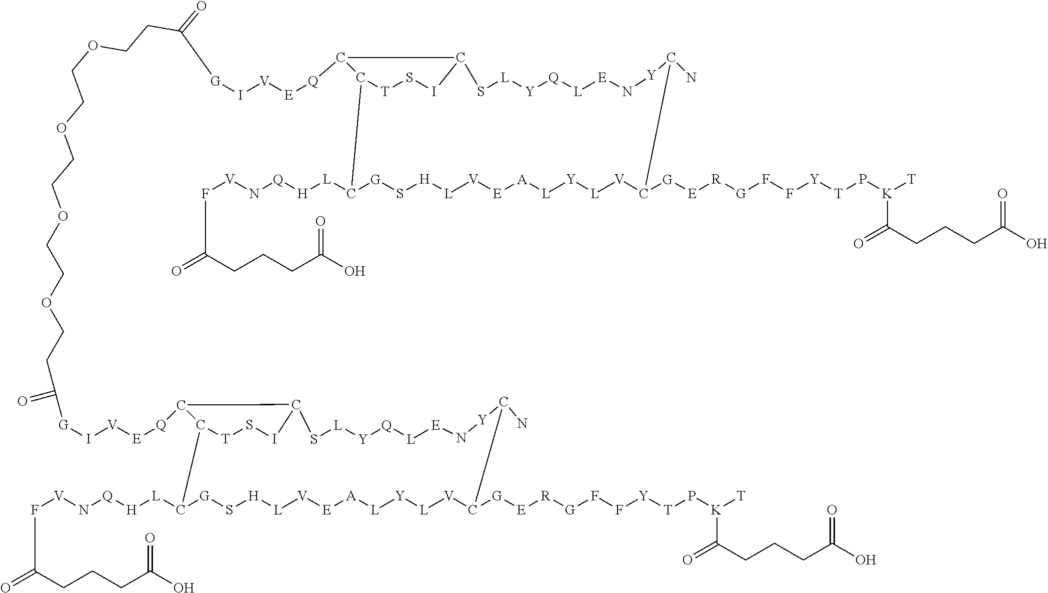 | D | 3.84 | 1762.23 |
| Di- mer 87 | 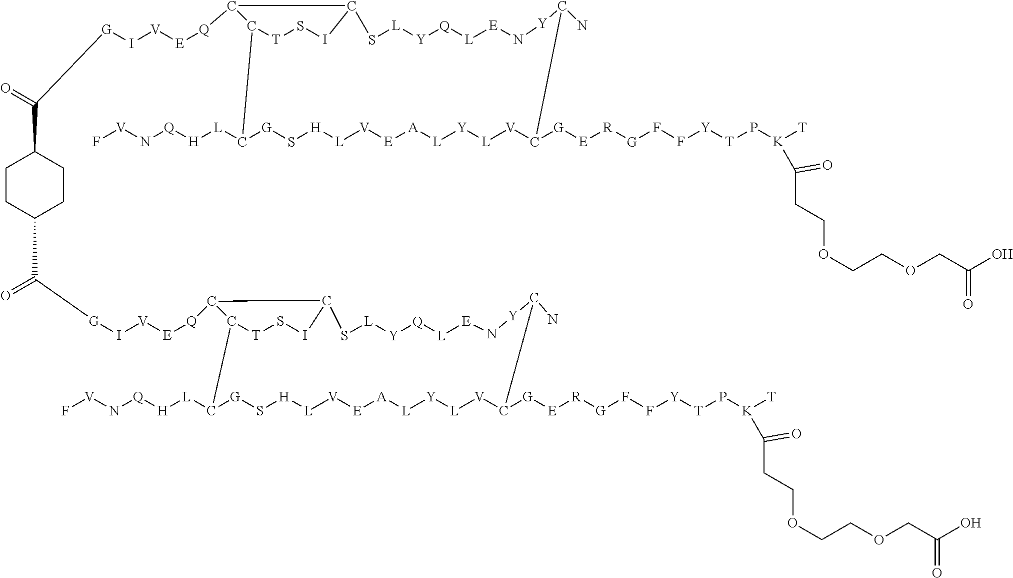 | D | 3.70 | 1725.19 |
| Di- mer 88 | 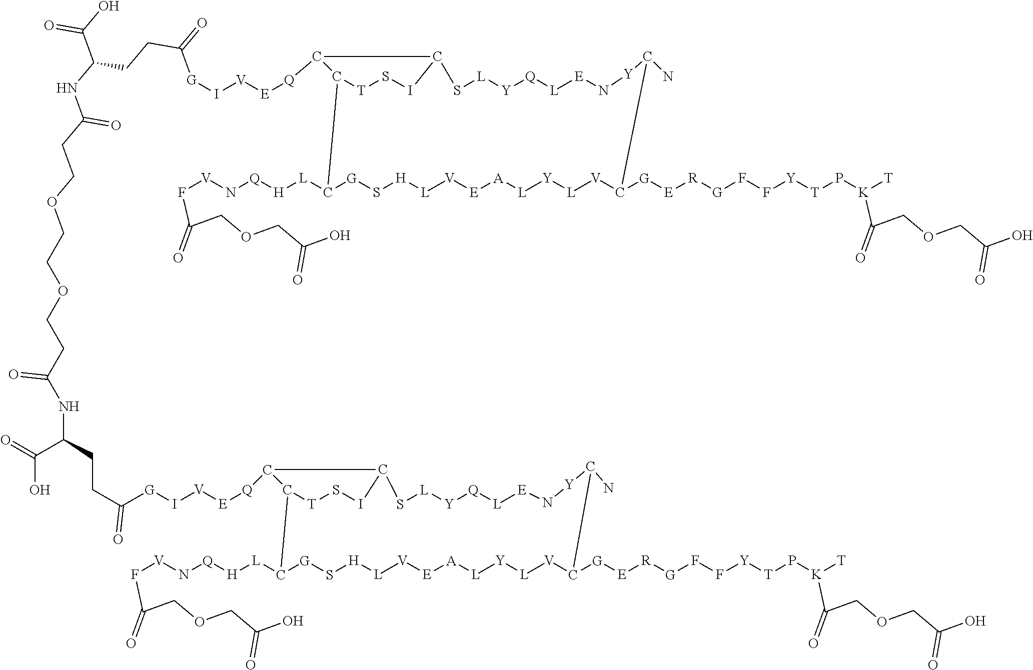 | D | 3.84 | 1787.61 |
| Di- mer 89 | 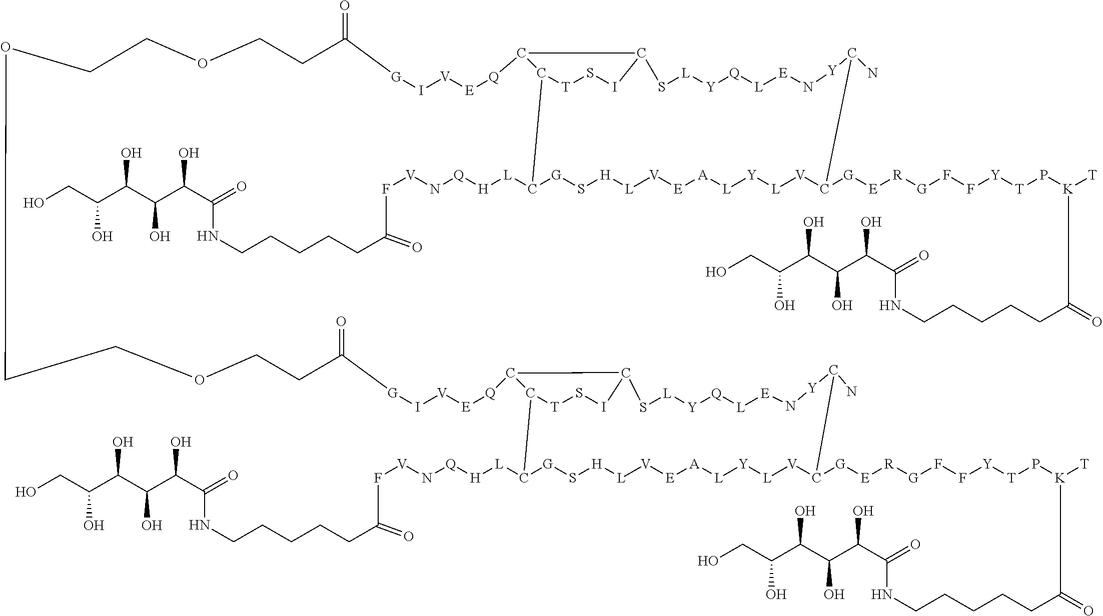 | D | 3.63 | 1856.79 1656.68 |
| Di- mer 90 | 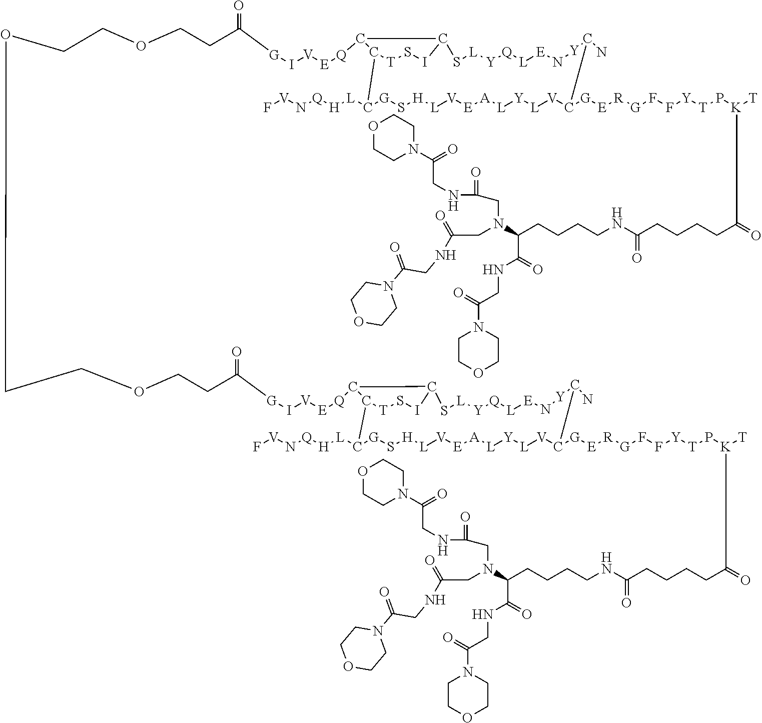 | D | 3.56 | 1640.07 |
| Di- mer 91 | 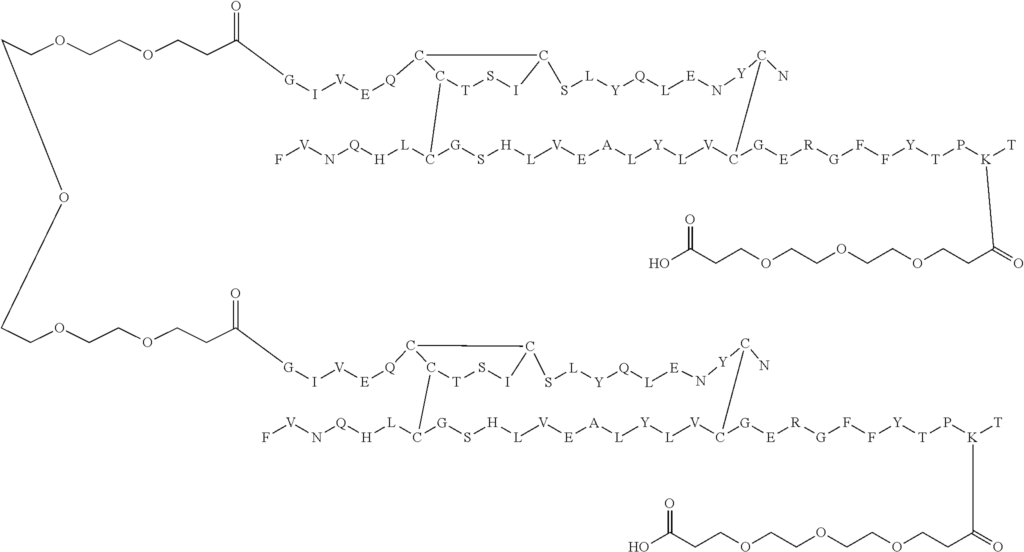 | D | 3.73 | 1548.57 1769.50 |
| Di- mer 92 | 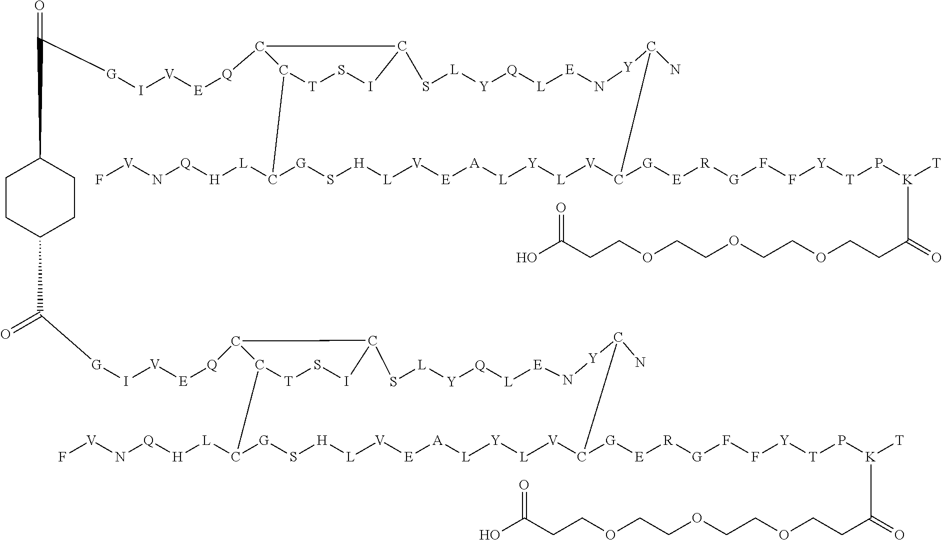 | D | 3.71 | 1527.76 1745.60 |
| Di- mer 93 | 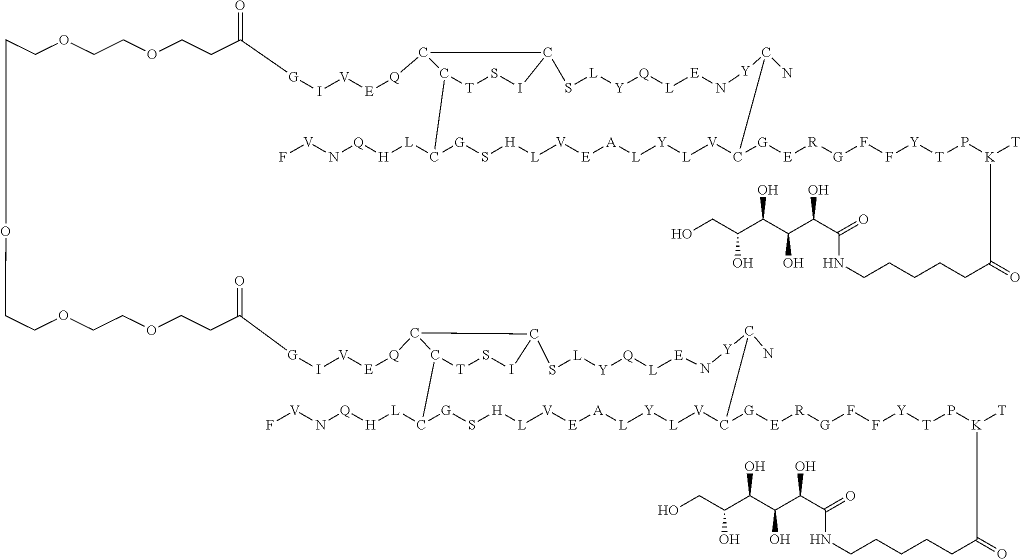 | D | 3.58 | 1563.25 1786.48 |
| Di- mer 94 | 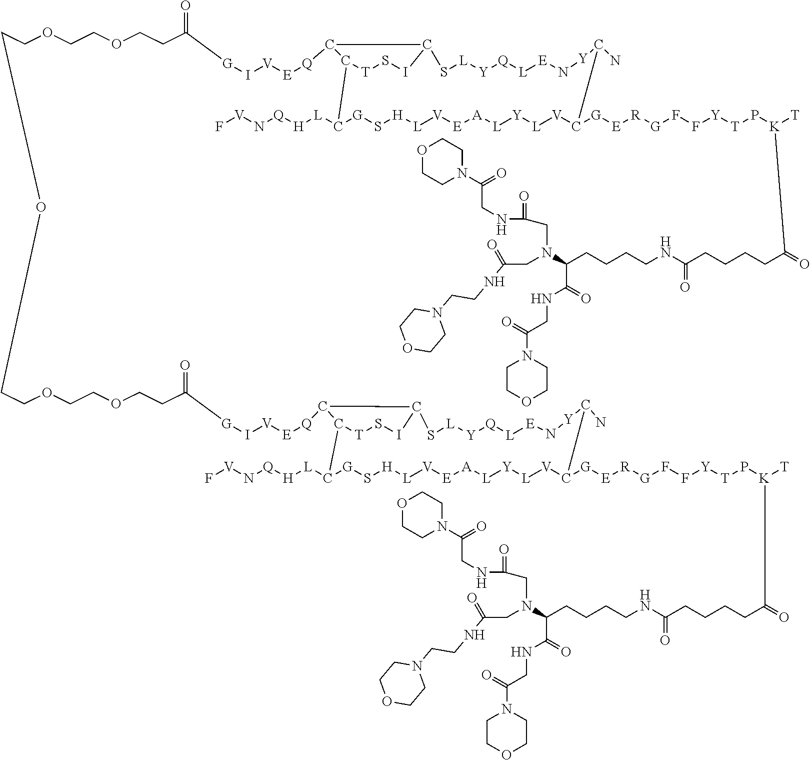 | D | 3.66 | 1491.89 1678.50 |
| Di- mer 95 | 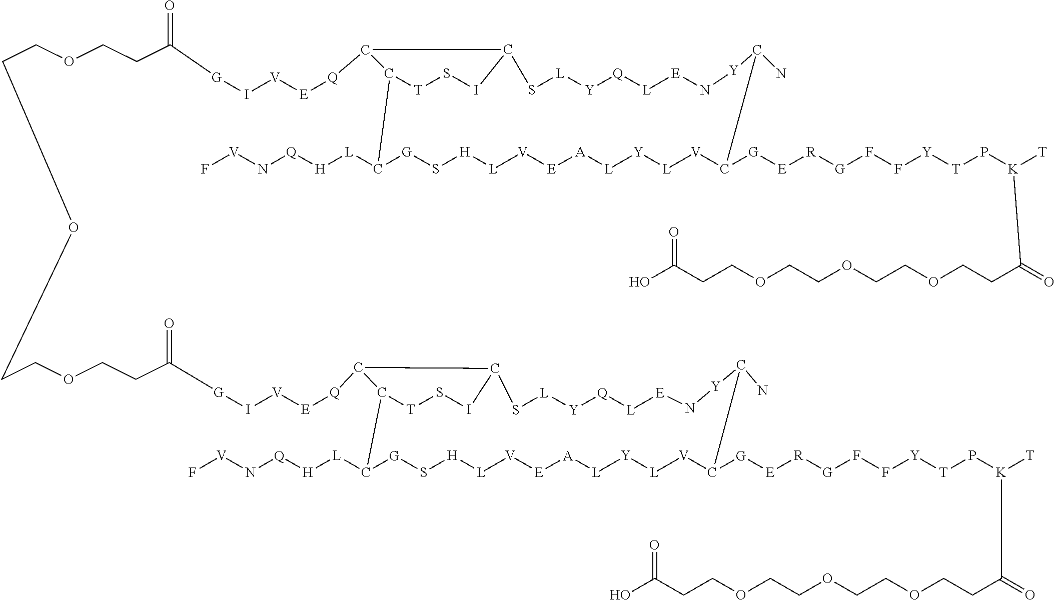 | D | 3.71 | 1537.41 1756.90 |
| Di- mer 96 | 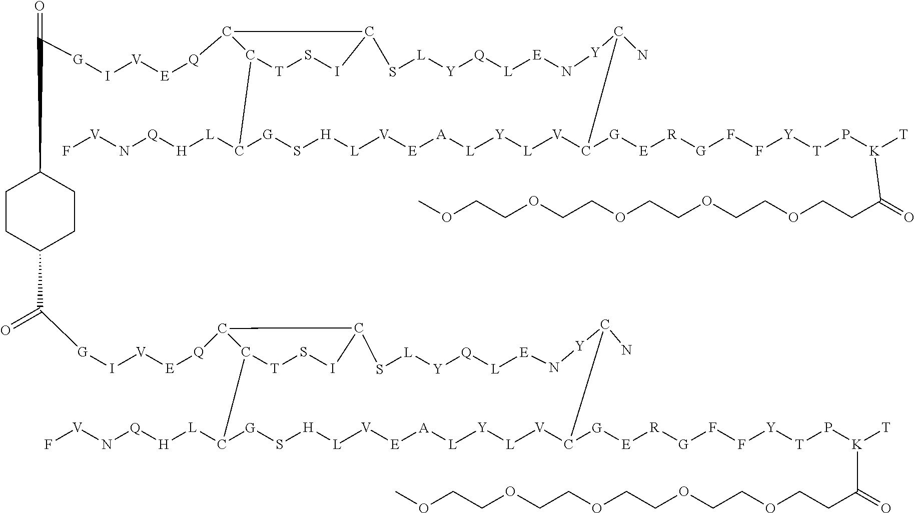 | D | 3.74 | 1535.39 1754.84 |
| Di- mer 97 | 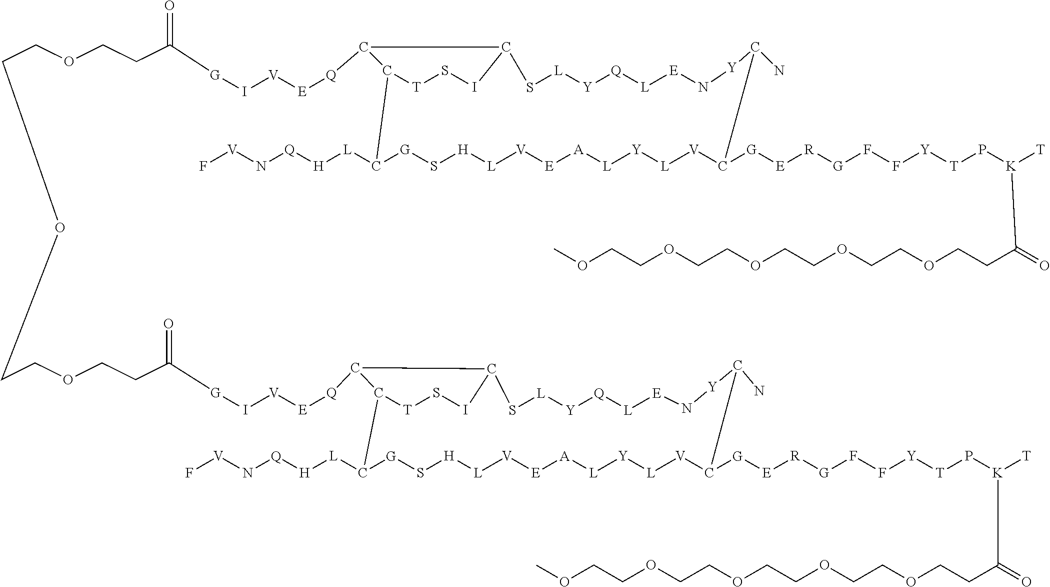 | C | 0.86 | 1546.04 1766.44 |
| Di- mer 98 | 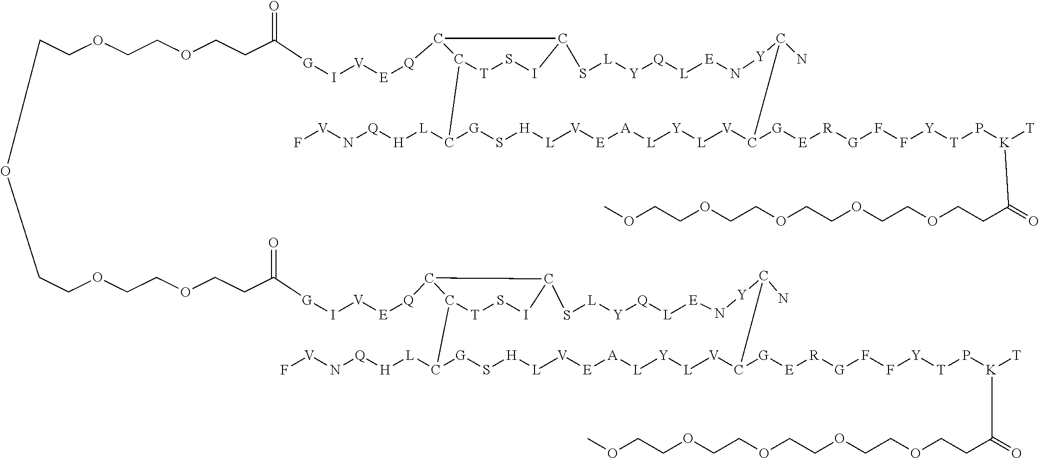 | D | 3.79 | 1556.18 1778.18 |
| Di- mer 99 | 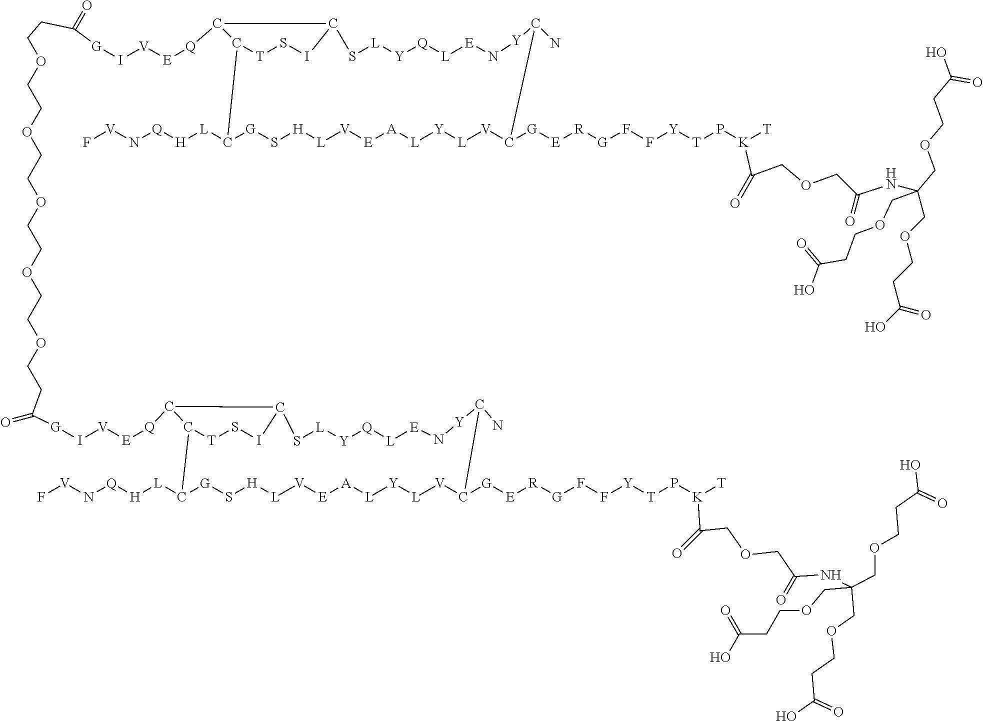 | D | 3.62 | 1827.72 |
| Di- mer 100 | 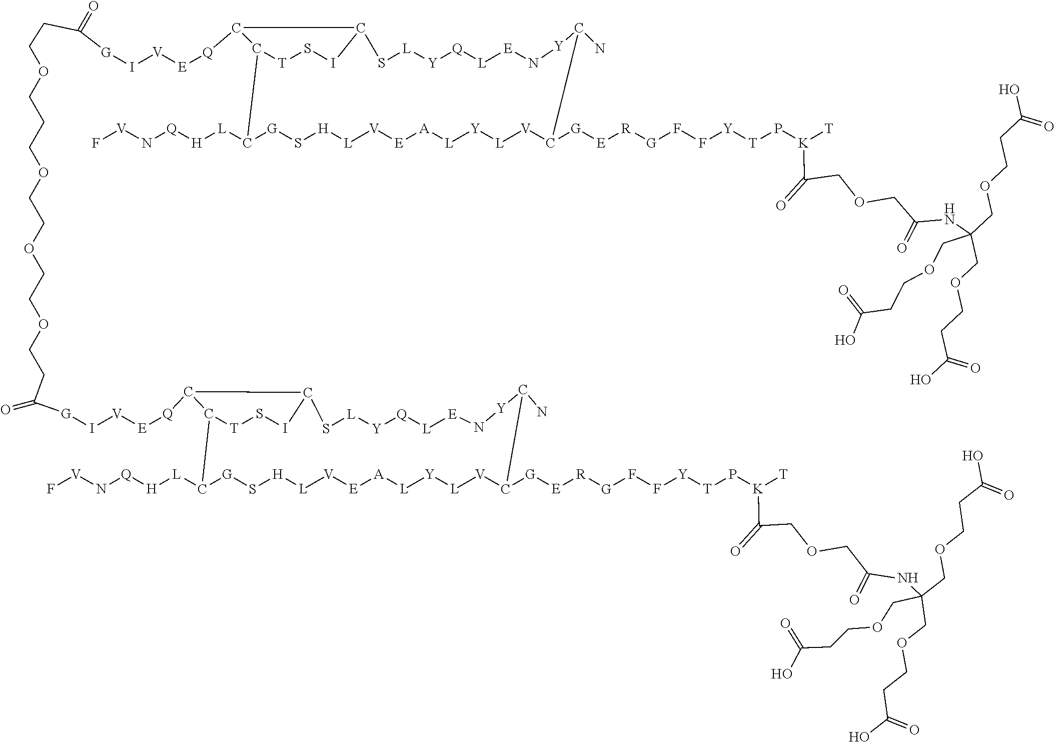 | D | 3.69 | 1821.38 |
| Di- mer 101 | 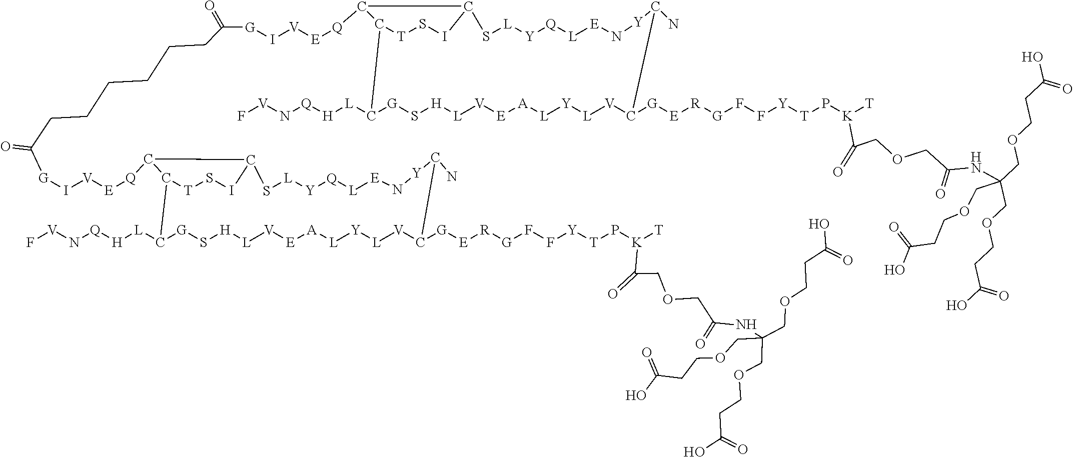 | D | 3.70 | 1804.12 |
| Di- mer 102 | 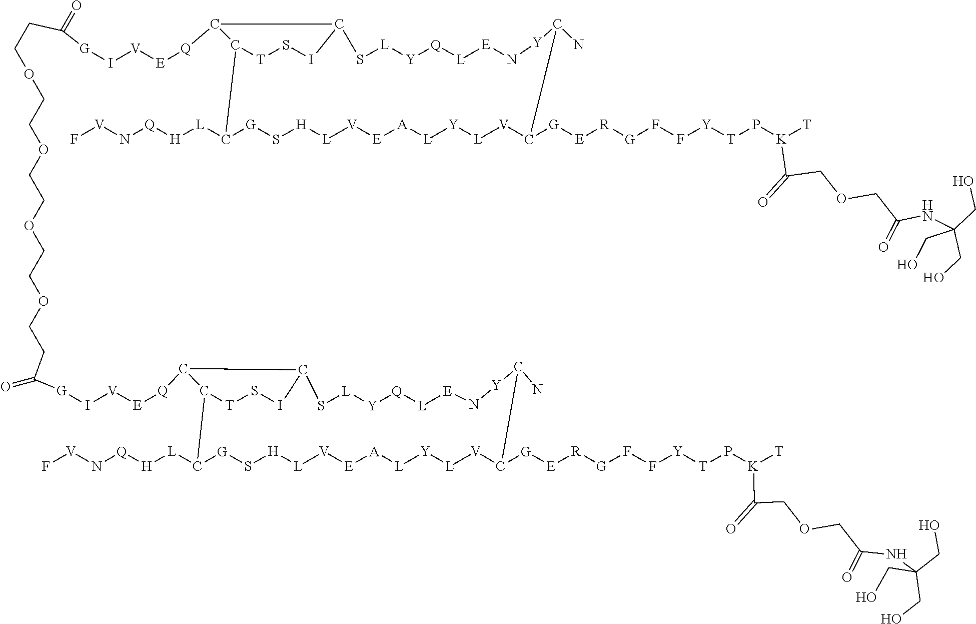 | D | 3.45 | 1709.67 |
| Di- mer 103 | 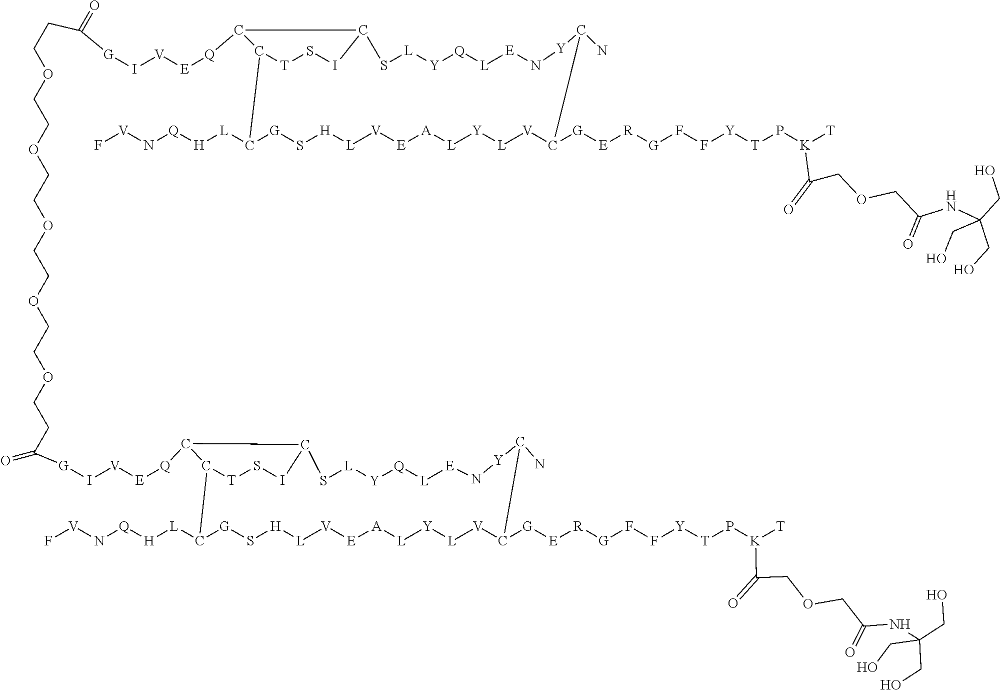 | D | 3.63 | 1765.82 |
| Di- mer 104 | 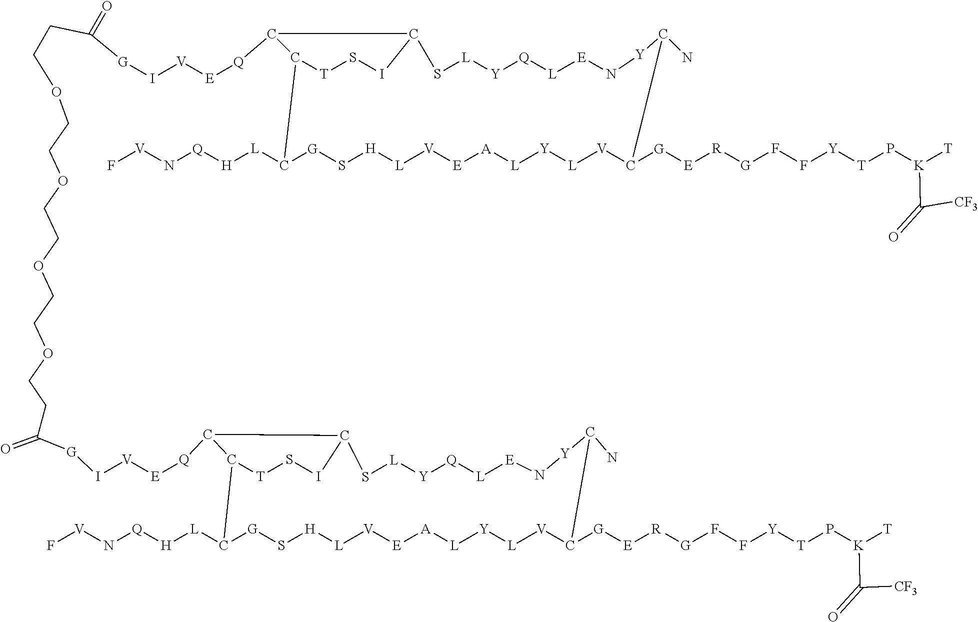 | D | 3.82 | 1725.30 |
| Di- mer 105 | 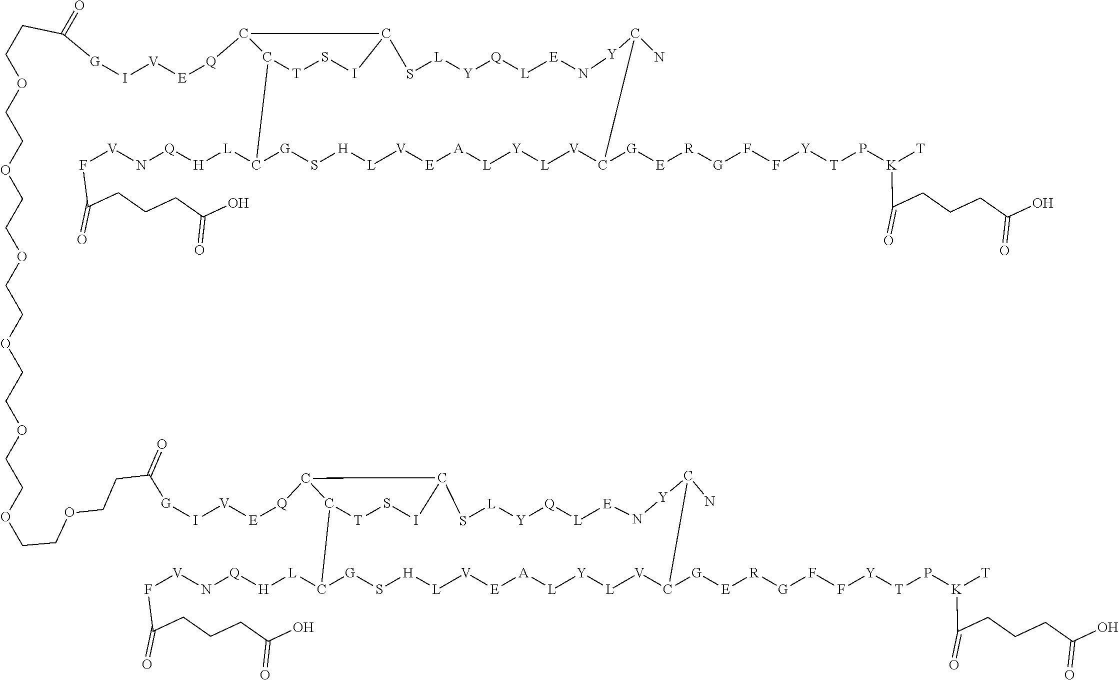 | C | 1.24 | 1780.83 |
| Di- mer 106 | 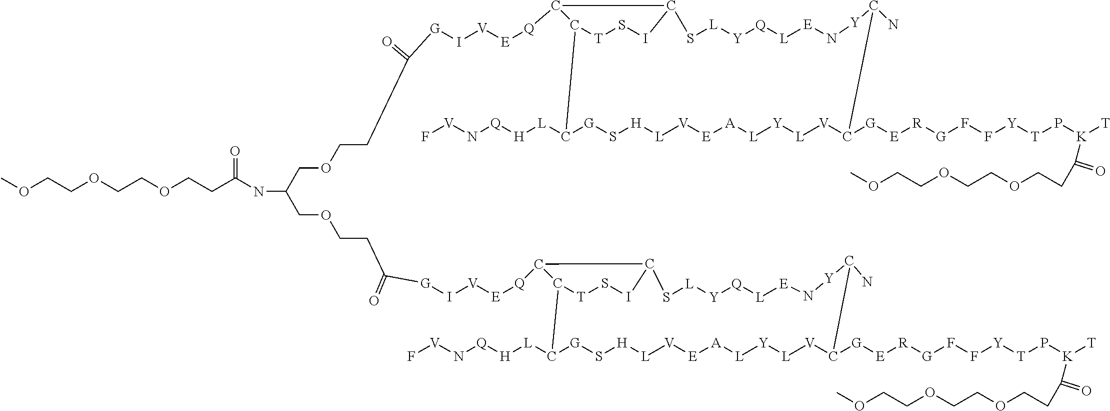 | D | 3.72 | 1763.6 |
| Di- mer 107 | 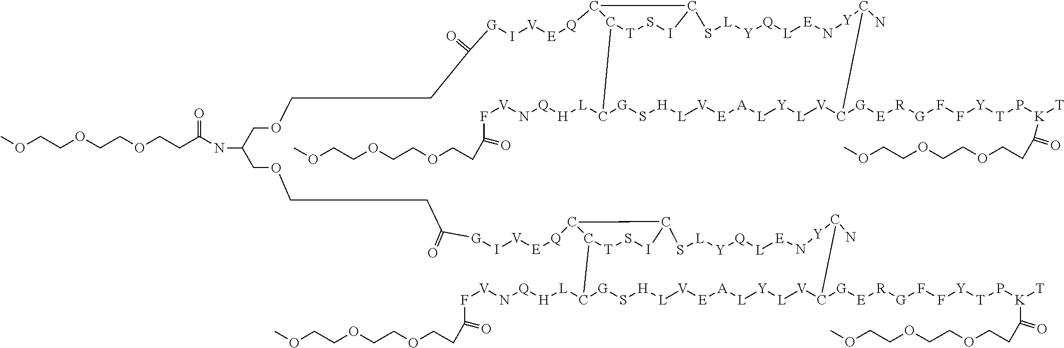 | D | 3.95 | 1812.8 |
| Di- mer 108 | 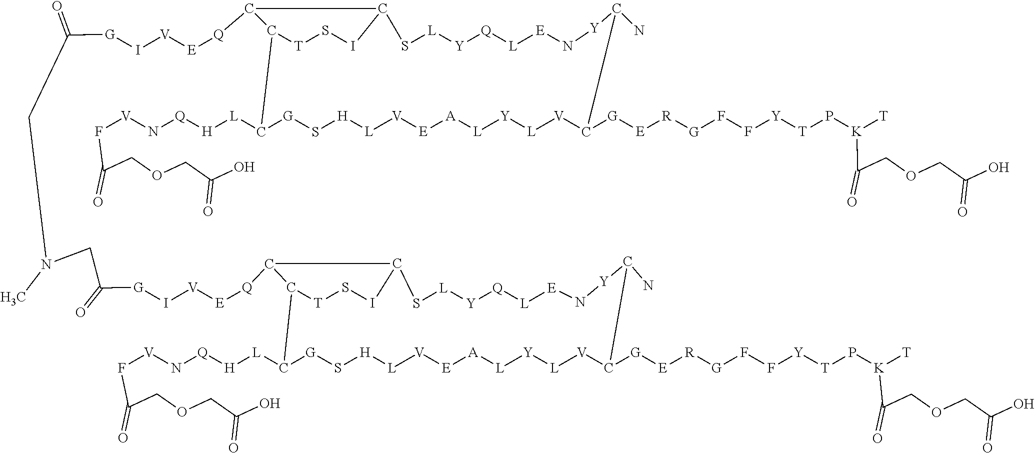 | A | 0.89 | 1743.45 |
| Di- mer 109 | 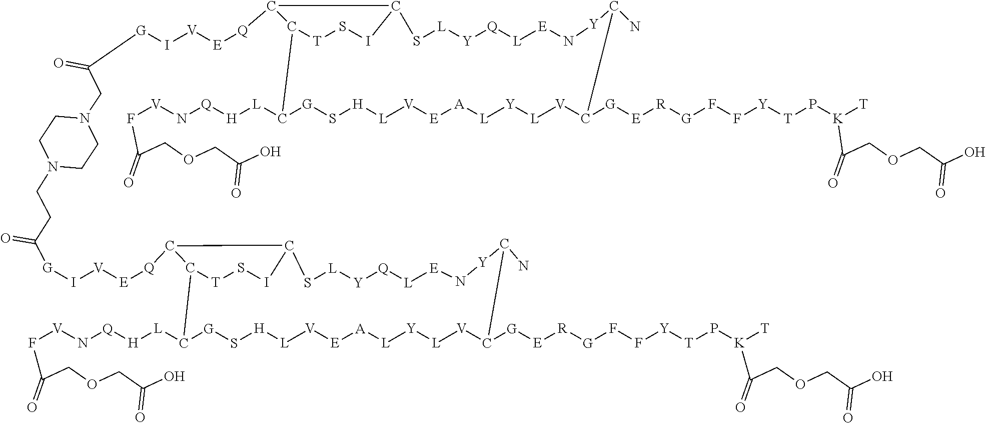 | A | 0.88 | 1753.17 |

### Example 16

General Method E: Synthesis of A1-A1′ dimers where B1, B1′, B29, B29′ are not blocked with caping groups.

### Step 1 Dimerization at A1-A1′ positions of Analog 4, N^{6,29B}-Boc RHI

N^{6,29B}-Boc RHI (Analog 4) is suspended at room temperature in an organic solvent or mixed aqueous (aq)/organic solvents, e.g., DMSO, in the presence of a base, e.g., TEA. The mixture is allowed to stir gently until insulin is completely dissolved. To the resulting solution is added a bis-functional activated ester intermediate (linking reagent) in solution of organic solvents, such as DMSO or DMF. After UPLC chromatogram shows that a substantial portion of the reaction mixture has converted into N^{2,1A},N^{2,1A′}-insulin dimer, the reaction mixture may be added dropwise to 50-100 volumes of IPAC, MTBE, or IPAC and MTBE mixture (e.g. 5:1), or IPAC and t-amyl alcohol mixture (e.g. 3:1), which causes the product of the reaction to form a precipitate. The precipitate is collected by filtration or centrifugation and dried using vacuum or a stream of nitrogen. Alternatively, the reaction mixture can be subjected directly to reverse phase HPLC purification (KROMASIL C8 250×50 mm, 10 μm, 100 Å column; Buffer A: 0.05-0.1% TFA in deionized water; Buffer B: 0.05-0.1% TFA in AcCN), or the reaction may be quenched by careful dilution with cold acidic H2O (20×, pH about 3.0) at 0° C. and its pH is adjusted to a final pH of 2.5 using 1 N HCl (and 0.1 N NaOH if needed). The solution may first be concentrated by ultrafiltration, either through a tangential flow filtration (TFF) system or using Amicon Ultra-15 Centrifugal Units, with 1K, 3K or 10K MWCO membrane. The concentrated solution is then subjected to reverse phase HPLC purification (KROMASIL C8 250×50 mm, 10 μm, 100 Å column; Buffer A: 0.05-0.1% TFA in deionized water; Buffer B: 0.05-0.1% TFA in AcCN). Fractions containing the desired insulin dimer are combined and freeze-dried or buffer exchanged using TFF system and/or Amicon Ultra-15 to give the N^{2,1A},N^{2,1A′} insulin dimers, in which N^{6,29A} and N^{6,29A′} positions are protected with Boc groups.

### Step 2. Removal of Boc Protective Groups from N^{6,29A} and N^{6,29A′} Positions of the Dimer

The product of Step 1 is dissolved in an appropriate volume of trifluoroacetic acid, and removal of Boc protective groups is confirmed by UPLC typically after 30 min of reaction time. The reaction mixture is added dropwise to 50-100 volumes of IPAC, MTBE, or IPAC and MTBE mixture (e.g. 5:1), which causes the product of the reaction to form a precipitate. The precipitate is collected by filtration or centrifugation, dried using vacuum or a stream of nitrogen, and can be first subjected to ion exchange chromatography (PolySULFOETHYL A column, PolyLC Inc., 250×21 mm, 5 μm, 1000 Å; Buffer A: 0.1%(v/v)H3PO4/25% AcCN; Buffer B: 0.1%(v/v)H3PO4/25% AcCN/0.5 M NaCl). The desired fractions are combined and concentrated using TFF system or Amicon Ultra-15. The concentrated solution is then subjected to reverse phase HPLC purification (KROMASIL C8 250×50 mm, 10 μm, 100 Å column; Buffer A: 0.05-0.1% TFA in deionized water; Buffer B: 0.05-0.1% TFA in AcCN). Fractions containing the desired insulin dimer are combined and freeze-dried or buffer exchanged using TFF system and/or Amicon Ultra-15 to give the N^{2,1A},N^{2,1A′} Insulin dimers.

Alternatively, the crude reaction mixture can be added dropwise into a mixture of cold water and AcN while maintaining pH of the resulting solution at 2.5. The solution is concentrated using TFF system or Amicon Ultra-15 and purified as described earlier.

### Example 17

### Dimer 110 Synthesis Using General Method E

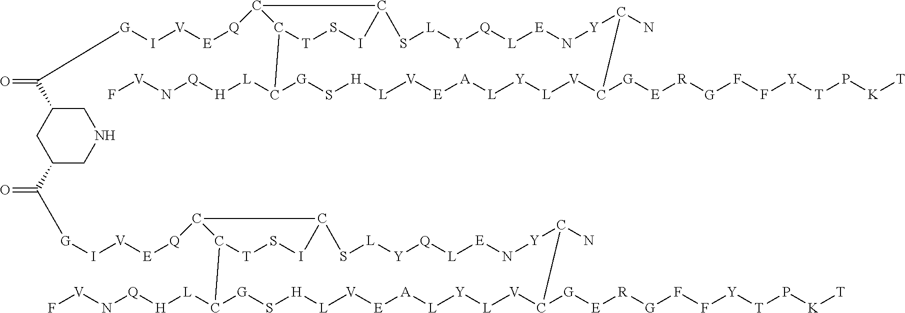

### Step 1 Dimerization at A1-A1′ Positions

Analog 4 (193 mg; 0.033 mmol) is suspended at room temperature in 2.0 mL of DMSO, in the presence of TMP (150 μL; 0.889 mmol). The mixture is allowed to stir gently until insulin is completely dissolved. To the resulting solution is added 1-(tert-butyl) 3,5-bis(2,5-dioxopyrrolidin-1-yl) (3R,5S)-piperidine-1,3,5-tricarboxylate (Linking Reagent 4, 7.65 mg; 0.016 mmol) in 80 μL of DMSO. After UPLC chromatogram shows that a substantial portion of the reaction mixture has converted into N^{2,1A},N^{2,1A′}-insulin dimer, the reaction mixture is added dropwise to 50 mL of IPAC/MTBE (4:1) solvent mixture, which causes the product of the reaction to form a precipitate. The precipitate is collected by filtration and dried using vacuum and a stream of nitrogen. The crude precipitate is then re-dissolved in 20 mL of 20% CH3CN/water and its pH is adjusted to a final pH of 3 using 1 N HCl (and 0.1 N NaOH if needed). The acidified solution of the crude mixture is subjected to reverse phase HPLC purification (KROMASIL C8 250×50 mm, 10 μm, 100 Å column; Buffer A: 0.05% TFA in deionized water; Buffer B: 0.05% TFA in AcCN). Fractions containing the desired insulin dimer are combined and freeze-dried to give the N^{2,1A},N^{2,1A′} insulin dimer, in which N^{6,29A},N^{6,29A′} and the linker's piperidine ring N position are protected with Boc groups. UPLC-MS Method D: Rt=3.72 min, m/z=1721.34 [(M+7)/7].

### Step 2 Removal of Boc Protective Groups from N^{6,29A}, N^{6,29A′} and from the Linker

The product of Step 1 was processed according to the procedure described in Step 2 of General Method E. The title material was isolated as a white solid after lyophilization of fractions of preparative HPLC chromatography (KROMASIL C8 250×50 mm, 10 μm, 100 Å column; Buffer A: 0.05% TFA in deionized water; Buffer B: 0.05% TFA in AcCN). UPLC-MS Method D: Rt=3.39 min, m/z=1959.66 [(M+6)/6].

Variation of General Method E: Synthesis of A1-A1′ dimers where B1, B1′, B29, B29′ are not blocked with capping groups, using Analog 31 as the starting material.

### Example 18

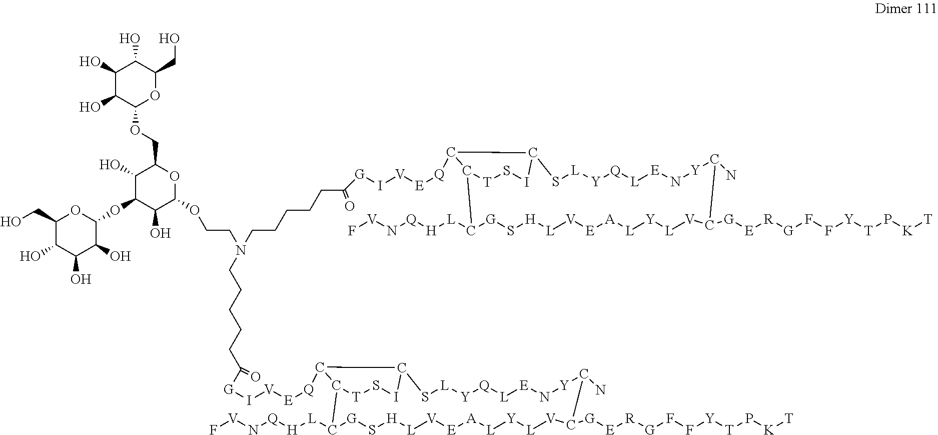

B1,B29-Bis-phenylacetamide insulin (Analog 31) (150 mg, 0.025 mmol) was dissolved in DMSO (1.0 mL) and treated with Et3N (0.086 mL, 0.619 mmol). Linking reagent, bis(2,5-dioxopyrrolidin-1-yl) 6,6′-((2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)azanediyl)dihexanoate (Linking reagent 44) (12 mg, 0.012 mmol) was dissolved in DMSO (0.05 mL) and added to the reaction mixture and stirring proceeded for 2 hours. The reaction mixture was added to acetonitrile (40 mL) to form a white precipitate which was isolated by centrifugation. The pellet was re-suspended in acetonitrile (40 mL) and a cycle of centrifugation was repeated. The supernatent was decanted and solid pellet dried under a stream of dry nitrogen.

The pellet was dissolved in 0.1M tris HCl buffer with pH adjusted to 8.0 by the addition of 1N NaOH. Enzyme PGA-005 [SEQ ID NO: 6, disclosed in US2020/0115696 (40 mg)] dissolved in 0.1M tris HCl buffer (1 mL) and 0.6 mL of this solution added to the insulin mixture and shaken (300 rpm) for 3 hours. The product was isolated by ion-exchange chromatography (IEC) (PolySULFOETHYL A column, PolyLC Inc., 250×21 mm, 5 μm, 1000 Å; Buffer A: 0.1%(v/v)H₃PO₄/25% AcCN; Buffer B: 0.1%(v/v) H₃PO₄/25% AcCN/0.5 M NaCl), gradient 10-60% of Solvent B in 24 min. Fractions containing the desired material were combined, and the product was re-purified by reverse-phase chromatography (KROMASIL C8 250×50 mm, 10 μm, 100 Å column; Buffer A: 0.05% TFA in deionized water; Buffer B: 0.05% TFA in AcCN, flow 85 mL/min, gradient from 28% to 34% in 30 min). Product-containing fractions were lyophilized to give the title material as a solid. UPLC-MS Method D: Rt=3.32 min, m/z=1765.64[(M+7)/7].

### Example 19

Table III insulin dimers 112 through 127 (backbone depicted by Formula I) in Table X below, were prepared by General Method E, wherein A1-A1′ dimers do not have capping groups on 1B1,1B29,B31′, B29′.

TABLE X

| | | | | (M + 6)/6 |
|---|---|---|---|---|
| | | UPLC | Rt | or |
| Dimer # | Linker Chemdraw | Method | (min) | ((M + 7)/7) |
| Dimer 112 | 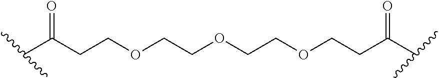 [SMILES: *C(=O)CCOCCOCCOCCC(*)=O] | D | 4.50 | 1972.21 |
| Dimer 113 | 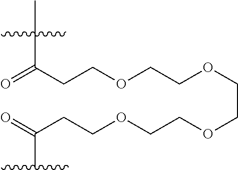 [SMILES: *C(=O)CCOCCOCCOCCOCCC(*)=O] | D | 3.32 | 1979.679 |
| Dimer 114 | 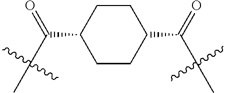 | D | 3.36 | 1959.51 |
| Dimer 115 | 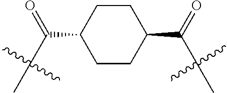 [SMILES: *C(=O)[C@H]1CC[C@H](C(*)=O)CC1] | D | 3.43 | 1959.28 |
| Dimer 116 | 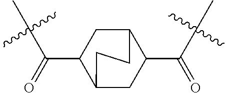 [SMILES: *C(=O)C1CC2CCC1CC2C(*)=O] | D | 3.76 | 1964.18 |
| Dimer 117 | 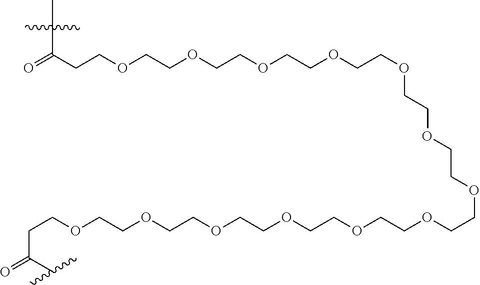 [SMILES: *C(=O)CCOCCOCCOCCOCCOCCOCCOCCOCCOCCOCCOCCOCCOCCC(*)=O] | D | 3.60 | 1753.70 |
| Dimer 118 | 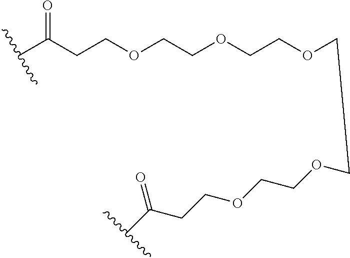 [SMILES: *C(=O)CCOCCOCCOCCOCCOCCC(*)=O] | D | 3.58 | 1703.52 |
| Dimer 119 | 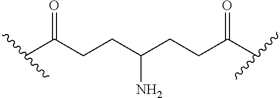 [SMILES: *C(=O)CCC(N)CCC(*)=O] | D | 3.43 | 1959.9; 1680.0 |
| Dimer 120 | 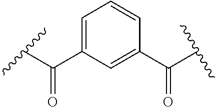 [SMILES: *C(=O)c1cccc(C(*)=O)c1] | D | 3.41 | 1959.8, 1680.1 |
| Dimer 121 | 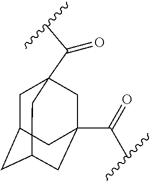 [SMILES: *C(=O)C12CC3CC(C1)CC(C(*)=O)(C3)C2] | D | 4.41 | 1967.9 1686.9 |
| Dimer 122 | 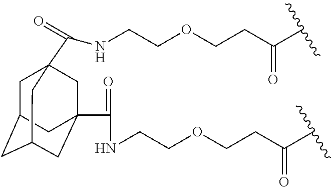 [SMILES: *C(=O)CCOCCNC(=O)C12CC3CC(C1)CC(C(=O)NCCOCCC(*)=O)(C3)C2] | D | 3.64 | 1719.9 |
| Dimer 123 | 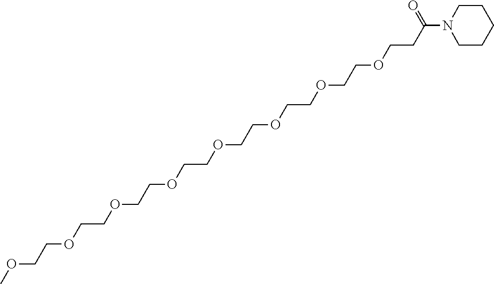 [SMILES: COCCOCCOCCOCCOCCOCCOCCOCCC(=O)N1CCCCC1] | D | 3.6 | 1740.8302 |
| Dimer 124 | 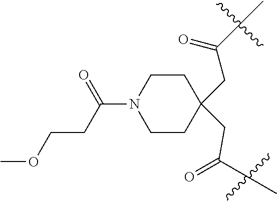 [SMILES: *C(=O)CC1(CC(*)=O)CCN(C(=O)CCOC)CC1] | D | 3.55 | 1978.41 |
| Dimer 125 | 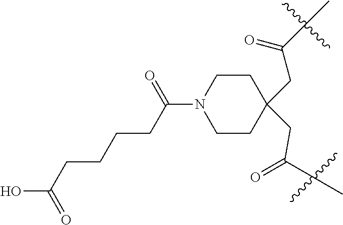 [SMILES: *C(=O)CC1(CC(*)=O)CCN(C(=O)CCCCC(=O)O)CC1] | D | 3.54 | 1985.7452 |
| Dimer 126 | 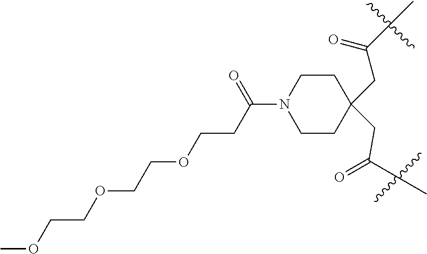 [SMILES: *C(=O)CC1(CC(*)=O)CCN(C(=O)CCOCCOCCOC)CC1] | D | 3.55 | 1993.019 |
| Dimer 127 | 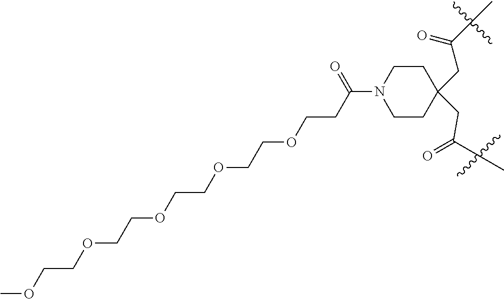 [SMILES: *C(=O)CC1(CC(*)=O)CCN(C(=O)CCOCCOCCOCCOCCOC)CC1] | D | 3.56 | 1721.09 |

### Example 20

Table III insulin dimers 128 through 130 (backbone depicted by Formula I), in Table XI below, were prepared by General Method E, wherein A1-A1′ dimers do not have capping groups on B1,B29,B1′, B29′.

TABLE XI

| Di- | | | | (M + 6)/ |
|---|---|---|---|---|
| mer | | UPLC | | 6 or |
| Num- | | Meth- | Rt | (M + 7)/ |
| ber | Composite structure + Chemdraw Structure | od | (min) | 7 |
| Di- mer 128 | 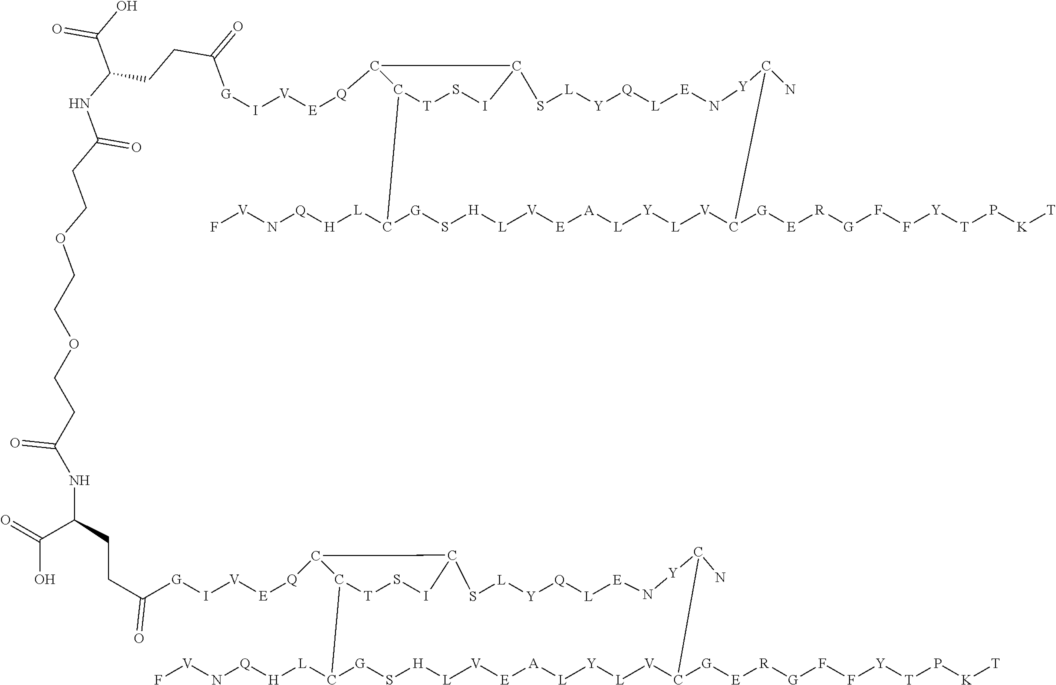 | D | 3.6 | 1721.19 |
| Di- mer 129 | 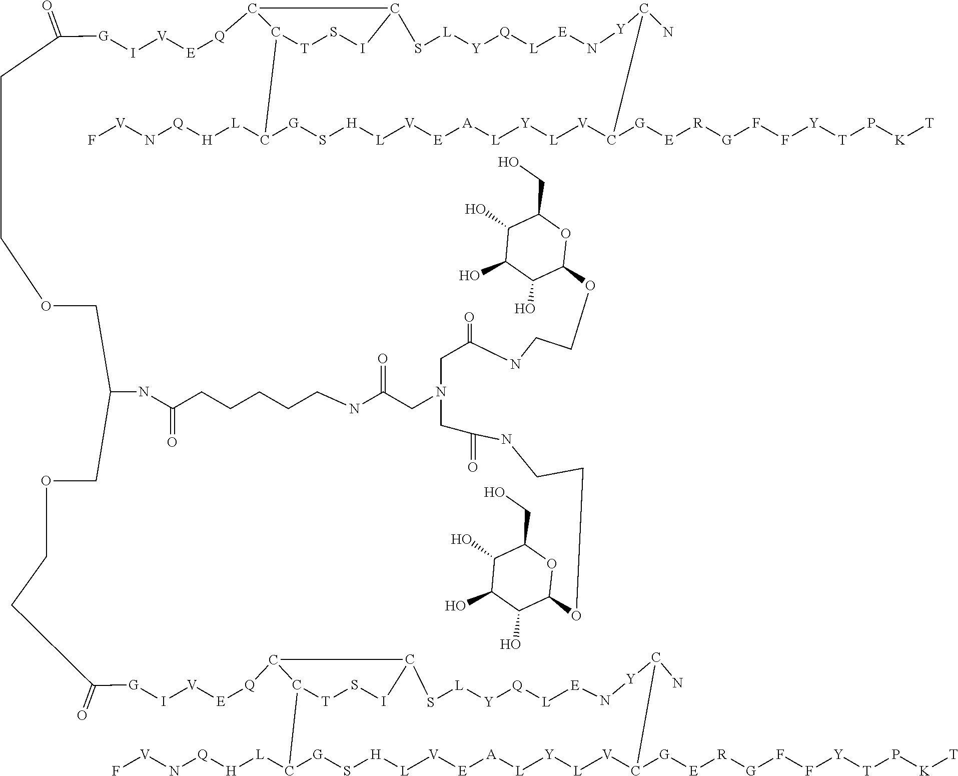 | A | 0.84 | 1788.35 |
| Di- mer 130 | 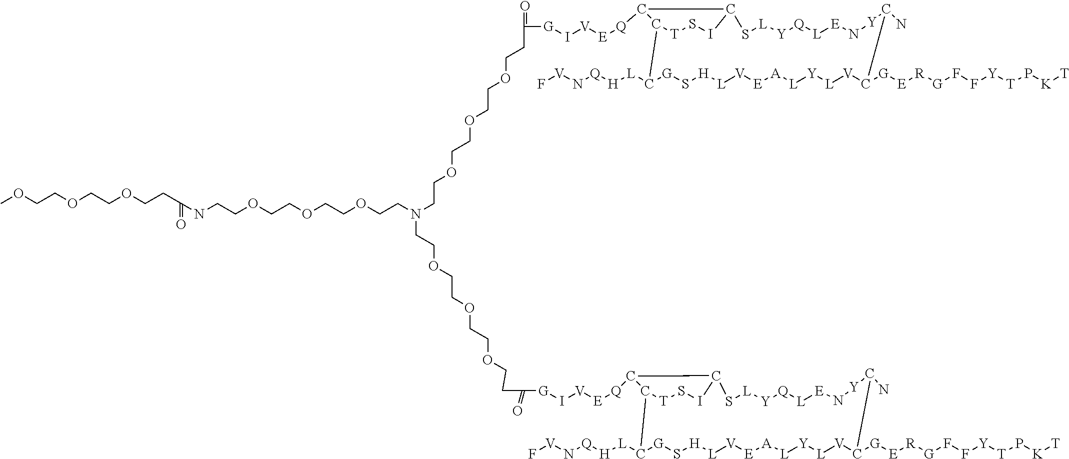 | D | 3.42 | 1765.9136 |

### General Method F: Synthesis of Insulin Dimers Using Cu²⁺-Catalyzed Click Chemistry

### Example 21

In an appropriately sized container, appropriate acetylene containing insulin intermediate (Analog) was dissolved, with gentle stirring, at room temperature in a mixed solvent of DMSO and aq. triethylammonium acetate buffer (pH 7.0, concentration 0.2 mM). In another appropriately sized container, appropriate azido containing insulin intermediate (Analog) was dissolved, with gentle stirring, at rt in a mixed solvent of DMSO and water. Both solutions were combined, thoroughly mixed, and degassed by gentle bubbling of nitrogen. To the resulting solution was added freshly prepared sodium ascorbate or ascorbic acid solution (final concentration is 0.5 mM) and, after thoroughly mixing, a solution of 10 mM of CuSO₄ and tris[(1-benzyl-1H-1,2,3-triazol-4-yl)methyl]amine (i.e., TBTA ligand) in 55% DMSO. After degassing by gentle bubbling N₂ and thorough mixing, the mixture was stored at rt, with occasional mixing, overnight. The reaction mixture was carefully diluted with a mixed solvent (v/v 7:3 AcCN/water with 0.05% TFA) at 0° C. and pH was adjusted to 2.50 using 0.1, 1.0 N HCl (and 0.1 N NaOH if needed). The solution was first concentrated by ultrafiltration, either through a tangential flow filtration (TFF) system or using Amicon Ultra-15 Centrifugal Units, with 1K, 3K, or 10K MWCO membrane. The concentrated solution was usually first subjected to ion exchange chromatography (PolySULFOETHYL A column, PolyLC Inc., 250×21 mm, 5 □m, 1000 Å; Buffer A: 0.1%(v/v)H₃PO₄/25% AcCN; Buffer B: 0.1%(v/v)H₃PO₄/25% AcCN/0.5 M NaCl). Fractions containing desired product with desired purity were combined and concentrated using TFF system or Amicon Ultra-15. The resulting solution was then further purified by reverse phase HPLC (Waters C4 250×50 mm column, 10 □m, 1000 Å column or KROMASIL C8 250×50 mm, 10 □m, 100 Å column; Buffer A: 0.05-0.1% TFA in water; Buffer B: 0.05-0.1% TFA in AcCN). Fractions containing the desired product with desired purity were combined and freeze-dried or buffer exchanged using TFF system and/or Amicon Ultra-15 to give the insulin dimers.

### Example 22

Table III insulin dimers 131 and 132 (backbone depicted by Formula I) and dimer 133 (backbone depicted by Formula Ic), disclosed in Table XII below, were prepared from appropriate alkyne and azide precursors (Analogs) using conditions of General Method F, wherein A1-A1′ dimers do not have capping groups on B1,B29,B1′, B29′.

TABLE XII
Dimers prepared by General Method F (Click Chemistry).

| | | | (M + 6)/6 | |
|---|---|---|---|---|
| | | Rt | or | UPLC |
| Dimer | Description | (min) | ((M +7)/7) | Method |
| Dimer 131 (from Analog 36 + 37 | 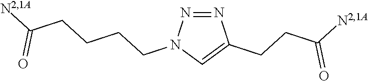 | 3.83 | 1970.97 | D |
| Dimer 132 (from Analog 38 + 39) | 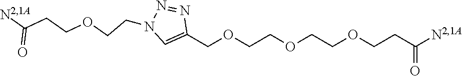 | 3.30 | 1993.13 | D |
| Dimer 133 (from Analog 37 + 40) | 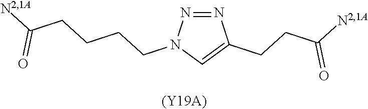 | 4.50 | 1938.935 | D |

### General Method G: Synthesis of B1,B1=Capping Group Installed, A1,A1′=No Capping Group Installed

B29-Boc-RHI is dimerized at A1,A1′ positions using an appropriate linking reagent and conditions of the general method D. The resulting dimer is isolated, re-dissolved in DMSO, and acylated with a capping reagent in the presence of an organic base (e.g. triethylamine or TMP). The product, B1,B1′-capped, B29,B29′-Boc-protected dimer is isolated by precipitation, after dilution of the reaction mixture with a weaker solvent (ether, MTBE, IPAC, etc.) and optionally purified by preparative reverse-phase chromatography. Finally, B29 and B29′ sites are deprotected by treatment of the material with TFA containing 2.5% of water.

### Example 23

### General Method G: Synthesis of Dimer 134

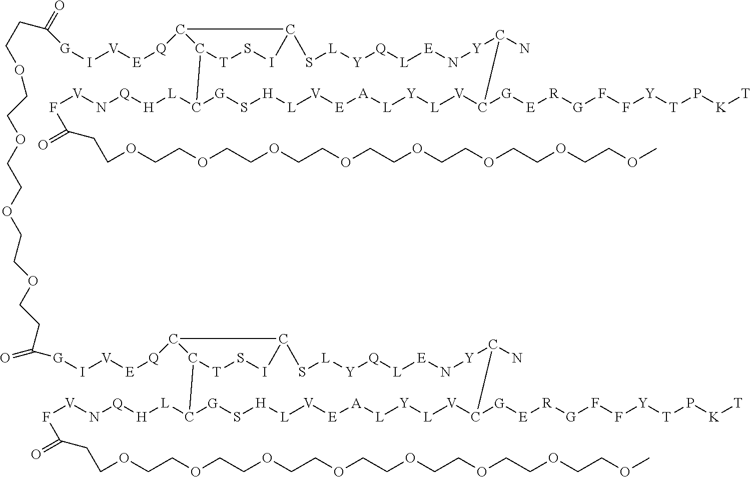

Step 1 Coupling: Starting from Analog 4 B29-Boc-RHI and Linking Reagent 32 and using procedures of general method D, the B29,B29′-bis-Boc-protected dimer was obtained. UPLC-MS Method D: Rt=3.92 min, m/z=1726.42 [(M+7)/7].

Step 2. Acylation at B1,B1′ positions.

To a solution of the material of Step 1 (89 mg, 7.37 μmol) in DMSO (434 μl) was added triethylamine (41.1 μl, 0.295 mmol) followed by a solution of the capping reagent 2,5-dioxopyrrolidin-1-yl 2,5,8,11,14,17,20,23-octaoxahexacosan-26-oate (Capping Reagent 6) (9.39 mg, 0.018 mmol) in a small amount of DMSO (100 μL). Stirred the mixture overnight. Precipitated by adding the reaction mixture to ice-cold MTBE (30 mL) and collected precipitate by centrifugation. Removed trace solvent from precipitate by pumping on rotovap. UPLC-MS Method D: Rt=4.15 min, m/z=1839.39[(M+7)/7].

Step 3. Cleavage of Boc protective groups.

Treated the precipitate with 1.5 mL of TFA and let the mixture stand for 1.5 hr. Added the reaction mixture to ice-cold MTBE (30 mL) and collected ppt by centrifugation. Removed trace solvent from precipitate by pumping on rotovap. The product was purified by reverse-phase chromatography on C-8 phase (Column Kromasil, C8 10 μM 100 A, size 250×50 mm; solvent A=water/0.05% TFA, solvent B=AcN/0.05% TFA), Flow=85 mL/min, gradient B in A 27-38% in 30 min (Gilson C). UPLC-MS Method D: Rt=3.98 min m/z=1810.85 [(M+7)/7].

### Example 24

TABLE III
insulin dimers 135 through 142 in Table XIII below, were prepared using conditions of General Method G.

| | | | | (M + 6)/6 |
|---|---|---|---|---|
| Dimer | | UPLC | Rt | or |
| Number | Composite structure + Chemdraw Structure | Method | (min) | (M + 7)/7 |
| Dimer 135 | 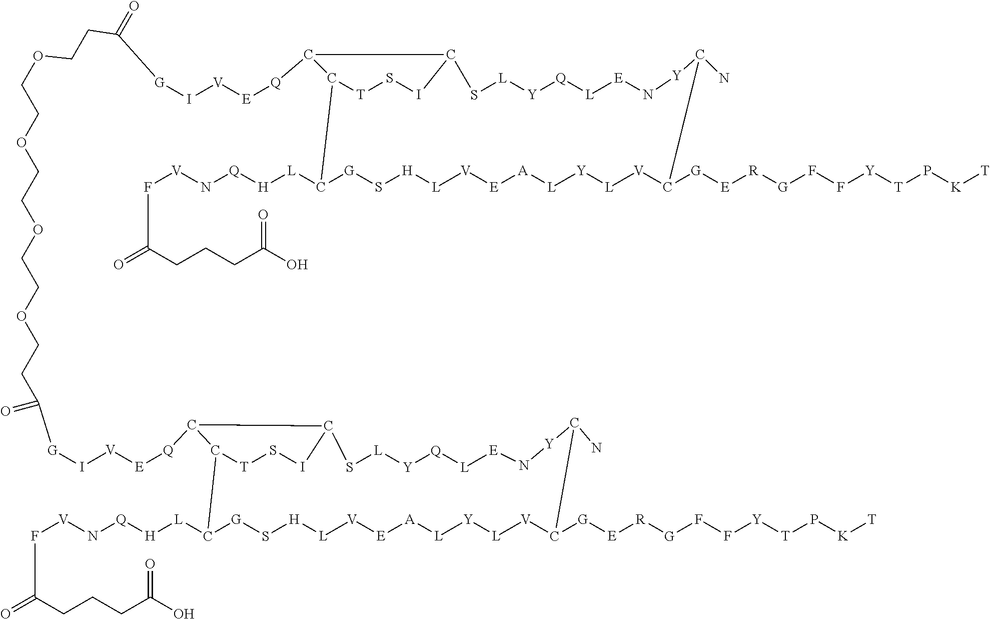 | D | 3.67 | 1729.79 |
| Dimer 136 | 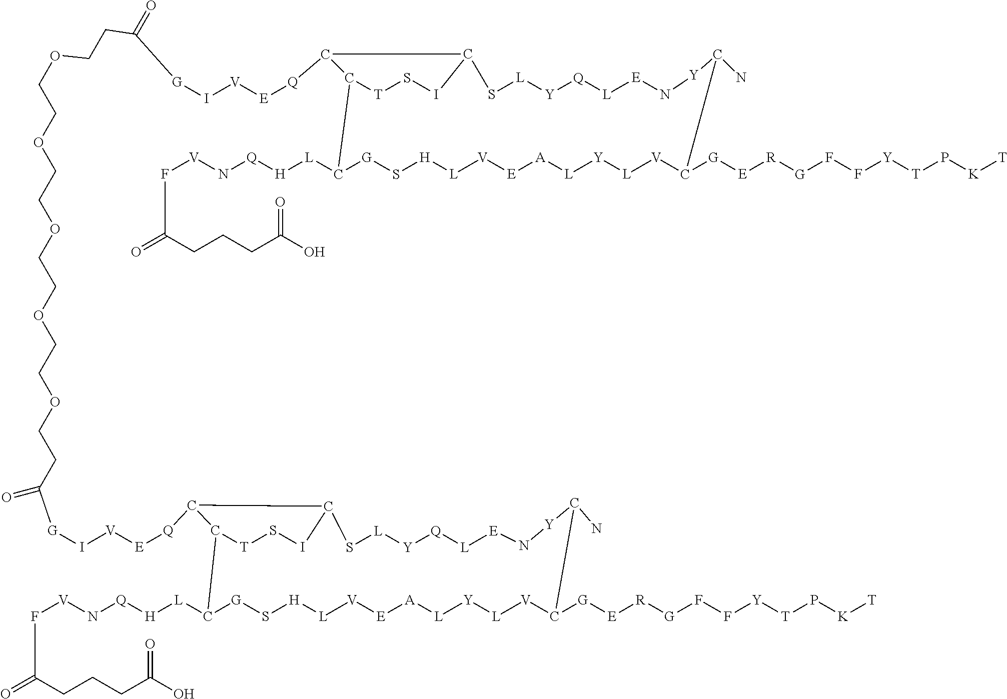 | D | 3.62 | 1737.47 |
| Dimer 137 | 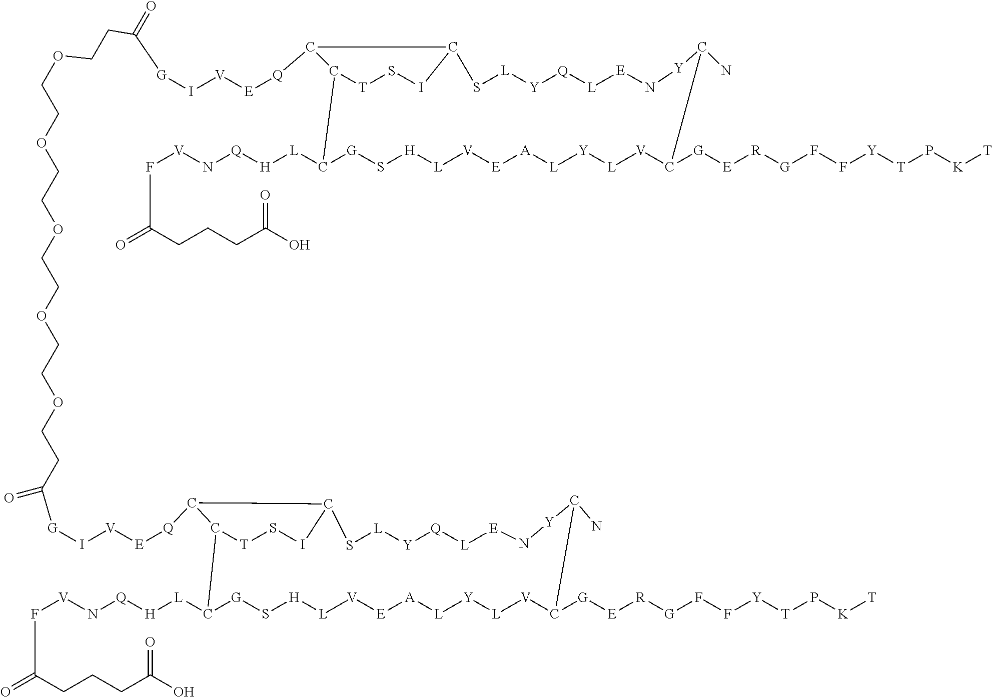 | D | 3.71 | 1736.97 |
| Dimer 138 | 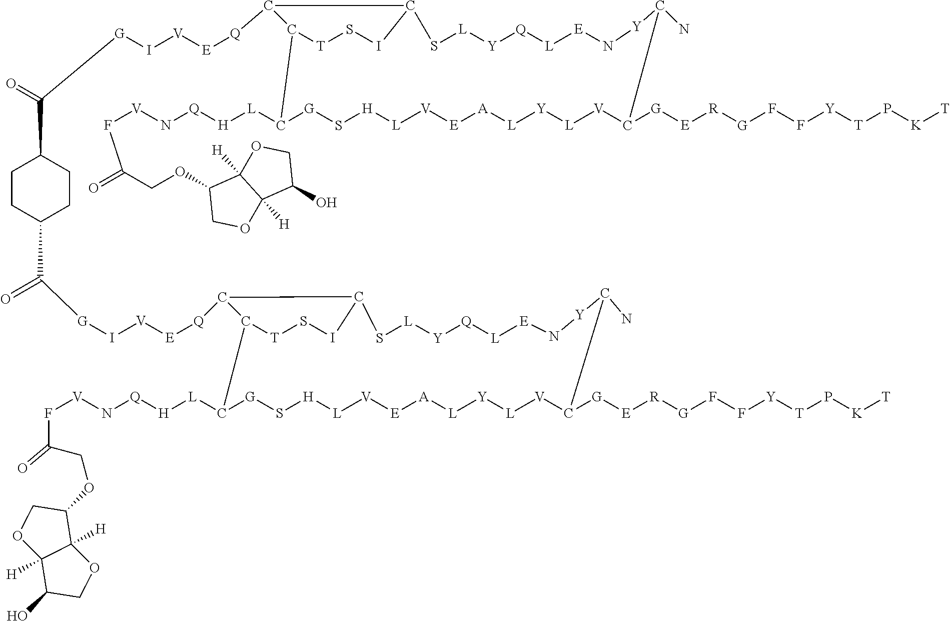 | D | 3.67 | 1733.32 |
| Dimer 139 | 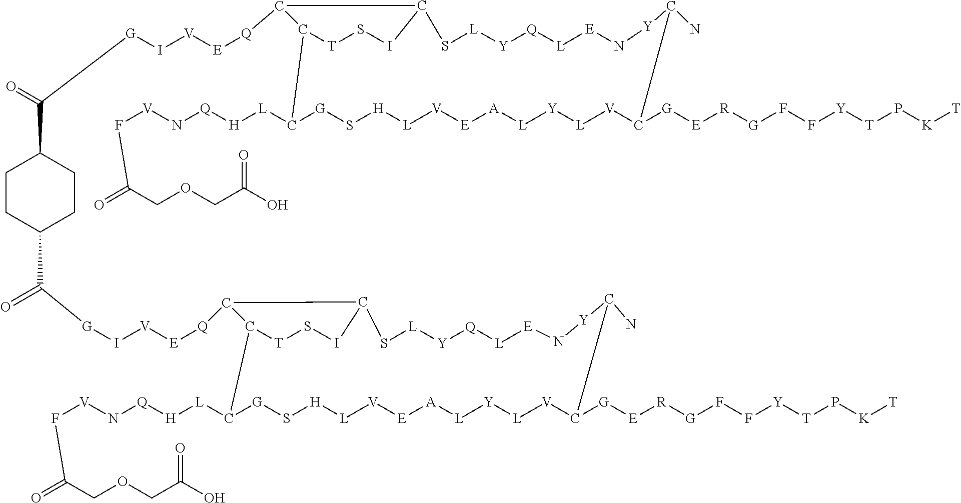 | D | 3.72 | 1712.51 |
| Dimer 140 | 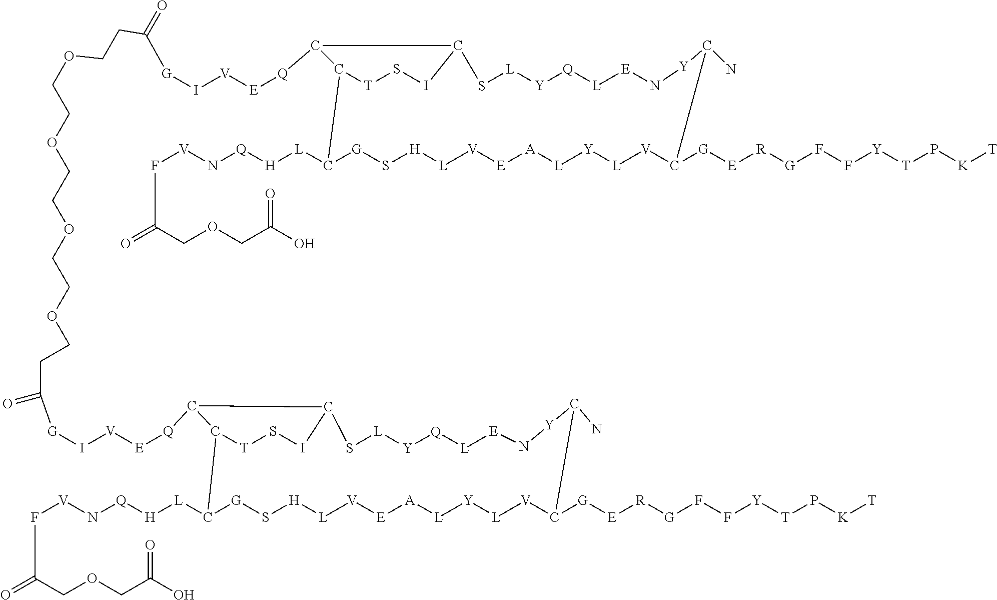 | D | 3.71 | 1731.33 |
| Dimer 141 | 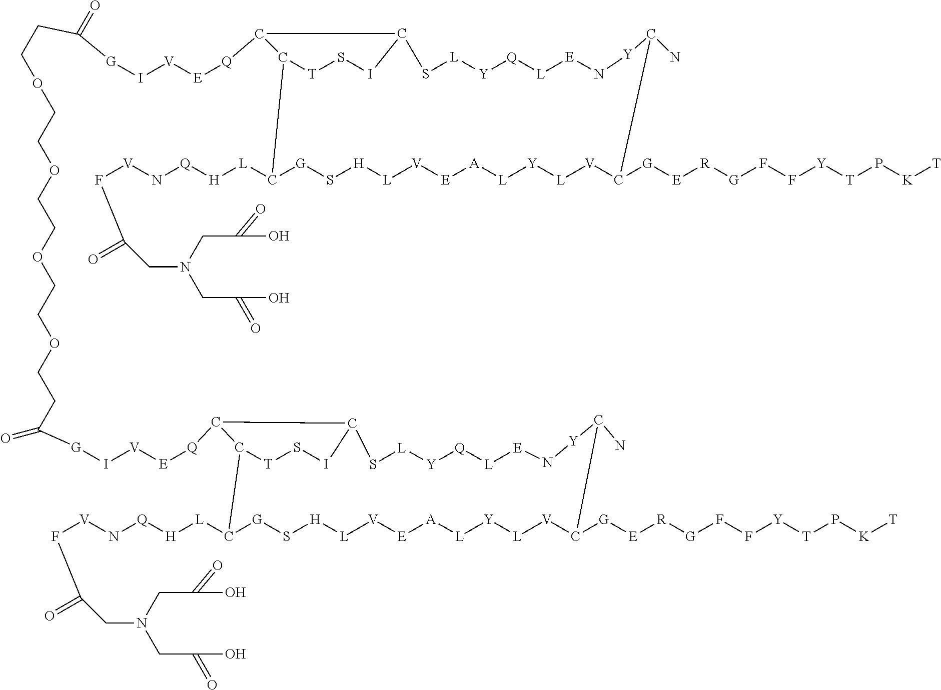 | D | 3.69 | 1747.30 |
| Dimer 142 | 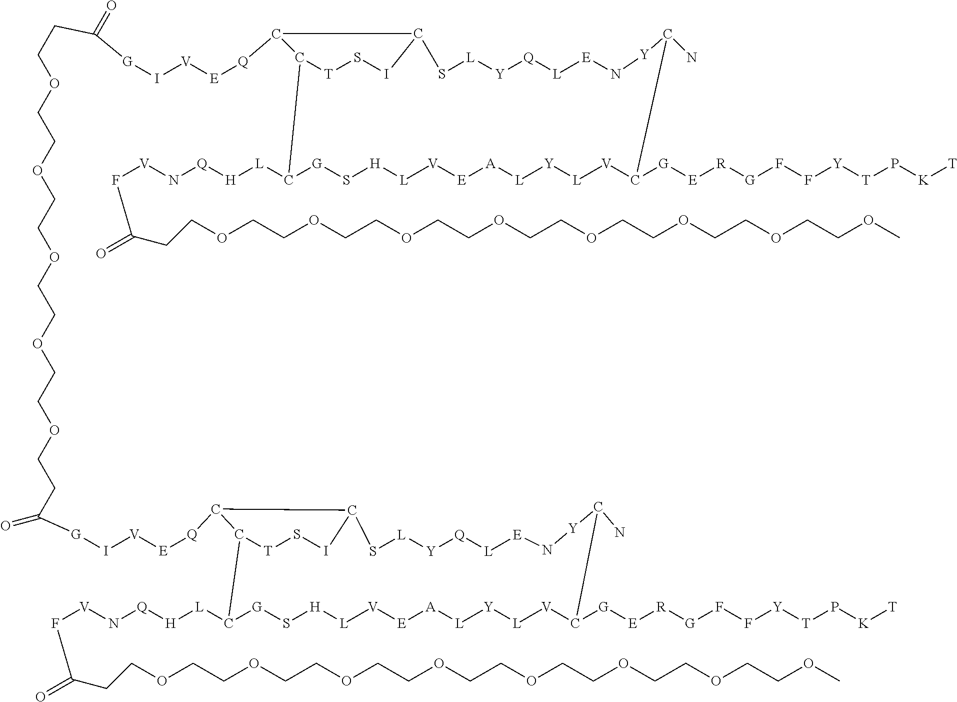 | D | 3.64 | 1816.31 |

### General Method H

In some cases, the linking reagent bears protective groups which can be cleaved after formation of the dimer using base, which as ammonium hydroxide. General method H involves treatment of the crude insulin dimer with ammonium hydroxide to remove the protective groups from the linker.

### Example 25

### Illustration of General Method H. Synthesis of Dimer 143

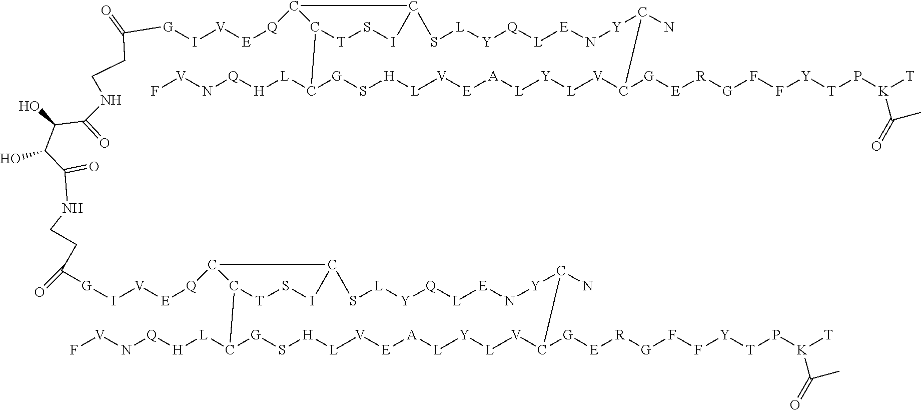

To a solution of B29-Ac-RHI (Analog 7) (192 mg, 0.033 mmol) in DMSO was added triethylamine (183 μl, 1.313 mmol) followed by the linking reagent bis(2,5-dioxopyrrolidin-1-yl) 3,3′-(((2R,3R)-2,3-diacetoxysuccinyl)bis(azanediyl))dipropionate (Linking reagent 37, 0.1M/DMF) (181 μl, 0.018 mmol). Stirred the mixture for 1 hr. Isolated the crude dimer by precipitation of the reaction mixture into 30 mL of ice-cold MTBE and centrifugation. Dried the pellet in a stream of nitrogen. Dissolved the pellet in 1.5 mL of concentrated (29%) commercial ammonium hydroxide and allowed to stand for 1 hr to remove the protective acetyl groups. Removed most of the ammonium hydroxide by diluting the reaction mixture with water to total volume of 15 mL and doing 3 cycles of diafiltration in Amicon Tubes reducing the volume to ˜3 mL per cycle, and adding fresh water. After last cycle and shortly before the injection on HPLC column, adjusted pH to 2.5 with 1M HCl. The product was isolated by reverse-phase chromatography on C-8 phase (Column Kromasil, C8 10 uM 100 Å, size 250×50 mm; solvent A=water/0.05% TFA, solvent B=AcN/0.05% TFA), Flow=85 m/min, gradient B in A 27-36% in 30 min. UPLC-MS Method D: Rt=3.58 min, m/z=1709.57[(M+7)/7].

### Example 26

### Synthesis of Dimer 144

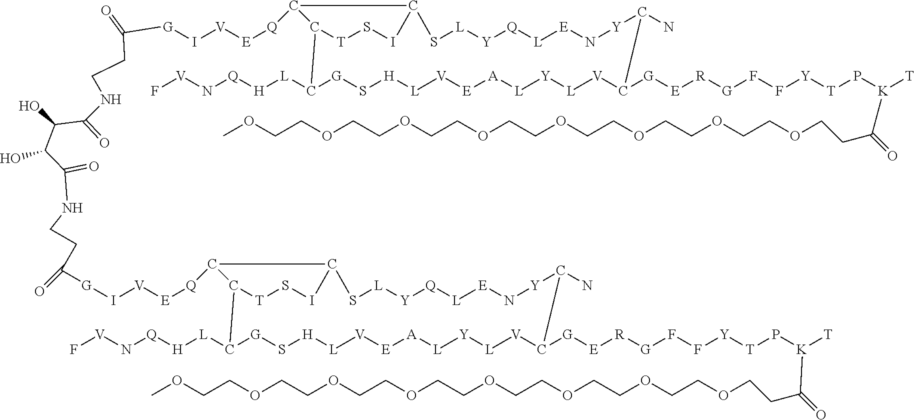

Dimer 144 was synthesized from Analog 5 and Linking reagent 37 according to general method H. UPLC-MS Method D: Rt=3.70 min, m/z=1810.81[(M+7)/7].

### Example 27

A. Insulin Receptor Binding Assays were performed as follows.

IR binding assay was run in a scintillation proximity assay (SPA) in 384-well format using cell membranes prepared from CHO cells overexpressing human IR(B) grown in F12 media containing 10% FBS and antibiotics (G418, Penicillin/Strepavidin). Cell membranes were prepared in 50 mM Tris buffer, pH 7.8 containing 5 mM MgCl₂. The assay buffer contained 50 mM Tris buffer, pH 7.5, 150 mM NaCl, 1 mM CaCl₂, 5 mM MgCl₂, 0.1% BSA and protease inhibitors (Complete-Mini-Roche). Cell membranes were added to WGA PVT PEI SPA beads (5 mg/mL final concentration) followed by addition of insulin dimer molecules at appropriate concentrations. After 5-15 min incubation at room temperature, ¹²⁵[I]-insulin was added at 0.015 nM final concentration for a final total volume of 50 μL. The mixture was incubated with shaking at room temperature for 1 to 12 hours followed by scintillation counting to determine ¹²⁵[I]-insulin binding to IR and the titration effects of insulin dimer molecules on this interaction.

B. Insulin Receptor (IR) AKT-Phosphorylation Assays were performed as follows.

Insulin receptor activation can be assessed by measuring phosphorylation of the Akt protein, a key step in the insulin receptor signaling cascade. CHO cell lines overexpressing human IR were utilized in an HTRF sandwich ELISA assay kit (Cisbio “Phospho-AKT (Ser473) and Phospho-AKT(Thr308) Cellular Assay Kits”). Cells were grown in F12 media supplemented with 10% FBS, 400 μg/mL G418 and 10 mM HEPES. Prior to assay, the cells were incubated in serum free media for 2 to 4 hr. Alternatively, the cells could be frozen and aliquoted ahead of time in media containing 20% DMSO and used in the assay upon thawing, spin down and re-suspension. Cells were plated at 10,000 cells per well in 20 μL of the serum free F12 media in 384-well plates. Humulin and insulin glargine controls were run on each plate of test compounds. The titrated compounds were added to the cells (2 μL per well, final concentrations=1000 nM titrated down to 0.512 μM in 1:5 fold dilutions) and incubated at 37° C. for 30 min. The cells were lysed with 8 μL of the prepared lysis buffer provided in the CisBio kit and incubated at 25° C. for 1 hr. The diluted antibody reagents (anti-AKT-d2 and anti-pAKT-Eu3/cryptate) were prepared according to the kit instructions and then 10 μL was added to each well of cell lysate followed by incubation at 25° C. for 3.5 to 5 hr. The plate was read by in an Envision plate reader (Excitation=320 nm; Emission=665 nm) to determine the IR pAkt agonist activity with regard to both potency and maximum response for each compound.

Table XIV shows the in vitro biological activity of the insulin dimers towards the insulin receptor (IR). The activities were measured by either ligand competition assays as described in Example 27A or functional Akt-phosphorylation assays as described in Example 27B.

### In Vitro Biological Activity.

| Dimer | IR Binding | IR pAkt | IR pAkt |
|---|---|---|---|
| No. | IC₅₀ (nM) | EC₅₀ (nM) | % Max |
| 1 | 14.7 | 7.3 | 35 |
| 2 | 2500.0 | 59330.0 | 65 |
| 3 | 98.0 | 11.3 | 78 |
| 4 | 432.2 | 68.0 | 49 |
| 5 | 183.9 | 19.0 | 93 |
| 6 | 5.2 | 0.1 | 29 |
| 7 | 7.8 | 29.5 | 44 |
| 8 | 4.4 | 0.3 | 20 |
| 9 | 0.3 | 0.9 | 59 |
| 10 | 5.4 | 0.3 | 43 |
| 11 | 0.5 | 0.6 | 46 |
| 12 | 5.9 | 1.2 | 31 |
| 13 | 0.5 | 0.5 | 25 |
| 14 | 7.2 | 0.8 | 30 |
| 15 | 11.0 | 0.3 | 26 |
| 16 | 4.7 | 0.7 | 21 |
| 17 | 0.2 | 0.6 | 20 |
| 18 | 3.5 | 0.8 | 19 |
| 19 | 1.6 | 0.8 | 38 |
| 20 | 5.9 | 0.1 | 27 |
| 21 | 5.4 | 1.1 | 23 |
| 22 | 4.1 | 0.5 | 35 |
| 23 | 4.2 | 0.2 | 24 |
| 24 | 0.6 | 0.3 | 24 |
| 25 | 1.8 | 0.6 | 30 |
| 26 | 2.4 | 0.1 | 22 |
| 27 | 2.5 | 0.1 | 37 |
| 28 | 0.8 | 1.0 | 29 |
| 29 | 10.8 | 0.7 | 21 |
| 30 | 0.1 | 0.1 | 13 |
| 31 | 7.2 | 0.5 | 43 |
| 32 | 8.3 | 0.3 | 31 |
| 33 | 0.8 | 1.4 | 40 |
| 34 | 1.4 | 1.8 | 38 |
| 35 | 0.1 | 0.8 | 25 |
| 36 | 10.0 | 0.1 | 34 |
| 37 | 10.0 | 0.1 | 27 |
| 38 | 0.9 | 0.1 | 29 |
| 39 | 1.2 | 0.6 | 17 |
| 40 | 3.9 | 0.3 | 20 |
| 41 | 5.3 | 0.9 | 13 |
| 42 | 2.0 | 0.8 | 17 |
| 43 | 1.5 | 1.2 | 19 |
| 44 | 0.9 | 0.2 | 20 |
| 45 | 0.6 | 0.1 | 17 |
| 46 | 1.3 | 0.3 | 12 |
| 47 | 2.5 | 1.4 | 41 |
| 48 | 0.6 | 0.5 | 48 |
| 49 | 0.9 | 0.1 | 6 |
| 50 | 1.4 | 1.2 | 50 |
| 51 | 0.8 | 0.4 | 30 |
| 52 | 2.2 | 0.1 | 22 |
| 53 | 0.7 | 0.9 | 34 |
| 54 | 4.8 | 0.5 | 28 |
| 55 | 1.6 | 0.4 | 35 |
| 56 | 0.5 | 1.6 | 26 |
| 57 | 0.7 | 0.1 | 10 |
| 58 | 5.1 | 1.2 | 24 |
| 59 | 6.9 | 0.4 | 16 |
| 60 | 14.8 | 0.1 | 16 |
| 61 | 11.9 | 0.1 | 12 |
| 62 | 1.9 | 0.3 | 21 |
| 63 | 2.2 | 1.0 | 33 |
| 64 | 4.7 | 0.5 | 16 |
| 65 | 18.4 | 1.5 | 19 |
| 66 | 11.2 | 1.6 | 20 |
| 67 | 44.8 | 7.9 | 21 |
| 68 | 19.2 | 0.3 | 32 |
| 69 | 2.2 | 0.9 | 29 |
| 70 | 4.1 | 0.1 | 27 |
| 71 | 0.6 | 0.1 | 28 |
| 72 | 2.3 | 0.3 | 29 |
| 73 | 2.8 | 0.1 | 28 |
| 74 | 18.4 | 0.3 | 24 |
| 75 | 4362.0 | 0.2 | 17 |
| 76 | | 0.2 | 26 |
| 77 | 9.3 | 0.4 | 21 |
| 78 | 20.5 | 0.1 | 34 |
| 79 | 10.5 | 0.4 | 39 |
| 80 | | 0.1 | 18 |
| 81 | 2511.0 | 0.02 | 14 |
| 82 | 1.4 | 0.3 | 24 |
| 83 | 185.0 | 0.3 | 15 |
| 84 | 18.0 | 0.1 | 16 |
| 85 | 9.0 | 0.2 | 16 |
| 86 | 14.9 | 0.2 | 22 |
| 87 | 17.1 | 0.1 | 15 |
| 88 | 145.2 | 0.3 | 12 |
| 89 | 1.1 | 0.4 | 25 |
| 90 | 9.3 | 1.5 | 23 |
| 91 | 3.0 | 0.1 | 21 |
| 92 | 3.7 | 0.1 | 16 |
| 93 | 0.9 | 0.1 | 23 |
| 94 | 4.5 | 0.3 | 20 |
| 95 | 10.3 | 0.1 | 26 |
| 96 | 5.9 | 0.4 | 22 |
| 97 | 4.3 | 0.2 | 30 |
| 98 | 28.8 | 0.1 | 27 |
| 99 | 2511 | 0.1 | 21 |
| 100 | 2511 | 0.1 | 26 |
| 101 | 2511 | 0.5 | 35 |
| 102 | 2.0 | 0.2 | 31 |
| 103 | 2.6 | 0.5 | 29 |
| 104 | 2.1 | 0.4 | 29 |
| 105 | 3.2 | 0.1 | 10 |
| 106 | 0.4 | 0.8 | 32 |
| 107 | 0.9 | 0.6 | 25 |
| 108 | 12.6 | 0.5 | 23 |
| 109 | 1.2 | 0.3 | 26 |
| 110 | 1.2 | 0.2 | 33 |
| 111 | 1.7 | 0.1 | 18 |
| 112 | 2.5 | 2.1 | 34 |
| 113 | 3.8 | 0.3 | 14 |
| 114 | 3.0 | 1.0 | 21 |
| 115 | 3.5 | 1.0 | 16 |
| 116 | 4.5 | 4.6 | 23 |
| 117 | 13.8 | 0.3 | 40 |
| 118 | 1.6 | 0.1 | 32 |
| 119 | 0.6 | 0.8 | 32 |
| 120 | 18.0 | 2.3 | 29 |
| 121 | 1.6 | 0.1 | 38 |
| 122 | 0.7 | 0.1 | 32 |
| 123 | 1.0 | 0.5 | 33 |
| 124 | 2.6 | 0.8 | 33 |
| 125 | 1.2 | 0.2 | 26 |
| 126 | 1.4 | 0.7 | 27 |
| 127 | 1.6 | 0.4 | 14 |
| 128 | 4.8 | 0.3 | 22 |
| 129 | 1.2 | 2.7 | 33 |
| 130 | 6.4 | 0.5 | 38 |
| 131 | 1.5 | 53.0 | 88 |
| 132 | 6.7 | 2.6 | 34 |
| 133 | 0.7 | 0.3 | 32 |
| 134 | 1.1 | 0.3 | 19 |
| 135 | 0.8 | 0.1 | 17 |
| 136 | 1.2 | 0.1 | 17 |
| 137 | 1.2 | 0.04 | 14 |
| 138 | 0.5 | 0.2 | 17 |
| 139 | 0.3 | 0.02 | 13 |
| 140 | 1.0 | 0.1 | 19 |
| 141 | 1.1 | 0.2 | 20 |
| 142 | 1.8 | 0.2 | 20 |
| 143 | 0.8 | 0.7 | 28 |
| 144 | 1.6 | 0.6 | 27 |

### Example 27

The glucose lowering effect of Dimers 17, 30, 44, 49, 57, 62, 84, 85, 86, 132, 134, and 139 were compared to RHI in Diabetic Yucatan miniature pigs (D minipigs) as follows.

Yucatan minipigs were rendered Type 1 diabetic by Alloxan injections following a proprietary protocol developed by Sinclair Research Center (Auxvasse, MO). Induction is considered successful if basal glucose levels exceed 150 mg/dL. Diabetic(D) minipigs with plasma glucose levels of approximately 300 mg/dl were utilized in these experiments.

Male Yucatan minipigs, instrumented with two Jugular vein vascular access ports (VAP), were used in these studies. On the day of the study after an overnight fast, minipigs were placed in slings, and VAPs were accessed for infusion and sampling. At t=0 min, and after collecting two baseline blood samples for plasma glucose measurement (t=−30 minutes and t=0 minutes), minipigs were administered Humulin (recombinant human insulin, RHI) or insulin dimer (i.e., 17, 30, 44, 49, 57, 62, 84, 85, 86, 132, 134, or 139) as a single bolus IV, at 0.69 nmol/kg. Humulin and the immediately preceding aforementioned insulin dimers were formulated at 69 nmol/ml in a buffer containing Glycerin, 16 mg/mL; Metacresol, 1.6 mg/mL; Phenol, 0.65 mg/mL; Anhydrous Sodium Phosphate, Dibasic, 3.8 mg/mL; pH adjusted to 7.4 with HCl. After dosing, sampling continued for 480 minutes; time points for sample collection were −30 min, 0 min, 8 min, 15 min, 30 min, 45 min, 60 min, 90 min, 120 min, 150 min, 180 min, 210 min, 240 min, 270 min, 300 min, 330 min, 360 min, 420 min, 480 min. Blood was collected in K3-EDTA tubes, supplemented with 10 μg/mL aprotinin, and kept on ice until processing, which occurred within 30 minutes of collection. After centrifugation at 3000 rpm, 4° C., for 8 min, plasma was collected and aliquoted for glucose measurement using a Beckman Coulter AU480 Chemistry analyzer and for compound levels measurement.

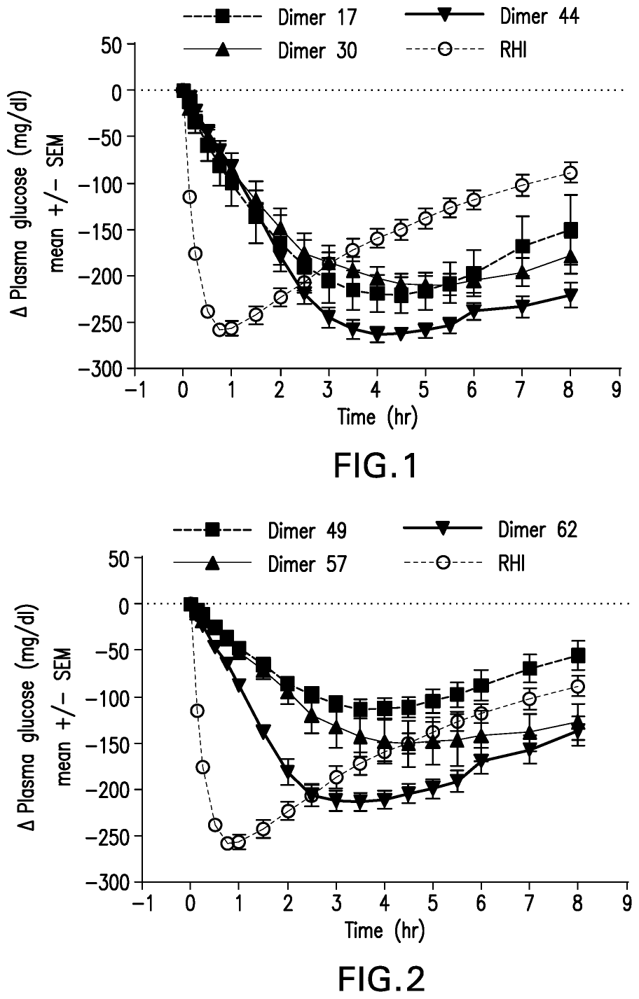

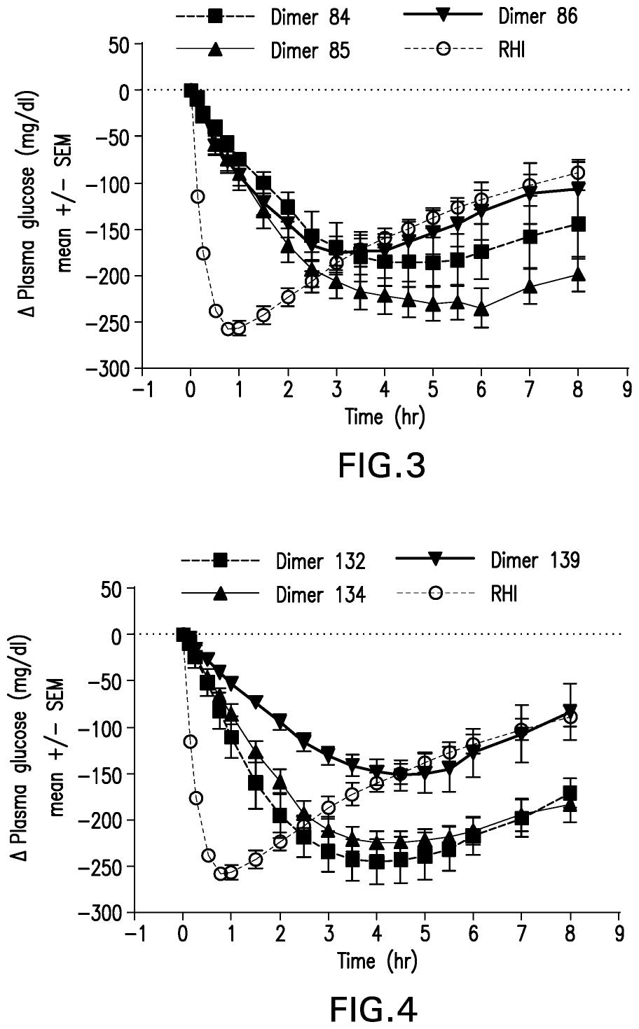

The results are shown in FIGS. 1-4. The results are presented as the change of glucose at any given time point to time 0 and show that the insulin dimers present less risk of promoting hypoglycemia than RHI.

Below are representable sequences useful in the invention.

Table of Sequences

| SEQ | | |
|---|---|---|
| ID | | |
| NO: | Description | Sequence |
| 1 | Homo sapiens insulin A chain | GIVEQCCTSICSLYQLENYCN |
| 2 | Homo sapiens insulin B chain | FVNQHLCGSHLVEALYLVC |
| | | GERGFFYTPKT |
| 3 | Artificial sequence insulin A chain | GX₂X₃EQCCX_{&}SICSLYQLX₁₇ |
| | X₂ is isoleucine or threonine; | NX₁₉CX₂₃ |
| | X₃ is valine, glycine, or leucine; | |
| | X₈ is threonine or histidine; | |
| | X₁₇ is glutamic acid or glutamine; | |
| | X₁₉ is tyrosine, 4-methoxy- | |
| | phenylalanine, alanine, or 4-amino | |
| | phenylalanine; | |
| | X₂₃ is asparagine or glycine; | |
| 4 | Artificial sequence insulin B chain | X₂₅LCGX₂₉X₃₀LVEALYLVC |
| | X₂₅ is histidine or threonine; | GERGFX₂₇YTX₃₁X₃₂ |
| | X₂₇ is phenylalanine or aspartic | |
| | acid; | |
| | X₂₉ is alanine, glycine or serine; | |
| | X₃₀ is histidine, aspartic acid, | |
| | glutamic acid, homocysteic acid, or | |
| | cysteic acid; | |
| | X₃₁ is aspartic acid, proline or | |
| | lysine; and | |
| | X₃₂ is proline or lysine, with the | |
| | proviso that at least one of X₃₁ or | |
| | X₃₂ is lysine | |
| 5 | Artificial sequence insulin B chain | X₂₂VNQX₂₅X₂₆CGX₂₉X₃₀L |
| | X₂₂ is phenylalanine or desamino- | VEALYLVCGERGFX₂₇YTX₃₁ |
| | phenylalanine; | X₃₂X₃₃X₃₄X₃₅ |
| | X₂₅ is histidine or threonine; | |
| | X₂₆ is glycine or leucine; | |
| | X₂₇ is phenylalanine or aspartic | |
| | acid; | |
| | X₂₉ is alanine, glycine, or serine; | |
| | X₃₀ is histidine, aspartic acid, | |
| | glutamic acid, homocysteic acid, or | |
| | cysteic acid; | |
| | X₃₁ is aspartic acid, proline, or | |
| | lysine; | |
| | X₃₂ is lysine or proline; | |
| | X₃₃ is threonine, alanine, or absent; | |
| | X₃₄ is arginine or absent; and | |
| | X₃₅ is arginine or absent; | |
| | With the proviso at least one of | |
| | X₃₁ or X₃₂ is lysine | |
| 6 | Artificial sequence | FVNQHLCGSHLVEALYLVC |
| | insulin lispro B chain | GERGFFYTKPT |
| 7 | Artificial sequence | GIVEQCCTSICSLYQLENYCG |
| | insulin glargine A chain | |
| 8 | Artificial sequence | FVNQHLCGSHLVEALYLVC |
| | Insulin glargine B chain | GERGFFYTPKTRR |
| 9 | Artificial sequence | FVNQHLCGSHLVEALYLVC |
| | Insulin aspart B chain | GERGFFYTDKT |
| 10 | Artificial sequence A:Y19A | GIVEQCCTSICSLYQLENACN |
| 11 | Artificial sequence | FVNQHLCGSHLVEALYLVC |
| | Insulin degludec B chain | GERGFFYTPK |
| 12 | Artificial sequence B: DesB30- | FVNQHLCGSHLVEALYLVC |
| | K29R | GERGFFYTPR |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. An insulin dimer comprising:
two insulin molecules dimerized by covalently linking the α-amino groups of the A1 residue of each insulin via a linker moiety, wherein the linker moiety is selected from the group consisting Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, Linker 23, Linker 24, Linker 25, Linker 26, Linker 27, Linker 28, Linker 29, Linker 30, Linker 31, Linker 32, Linker 33, Linker 34, Linker 35, Linker 36, Linker 37, Linker 38, Linker 39, Linker 40, Linker 41, Linker 42, Linker 43, Linker 44, Linker 45, and Linker 46.

2. The insulin dimer of claim 1 wherein each of the amines of the B-29 lysine or B-28 and the N-terminal amino acids of each of the B-chain of the two insulins optionally and independently are conjugated with a capping group.

3. The insulin dimer of claim 1, wherein the capping group comprises acyl moieties bearing carbamates, PEG-containing chains, sugar-containing groups, carboxylic acid containing groups, amines, amides, hydroxyls, phosphonate groups, and heterocycles, or a mixture thereof.

4. The insulin dimer of claim 1, wherein the capping group is a linear or branch C₁₋₆ alkyl, or has the general formula RC(O)—, where R is:
a) a peptide,
b) PEG,
c) linear or branched C₁₋₆ alkyl chain,
d) R′NH—, or
e) R′O—,
wherein R′ is H (when R is R′NH—), peptide, PEG, or linear or branched alkyl chain, and wherein each said peptide, PEG and linear or branched alkyl may be unsubstituted or substituted with 1 to 3 groups selected from amino-, phosphono-, hydroxy-, carboxylic acid, amino acid, PEG, and saccharides.

5. The insulin dimer of claim 1, wherein the capping group is
a) dimethyl,
b) isobutyl, or
c) RC(O)— which is selected from acetyl, phenylacetyl, methoxy acetyl, 2-(carboxymethoxy)acetyl, 2-[bis(carboxymethylamino)]acetyl, carbamoyl, N-alkyl carbamoyl, glutaryl, trifluoroacetyl, glycyl, aminoethylglucose (AEG), AEG-C6, PEG1, PEG2, PEG3, PEG4, PEG5, PEG8, PEG24, and alkoxycarbonyl.

6. The insulin dimer of claim 2 wherein the capping group is selected from Capping Group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31.

7. An insulin dimer comprising two insulin molecules dimerized by covalently linking the α-amino groups of the A1 residue of each insulin via a linker moiety, wherein the linker moiety is selected from the group consisting of Linking moiety Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, Linker 23, Linker 24, Linker 25, Linker 26, Linker 27, Linker 28, Linker 29, Linker 30, Linker 31, Linker 32, Linker 33, Linker 34, Linker 35, Linker 36, Linker 37, Linker 38, Linker 39, Linker 40, Linker 41, Linker 42, Linker 43, Linker 44, Linker 45, and Linker 46, and wherein each of the F-amines of the B-29 or B-28 lysine and the N-terminal amino acids of each of the B-chain of the two insulins independently are conjugated with 0-4 capping groups selected from Capping Group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31.

8. The insulin dimer of claim 1, wherein the first insulin and the second insulin heterodimers are independently native human insulin, insulin lispro, insulin aspart, desB30 insulin, or insulin glargine.

9. The insulin dimer of claim 1, wherein each A-chain polypeptide independently comprises the amino acid sequence GX₂X₃EQCCX₈SICSLYQLX₁₇NX₁₉CX₂₃ (SEQ ID NO:3) and each B-chain polypeptide independently comprises the amino acid sequence
X₂₅LCGX₂₉X₃₀LVEALYLVCGERGFX₂₇YTX₃₁X₃₂ (SEQ ID NO:4) or
X₂₂VNQX₂₅X₂₆CGX₂₉X₃₀LVEALYLVCGERGFX₂₇YTX₃₁X₃₂X₃₃X₃₄X₃₅ (SEQ ID NO:5) wherein X₂ is isoleucine or threonine; X₃ is valine, glycine, or leucine; X₈ is threonine or histidine; X₁₇ is glutamic acid or glutamine; X₁₉ is tyrosine, 4-methoxy-phenylalanine, alanine, or 4-amino phenylalanine; X₂₃ is asparagine or glycine; X₂₂ is or phenylalanine and desamino-phenylalanine; X₂₅ is histidine or threonine; X₂₆ is leucine or glycine; X₂₇ is phenylalanine or aspartic acid; X₂₉ is alanine, glycine, or serine; X₃₀ is histidine, aspartic acid, glutamic acid, homocysteic acid, or cysteic acid; X₃₁ is aspartic acid, proline, or lysine; X₃₂ is lysine or proline; X₃₃ is threonine, alanine, or absent; X₃₄ is arginine or absent; and X₃₅ is arginine or absent; with the proviso at least one of X₃₁ or X₃₂ is lysine.

10. The insulin dimer according to claim 1 wherein each A chain polypeptide independently comprises the amino acid sequence GIVEQCCTSICSLYQLENYCG (SEQ ID NO: 7) or GIVEQCCTSICSLYQLENACN (SEQ ID NO:10) and each B chain independently comprises the amino acid sequence FVNQHLCGSHLVEALYLVCGERGFFYTKPT (SEQ ID NO: 6), FVNQHLCGSHLVEALYLVCGERGFFYTPKTRR (SEQ ID NO: 8) or FVNQHLCGSHLVEALYLVCGERGFFYTDKT (SEQ ID NO: 9).

11. The insulin dimer of according to claim 1, wherein each A-chain polypeptide independently comprises the amino acid sequence GIVEQCCTSICSLYQLENYCN (SEQ ID NO: 1) and each B chain independently comprises amino acid sequence FVNQHLCGSH LVEALYLVCGERGFFYTPKT (SEQ ID NO: 2), FVNQHLCGSHLVEALYLVCGERGFFYTPK (SEQ ID: 11), or FVNQHLCGSHLVEALYLVCGERGFFYTPR (SEQ ID NO:12)

12. A composition comprising:
a first insulin or insulin analog heterodimer and a second insulin or insulin analog heterodimer each heterodimer having an A-chain polypeptide and a B-chain polypeptide, wherein the A-chain polypeptide and the B-chain polypeptide are linked together through interchain disulfide bonds; wherein the first and second insulin or insulin analog heterodimers are covalently linked together through a linking moiety joining the α-amino groups at the A1 position of the two A-chain polypeptides, wherein the linking moiety is selected from the group consisting of Linking moiety Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, Linker 23, Linker 24, Linker 25, Linker 26, Linker 27, Linker 28, Linker 29, Linker 30, Linker 31, Linker 32, Linker 33, Linker 34, Linker 35, Linker 36, Linker 37, Linker 38, Linker 39, Linker 40, Linker 41, Linker 42, Linker 43, Linker 44, Linker 45, and Linker 46; wherein the insulin analog is selected from insulin lispro, insulin aspart, and insulin glargine; and wherein each of the ε-amines of the B-29 or B-28 lysine and the N-terminal amino acids of each of the B-chain of the two insulins optionally and independently are conjugated with 0-4 capping groups.

13. The composition of claim 12, wherein the capping group is a linear or branch C₁₋₆ alkyl, or has the general formula RC(O)—, where R is:
a) a peptide,
b) PEG,
c) linear or branched C₁₋₆ alkyl chain,
d) R′NH—, or
e) R′O—,
wherein R′ is H (when R is R′NH—), peptide, PEG, or linear or branched alkyl chain, and wherein each said peptide, PEG and linear or branched alkyl may be unsubstituted or substituted with 1 to 3 groups selected from amino-, phosphono-, hydroxy-, carboxylic acid, amino acid, PEG, and saccharides.

14. The composition of claim 12, wherein the capping group is from selected from Capping Group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31.

15. The composition of claim 12, wherein the first and second insulin or insulin analog heterodimers are the same, or wherein the first and second insulin or insulin analog heterodimers are different.

16. A composition comprising an insulin dimer selected from Table III.

17. The composition of claim 16, wherein the composition further comprises a pharmaceutically acceptable carrier.

18. The composition of claim 16 wherein the composition further comprises a GLP-1 receptor agonist.

19. A method for treating diabetes comprising administering to an individual with diabetes a therapeutically effective amount of a composition comprising the insulin receptor partial agonist of claim 1.

20. The method of claim 19, wherein the diabetes is Type 1 diabetes, Type 2 diabetes, or gestational diabetes.

21. A composition for the treatment of diabetes comprising the insulin receptor partial agonist of claim 1.

22. The composition of claim 20, wherein the diabetes is Type 1 diabetes, Type 2 diabetes, or gestational diabetes.

23. (canceled)

24. (canceled)
